SEQUENCE LISTING (1448 sequences)

SEQ ID NO: 1 - RNA, 19 nt, Artificial Sequence
  Description of Artificial Sequence Synthetic oligonucleotide
aaugcuaucauccagaacu

SEQ ID NO: 2 - RNA, 19 nt, Artificial Sequence
  Description of Artificial Sequence Synthetic oligonucleotide
agagaugcgacccaguucc

SEQ ID NO: 3 - RNA, 19 nt, Artificial Sequence
  Description of Artificial Sequence Synthetic oligonucleotide
uuauuuaagggaacgacag

SEQ ID NO: 4 - RNA, 19 nt, Artificial Sequence
  Description of Artificial Sequence Synthetic oligonucleotide
aauucuagauccaaacugc

SEQ ID NO: 5 - RNA, 19 nt, Artificial Sequence
  Description of Artificial Sequence Synthetic oligonucleotide
aauuccaaguucgagaagg

SEQ ID NO: 6 - RNA, 19 nt, Artificial Sequence
  Description of Artificial Sequence Synthetic oligonucleotide
uuucuaacuaaacauaaga

SEQ ID NO: 7 - RNA, 19 nt, Artificial Sequence
  Description of Artificial Sequence Synthetic oligonucleotide
ucuaacuaaacauaagaca

SEQ ID NO: 8 - RNA, 19 nt, Artificial Sequence
  Description of Artificial Sequence Synthetic oligonucleotide
aaguguucaucagagaagc

SEQ ID NO: 9 - RNA, 19 nt, Artificial Sequence
  Description of Artificial Sequence Synthetic oligonucleotide
uggacucuagagaagagcg

SEQ ID NO: 10 - RNA, 19 nt, Artificial Sequence
  Description of Artificial Sequence Synthetic oligonucleotide
ucuagauccaaacugcaug

SEQ ID NO: 11 - RNA, 19 nt, Artificial Sequence
  Description of Artificial Sequence Synthetic oligonucleotide
aaaauuccaaguucgagaa

SEQ ID NO: 12 - RNA, 19 nt, Artificial Sequence
  Description of Artificial Sequence Synthetic oligonucleotide
aaccgacuccucuagacgc

SEQ ID NO: 13 - RNA, 19 nt, Artificial Sequence
  Description of Artificial Sequence Synthetic oligonucleotide
ugugaaaucagggugacug

SEQ ID NO: 14 - RNA, 19 nt, Artificial Sequence
  Description of Artificial Sequence Synthetic oligonucleotide
aaggcuacugaacauaacu

SEQ ID NO: 15 - RNA, 19 nt, Artificial Sequence
  Description of Artificial Sequence Synthetic oligonucleotide
uuugcuaccacugauacaa

SEQ ID NO: 16 - RNA, 19 nt, Artificial Sequence
  Description of Artificial Sequence Synthetic oligonucleotide
caucuaaaggaauaugacg

SEQ ID NO: 17 - RNA, 19 nt, Artificial Sequence
  Description of Artificial Sequence Synthetic oligonucleotide
uugacugcaacacucagga

SEQ ID NO: 18 - RNA, 19 nt, Artificial Sequence
  Description of Artificial Sequence Synthetic oligonucleotide
uuagaaacguauuuccaag

SEQ ID NO: 19 - RNA, 19 nt, Artificial Sequence
  Description of Artificial Sequence Synthetic oligonucleotide
ucuaagauugcacagcaaa

SEQ ID NO: 20 - RNA, 19 nt, Artificial Sequence
  Description of Artificial Sequence Synthetic oligonucleotide
ucuaccuuaguaacacucg

SEQ ID NO: 21 - RNA, 19 nt, Artificial Sequence
  Description of Artificial Sequence Synthetic oligonucleotide
cuugguauuguuuucuuag

SEQ ID NO: 22 - RNA, 19 nt, Artificial Sequence
  Description of Artificial Sequence Synthetic oligonucleotide
ucaaaacuuccuuugauac

SEQ ID NO: 23 - RNA, 19 nt, Artificial Sequence
  Description of Artificial Sequence Synthetic oligonucleotide
uucuaacuaaacauaagac

SEQ ID NO: 24 - RNA, 19 nt, Artificial Sequence
  Description of Artificial Sequence Synthetic oligonucleotide
ucuaagguaggauuuggag

SEQ ID NO: 25 - RNA, 19 nt, Artificial Sequence
  Description of Artificial Sequence Synthetic oligonucleotide
ugaacuaaguggcauauuc

SEQ ID NO: 26 - RNA, 19 nt, Artificial Sequence
  Description of Artificial Sequence Synthetic oligonucleotide
aagggcugaacuaaguggc

SEQ ID NO: 27 - RNA, 19 nt, Artificial Sequence
  Description of Artificial Sequence Synthetic oligonucleotide
aacgacagguuuugaaagg

SEQ ID NO: 28 - RNA, 19 nt, Artificial Sequence
  Description of Artificial Sequence Synthetic oligonucleotide
uccguuuagucacaaaagc

SEQ ID NO: 29 - RNA, 19 nt, Artificial Sequence
  Description of Artificial Sequence Synthetic oligonucleotide
gacgucaauaaacugauga

SEQ ID NO: 30 - RNA, 19 nt, Artificial Sequence
  Description of Artificial Sequence Synthetic oligonucleotide
ucuuguaagaucucacagc

SEQ ID NO: 31 - RNA, 19 nt, Artificial Sequence
  Description of Artificial Sequence Synthetic oligonucleotide
aauaugacgaucagcaaga

SEQ ID NO: 32 - RNA, 19 nt, Artificial Sequence
  Description of Artificial Sequence Synthetic oligonucleotide
cuagauccaaacugcaugg

SEQ ID NO: 33 - RNA, 19 nt, Artificial Sequence
  Description of Artificial Sequence Synthetic oligonucleotide
aacauaacuugagugucac

SEQ ID NO: 34 - RNA, 19 nt, Artificial Sequence
  Description of Artificial Sequence Synthetic oligonucleotide
uguuauuggggccccgucg

SEQ ID NO: 35 - RNA, 19 nt, Artificial Sequence
  Description of Artificial Sequence Synthetic oligonucleotide
uauuuaagggaacgacagg

SEQ ID NO: 36 - RNA, 19 nt, Artificial Sequence
  Description of Artificial Sequence Synthetic oligonucleotide
uaccaaaccauaaguaaug

SEQ ID NO: 37 - RNA, 19 nt, Artificial Sequence
  Description of Artificial Sequence Synthetic oligonucleotide
uagaaacguauuuccaagg

SEQ ID NO: 38 - RNA, 19 nt, Artificial Sequence
  Description of Artificial Sequence Synthetic oligonucleotide
aucuaaaggaauaugacga

SEQ ID NO: 39 - RNA, 19 nt, Artificial Sequence
  Description of Artificial Sequence Synthetic oligonucleotide
ugcccagauuauuaaaacg

SEQ ID NO: 40 - RNA, 19 nt, Artificial Sequence
  Description of Artificial Sequence Synthetic oligonucleotide
ggaauugagaucaauuugg

SEQ ID NO: 41 - RNA, 19 nt, Artificial Sequence
  Description of Artificial Sequence Synthetic oligonucleotide
aaaggaauaugacgaucag

SEQ ID NO: 42 - RNA, 19 nt, Artificial Sequence
  Description of Artificial Sequence Synthetic oligonucleotide
agagcgacaaaauaucaga

SEQ ID NO: 43 - RNA, 19 nt, Artificial Sequence
  Description of Artificial Sequence Synthetic oligonucleotide
cuacagagcagcaaaugcc

SEQ ID NO: 44 - RNA, 19 nt, Artificial Sequence
  Description of Artificial Sequence Synthetic oligonucleotide
gaugcuggaacuucuugcc

SEQ ID NO: 45 - RNA, 19 nt, Artificial Sequence
  Description of Artificial Sequence Synthetic oligonucleotide
uaaacauaagacacgucuc

SEQ ID NO: 46 - RNA, 19 nt, Artificial Sequence
  Description of Artificial Sequence Synthetic oligonucleotide
ugcucaucggcaccgaucc

SEQ ID NO: 47 - RNA, 19 nt, Artificial Sequence
  Description of Artificial Sequence Synthetic oligonucleotide
cuaccacugauacaaacgc

SEQ ID NO: 48 - RNA, 19 nt, Artificial Sequence
  Description of Artificial Sequence Synthetic oligonucleotide
ucuuauuuaagggaacgac

SEQ ID NO: 49 - RNA, 19 nt, Artificial Sequence
  Description of Artificial Sequence Synthetic oligonucleotide
aacaaacacugauaguuaa

SEQ ID NO: 50 - RNA, 19 nt, Artificial Sequence
  Description of Artificial Sequence Synthetic oligonucleotide
auuuaagggaacgacaggu

SEQ ID NO: 51 - RNA, 19 nt, Artificial Sequence
  Description of Artificial Sequence Synthetic oligonucleotide
uccucuuucaauucuuuac

SEQ ID NO: 52 - RNA, 19 nt, Artificial Sequence
  Description of Artificial Sequence Synthetic oligonucleotide
gaggaagaaacuauugccc

SEQ ID NO: 53 - RNA, 19 nt, Artificial Sequence
  Description of Artificial Sequence Synthetic oligonucleotide
agaagagcgacaaaauauc

SEQ ID NO: 54 - RNA, 19 nt, Artificial Sequence
  Description of Artificial Sequence Synthetic oligonucleotide
aaacgccaaagcaagcuac

SEQ ID NO: 55 - RNA, 19 nt, Artificial Sequence
  Description of Artificial Sequence Synthetic oligonucleotide
guaaaaugagcuucucacc

SEQ ID NO: 56 - RNA, 19 nt, Artificial Sequence
  Description of Artificial Sequence Synthetic oligonucleotide
ugaaaucagggugacugcg

SEQ ID NO: 57 - RNA, 19 nt, Artificial Sequence
  Description of Artificial Sequence Synthetic oligonucleotide
uguucaucuguaagaaaca

SEQ ID NO: 58 - RNA, 19 nt, Artificial Sequence
  Description of Artificial Sequence Synthetic oligonucleotide
aacgcuggcaauucugcug

SEQ ID NO: 59 - RNA, 19 nt, Artificial Sequence
  Description of Artificial Sequence Synthetic oligonucleotide
acaguagcaccccaagagg

SEQ ID NO: 60 - RNA, 19 nt, Artificial Sequence
  Description of Artificial Sequence Synthetic oligonucleotide
aagacacgucucuugacug

SEQ ID NO: 61 - RNA, 19 nt, Artificial Sequence
  Description of Artificial Sequence Synthetic oligonucleotide
uguuaagggccuccgugcg

SEQ ID NO: 62 - RNA, 19 nt, Artificial Sequence
  Description of Artificial Sequence Synthetic oligonucleotide
cuuguaagaucucacagca

SEQ ID NO: 63 - RNA, 19 nt, Artificial Sequence
  Description of Artificial Sequence Synthetic oligonucleotide
aaccccucguggcaaagac

SEQ ID NO: 64 - RNA, 19 nt, Artificial Sequence
  Description of Artificial Sequence Synthetic oligonucleotide
accaauaacucugucauca

SEQ ID NO: 65 - RNA, 19 nt, Artificial Sequence
  Description of Artificial Sequence Synthetic oligonucleotide
gaacuaaguggcauauucg

SEQ ID NO: 66 - RNA, 19 nt, Artificial Sequence
  Description of Artificial Sequence Synthetic oligonucleotide
cuuauuuaagggaacgaca

SEQ ID NO: 67 - RNA, 19 nt, Artificial Sequence
  Description of Artificial Sequence Synthetic oligonucleotide
aucaccaauaacucuguca

SEQ ID NO: 68 - RNA, 19 nt, Artificial Sequence
  Description of Artificial Sequence Synthetic oligonucleotide
aauugagaucaauuuggaa

SEQ ID NO: 69 - RNA, 19 nt, Artificial Sequence
  Description of Artificial Sequence Synthetic oligonucleotide
aggauugcaauguuugcug

SEQ ID NO: 70 - RNA, 19 nt, Artificial Sequence
  Description of Artificial Sequence Synthetic oligonucleotide
gaucucacagcauccuggg

SEQ ID NO: 71 - RNA, 19 nt, Artificial Sequence
  Description of Artificial Sequence Synthetic oligonucleotide
uaaguggcauauucgagcu

SEQ ID NO: 72 - RNA, 19 nt, Artificial Sequence
  Description of Artificial Sequence Synthetic oligonucleotide
ugucacacaguguuguuac

SEQ ID NO: 73 - RNA, 19 nt, Artificial Sequence
  Description of Artificial Sequence Synthetic oligonucleotide
cagucuuccguuuagucac

SEQ ID NO: 74 - RNA, 19 nt, Artificial Sequence
  Description of Artificial Sequence Synthetic oligonucleotide
guucgagaaggguugauug

SEQ ID NO: 75 - RNA, 19 nt, Artificial Sequence
  Description of Artificial Sequence Synthetic oligonucleotide
gacgggaguaaccagccag

SEQ ID NO: 76 - RNA, 19 nt, Artificial Sequence
  Description of Artificial Sequence Synthetic oligonucleotide
acucaggacgucaauaaac

SEQ ID NO: 77 - RNA, 19 nt, Artificial Sequence
  Description of Artificial Sequence Synthetic oligonucleotide
auauucgagcucuucuuag

SEQ ID NO: 78 - RNA, 19 nt, Artificial Sequence
  Description of Artificial Sequence Synthetic oligonucleotide
agaaacguauuuccaagga

SEQ ID NO: 79 - RNA, 19 nt, Artificial Sequence
  Description of Artificial Sequence Synthetic oligonucleotide
gauccaaacugcauggccc

SEQ ID NO: 80 - RNA, 19 nt, Artificial Sequence
  Description of Artificial Sequence Synthetic oligonucleotide
cauaaguaauggcuagagg

SEQ ID NO: 81 - RNA, 19 nt, Artificial Sequence
  Description of Artificial Sequence Synthetic oligonucleotide
ugcaaggcguaaaaugagc

SEQ ID NO: 82 - RNA, 19 nt, Artificial Sequence
  Description of Artificial Sequence Synthetic oligonucleotide
uccucuagacgcaaaccag

SEQ ID NO: 83 - RNA, 19 nt, Artificial Sequence
  Description of Artificial Sequence Synthetic oligonucleotide
aagagcgacaaaauaucag

SEQ ID NO: 84 - RNA, 19 nt, Artificial Sequence
  Description of Artificial Sequence Synthetic oligonucleotide
uuaaaacgccaaagcaagc

SEQ ID NO: 85 - RNA, 19 nt, Artificial Sequence
  Description of Artificial Sequence Synthetic oligonucleotide
caauaaacugaugaacuac

SEQ ID NO: 86 - RNA, 19 nt, Artificial Sequence
  Description of Artificial Sequence Synthetic oligonucleotide
accgaucccagugagaauc

SEQ ID NO: 87 - RNA, 19 nt, Artificial Sequence
  Description of Artificial Sequence Synthetic oligonucleotide
cuugaauggggaacgccgg

SEQ ID NO: 88 - RNA, 19 nt, Artificial Sequence
  Description of Artificial Sequence Synthetic oligonucleotide
gaggcuugauaauauauaa

SEQ ID NO: 89 - RNA, 19 nt, Artificial Sequence
  Description of Artificial Sequence Synthetic oligonucleotide
acauaagacacgucucuug

SEQ ID NO: 90 - RNA, 19 nt, Artificial Sequence
  Description of Artificial Sequence Synthetic oligonucleotide
cauuugcuaccacugauac

SEQ ID NO: 91 - RNA, 19 nt, Artificial Sequence
  Description of Artificial Sequence Synthetic oligonucleotide
ugggucacuaaccaaccga

SEQ ID NO: 92 - RNA, 19 nt, Artificial Sequence
  Description of Artificial Sequence Synthetic oligonucleotide
uaugacgaucagcaagagc

SEQ ID NO: 93 - RNA, 19 nt, Artificial Sequence
  Description of Artificial Sequence Synthetic oligonucleotide
aacaccucucaaaugaaga

SEQ ID NO: 94 - RNA, 19 nt, Artificial Sequence
  Description of Artificial Sequence Synthetic oligonucleotide
uccuuucuaacuaaacaua

SEQ ID NO: 95 - RNA, 19 nt, Artificial Sequence
  Description of Artificial Sequence Synthetic oligonucleotide
ugcuuauauacuuuccgau

SEQ ID NO: 96 - RNA, 19 nt, Artificial Sequence
  Description of Artificial Sequence Synthetic oligonucleotide
gaugacgggaguaaccagc

SEQ ID NO: 97 - RNA, 19 nt, Artificial Sequence
  Description of Artificial Sequence Synthetic oligonucleotide
uuaagggaacgacagguuu

SEQ ID NO: 98 - RNA, 19 nt, Artificial Sequence
  Description of Artificial Sequence Synthetic oligonucleotide
agaaacaucgcaagggucu

SEQ ID NO: 99 - RNA, 19 nt, Artificial Sequence
  Description of Artificial Sequence Synthetic oligonucleotide
uuaucagaugcuuucucau

SEQ ID NO: 100 - RNA, 19 nt, Artificial Sequence
  Description of Artificial Sequence Synthetic oligonucleotide
cauugcaauuuccucauuc

SEQ ID NO: 101 - RNA, 19 nt, Artificial Sequence
  Description of Artificial Sequence Synthetic oligonucleotide
gcgacaaaauaucagauuc

SEQ ID NO: 102 - RNA, 19 nt, Artificial Sequence
  Description of Artificial Sequence Synthetic oligonucleotide
ucacgacugagguuccugg

SEQ ID NO: 103 - RNA, 19 nt, Artificial Sequence
  Description of Artificial Sequence Synthetic oligonucleotide
aaaacgccaaagcaagcua

SEQ ID NO: 104 - RNA, 19 nt, Artificial Sequence
  Description of Artificial Sequence Synthetic oligonucleotide
ugugacucggucuggcacc

SEQ ID NO: 105 - RNA, 19 nt, Artificial Sequence
  Description of Artificial Sequence Synthetic oligonucleotide
ucuaaaggaauaugacgau

SEQ ID NO: 106 - RNA, 19 nt, Artificial Sequence
  Description of Artificial Sequence Synthetic oligonucleotide
ucaucaagacccugggcca

SEQ ID NO: 107 - RNA, 19 nt, Artificial Sequence
  Description of Artificial Sequence Synthetic oligonucleotide
cuuaguucaucuaaugucu

SEQ ID NO: 108 - RNA, 19 nt, Artificial Sequence
  Description of Artificial Sequence Synthetic oligonucleotide
caaugggcucaucuaaagg

SEQ ID NO: 109 - RNA, 19 nt, Artificial Sequence
  Description of Artificial Sequence Synthetic oligonucleotide
aaccaaccgacuccucuag

SEQ ID NO: 110 - RNA, 19 nt, Artificial Sequence
  Description of Artificial Sequence Synthetic oligonucleotide
uuuugcaauucuagaucca

SEQ ID NO: 111 - RNA, 19 nt, Artificial Sequence
  Description of Artificial Sequence Synthetic oligonucleotide
acugauacaaacgcuggca

SEQ ID NO: 112 - RNA, 19 nt, Artificial Sequence
  Description of Artificial Sequence Synthetic oligonucleotide
cauggaaucaacaccucca

SEQ ID NO: 113 - RNA, 19 nt, Artificial Sequence
  Description of Artificial Sequence Synthetic oligonucleotide
aacaucgcaagggucucuc

SEQ ID NO: 114 - RNA, 19 nt, Artificial Sequence
  Description of Artificial Sequence Synthetic oligonucleotide
cuggaagcgaacacgauga

SEQ ID NO: 115 - RNA, 19 nt, Artificial Sequence
  Description of Artificial Sequence Synthetic oligonucleotide
aaauacgcugcuguucugu

SEQ ID NO: 116 - RNA, 19 nt, Artificial Sequence
  Description of Artificial Sequence Synthetic oligonucleotide
cuguuaagggccuccgugc

SEQ ID NO: 117 - RNA, 19 nt, Artificial Sequence
  Description of Artificial Sequence Synthetic oligonucleotide
caagaggacucuucggaag

SEQ ID NO: 118 - RNA, 19 nt, Artificial Sequence
  Description of Artificial Sequence Synthetic oligonucleotide
ucauuugcuaccacugaua

SEQ ID NO: 119 - RNA, 19 nt, Artificial Sequence
  Description of Artificial Sequence Synthetic oligonucleotide
uuugcaauucuagauccaa

SEQ ID NO: 120 - RNA, 19 nt, Artificial Sequence
  Description of Artificial Sequence Synthetic oligonucleotide
uugcccagauuauuaaaac

SEQ ID NO: 121 - RNA, 19 nt, Artificial Sequence
  Description of Artificial Sequence Synthetic oligonucleotide
gucgucuaagauugcacag

SEQ ID NO: 122 - RNA, 19 nt, Artificial Sequence
  Description of Artificial Sequence Synthetic oligonucleotide
aaaccauaaguaauggcua

SEQ ID NO: 123 - RNA, 19 nt, Artificial Sequence
  Description of Artificial Sequence Synthetic oligonucleotide
aagacccugggccacagua

SEQ ID NO: 124 - RNA, 19 nt, Artificial Sequence
  Description of Artificial Sequence Synthetic oligonucleotide
gguuagaaaguucauccac

SEQ ID NO: 125 - RNA, 19 nt, Artificial Sequence
  Description of Artificial Sequence Synthetic oligonucleotide
aacaugaccggaaaacaaa

SEQ ID NO: 126 - RNA, 19 nt, Artificial Sequence
  Description of Artificial Sequence Synthetic oligonucleotide
ucuagacgcaaaccagaca

SEQ ID NO: 127 - RNA, 19 nt, Artificial Sequence
  Description of Artificial Sequence Synthetic oligonucleotide
uucgagaaggguugauuga

SEQ ID NO: 128 - RNA, 19 nt, Artificial Sequence
  Description of Artificial Sequence Synthetic oligonucleotide
cagauuauuaaaacgccaa

SEQ ID NO: 129 - RNA, 19 nt, Artificial Sequence
  Description of Artificial Sequence Synthetic oligonucleotide
acuccucuagacgcaaacc

SEQ ID NO: 130 - RNA, 19 nt, Artificial Sequence
  Description of Artificial Sequence Synthetic oligonucleotide
ugguauuguuuucuuaguu

SEQ ID NO: 131 - RNA, 19 nt, Artificial Sequence
  Description of Artificial Sequence Synthetic oligonucleotide
ucaggacgucaauaaacug

SEQ ID NO: 132 - RNA, 19 nt, Artificial Sequence
  Description of Artificial Sequence Synthetic oligonucleotide
cuuccguuuagucacaaaa

SEQ ID NO: 133 - RNA, 19 nt, Artificial Sequence
  Description of Artificial Sequence Synthetic oligonucleotide
uauucgagcucuucuuagu

SEQ ID NO: 134 - RNA, 19 nt, Artificial Sequence
  Description of Artificial Sequence Synthetic oligonucleotide
ugaucaccaauaacucugu

SEQ ID NO: 135 - RNA, 19 nt, Artificial Sequence
  Description of Artificial Sequence Synthetic oligonucleotide
uaggauuuggagccuucca

SEQ ID NO: 136 - RNA, 19 nt, Artificial Sequence
  Description of Artificial Sequence Synthetic oligonucleotide
aaaauacgcugcuguucug

SEQ ID NO: 137 - RNA, 19 nt, Artificial Sequence
  Description of Artificial Sequence Synthetic oligonucleotide
acgucaauaaacugaugaa

SEQ ID NO: 138 - RNA, 19 nt, Artificial Sequence
  Description of Artificial Sequence Synthetic oligonucleotide
uuggaaaauuccagaugcu

SEQ ID NO: 139 - RNA, 19 nt, Artificial Sequence
  Description of Artificial Sequence Synthetic oligonucleotide
aaacgcuggcaauucugcu

SEQ ID NO: 140 - RNA, 19 nt, Artificial Sequence
  Description of Artificial Sequence Synthetic oligonucleotide
ggcuacugaacauaacuug

SEQ ID NO: 141 - RNA, 19 nt, Artificial Sequence
  Description of Artificial Sequence Synthetic oligonucleotide
augacgggaguaaccagcc

SEQ ID NO: 142 - RNA, 19 nt, Artificial Sequence
  Description of Artificial Sequence Synthetic oligonucleotide
caaccgacuccucuagacg

SEQ ID NO: 143 - RNA, 19 nt, Artificial Sequence
  Description of Artificial Sequence Synthetic oligonucleotide
uauugcccagauuauuaaa

SEQ ID NO: 144 - RNA, 19 nt, Artificial Sequence
  Description of Artificial Sequence Synthetic oligonucleotide
uggcaauucugcugcagug

SEQ ID NO: 145 - RNA, 19 nt, Artificial Sequence
  Description of Artificial Sequence Synthetic oligonucleotide
auacaaacgcuggcaauuc

SEQ ID NO: 146 - RNA, 19 nt, Artificial Sequence
  Description of Artificial Sequence Synthetic oligonucleotide
uucuagauccaaacugcau

SEQ ID NO: 147 - RNA, 19 nt, Artificial Sequence
  Description of Artificial Sequence Synthetic oligonucleotide
cuucggggcaucuccacgc

SEQ ID NO: 148 - RNA, 19 nt, Artificial Sequence
  Description of Artificial Sequence Synthetic oligonucleotide
agguuagaaaguucaucca

SEQ ID NO: 149 - RNA, 19 nt, Artificial Sequence
  Description of Artificial Sequence Synthetic oligonucleotide
aucgcaagggucucucuug

SEQ ID NO: 150 - RNA, 19 nt, Artificial Sequence
  Description of Artificial Sequence Synthetic oligonucleotide
gucccuaccaaaccauaag

SEQ ID NO: 151 - RNA, 19 nt, Artificial Sequence
  Description of Artificial Sequence Synthetic oligonucleotide
aagcaagcuacgccggcca

SEQ ID NO: 152 - RNA, 19 nt, Artificial Sequence
  Description of Artificial Sequence Synthetic oligonucleotide
uuacaggguggaaucacau

SEQ ID NO: 153 - RNA, 19 nt, Artificial Sequence
  Description of Artificial Sequence Synthetic oligonucleotide
agcaucuuauuuaagggaa

SEQ ID NO: 154 - RNA, 19 nt, Artificial Sequence
  Description of Artificial Sequence Synthetic oligonucleotide
aguuccauugcaauuuccu

SEQ ID NO: 155 - RNA, 19 nt, Artificial Sequence
  Description of Artificial Sequence Synthetic oligonucleotide
ucgagaaggguugauugac

SEQ ID NO: 156 - RNA, 19 nt, Artificial Sequence
  Description of Artificial Sequence Synthetic oligonucleotide
gucaauaaacugaugaacu

SEQ ID NO: 157 - RNA, 19 nt, Artificial Sequence
  Description of Artificial Sequence Synthetic oligonucleotide
uuaagggccuccgugcgug

SEQ ID NO: 158 - RNA, 19 nt, Artificial Sequence
  Description of Artificial Sequence Synthetic oligonucleotide
ucguaccccacuucggggc

SEQ ID NO: 159 - RNA, 19 nt, Artificial Sequence
  Description of Artificial Sequence Synthetic oligonucleotide
gagaagagcgacaaaauau

SEQ ID NO: 160 - RNA, 19 nt, Artificial Sequence
  Description of Artificial Sequence Synthetic oligonucleotide
gaaucaacaccuccagaaa

SEQ ID NO: 161 - RNA, 19 nt, Artificial Sequence
  Description of Artificial Sequence Synthetic oligonucleotide
agccacauucagaagauca

SEQ ID NO: 162 - RNA, 19 nt, Artificial Sequence
  Description of Artificial Sequence Synthetic oligonucleotide
agcgacaaaauaucagauu

SEQ ID NO: 163 - RNA, 19 nt, Artificial Sequence
  Description of Artificial Sequence Synthetic oligonucleotide
ccguuuagucacaaaagcc

SEQ ID NO: 164 - RNA, 19 nt, Artificial Sequence
  Description of Artificial Sequence Synthetic oligonucleotide
gaagagcgacaaaauauca

SEQ ID NO: 165 - RNA, 19 nt, Artificial Sequence
  Description of Artificial Sequence Synthetic oligonucleotide
ucaucggcaccgaucccag

SEQ ID NO: 166 - RNA, 19 nt, Artificial Sequence
  Description of Artificial Sequence Synthetic oligonucleotide
uuaguaacacucggcccag

SEQ ID NO: 167 - RNA, 19 nt, Artificial Sequence
  Description of Artificial Sequence Synthetic oligonucleotide
aguguuguuacagggugga

SEQ ID NO: 168 - RNA, 19 nt, Artificial Sequence
  Description of Artificial Sequence Synthetic oligonucleotide
aauacgcugcuguucuguu

SEQ ID NO: 169 - RNA, 19 nt, Artificial Sequence
  Description of Artificial Sequence Synthetic oligonucleotide
aguagcaccccaagaggac

SEQ ID NO: 170 - RNA, 19 nt, Artificial Sequence
  Description of Artificial Sequence Synthetic oligonucleotide
auugcccagauuauuaaaa

SEQ ID NO: 171 - RNA, 19 nt, Artificial Sequence
  Description of Artificial Sequence Synthetic oligonucleotide
gaucccagugagaaucacc

SEQ ID NO: 172 - RNA, 19 nt, Artificial Sequence
  Description of Artificial Sequence Synthetic oligonucleotide
cucucaaaugaagaacaug

SEQ ID NO: 173 - RNA, 19 nt, Artificial Sequence
  Description of Artificial Sequence Synthetic oligonucleotide
gaauggggaacgccggaac

SEQ ID NO: 174 - RNA, 19 nt, Artificial Sequence
  Description of Artificial Sequence Synthetic oligonucleotide
gacucggucuggcaccucc

SEQ ID NO: 175 - RNA, 19 nt, Artificial Sequence
  Description of Artificial Sequence Synthetic oligonucleotide
gugaaaucagggugacugc

SEQ ID NO: 176 - RNA, 19 nt, Artificial Sequence
  Description of Artificial Sequence Synthetic oligonucleotide
cuuuccgauucuucuucag

SEQ ID NO: 177 - RNA, 19 nt, Artificial Sequence
  Description of Artificial Sequence Synthetic oligonucleotide
uacaaacgcuggcaauucu

SEQ ID NO: 178 - RNA, 19 nt, Artificial Sequence
  Description of Artificial Sequence Synthetic oligonucleotide
ucgguugucucuguguuuc

SEQ ID NO: 179 - RNA, 19 nt, Artificial Sequence
  Description of Artificial Sequence Synthetic oligonucleotide
cugcuuauauacuuuccga

SEQ ID NO: 180 - RNA, 19 nt, Artificial Sequence
  Description of Artificial Sequence Synthetic oligonucleotide
aaguaauggcuagaggcuu

SEQ ID NO: 181 - RNA, 19 nt, Artificial Sequence
  Description of Artificial Sequence Synthetic oligonucleotide
gaagaacaugaccggaaaa

SEQ ID NO: 182 - RNA, 19 nt, Artificial Sequence
  Description of Artificial Sequence Synthetic oligonucleotide
cccagauuauuaaaacgcc

SEQ ID NO: 183 - RNA, 19 nt, Artificial Sequence
  Description of Artificial Sequence Synthetic oligonucleotide
gacgaucagcaagagcugc

SEQ ID NO: 184 - RNA, 19 nt, Artificial Sequence
  Description of Artificial Sequence Synthetic oligonucleotide
acgaugacgggaguaacca

SEQ ID NO: 185 - RNA, 19 nt, Artificial Sequence
  Description of Artificial Sequence Synthetic oligonucleotide
augcgacccaguuccauug

SEQ ID NO: 186 - RNA, 19 nt, Artificial Sequence
  Description of Artificial Sequence Synthetic oligonucleotide
aucuuauuuaagggaacga

SEQ ID NO: 187 - RNA, 19 nt, Artificial Sequence
  Description of Artificial Sequence Synthetic oligonucleotide
aacauaagacacgucucuu

SEQ ID NO: 188 - RNA, 19 nt, Artificial Sequence
  Description of Artificial Sequence Synthetic oligonucleotide
cucguaccccacuucgggg

SEQ ID NO: 189 - RNA, 19 nt, Artificial Sequence
  Description of Artificial Sequence Synthetic oligonucleotide
uaaccaaccgacuccucua

SEQ ID NO: 190 - RNA, 19 nt, Artificial Sequence
  Description of Artificial Sequence Synthetic oligonucleotide
ccaaguucgagaaggguug

SEQ ID NO: 191 - RNA, 19 nt, Artificial Sequence
  Description of Artificial Sequence Synthetic oligonucleotide
acuauugcccagauuauua

SEQ ID NO: 192 - RNA, 19 nt, Artificial Sequence
  Description of Artificial Sequence Synthetic oligonucleotide
cuccucuagacgcaaacca

SEQ ID NO: 193 - RNA, 19 nt, Artificial Sequence
  Description of Artificial Sequence Synthetic oligonucleotide
acaaacgcuggcaauucug

SEQ ID NO: 194 - RNA, 19 nt, Artificial Sequence
  Description of Artificial Sequence Synthetic oligonucleotide
gcuguuaagggccuccgug

SEQ ID NO: 195 - RNA, 19 nt, Artificial Sequence
  Description of Artificial Sequence Synthetic oligonucleotide
cgugcauccgacuuguagg

SEQ ID NO: 196 - RNA, 19 nt, Artificial Sequence
  Description of Artificial Sequence Synthetic oligonucleotide
aaauccucuuucaauucuu

SEQ ID NO: 197 - RNA, 19 nt, Artificial Sequence
  Description of Artificial Sequence Synthetic oligonucleotide
uugccacuugucugcaagc

SEQ ID NO: 198 - RNA, 19 nt, Artificial Sequence
  Description of Artificial Sequence Synthetic oligonucleotide
gaaacguauuuccaaggau

SEQ ID NO: 199 - RNA, 19 nt, Artificial Sequence
  Description of Artificial Sequence Synthetic oligonucleotide
ucuucggaagggaucucau

SEQ ID NO: 200 - RNA, 19 nt, Artificial Sequence
  Description of Artificial Sequence Synthetic oligonucleotide
ucgcaagggucucucuuga

SEQ ID NO: 201 - RNA, 19 nt, Artificial Sequence
  Description of Artificial Sequence Synthetic oligonucleotide
aguuaaaugacccauacac

SEQ ID NO: 202 - RNA, 19 nt, Artificial Sequence
  Description of Artificial Sequence Synthetic oligonucleotide
auaucaagcagauacaugc

SEQ ID NO: 203 - RNA, 19 nt, Artificial Sequence
  Description of Artificial Sequence Synthetic oligonucleotide
accaauaucaagcagauac

SEQ ID NO: 204 - RNA, 19 nt, Artificial Sequence
  Description of Artificial Sequence Synthetic oligonucleotide
aaugacaucaauauuuagc

SEQ ID NO: 205 - RNA, 19 nt, Artificial Sequence
  Description of Artificial Sequence Synthetic oligonucleotide
ucacauaguagauagaagg

SEQ ID NO: 206 - RNA, 19 nt, Artificial Sequence
  Description of Artificial Sequence Synthetic oligonucleotide
uucaagcuaaacuugaagg

SEQ ID NO: 207 - RNA, 19 nt, Artificial Sequence
  Description of Artificial Sequence Synthetic oligonucleotide
uucgauacgauuuucuugg

SEQ ID NO: 208 - RNA, 19 nt, Artificial Sequence
  Description of Artificial Sequence Synthetic oligonucleotide
ugcaugaucguaaagaaug

SEQ ID NO: 209 - RNA, 19 nt, Artificial Sequence
  Description of Artificial Sequence Synthetic oligonucleotide
augggauucaguuauggcc

SEQ ID NO: 210 - RNA, 19 nt, Artificial Sequence
  Description of Artificial Sequence Synthetic oligonucleotide
auguacaagcuacaaaugc

SEQ ID NO: 211 - RNA, 19 nt, Artificial Sequence
  Description of Artificial Sequence Synthetic oligonucleotide
acccguugaaaguagaugc

SEQ ID NO: 212 - RNA, 19 nt, Artificial Sequence
  Description of Artificial Sequence Synthetic oligonucleotide
uaaacuugaagguaagagc

SEQ ID NO: 213 - RNA, 19 nt, Artificial Sequence
  Description of Artificial Sequence Synthetic oligonucleotide
aguguaugcacccauguuc

SEQ ID NO: 214 - RNA, 19 nt, Artificial Sequence
  Description of Artificial Sequence Synthetic oligonucleotide
cuuacauggauuugcauag

SEQ ID NO: 215 - RNA, 19 nt, Artificial Sequence
  Description of Artificial Sequence Synthetic oligonucleotide
acuaacaguaaguuaaaug

SEQ ID NO: 216 - RNA, 19 nt, Artificial Sequence
  Description of Artificial Sequence Synthetic oligonucleotide
ucggcuauaacucucacuc

SEQ ID NO: 217 - RNA, 19 nt, Artificial Sequence
  Description of Artificial Sequence Synthetic oligonucleotide
uucaguuauggccaguucc

SEQ ID NO: 218 - RNA, 19 nt, Artificial Sequence
  Description of Artificial Sequence Synthetic oligonucleotide
uguaugauacuuccaacug

SEQ ID NO: 219 - RNA, 19 nt, Artificial Sequence
  Description of Artificial Sequence Synthetic oligonucleotide
auagaaggccucuggaacc

SEQ ID NO: 220 - RNA, 19 nt, Artificial Sequence
  Description of Artificial Sequence Synthetic oligonucleotide
uauuuagcucuuuugcccg

SEQ ID NO: 221 - RNA, 19 nt, Artificial Sequence
  Description of Artificial Sequence Synthetic oligonucleotide
aacaguaaguuaaaugguc

SEQ ID NO: 222 - RNA, 19 nt, Artificial Sequence
  Description of Artificial Sequence Synthetic oligonucleotide
gauauugacugccagugug

SEQ ID NO: 223 - RNA, 19 nt, Artificial Sequence
  Description of Artificial Sequence Synthetic oligonucleotide
uccaacuguuacuaggugg

SEQ ID NO: 224 - RNA, 19 nt, Artificial Sequence
  Description of Artificial Sequence Synthetic oligonucleotide
gauacuagcagaagcacag

SEQ ID NO: 225 - RNA, 19 nt, Artificial Sequence
  Description of Artificial Sequence Synthetic oligonucleotide
uucaucgaggauauggcag

SEQ ID NO: 226 - RNA, 19 nt, Artificial Sequence
  Description of Artificial Sequence Synthetic oligonucleotide
cauggauuugcauagauga

SEQ ID NO: 227 - RNA, 19 nt, Artificial Sequence
  Description of Artificial Sequence Synthetic oligonucleotide
uguacaagcuacaaaugcu

SEQ ID NO: 228 - RNA, 19 nt, Artificial Sequence
  Description of Artificial Sequence Synthetic oligonucleotide
aaagccuuagaugccuucc

SEQ ID NO: 229 - RNA, 19 nt, Artificial Sequence
  Description of Artificial Sequence Synthetic oligonucleotide
uuugcauagaugauccucu

SEQ ID NO: 230 - RNA, 19 nt, Artificial Sequence
  Description of Artificial Sequence Synthetic oligonucleotide
uguaccaacuguaucucag

SEQ ID NO: 231 - RNA, 19 nt, Artificial Sequence
  Description of Artificial Sequence Synthetic oligonucleotide
ucaagauaguuuauuuuca

SEQ ID NO: 232 - RNA, 19 nt, Artificial Sequence
  Description of Artificial Sequence Synthetic oligonucleotide
cuaaacuugaagguaagag

SEQ ID NO: 233 - RNA, 19 nt, Artificial Sequence
  Description of Artificial Sequence Synthetic oligonucleotide
cauugauacuagcagaagc

SEQ ID NO: 234 - RNA, 19 nt, Artificial Sequence
  Description of Artificial Sequence Synthetic oligonucleotide
uauaacucucacuccagag

SEQ ID NO: 235 - RNA, 19 nt, Artificial Sequence
  Description of Artificial Sequence Synthetic oligonucleotide
ucaagcuaaacuugaaggu

SEQ ID NO: 236 - RNA, 19 nt, Artificial Sequence
  Description of Artificial Sequence Synthetic oligonucleotide
acaguaaguuaaauggucc

SEQ ID NO: 237 - RNA, 19 nt, Artificial Sequence
  Description of Artificial Sequence Synthetic oligonucleotide
cuugaucugagacacuugc

SEQ ID NO: 238 - RNA, 19 nt, Artificial Sequence
  Description of Artificial Sequence Synthetic oligonucleotide
ugaucguaaagaaugcagu

SEQ ID NO: 239 - RNA, 19 nt, Artificial Sequence
  Description of Artificial Sequence Synthetic oligonucleotide
aaugcaguuaaaugaccca

SEQ ID NO: 240 - RNA, 19 nt, Artificial Sequence
  Description of Artificial Sequence Synthetic oligonucleotide
uaaaugacccauacacucc

SEQ ID NO: 241 - RNA, 19 nt, Artificial Sequence
  Description of Artificial Sequence Synthetic oligonucleotide
cauuucaaaacuaacagua

SEQ ID NO: 242 - RNA, 19 nt, Artificial Sequence
  Description of Artificial Sequence Synthetic oligonucleotide
caacauuagcuuuuggccc

SEQ ID NO: 243 - RNA, 19 nt, Artificial Sequence
  Description of Artificial Sequence Synthetic oligonucleotide
auacaugaagguuugcuca

SEQ ID NO: 244 - RNA, 19 nt, Artificial Sequence
  Description of Artificial Sequence Synthetic oligonucleotide
ucauaagcugccacauugg

SEQ ID NO: 245 - RNA, 19 nt, Artificial Sequence
  Description of Artificial Sequence Synthetic oligonucleotide
gaaguugauugccaagugc

SEQ ID NO: 246 - RNA, 19 nt, Artificial Sequence
  Description of Artificial Sequence Synthetic oligonucleotide
ggcaucuacacauugauac

SEQ ID NO: 247 - RNA, 19 nt, Artificial Sequence
  Description of Artificial Sequence Synthetic oligonucleotide
uagaugauccucucgggag

SEQ ID NO: 248 - RNA, 19 nt, Artificial Sequence
  Description of Artificial Sequence Synthetic oligonucleotide
aaaagccuuagaugccuuc

SEQ ID NO: 249 - RNA, 19 nt, Artificial Sequence
  Description of Artificial Sequence Synthetic oligonucleotide
cucgacaucacauaguaga

SEQ ID NO: 250 - RNA, 19 nt, Artificial Sequence
  Description of Artificial Sequence Synthetic oligonucleotide
uuuuagccaucuuaggugu

SEQ ID NO: 251 - RNA, 19 nt, Artificial Sequence
  Description of Artificial Sequence Synthetic oligonucleotide
uguugagauuuauuacagg

SEQ ID NO: 252 - RNA, 19 nt, Artificial Sequence
  Description of Artificial Sequence Synthetic oligonucleotide
aauccggucaaggccaucg

SEQ ID NO: 253 - RNA, 19 nt, Artificial Sequence
  Description of Artificial Sequence Synthetic oligonucleotide
aagguuugcucacuggacu

SEQ ID NO: 254 - RNA, 19 nt, Artificial Sequence
  Description of Artificial Sequence Synthetic oligonucleotide
aguggcgauccgcaaaacc

SEQ ID NO: 255 - RNA, 19 nt, Artificial Sequence
  Description of Artificial Sequence Synthetic oligonucleotide
uaugauauugacugccagu

SEQ ID NO: 256 - RNA, 19 nt, Artificial Sequence
  Description of Artificial Sequence Synthetic oligonucleotide
caggauaucaggucgcuag

SEQ ID NO: 257 - RNA, 19 nt, Artificial Sequence
  Description of Artificial Sequence Synthetic oligonucleotide
ugcuguaugauacuuccaa

SEQ ID NO: 258 - RNA, 19 nt, Artificial Sequence
  Description of Artificial Sequence Synthetic oligonucleotide
guagauagaaggccucugg

SEQ ID NO: 259 - RNA, 19 nt, Artificial Sequence
  Description of Artificial Sequence Synthetic oligonucleotide
gaaugcaguuaaaugaccc

SEQ ID NO: 260 - RNA, 19 nt, Artificial Sequence
  Description of Artificial Sequence Synthetic oligonucleotide
cucaccaacaugacuacag

SEQ ID NO: 261 - RNA, 19 nt, Artificial Sequence
  Description of Artificial Sequence Synthetic oligonucleotide
guaguaucugccuggcucg

SEQ ID NO: 262 - RNA, 19 nt, Artificial Sequence
  Description of Artificial Sequence Synthetic oligonucleotide
uauccucagauccaggaaa

SEQ ID NO: 263 - RNA, 19 nt, Artificial Sequence
  Description of Artificial Sequence Synthetic oligonucleotide
caaacauucccugaugccc

SEQ ID NO: 264 - RNA, 19 nt, Artificial Sequence
  Description of Artificial Sequence Synthetic oligonucleotide
augauauugacugccagug

SEQ ID NO: 265 - RNA, 19 nt, Artificial Sequence
  Description of Artificial Sequence Synthetic oligonucleotide
acuugaagguaagagcuac

SEQ ID NO: 266 - RNA, 19 nt, Artificial Sequence
  Description of Artificial Sequence Synthetic oligonucleotide
cuuuacacugagucgacac

SEQ ID NO: 267 - RNA, 19 nt, Artificial Sequence
  Description of Artificial Sequence Synthetic oligonucleotide
ugugcaugaucguaaagaa

SEQ ID NO: 268 - RNA, 19 nt, Artificial Sequence
  Description of Artificial Sequence Synthetic oligonucleotide
gauacgauuuucuuggcua

SEQ ID NO: 269 - RNA, 19 nt, Artificial Sequence
  Description of Artificial Sequence Synthetic oligonucleotide
acugagucgacacacugcu

SEQ ID NO: 270 - RNA, 19 nt, Artificial Sequence
  Description of Artificial Sequence Synthetic oligonucleotide
aaaccuguuccaauggcag

SEQ ID NO: 271 - RNA, 19 nt, Artificial Sequence
  Description of Artificial Sequence Synthetic oligonucleotide
auaacucucacuccagagu

SEQ ID NO: 272 - RNA, 19 nt, Artificial Sequence
  Description of Artificial Sequence Synthetic oligonucleotide
cauaaaagggagugacccg

SEQ ID NO: 273 - RNA, 19 nt, Artificial Sequence
  Description of Artificial Sequence Synthetic oligonucleotide
uuaagggacauugcaaaca

SEQ ID NO: 274 - RNA, 19 nt, Artificial Sequence
  Description of Artificial Sequence Synthetic oligonucleotide
aauaucaagcagauacaug

SEQ ID NO: 275 - RNA, 19 nt, Artificial Sequence
  Description of Artificial Sequence Synthetic oligonucleotide
uuuacacugagucgacaca

SEQ ID NO: 276 - RNA, 19 nt, Artificial Sequence
  Description of Artificial Sequence Synthetic oligonucleotide
auugauacuagcagaagca

SEQ ID NO: 277 - RNA, 19 nt, Artificial Sequence
  Description of Artificial Sequence Synthetic oligonucleotide
gguaagagcuacaagaaug

SEQ ID NO: 278 - RNA, 19 nt, Artificial Sequence
  Description of Artificial Sequence Synthetic oligonucleotide
uuuaagcuucguauccuca

SEQ ID NO: 279 - RNA, 19 nt, Artificial Sequence
  Description of Artificial Sequence Synthetic oligonucleotide
aacguaguaucugccuggc

SEQ ID NO: 280 - RNA, 19 nt, Artificial Sequence
  Description of Artificial Sequence Synthetic oligonucleotide
caguaaguuaaaugguccc

SEQ ID NO: 281 - RNA, 19 nt, Artificial Sequence
  Description of Artificial Sequence Synthetic oligonucleotide
gacugcauaaaagggagug

SEQ ID NO: 282 - RNA, 19 nt, Artificial Sequence
  Description of Artificial Sequence Synthetic oligonucleotide
auaagcugccacauuggcc

SEQ ID NO: 283 - RNA, 19 nt, Artificial Sequence
  Description of Artificial Sequence Synthetic oligonucleotide
acuuuugacugcauaaaag

SEQ ID NO: 284 - RNA, 19 nt, Artificial Sequence
  Description of Artificial Sequence Synthetic oligonucleotide
agggucaguacauccacuc

SEQ ID NO: 285 - RNA, 19 nt, Artificial Sequence
  Description of Artificial Sequence Synthetic oligonucleotide
agaugauccucucgggagg

SEQ ID NO: 286 - RNA, 19 nt, Artificial Sequence
  Description of Artificial Sequence Synthetic oligonucleotide
gaggcaacguaguaucugc

SEQ ID NO: 287 - RNA, 19 nt, Artificial Sequence
  Description of Artificial Sequence Synthetic oligonucleotide
gaugggauucaguuauggc

SEQ ID NO: 288 - RNA, 19 nt, Artificial Sequence
  Description of Artificial Sequence Synthetic oligonucleotide
ugcccaauuauuucaagau

SEQ ID NO: 289 - RNA, 19 nt, Artificial Sequence
  Description of Artificial Sequence Synthetic oligonucleotide
agcuacaaaugcuugcuca

SEQ ID NO: 290 - RNA, 19 nt, Artificial Sequence
  Description of Artificial Sequence Synthetic oligonucleotide
ugcauaaaagggagugacc

SEQ ID NO: 291 - RNA, 19 nt, Artificial Sequence
  Description of Artificial Sequence Synthetic oligonucleotide
uuagaaucgucaguggcga

SEQ ID NO: 292 - RNA, 19 nt, Artificial Sequence
  Description of Artificial Sequence Synthetic oligonucleotide
ugagucgacacacugcuuu

SEQ ID NO: 293 - RNA, 19 nt, Artificial Sequence
  Description of Artificial Sequence Synthetic oligonucleotide
uaaaagggagugacccggg

SEQ ID NO: 294 - RNA, 19 nt, Artificial Sequence
  Description of Artificial Sequence Synthetic oligonucleotide
aggaauacacuucauuaau

SEQ ID NO: 295 - RNA, 19 nt, Artificial Sequence
  Description of Artificial Sequence Synthetic oligonucleotide
uccgacaaggccuggacga

SEQ ID NO: 296 - RNA, 19 nt, Artificial Sequence
  Description of Artificial Sequence Synthetic oligonucleotide
augaucguaaagaaugcag

SEQ ID NO: 297 - RNA, 19 nt, Artificial Sequence
  Description of Artificial Sequence Synthetic oligonucleotide
auagaugauccucucggga

SEQ ID NO: 298 - RNA, 19 nt, Artificial Sequence
  Description of Artificial Sequence Synthetic oligonucleotide
ggauauggcagucaaacuc

SEQ ID NO: 299 - RNA, 19 nt, Artificial Sequence
  Description of Artificial Sequence Synthetic oligonucleotide
guuaguaugucugaaaagu

SEQ ID NO: 300 - RNA, 19 nt, Artificial Sequence
  Description of Artificial Sequence Synthetic oligonucleotide
gcuucguauccucagaucc

SEQ ID NO: 301 - RNA, 19 nt, Artificial Sequence
  Description of Artificial Sequence Synthetic oligonucleotide
agugugucccuucuuuaag

SEQ ID NO: 302 - RNA, 19 nt, Artificial Sequence
  Description of Artificial Sequence Synthetic oligonucleotide
caacguaguaucugccugg

SEQ ID NO: 303 - RNA, 19 nt, Artificial Sequence
  Description of Artificial Sequence Synthetic oligonucleotide
aaggccaucgcauguuggu

SEQ ID NO: 304 - RNA, 19 nt, Artificial Sequence
  Description of Artificial Sequence Synthetic oligonucleotide
aacccguugaaaguagaug

SEQ ID NO: 305 - RNA, 19 nt, Artificial Sequence
  Description of Artificial Sequence Synthetic oligonucleotide
caucaucuggacuccauug

SEQ ID NO: 306 - RNA, 19 nt, Artificial Sequence
  Description of Artificial Sequence Synthetic oligonucleotide
cuaugauauugacugccag

SEQ ID NO: 307 - RNA, 19 nt, Artificial Sequence
  Description of Artificial Sequence Synthetic oligonucleotide
ucgggaggcacuccaaggc

SEQ ID NO: 308 - RNA, 19 nt, Artificial Sequence
  Description of Artificial Sequence Synthetic oligonucleotide
gaaucgucaguggcgaucc

SEQ ID NO: 309 - RNA, 19 nt, Artificial Sequence
  Description of Artificial Sequence Synthetic oligonucleotide
gauaucaggucgcuaggca

SEQ ID NO: 310 - RNA, 19 nt, Artificial Sequence
  Description of Artificial Sequence Synthetic oligonucleotide
cauaagcugccacauuggc

SEQ ID NO: 311 - RNA, 19 nt, Artificial Sequence
  Description of Artificial Sequence Synthetic oligonucleotide
agguuugcucacuggacuc

SEQ ID NO: 312 - RNA, 19 nt, Artificial Sequence
  Description of Artificial Sequence Synthetic oligonucleotide
cauacacuccaucauucac

SEQ ID NO: 313 - RNA, 19 nt, Artificial Sequence
  Description of Artificial Sequence Synthetic oligonucleotide
ucuagagaugggauucagu

SEQ ID NO: 314 - RNA, 19 nt, Artificial Sequence
  Description of Artificial Sequence Synthetic oligonucleotide
uaagugugacccaucuccu

SEQ ID NO: 315 - RNA, 19 nt, Artificial Sequence
  Description of Artificial Sequence Synthetic oligonucleotide
accuucauuaacucaacuu

SEQ ID NO: 316 - RNA, 19 nt, Artificial Sequence
  Description of Artificial Sequence Synthetic oligonucleotide
uacaugaagguuugcucac

SEQ ID NO: 317 - RNA, 19 nt, Artificial Sequence
  Description of Artificial Sequence Synthetic oligonucleotide
uacauggauuugcauagau

SEQ ID NO: 318 - RNA, 19 nt, Artificial Sequence
  Description of Artificial Sequence Synthetic oligonucleotide
gaguaauacuucucaucug

SEQ ID NO: 319 - RNA, 19 nt, Artificial Sequence
  Description of Artificial Sequence Synthetic oligonucleotide
aacuuuacacugagucgac

SEQ ID NO: 320 - RNA, 19 nt, Artificial Sequence
  Description of Artificial Sequence Synthetic oligonucleotide
ucucggugugccuacaaaa

SEQ ID NO: 321 - RNA, 19 nt, Artificial Sequence
  Description of Artificial Sequence Synthetic oligonucleotide
ucuggcgggaaguacuugu

SEQ ID NO: 322 - RNA, 19 nt, Artificial Sequence
  Description of Artificial Sequence Synthetic oligonucleotide
aagcuaaacuugaagguaa

SEQ ID NO: 323 - RNA, 19 nt, Artificial Sequence
  Description of Artificial Sequence Synthetic oligonucleotide
uugcuuacauggauuugca

SEQ ID NO: 324 - RNA, 19 nt, Artificial Sequence
  Description of Artificial Sequence Synthetic oligonucleotide
aaacauucccugaugccca

SEQ ID NO: 325 - RNA, 19 nt, Artificial Sequence
  Description of Artificial Sequence Synthetic oligonucleotide
ucgaggauauggcagucaa

SEQ ID NO: 326 - RNA, 19 nt, Artificial Sequence
  Description of Artificial Sequence Synthetic oligonucleotide
uccgcaaaaccaacuuggc

SEQ ID NO: 327 - RNA, 19 nt, Artificial Sequence
  Description of Artificial Sequence Synthetic oligonucleotide
acaguguaugcacccaugu

SEQ ID NO: 328 - RNA, 19 nt, Artificial Sequence
  Description of Artificial Sequence Synthetic oligonucleotide
ucuggcgaccuucauuaac

SEQ ID NO: 329 - RNA, 19 nt, Artificial Sequence
  Description of Artificial Sequence Synthetic oligonucleotide
ucaguggcgauccgcaaaa

SEQ ID NO: 330 - RNA, 19 nt, Artificial Sequence
  Description of Artificial Sequence Synthetic oligonucleotide
cugcauaaaagggagugac

SEQ ID NO: 331 - RNA, 19 nt, Artificial Sequence
  Description of Artificial Sequence Synthetic oligonucleotide
uggaacccguugaaaguag

SEQ ID NO: 332 - RNA, 19 nt, Artificial Sequence
  Description of Artificial Sequence Synthetic oligonucleotide
gcugacaccaaugacauca

SEQ ID NO: 333 - RNA, 19 nt, Artificial Sequence
  Description of Artificial Sequence Synthetic oligonucleotide
auaguagauagaaggccuc

SEQ ID NO: 334 - RNA, 19 nt, Artificial Sequence
  Description of Artificial Sequence Synthetic oligonucleotide
uuacuagguggugaugcag

SEQ ID NO: 335 - RNA, 19 nt, Artificial Sequence
  Description of Artificial Sequence Synthetic oligonucleotide
ugauauugacugccagugu

SEQ ID NO: 336 - RNA, 19 nt, Artificial Sequence
  Description of Artificial Sequence Synthetic oligonucleotide
agcuucguauccucagauc

SEQ ID NO: 337 - RNA, 19 nt, Artificial Sequence
  Description of Artificial Sequence Synthetic oligonucleotide
agguaagugugacccaucu

SEQ ID NO: 338 - RNA, 19 nt, Artificial Sequence
  Description of Artificial Sequence Synthetic oligonucleotide
ugaggcaacguaguaucug

SEQ ID NO: 339 - RNA, 19 nt, Artificial Sequence
  Description of Artificial Sequence Synthetic oligonucleotide
ucucgggaggcacuccaag

SEQ ID NO: 340 - RNA, 19 nt, Artificial Sequence
  Description of Artificial Sequence Synthetic oligonucleotide
aucgucaguggcgauccgc

SEQ ID NO: 341 - RNA, 19 nt, Artificial Sequence
  Description of Artificial Sequence Synthetic oligonucleotide
aucacauaguagauagaag

SEQ ID NO: 342 - RNA, 19 nt, Artificial Sequence
  Description of Artificial Sequence Synthetic oligonucleotide
ucgacacacugcuuuagaa

SEQ ID NO: 343 - RNA, 19 nt, Artificial Sequence
  Description of Artificial Sequence Synthetic oligonucleotide
cacugagucgacacacugc

SEQ ID NO: 344 - RNA, 19 nt, Artificial Sequence
  Description of Artificial Sequence Synthetic oligonucleotide
cacauugauacuagcagaa

SEQ ID NO: 345 - RNA, 19 nt, Artificial Sequence
  Description of Artificial Sequence Synthetic oligonucleotide
uugcuggcggcauacuuga

SEQ ID NO: 346 - RNA, 19 nt, Artificial Sequence
  Description of Artificial Sequence Synthetic oligonucleotide
auacacuccaucauucaca

SEQ ID NO: 347 - RNA, 19 nt, Artificial Sequence
  Description of Artificial Sequence Synthetic oligonucleotide
cacaauccggucaaggcca

SEQ ID NO: 348 - RNA, 19 nt, Artificial Sequence
  Description of Artificial Sequence Synthetic oligonucleotide
cugugcaggaagugugucc

SEQ ID NO: 349 - RNA, 19 nt, Artificial Sequence
  Description of Artificial Sequence Synthetic oligonucleotide
augacgguccaucccgcuc

SEQ ID NO: 350 - RNA, 19 nt, Artificial Sequence
  Description of Artificial Sequence Synthetic oligonucleotide
cuacaagaauggcaucuac

SEQ ID NO: 351 - RNA, 19 nt, Artificial Sequence
  Description of Artificial Sequence Synthetic oligonucleotide
cucgggaggcacuccaagg

SEQ ID NO: 352 - RNA, 19 nt, Artificial Sequence
  Description of Artificial Sequence Synthetic oligonucleotide
auacgauuuucuuggcuau

SEQ ID NO: 353 - RNA, 19 nt, Artificial Sequence
  Description of Artificial Sequence Synthetic oligonucleotide
aacuaacaguaaguuaaau

SEQ ID NO: 354 - RNA, 19 nt, Artificial Sequence
  Description of Artificial Sequence Synthetic oligonucleotide
aggucucagggucaguaca

SEQ ID NO: 355 - RNA, 19 nt, Artificial Sequence
  Description of Artificial Sequence Synthetic oligonucleotide
ugacguuccucaccaacau

SEQ ID NO: 356 - RNA, 19 nt, Artificial Sequence
  Description of Artificial Sequence Synthetic oligonucleotide
acauugauacuagcagaag

SEQ ID NO: 357 - RNA, 19 nt, Artificial Sequence
  Description of Artificial Sequence Synthetic oligonucleotide
aaguacuuguccagagcug

SEQ ID NO: 358 - RNA, 19 nt, Artificial Sequence
  Description of Artificial Sequence Synthetic oligonucleotide
gcaguuaaaugacccauac

SEQ ID NO: 359 - RNA, 19 nt, Artificial Sequence
  Description of Artificial Sequence Synthetic oligonucleotide
aaacuaacaguaaguuaaa

SEQ ID NO: 360 - RNA, 19 nt, Artificial Sequence
  Description of Artificial Sequence Synthetic oligonucleotide
aaaugcuugcucaguggcu

SEQ ID NO: 361 - RNA, 19 nt, Artificial Sequence
  Description of Artificial Sequence Synthetic oligonucleotide
ucguauccucagauccagg

SEQ ID NO: 362 - RNA, 19 nt, Artificial Sequence
  Description of Artificial Sequence Synthetic oligonucleotide
caaggccaucgcauguugg

SEQ ID NO: 363 - RNA, 19 nt, Artificial Sequence
  Description of Artificial Sequence Synthetic oligonucleotide
aguagauagaaggccucug

SEQ ID NO: 364 - RNA, 19 nt, Artificial Sequence
  Description of Artificial Sequence Synthetic oligonucleotide
ggcuauaacucucacucca

SEQ ID NO: 365 - RNA, 19 nt, Artificial Sequence
  Description of Artificial Sequence Synthetic oligonucleotide
gcuuacauggauuugcaua

SEQ ID NO: 366 - RNA, 19 nt, Artificial Sequence
  Description of Artificial Sequence Synthetic oligonucleotide
gcuuuagaaucgucagugg

SEQ ID NO: 367 - RNA, 19 nt, Artificial Sequence
  Description of Artificial Sequence Synthetic oligonucleotide
ggcuaugauauugacugcc

SEQ ID NO: 368 - RNA, 19 nt, Artificial Sequence
  Description of Artificial Sequence Synthetic oligonucleotide
gaugauccucucgggaggc

SEQ ID NO: 369 - RNA, 19 nt, Artificial Sequence
  Description of Artificial Sequence Synthetic oligonucleotide
auacaugcugaaaccaacu

SEQ ID NO: 370 - RNA, 19 nt, Artificial Sequence
  Description of Artificial Sequence Synthetic oligonucleotide
acgauuuucuuggcuauga

SEQ ID NO: 371 - RNA, 19 nt, Artificial Sequence
  Description of Artificial Sequence Synthetic oligonucleotide
aaucgucaguggcgauccg

SEQ ID NO: 372 - RNA, 19 nt, Artificial Sequence
  Description of Artificial Sequence Synthetic oligonucleotide
ucagaacgucuccaagguc

SEQ ID NO: 373 - RNA, 19 nt, Artificial Sequence
  Description of Artificial Sequence Synthetic oligonucleotide
aucguaaagaaugcaguua

SEQ ID NO: 374 - RNA, 19 nt, Artificial Sequence
  Description of Artificial Sequence Synthetic oligonucleotide
guaugauacuuccaacugu

SEQ ID NO: 375 - RNA, 19 nt, Artificial Sequence
  Description of Artificial Sequence Synthetic oligonucleotide
gcugaggcaacguaguauc

SEQ ID NO: 376 - RNA, 19 nt, Artificial Sequence
  Description of Artificial Sequence Synthetic oligonucleotide
agcuaggguguucacuaca

SEQ ID NO: 377 - RNA, 19 nt, Artificial Sequence
  Description of Artificial Sequence Synthetic oligonucleotide
cuaggguguucacuacagc

SEQ ID NO: 378 - RNA, 19 nt, Artificial Sequence
  Description of Artificial Sequence Synthetic oligonucleotide
gucaguacauccacucccc

SEQ ID NO: 379 - RNA, 19 nt, Artificial Sequence
  Description of Artificial Sequence Synthetic oligonucleotide
caguacauccacuccccac

SEQ ID NO: 380 - RNA, 19 nt, Artificial Sequence
  Description of Artificial Sequence Synthetic oligonucleotide
agaacgucuccaagguccg

SEQ ID NO: 381 - RNA, 19 nt, Artificial Sequence
  Description of Artificial Sequence Synthetic oligonucleotide
gcaacguaguaucugccug

SEQ ID NO: 382 - RNA, 19 nt, Artificial Sequence
  Description of Artificial Sequence Synthetic oligonucleotide
auauugacugccaguguga

SEQ ID NO: 383 - RNA, 19 nt, Artificial Sequence
  Description of Artificial Sequence Synthetic oligonucleotide
augcaguuaaaugacccau

SEQ ID NO: 384 - RNA, 19 nt, Artificial Sequence
  Description of Artificial Sequence Synthetic oligonucleotide
augacguuccucaccaaca

SEQ ID NO: 385 - RNA, 19 nt, Artificial Sequence
  Description of Artificial Sequence Synthetic oligonucleotide
ucgauacgauuuucuuggc

SEQ ID NO: 386 - RNA, 19 nt, Artificial Sequence
  Description of Artificial Sequence Synthetic oligonucleotide
cacaugugcaugaucguaa

SEQ ID NO: 387 - RNA, 19 nt, Artificial Sequence
  Description of Artificial Sequence Synthetic oligonucleotide
auuuagcucuuuugcccgu

SEQ ID NO: 388 - RNA, 19 nt, Artificial Sequence
  Description of Artificial Sequence Synthetic oligonucleotide
uccauugcuggcggcauac

SEQ ID NO: 389 - RNA, 19 nt, Artificial Sequence
  Description of Artificial Sequence Synthetic oligonucleotide
ccacaauccggucaaggcc

SEQ ID NO: 390 - RNA, 19 nt, Artificial Sequence
  Description of Artificial Sequence Synthetic oligonucleotide
uacgauuuucuuggcuaug

SEQ ID NO: 391 - RNA, 19 nt, Artificial Sequence
  Description of Artificial Sequence Synthetic oligonucleotide
aggcaacguaguaucugcc

SEQ ID NO: 392 - RNA, 19 nt, Artificial Sequence
  Description of Artificial Sequence Synthetic oligonucleotide
aucugugccaagcccuacu

SEQ ID NO: 393 - RNA, 19 nt, Artificial Sequence
  Description of Artificial Sequence Synthetic oligonucleotide
gucacaacauuagcuuuug

SEQ ID NO: 394 - RNA, 19 nt, Artificial Sequence
  Description of Artificial Sequence Synthetic oligonucleotide
uuagcucuuuugcccguuc

SEQ ID NO: 395 - RNA, 19 nt, Artificial Sequence
  Description of Artificial Sequence Synthetic oligonucleotide
cuauaacucucacuccaga

SEQ ID NO: 396 - RNA, 19 nt, Artificial Sequence
  Description of Artificial Sequence Synthetic oligonucleotide
acccauacacuccaucauu

SEQ ID NO: 397 - RNA, 19 nt, Artificial Sequence
  Description of Artificial Sequence Synthetic oligonucleotide
cuucguauccucagaucca

SEQ ID NO: 398 - RNA, 19 nt, Artificial Sequence
  Description of Artificial Sequence Synthetic oligonucleotide
aguugauugccaagugcug

SEQ ID NO: 399 - RNA, 19 nt, Artificial Sequence
  Description of Artificial Sequence Synthetic oligonucleotide
agcuaaacuugaagguaag

SEQ ID NO: 400 - RNA, 19 nt, Artificial Sequence
  Description of Artificial Sequence Synthetic oligonucleotide
ucgcauguugguccccaga

SEQ ID NO: 401 - RNA, 19 nt, Artificial Sequence
  Description of Artificial Sequence Synthetic oligonucleotide
cuagaaacggccuagcacg

SEQ ID NO: 402 - RNA, 19 nt, Artificial Sequence
  Description of Artificial Sequence Synthetic oligonucleotide
aguacucuagucuggagcc

SEQ ID NO: 403 - RNA, 19 nt, Artificial Sequence
  Description of Artificial Sequence Synthetic oligonucleotide
guaacguaagcucacacgg

SEQ ID NO: 404 - RNA, 19 nt, Artificial Sequence
  Description of Artificial Sequence Synthetic oligonucleotide
augacugccauauucuccc

SEQ ID NO: 405 - RNA, 19 nt, Artificial Sequence
  Description of Artificial Sequence Synthetic oligonucleotide
acgcuuaucucauugacac

SEQ ID NO: 406 - RNA, 19 nt, Artificial Sequence
  Description of Artificial Sequence Synthetic oligonucleotide
auaugcuugggcuaucugg

SEQ ID NO: 407 - RNA, 19 nt, Artificial Sequence
  Description of Artificial Sequence Synthetic oligonucleotide
cugaaugcgagucauguag

SEQ ID NO: 408 - RNA, 19 nt, Artificial Sequence
  Description of Artificial Sequence Synthetic oligonucleotide
cauucaauaacuuucagcu

SEQ ID NO: 409 - RNA, 19 nt, Artificial Sequence
  Description of Artificial Sequence Synthetic oligonucleotide
aagcgauuccccuuucugg

SEQ ID NO: 410 - RNA, 19 nt, Artificial Sequence
  Description of Artificial Sequence Synthetic oligonucleotide
acgacaucagcguagcaac

SEQ ID NO: 411 - RNA, 19 nt, Artificial Sequence
  Description of Artificial Sequence Synthetic oligonucleotide
auauaagcaaccuccccuc

SEQ ID NO: 412 - RNA, 19 nt, Artificial Sequence
  Description of Artificial Sequence Synthetic oligonucleotide
aucgucaugccguugacag

SEQ ID NO: 413 - RNA, 19 nt, Artificial Sequence
  Description of Artificial Sequence Synthetic oligonucleotide
gacggaaguaggcagcucg

SEQ ID NO: 414 - RNA, 19 nt, Artificial Sequence
  Description of Artificial Sequence Synthetic oligonucleotide
ucccgauuuaagaagaggu

SEQ ID NO: 415 - RNA, 19 nt, Artificial Sequence
  Description of Artificial Sequence Synthetic oligonucleotide
aguaucagagacaguaccc

SEQ ID NO: 416 - RNA, 19 nt, Artificial Sequence
  Description of Artificial Sequence Synthetic oligonucleotide
ugugcguccaugcuucagg

SEQ ID NO: 417 - RNA, 19 nt, Artificial Sequence
  Description of Artificial Sequence Synthetic oligonucleotide
augcgagucauguagagca

SEQ ID NO: 418 - RNA, 19 nt, Artificial Sequence
  Description of Artificial Sequence Synthetic oligonucleotide
ucuucgacaaugcuuggcu

SEQ ID NO: 419 - RNA, 19 nt, Artificial Sequence
  Description of Artificial Sequence Synthetic oligonucleotide
cucaccucguagaacaugg

SEQ ID NO: 420 - RNA, 19 nt, Artificial Sequence
  Description of Artificial Sequence Synthetic oligonucleotide
uguacacauauaugcuugg

SEQ ID NO: 421 - RNA, 19 nt, Artificial Sequence
  Description of Artificial Sequence Synthetic oligonucleotide
gaguacucuagucuggagc

SEQ ID NO: 422 - RNA, 19 nt, Artificial Sequence
  Description of Artificial Sequence Synthetic oligonucleotide
uugagguguaagaacggag

SEQ ID NO: 423 - RNA, 19 nt, Artificial Sequence
  Description of Artificial Sequence Synthetic oligonucleotide
uguccaucgauauaagcaa

SEQ ID NO: 424 - RNA, 19 nt, Artificial Sequence
  Description of Artificial Sequence Synthetic oligonucleotide
agugaggaugaacugacca

SEQ ID NO: 425 - RNA, 19 nt, Artificial Sequence
  Description of Artificial Sequence Synthetic oligonucleotide
gucgugguuacuucggugg

SEQ ID NO: 426 - RNA, 19 nt, Artificial Sequence
  Description of Artificial Sequence Synthetic oligonucleotide
ugacauaugugcacugggc

SEQ ID NO: 427 - RNA, 19 nt, Artificial Sequence
  Description of Artificial Sequence Synthetic oligonucleotide
guaaguaucagagacagua

SEQ ID NO: 428 - RNA, 19 nt, Artificial Sequence
  Description of Artificial Sequence Synthetic oligonucleotide
uccaucgauauaagcaacc

SEQ ID NO: 429 - RNA, 19 nt, Artificial Sequence
  Description of Artificial Sequence Synthetic oligonucleotide
ugauccuuagugaacugcu

SEQ ID NO: 430 - RNA, 19 nt, Artificial Sequence
  Description of Artificial Sequence Synthetic oligonucleotide
ugccacguugaacaaauga

SEQ ID NO: 431 - RNA, 19 nt, Artificial Sequence
  Description of Artificial Sequence Synthetic oligonucleotide
cuggcgagcagacucaagg

SEQ ID NO: 432 - RNA, 19 nt, Artificial Sequence
  Description of Artificial Sequence Synthetic oligonucleotide
uucgacaaugcuuggcugc

SEQ ID NO: 433 - RNA, 19 nt, Artificial Sequence
  Description of Artificial Sequence Synthetic oligonucleotide
ucgauauaagcaaccuccc

SEQ ID NO: 434 - RNA, 19 nt, Artificial Sequence
  Description of Artificial Sequence Synthetic oligonucleotide
uguuggcccacuaaagagu

SEQ ID NO: 435 - RNA, 19 nt, Artificial Sequence
  Description of Artificial Sequence Synthetic oligonucleotide
aguggagccaaaucgcucu

SEQ ID NO: 436 - RNA, 19 nt, Artificial Sequence
  Description of Artificial Sequence Synthetic oligonucleotide
ugaucacuggucuucgaca

SEQ ID NO: 437 - RNA, 19 nt, Artificial Sequence
  Description of Artificial Sequence Synthetic oligonucleotide
uugaucacuggucuucgac

SEQ ID NO: 438 - RNA, 19 nt, Artificial Sequence
  Description of Artificial Sequence Synthetic oligonucleotide
uaagcucacacgguacugg

SEQ ID NO: 439 - RNA, 19 nt, Artificial Sequence
  Description of Artificial Sequence Synthetic oligonucleotide
cagagaguacucuagucug

SEQ ID NO: 440 - RNA, 19 nt, Artificial Sequence
  Description of Artificial Sequence Synthetic oligonucleotide
gaaugcgagucauguagag

SEQ ID NO: 441 - RNA, 19 nt, Artificial Sequence
  Description of Artificial Sequence Synthetic oligonucleotide
aacguaagcucacacggua

SEQ ID NO: 442 - RNA, 19 nt, Artificial Sequence
  Description of Artificial Sequence Synthetic oligonucleotide
auucaauaacuuucagcug

SEQ ID NO: 443 - RNA, 19 nt, Artificial Sequence
  Description of Artificial Sequence Synthetic oligonucleotide
aacggccuagcacgguggc

SEQ ID NO: 444 - RNA, 19 nt, Artificial Sequence
  Description of Artificial Sequence Synthetic oligonucleotide
ugaaugcgagucauguaga

SEQ ID NO: 445 - RNA, 19 nt, Artificial Sequence
  Description of Artificial Sequence Synthetic oligonucleotide
gaugacugccauauucucc

SEQ ID NO: 446 - RNA, 19 nt, Artificial Sequence
  Description of Artificial Sequence Synthetic oligonucleotide
uaagcaaccuccccucgca

SEQ ID NO: 447 - RNA, 19 nt, Artificial Sequence
  Description of Artificial Sequence Synthetic oligonucleotide
cuugacggaaguaggcagc

SEQ ID NO: 448 - RNA, 19 nt, Artificial Sequence
  Description of Artificial Sequence Synthetic oligonucleotide
ugggaggugccacguaacg

SEQ ID NO: 449 - RNA, 19 nt, Artificial Sequence
  Description of Artificial Sequence Synthetic oligonucleotide
uucccgauuuaagaagagg

SEQ ID NO: 450 - RNA, 19 nt, Artificial Sequence
  Description of Artificial Sequence Synthetic oligonucleotide
aagccacaaagucgcgaac

SEQ ID NO: 451 - RNA, 19 nt, Artificial Sequence
  Description of Artificial Sequence Synthetic oligonucleotide
caucggaugcaucaccacc

SEQ ID NO: 452 - RNA, 19 nt, Artificial Sequence
  Description of Artificial Sequence Synthetic oligonucleotide
agucuucccgauuuaagaa

SEQ ID NO: 453 - RNA, 19 nt, Artificial Sequence
  Description of Artificial Sequence Synthetic oligonucleotide
agcacgacgacaucagcgu

SEQ ID NO: 454 - RNA, 19 nt, Artificial Sequence
  Description of Artificial Sequence Synthetic oligonucleotide
aagucgcgaacgcugggug

SEQ ID NO: 455 - RNA, 19 nt, Artificial Sequence
  Description of Artificial Sequence Synthetic oligonucleotide
agcgauuccccuuucugga

SEQ ID NO: 456 - RNA, 19 nt, Artificial Sequence
  Description of Artificial Sequence Synthetic oligonucleotide
agcaacacucugccgcucg

SEQ ID NO: 457 - RNA, 19 nt, Artificial Sequence
  Description of Artificial Sequence Synthetic oligonucleotide
aggugucgugguuacuucg

SEQ ID NO: 458 - RNA, 19 nt, Artificial Sequence
  Description of Artificial Sequence Synthetic oligonucleotide
ugccacguaacguaagcuc

SEQ ID NO: 459 - RNA, 19 nt, Artificial Sequence
  Description of Artificial Sequence Synthetic oligonucleotide
gugguuacuucgguggugg

SEQ ID NO: 460 - RNA, 19 nt, Artificial Sequence
  Description of Artificial Sequence Synthetic oligonucleotide
acgucuuguccauagccug

SEQ ID NO: 461 - RNA, 19 nt, Artificial Sequence
  Description of Artificial Sequence Synthetic oligonucleotide
gacacggggcaucggaugc

SEQ ID NO: 462 - RNA, 19 nt, Artificial Sequence
  Description of Artificial Sequence Synthetic oligonucleotide
ugcacugagucggcaagcg

SEQ ID NO: 463 - RNA, 19 nt, Artificial Sequence
  Description of Artificial Sequence Synthetic oligonucleotide
uaacguaagcucacacggu

SEQ ID NO: 464 - RNA, 19 nt, Artificial Sequence
  Description of Artificial Sequence Synthetic oligonucleotide
gggaaccaagcucaggccg

SEQ ID NO: 465 - RNA, 19 nt, Artificial Sequence
  Description of Artificial Sequence Synthetic oligonucleotide
aaagucgcgaacgcugggu

SEQ ID NO: 466 - RNA, 19 nt, Artificial Sequence
  Description of Artificial Sequence Synthetic oligonucleotide
caccucguagaacauggag

SEQ ID NO: 467 - RNA, 19 nt, Artificial Sequence
  Description of Artificial Sequence Synthetic oligonucleotide
ucaccagcguagcaucccc

SEQ ID NO: 468 - RNA, 19 nt, Artificial Sequence
  Description of Artificial Sequence Synthetic oligonucleotide
acgacgacaucagcguagc

SEQ ID NO: 469 - RNA, 19 nt, Artificial Sequence
  Description of Artificial Sequence Synthetic oligonucleotide
cauugaucacuggucuucg

SEQ ID NO: 470 - RNA, 19 nt, Artificial Sequence
  Description of Artificial Sequence Synthetic oligonucleotide
caucgauauaagcaaccuc

SEQ ID NO: 471 - RNA, 19 nt, Artificial Sequence
  Description of Artificial Sequence Synthetic oligonucleotide
agagaguacucuagucugg

SEQ ID NO: 472 - RNA, 19 nt, Artificial Sequence
  Description of Artificial Sequence Synthetic oligonucleotide
caggucaccuaccauggug

SEQ ID NO: 473 - RNA, 19 nt, Artificial Sequence
  Description of Artificial Sequence Synthetic oligonucleotide
agucgcgaacgcugggugg

SEQ ID NO: 474 - RNA, 19 nt, Artificial Sequence
  Description of Artificial Sequence Synthetic oligonucleotide
aagcaaccuccccucgcag

SEQ ID NO: 475 - RNA, 19 nt, Artificial Sequence
  Description of Artificial Sequence Synthetic oligonucleotide
acauauaugcuugggcuau

SEQ ID NO: 476 - RNA, 19 nt, Artificial Sequence
  Description of Artificial Sequence Synthetic oligonucleotide
gaagauccgccgaggauug

SEQ ID NO: 477 - RNA, 19 nt, Artificial Sequence
  Description of Artificial Sequence Synthetic oligonucleotide
agagauggcgauagccgac

SEQ ID NO: 478 - RNA, 19 nt, Artificial Sequence
  Description of Artificial Sequence Synthetic oligonucleotide
gaugacucggcguggucuc

SEQ ID NO: 479 - RNA, 19 nt, Artificial Sequence
  Description of Artificial Sequence Synthetic oligonucleotide
gaugccugucuagguaccg

SEQ ID NO: 480 - RNA, 19 nt, Artificial Sequence
  Description of Artificial Sequence Synthetic oligonucleotide
cuucccgauuuaagaagag

SEQ ID NO: 481 - RNA, 19 nt, Artificial Sequence
  Description of Artificial Sequence Synthetic oligonucleotide
ucgacaaugcuuggcugcc

SEQ ID NO: 482 - RNA, 19 nt, Artificial Sequence
  Description of Artificial Sequence Synthetic oligonucleotide
cacguucauggcagcaccg

SEQ ID NO: 483 - RNA, 19 nt, Artificial Sequence
  Description of Artificial Sequence Synthetic oligonucleotide
ucaggugucgugguuacuu

SEQ ID NO: 484 - RNA, 19 nt, Artificial Sequence
  Description of Artificial Sequence Synthetic oligonucleotide
cugcuaagugugcugcccc

SEQ ID NO: 485 - RNA, 19 nt, Artificial Sequence
  Description of Artificial Sequence Synthetic oligonucleotide
ugcgcagacugggaggugc

SEQ ID NO: 486 - RNA, 19 nt, Artificial Sequence
  Description of Artificial Sequence Synthetic oligonucleotide
guggauucgggguggcaag

SEQ ID NO: 487 - RNA, 19 nt, Artificial Sequence
  Description of Artificial Sequence Synthetic oligonucleotide
ccuagaaacggccuagcac

SEQ ID NO: 488 - RNA, 19 nt, Artificial Sequence
  Description of Artificial Sequence Synthetic oligonucleotide
ugccggagcacgacgacau

SEQ ID NO: 489 - RNA, 19 nt, Artificial Sequence
  Description of Artificial Sequence Synthetic oligonucleotide
cacgaugccaucgcaggcc

SEQ ID NO: 490 - RNA, 19 nt, Artificial Sequence
  Description of Artificial Sequence Synthetic oligonucleotide
cacuggucuucgacaaugc

SEQ ID NO: 491 - RNA, 19 nt, Artificial Sequence
  Description of Artificial Sequence Synthetic oligonucleotide
cauagguguccuccuggag

SEQ ID NO: 492 - RNA, 19 nt, Artificial Sequence
  Description of Artificial Sequence Synthetic oligonucleotide
uugacggaaguaggcagcu

SEQ ID NO: 493 - RNA, 19 nt, Artificial Sequence
  Description of Artificial Sequence Synthetic oligonucleotide
acuacgcagggguacccca

SEQ ID NO: 494 - RNA, 19 nt, Artificial Sequence
  Description of Artificial Sequence Synthetic oligonucleotide
ggaccucucccuuuccagg

SEQ ID NO: 495 - RNA, 19 nt, Artificial Sequence
  Description of Artificial Sequence Synthetic oligonucleotide
ucgugguuacuucgguggu

SEQ ID NO: 496 - RNA, 19 nt, Artificial Sequence
  Description of Artificial Sequence Synthetic oligonucleotide
ggugccacguaacguaagc

SEQ ID NO: 497 - RNA, 19 nt, Artificial Sequence
  Description of Artificial Sequence Synthetic oligonucleotide
cacgcuuaucucauugaca

SEQ ID NO: 498 - RNA, 19 nt, Artificial Sequence
  Description of Artificial Sequence Synthetic oligonucleotide
gguuacuucggugguggcg

SEQ ID NO: 499 - RNA, 19 nt, Artificial Sequence
  Description of Artificial Sequence Synthetic oligonucleotide
ccuuucuggacggaggacg

SEQ ID NO: 500 - RNA, 19 nt, Artificial Sequence
  Description of Artificial Sequence Synthetic oligonucleotide
caugcggauguacaugcca

SEQ ID NO: 501 - RNA, 19 nt, Artificial Sequence
  Description of Artificial Sequence Synthetic oligonucleotide
uuuagcccagcugcagaug

SEQ ID NO: 502 - RNA, 19 nt, Artificial Sequence
  Description of Artificial Sequence Synthetic oligonucleotide
auagccgacacgaugccau

SEQ ID NO: 503 - RNA, 19 nt, Artificial Sequence
  Description of Artificial Sequence Synthetic oligonucleotide
caaagucgcgaacgcuggg

SEQ ID NO: 504 - RNA, 19 nt, Artificial Sequence
  Description of Artificial Sequence Synthetic oligonucleotide
agcgcucuagguccccauc

SEQ ID NO: 505 - RNA, 19 nt, Artificial Sequence
  Description of Artificial Sequence Synthetic oligonucleotide
ugcgagucauguagagcac

SEQ ID NO: 506 - RNA, 19 nt, Artificial Sequence
  Description of Artificial Sequence Synthetic oligonucleotide
agggugccggagcacgacg

SEQ ID NO: 507 - RNA, 19 nt, Artificial Sequence
  Description of Artificial Sequence Synthetic oligonucleotide
guggcuagggauuguccac

SEQ ID NO: 508 - RNA, 19 nt, Artificial Sequence
  Description of Artificial Sequence Synthetic oligonucleotide
uaccgguggugcaggguag

SEQ ID NO: 509 - RNA, 19 nt, Artificial Sequence
  Description of Artificial Sequence Synthetic oligonucleotide
gcgauagccgacacgaugc

SEQ ID NO: 510 - RNA, 19 nt, Artificial Sequence
  Description of Artificial Sequence Synthetic oligonucleotide
auggggagcauggucugag

SEQ ID NO: 511 - RNA, 19 nt, Artificial Sequence
  Description of Artificial Sequence Synthetic oligonucleotide
ggaaccaagcucaggccgg

SEQ ID NO: 512 - RNA, 19 nt, Artificial Sequence
  Description of Artificial Sequence Synthetic oligonucleotide
ugucuagguaccgguggug

SEQ ID NO: 513 - RNA, 19 nt, Artificial Sequence
  Description of Artificial Sequence Synthetic oligonucleotide
cacauauaugcuugggcua

SEQ ID NO: 514 - RNA, 19 nt, Artificial Sequence
  Description of Artificial Sequence Synthetic oligonucleotide
cccgauuuaagaagaggug

SEQ ID NO: 515 - RNA, 19 nt, Artificial Sequence
  Description of Artificial Sequence Synthetic oligonucleotide
acgugacauaugugcacug

SEQ ID NO: 516 - RNA, 19 nt, Artificial Sequence
  Description of Artificial Sequence Synthetic oligonucleotide
gaugccccuggaugucugc

SEQ ID NO: 517 - RNA, 19 nt, Artificial Sequence
  Description of Artificial Sequence Synthetic oligonucleotide
aggggcgcacuacgcaggg

SEQ ID NO: 518 - RNA, 19 nt, Artificial Sequence
  Description of Artificial Sequence Synthetic oligonucleotide
gucuucgacaaugcuuggc

SEQ ID NO: 519 - RNA, 19 nt, Artificial Sequence
  Description of Artificial Sequence Synthetic oligonucleotide
agauccgccgaggauugug

SEQ ID NO: 520 - RNA, 19 nt, Artificial Sequence
  Description of Artificial Sequence Synthetic oligonucleotide
aggaggggcgcacuacgca

SEQ ID NO: 521 - RNA, 19 nt, Artificial Sequence
  Description of Artificial Sequence Synthetic oligonucleotide
cacacggcggaugguaccc

SEQ ID NO: 522 - RNA, 19 nt, Artificial Sequence
  Description of Artificial Sequence Synthetic oligonucleotide
uuccguacgucuuguccau

SEQ ID NO: 523 - RNA, 19 nt, Artificial Sequence
  Description of Artificial Sequence Synthetic oligonucleotide
aaccaagcucaggccggac

SEQ ID NO: 524 - RNA, 19 nt, Artificial Sequence
  Description of Artificial Sequence Synthetic oligonucleotide
agcauugaucacuggucuu

SEQ ID NO: 525 - RNA, 19 nt, Artificial Sequence
  Description of Artificial Sequence Synthetic oligonucleotide
ccuguccaucgauauaagc

SEQ ID NO: 526 - RNA, 19 nt, Artificial Sequence
  Description of Artificial Sequence Synthetic oligonucleotide
ugaugcggcagaggagccc

SEQ ID NO: 527 - RNA, 19 nt, Artificial Sequence
  Description of Artificial Sequence Synthetic oligonucleotide
agucggcaagcgauucccc

SEQ ID NO: 528 - RNA, 19 nt, Artificial Sequence
  Description of Artificial Sequence Synthetic oligonucleotide
cauaggaggggcgcacuac

SEQ ID NO: 529 - RNA, 19 nt, Artificial Sequence
  Description of Artificial Sequence Synthetic oligonucleotide
caccagcguagcauccccg

SEQ ID NO: 530 - RNA, 19 nt, Artificial Sequence
  Description of Artificial Sequence Synthetic oligonucleotide
agccgacacgaugccaucg

SEQ ID NO: 531 - RNA, 19 nt, Artificial Sequence
  Description of Artificial Sequence Synthetic oligonucleotide
gucugcuaagugugcugcc

SEQ ID NO: 532 - RNA, 19 nt, Artificial Sequence
  Description of Artificial Sequence Synthetic oligonucleotide
gagauggcgauagccgaca

SEQ ID NO: 533 - RNA, 19 nt, Artificial Sequence
  Description of Artificial Sequence Synthetic oligonucleotide
guggaagauccgccgagga

SEQ ID NO: 534 - RNA, 19 nt, Artificial Sequence
  Description of Artificial Sequence Synthetic oligonucleotide
gucgcgaacgcuggguggc

SEQ ID NO: 535 - RNA, 19 nt, Artificial Sequence
  Description of Artificial Sequence Synthetic oligonucleotide
guaccgguggugcagggua

SEQ ID NO: 536 - RNA, 19 nt, Artificial Sequence
  Description of Artificial Sequence Synthetic oligonucleotide
ggaggugccacguaacgua

SEQ ID NO: 537 - RNA, 19 nt, Artificial Sequence
  Description of Artificial Sequence Synthetic oligonucleotide
auccguagugugugugcca

SEQ ID NO: 538 - RNA, 19 nt, Artificial Sequence
  Description of Artificial Sequence Synthetic oligonucleotide
guccaucgauauaagcaac

SEQ ID NO: 539 - RNA, 19 nt, Artificial Sequence
  Description of Artificial Sequence Synthetic oligonucleotide
gaguggagccaaaucgcuc

SEQ ID NO: 540 - RNA, 19 nt, Artificial Sequence
  Description of Artificial Sequence Synthetic oligonucleotide
caggugucgugguuacuuc

SEQ ID NO: 541 - RNA, 19 nt, Artificial Sequence
  Description of Artificial Sequence Synthetic oligonucleotide
gaacuggcgagcagacuca

SEQ ID NO: 542 - RNA, 19 nt, Artificial Sequence
  Description of Artificial Sequence Synthetic oligonucleotide
ucgucaugccguugacagu

SEQ ID NO: 543 - RNA, 19 nt, Artificial Sequence
  Description of Artificial Sequence Synthetic oligonucleotide
agugcacugugcguccaug

SEQ ID NO: 544 - RNA, 19 nt, Artificial Sequence
  Description of Artificial Sequence Synthetic oligonucleotide
ccgaggauuguggugacag

SEQ ID NO: 545 - RNA, 19 nt, Artificial Sequence
  Description of Artificial Sequence Synthetic oligonucleotide
gcauugaucacuggucuuc

SEQ ID NO: 546 - RNA, 19 nt, Artificial Sequence
  Description of Artificial Sequence Synthetic oligonucleotide
acgcagggguaccccacag

SEQ ID NO: 547 - RNA, 19 nt, Artificial Sequence
  Description of Artificial Sequence Synthetic oligonucleotide
gcgagucauguagagcacg

SEQ ID NO: 548 - RNA, 19 nt, Artificial Sequence
  Description of Artificial Sequence Synthetic oligonucleotide
gaacgcuggguggcaugcg

SEQ ID NO: 549 - RNA, 19 nt, Artificial Sequence
  Description of Artificial Sequence Synthetic oligonucleotide
cuggguagggugcucucca

SEQ ID NO: 550 - RNA, 19 nt, Artificial Sequence
  Description of Artificial Sequence Synthetic oligonucleotide
cgauauaagcaaccucccc

SEQ ID NO: 551 - RNA, 19 nt, Artificial Sequence
  Description of Artificial Sequence Synthetic oligonucleotide
gccagagaguacucuaguc

SEQ ID NO: 552 - RNA, 19 nt, Artificial Sequence
  Description of Artificial Sequence Synthetic oligonucleotide
ccacuaaagagugcagcag

SEQ ID NO: 553 - RNA, 19 nt, Artificial Sequence
  Description of Artificial Sequence Synthetic oligonucleotide
ugcguccaugcuucagguc

SEQ ID NO: 554 - RNA, 19 nt, Artificial Sequence
  Description of Artificial Sequence Synthetic oligonucleotide
ccguacgucuuguccauag

SEQ ID NO: 555 - RNA, 19 nt, Artificial Sequence
  Description of Artificial Sequence Synthetic oligonucleotide
uuccgagccacgugacaua

SEQ ID NO: 556 - RNA, 19 nt, Artificial Sequence
  Description of Artificial Sequence Synthetic oligonucleotide
cauauaugcuugggcuauc

SEQ ID NO: 557 - RNA, 19 nt, Artificial Sequence
  Description of Artificial Sequence Synthetic oligonucleotide
cuguuuggcuauuuauuau

SEQ ID NO: 558 - RNA, 19 nt, Artificial Sequence
  Description of Artificial Sequence Synthetic oligonucleotide
cugagucggcaagcgauuc

SEQ ID NO: 559 - RNA, 19 nt, Artificial Sequence
  Description of Artificial Sequence Synthetic oligonucleotide
gcccacuaaagagugcagc

SEQ ID NO: 560 - RNA, 19 nt, Artificial Sequence
  Description of Artificial Sequence Synthetic oligonucleotide
aacuggcgagcagacucaa

SEQ ID NO: 561 - RNA, 19 nt, Artificial Sequence
  Description of Artificial Sequence Synthetic oligonucleotide
cuguaaccuguaguuccug

SEQ ID NO: 562 - RNA, 19 nt, Artificial Sequence
  Description of Artificial Sequence Synthetic oligonucleotide
ccucgcaggaccacacggc

SEQ ID NO: 563 - RNA, 19 nt, Artificial Sequence
  Description of Artificial Sequence Synthetic oligonucleotide
gggagugcacugugcgucc

SEQ ID NO: 564 - RNA, 19 nt, Artificial Sequence
  Description of Artificial Sequence Synthetic oligonucleotide
uggugaucgucaugccguu

SEQ ID NO: 565 - RNA, 19 nt, Artificial Sequence
  Description of Artificial Sequence Synthetic oligonucleotide
gccgacacgaugccaucgc

SEQ ID NO: 566 - RNA, 19 nt, Artificial Sequence
  Description of Artificial Sequence Synthetic oligonucleotide
gacucggcguggucucuca

SEQ ID NO: 567 - RNA, 19 nt, Artificial Sequence
  Description of Artificial Sequence Synthetic oligonucleotide
gcgggugcucaccucguag

SEQ ID NO: 568 - RNA, 19 nt, Artificial Sequence
  Description of Artificial Sequence Synthetic oligonucleotide
augucaaggcuccgcucuu

SEQ ID NO: 569 - RNA, 19 nt, Artificial Sequence
  Description of Artificial Sequence Synthetic oligonucleotide
gaaccaagcucaggccgga

SEQ ID NO: 570 - RNA, 19 nt, Artificial Sequence
  Description of Artificial Sequence Synthetic oligonucleotide
uagccgacacgaugccauc

SEQ ID NO: 571 - RNA, 19 nt, Artificial Sequence
  Description of Artificial Sequence Synthetic oligonucleotide
ccagagaguacucuagucu

SEQ ID NO: 572 - RNA, 19 nt, Artificial Sequence
  Description of Artificial Sequence Synthetic oligonucleotide
ggauguguuggcccacuaa

SEQ ID NO: 573 - RNA, 19 nt, Artificial Sequence
  Description of Artificial Sequence Synthetic oligonucleotide
ccgcacugcucagaaagcg

SEQ ID NO: 574 - RNA, 19 nt, Artificial Sequence
  Description of Artificial Sequence Synthetic oligonucleotide
acugagucggcaagcgauu

SEQ ID NO: 575 - RNA, 19 nt, Artificial Sequence
  Description of Artificial Sequence Synthetic oligonucleotide
ccccggaaugcacaccugc

SEQ ID NO: 576 - RNA, 19 nt, Artificial Sequence
  Description of Artificial Sequence Synthetic oligonucleotide
cugccgcucggcagugggc

SEQ ID NO: 577 - RNA, 19 nt, Artificial Sequence
  Description of Artificial Sequence Synthetic oligonucleotide
aggugccacguaacguaag

SEQ ID NO: 578 - RNA, 19 nt, Artificial Sequence
  Description of Artificial Sequence Synthetic oligonucleotide
ggaagauccgccgaggauu

SEQ ID NO: 579 - RNA, 19 nt, Artificial Sequence
  Description of Artificial Sequence Synthetic oligonucleotide
ggguagggugcucuccaac

SEQ ID NO: 580 - RNA, 19 nt, Artificial Sequence
  Description of Artificial Sequence Synthetic oligonucleotide
gucggcaagcgauuccccu

SEQ ID NO: 581 - RNA, 19 nt, Artificial Sequence
  Description of Artificial Sequence Synthetic oligonucleotide
ugucaaggcuccgcucuuu

SEQ ID NO: 582 - RNA, 19 nt, Artificial Sequence
  Description of Artificial Sequence Synthetic oligonucleotide
cuacuggugcgggugcuca

SEQ ID NO: 583 - RNA, 19 nt, Artificial Sequence
  Description of Artificial Sequence Synthetic oligonucleotide
gcaacucauggucugcacu

SEQ ID NO: 584 - RNA, 19 nt, Artificial Sequence
  Description of Artificial Sequence Synthetic oligonucleotide
caggaccacacggcggaug

SEQ ID NO: 585 - RNA, 19 nt, Artificial Sequence
  Description of Artificial Sequence Synthetic oligonucleotide
ggugcucaccucguagaac

SEQ ID NO: 586 - RNA, 19 nt, Artificial Sequence
  Description of Artificial Sequence Synthetic oligonucleotide
cacuuccguacgucuuguc

SEQ ID NO: 587 - RNA, 19 nt, Artificial Sequence
  Description of Artificial Sequence Synthetic oligonucleotide
ugacggaaguaggcagcuc

SEQ ID NO: 588 - RNA, 19 nt, Artificial Sequence
  Description of Artificial Sequence Synthetic oligonucleotide
ugacucggcguggucucuc

SEQ ID NO: 589 - RNA, 19 nt, Artificial Sequence
  Description of Artificial Sequence Synthetic oligonucleotide
ccauauccucucgggaacc

SEQ ID NO: 590 - RNA, 19 nt, Artificial Sequence
  Description of Artificial Sequence Synthetic oligonucleotide
agucgaugacugccauauu

SEQ ID NO: 591 - RNA, 19 nt, Artificial Sequence
  Description of Artificial Sequence Synthetic oligonucleotide
ccacacggcggaugguacc

SEQ ID NO: 592 - RNA, 19 nt, Artificial Sequence
  Description of Artificial Sequence Synthetic oligonucleotide
ggucaccagcguagcaucc

SEQ ID NO: 593 - RNA, 19 nt, Artificial Sequence
  Description of Artificial Sequence Synthetic oligonucleotide
guccacucaugcucuagau

SEQ ID NO: 594 - RNA, 19 nt, Artificial Sequence
  Description of Artificial Sequence Synthetic oligonucleotide
caugcgcagacugggaggu

SEQ ID NO: 595 - RNA, 19 nt, Artificial Sequence
  Description of Artificial Sequence Synthetic oligonucleotide
ggguaccaacaccugucca

SEQ ID NO: 596 - RNA, 19 nt, Artificial Sequence
  Description of Artificial Sequence Synthetic oligonucleotide
gcgcucuagguccccauca

SEQ ID NO: 597 - RNA, 19 nt, Artificial Sequence
  Description of Artificial Sequence Synthetic oligonucleotide
gcuagggauuguccacuca

SEQ ID NO: 598 - RNA, 19 nt, Artificial Sequence
  Description of Artificial Sequence Synthetic oligonucleotide
ggcuagggauuguccacuc

SEQ ID NO: 599 - RNA, 19 nt, Artificial Sequence
  Description of Artificial Sequence Synthetic oligonucleotide
accgaacuccagaguuuug

SEQ ID NO: 600 - RNA, 19 nt, Artificial Sequence
  Description of Artificial Sequence Synthetic oligonucleotide
gggcgcacuacgcaggggu

SEQ ID NO: 601 - RNA, 19 nt, Artificial Sequence
  Description of Artificial Sequence Synthetic oligonucleotide
aaaggaagguuccugauuc

SEQ ID NO: 602 - RNA, 19 nt, Artificial Sequence
  Description of Artificial Sequence Synthetic oligonucleotide
uugccaaaguauaagaugg

SEQ ID NO: 603 - RNA, 19 nt, Artificial Sequence
  Description of Artificial Sequence Synthetic oligonucleotide
uuuauugaacaacagauuu

SEQ ID NO: 604 - RNA, 19 nt, Artificial Sequence
  Description of Artificial Sequence Synthetic oligonucleotide
uauaagaugguuuccagcu

SEQ ID NO: 605 - RNA, 19 nt, Artificial Sequence
  Description of Artificial Sequence Synthetic oligonucleotide
guaguaaucaaacuuggcc

SEQ ID NO: 606 - RNA, 19 nt, Artificial Sequence
  Description of Artificial Sequence Synthetic oligonucleotide
ugauuaaagaagccaagag

SEQ ID NO: 607 - RNA, 19 nt, Artificial Sequence
  Description of Artificial Sequence Synthetic oligonucleotide
ugauucauacccacgauug

SEQ ID NO: 608 - RNA, 19 nt, Artificial Sequence
  Description of Artificial Sequence Synthetic oligonucleotide
guucacaagauccacaacc

SEQ ID NO: 609 - RNA, 19 nt, Artificial Sequence
  Description of Artificial Sequence Synthetic oligonucleotide
aaaaugucacuucgggagg

SEQ ID NO: 610 - RNA, 19 nt, Artificial Sequence
  Description of Artificial Sequence Synthetic oligonucleotide
auucuuacccacccaagcc

SEQ ID NO: 611 - RNA, 19 nt, Artificial Sequence
  Description of Artificial Sequence Synthetic oligonucleotide
gaagcgaacaacugcaugc

SEQ ID NO: 612 - RNA, 19 nt, Artificial Sequence
  Description of Artificial Sequence Synthetic oligonucleotide
caagauaccagucaccagc

SEQ ID NO: 613 - RNA, 19 nt, Artificial Sequence
  Description of Artificial Sequence Synthetic oligonucleotide
auugguaugcuuuguagag

SEQ ID NO: 614 - RNA, 19 nt, Artificial Sequence
  Description of Artificial Sequence Synthetic oligonucleotide
cuuauagagggccuggucc

SEQ ID NO: 615 - RNA, 19 nt, Artificial Sequence
  Description of Artificial Sequence Synthetic oligonucleotide
ugcauugauguucagucgc

SEQ ID NO: 616 - RNA, 19 nt, Artificial Sequence
  Description of Artificial Sequence Synthetic oligonucleotide
gaagaggcuacuuccuucg

SEQ ID NO: 617 - RNA, 19 nt, Artificial Sequence
  Description of Artificial Sequence Synthetic oligonucleotide
ugcuuuauugaacaacaga

SEQ ID NO: 618 - RNA, 19 nt, Artificial Sequence
  Description of Artificial Sequence Synthetic oligonucleotide
ugagauagcuggaccaggg

SEQ ID NO: 619 - RNA, 19 nt, Artificial Sequence
  Description of Artificial Sequence Synthetic oligonucleotide
agauccacaaccucgucag

SEQ ID NO: 620 - RNA, 19 nt, Artificial Sequence
  Description of Artificial Sequence Synthetic oligonucleotide
uguugcacaaccucagcag

SEQ ID NO: 621 - RNA, 19 nt, Artificial Sequence
  Description of Artificial Sequence Synthetic oligonucleotide
ugaguaguuggccuggucu

SEQ ID NO: 622 - RNA, 19 nt, Artificial Sequence
  Description of Artificial Sequence Synthetic oligonucleotide
guagaguuguuccagaauc

SEQ ID NO: 623 - RNA, 19 nt, Artificial Sequence
  Description of Artificial Sequence Synthetic oligonucleotide
auaacaggcauguaguaau

SEQ ID NO: 624 - RNA, 19 nt, Artificial Sequence
  Description of Artificial Sequence Synthetic oligonucleotide
uggacuugaagaugagagg

SEQ ID NO: 625 - RNA, 19 nt, Artificial Sequence
  Description of Artificial Sequence Synthetic oligonucleotide
uuggucaucugguagucag

SEQ ID NO: 626 - RNA, 19 nt, Artificial Sequence
  Description of Artificial Sequence Synthetic oligonucleotide
gauuaaagaagccaagagu

SEQ ID NO: 627 - RNA, 19 nt, Artificial Sequence
  Description of Artificial Sequence Synthetic oligonucleotide
ugaaugccacuuuucacag

SEQ ID NO: 628 - RNA, 19 nt, Artificial Sequence
  Description of Artificial Sequence Synthetic oligonucleotide
uccugauucauacccacga

SEQ ID NO: 629 - RNA, 19 nt, Artificial Sequence
  Description of Artificial Sequence Synthetic oligonucleotide
guggacuugaagaugagag

SEQ ID NO: 630 - RNA, 19 nt, Artificial Sequence
  Description of Artificial Sequence Synthetic oligonucleotide
acaaacucguaggcgaucc

SEQ ID NO: 631 - RNA, 19 nt, Artificial Sequence
  Description of Artificial Sequence Synthetic oligonucleotide
uucauucuuacccacccaa

SEQ ID NO: 632 - RNA, 19 nt, Artificial Sequence
  Description of Artificial Sequence Synthetic oligonucleotide
uucuuacccacccaagcca

SEQ ID NO: 633 - RNA, 19 nt, Artificial Sequence
  Description of Artificial Sequence Synthetic oligonucleotide
agugcauguuuucuuguag

SEQ ID NO: 634 - RNA, 19 nt, Artificial Sequence
  Description of Artificial Sequence Synthetic oligonucleotide
agugucacagaguugugca

SEQ ID NO: 635 - RNA, 19 nt, Artificial Sequence
  Description of Artificial Sequence Synthetic oligonucleotide
ucugaugguacuucuuaca

SEQ ID NO: 636 - RNA, 19 nt, Artificial Sequence
  Description of Artificial Sequence Synthetic oligonucleotide
guagcguacuuccacauac

SEQ ID NO: 637 - RNA, 19 nt, Artificial Sequence
  Description of Artificial Sequence Synthetic oligonucleotide
uucuugccaaaguauaaga

SEQ ID NO: 638 - RNA, 19 nt, Artificial Sequence
  Description of Artificial Sequence Synthetic oligonucleotide
uucauacccacgauuggca

SEQ ID NO: 639 - RNA, 19 nt, Artificial Sequence
  Description of Artificial Sequence Synthetic oligonucleotide
uuauugaacaacagauuuu

SEQ ID NO: 640 - RNA, 19 nt, Artificial Sequence
  Description of Artificial Sequence Synthetic oligonucleotide
ucuuucuugaagcgaacaa

SEQ ID NO: 641 - RNA, 19 nt, Artificial Sequence
  Description of Artificial Sequence Synthetic oligonucleotide
ucacagcucccucauaggc

SEQ ID NO: 642 - RNA, 19 nt, Artificial Sequence
  Description of Artificial Sequence Synthetic oligonucleotide
aaagugcauguuuucuugu

SEQ ID NO: 643 - RNA, 19 nt, Artificial Sequence
  Description of Artificial Sequence Synthetic oligonucleotide
aaaugucacuucgggaggg

SEQ ID NO: 644 - RNA, 19 nt, Artificial Sequence
  Description of Artificial Sequence Synthetic oligonucleotide
cauucuuacccacccaagc

SEQ ID NO: 645 - RNA, 19 nt, Artificial Sequence
  Description of Artificial Sequence Synthetic oligonucleotide
ggacuugaagaugagaggg

SEQ ID NO: 646 - RNA, 19 nt, Artificial Sequence
  Description of Artificial Sequence Synthetic oligonucleotide
ucuuacccacccaagccag

SEQ ID NO: 647 - RNA, 19 nt, Artificial Sequence
  Description of Artificial Sequence Synthetic oligonucleotide
uccguuuugggaucccagg

SEQ ID NO: 648 - RNA, 19 nt, Artificial Sequence
  Description of Artificial Sequence Synthetic oligonucleotide
uugucuugaguacauccac

SEQ ID NO: 649 - RNA, 19 nt, Artificial Sequence
  Description of Artificial Sequence Synthetic oligonucleotide
ucuccacaaacucguaggc

SEQ ID NO: 650 - RNA, 19 nt, Artificial Sequence
  Description of Artificial Sequence Synthetic oligonucleotide
ucauucuuacccacccaag

SEQ ID NO: 651 - RNA, 19 nt, Artificial Sequence
  Description of Artificial Sequence Synthetic oligonucleotide
uccacaaccucgucagggg

SEQ ID NO: 652 - RNA, 19 nt, Artificial Sequence
  Description of Artificial Sequence Synthetic oligonucleotide
aggacucugauuaaagaag

SEQ ID NO: 653 - RNA, 19 nt, Artificial Sequence
  Description of Artificial Sequence Synthetic oligonucleotide
ugcugcaagauaccaguca

SEQ ID NO: 654 - RNA, 19 nt, Artificial Sequence
  Description of Artificial Sequence Synthetic oligonucleotide
ugagugaguaguuggccug

SEQ ID NO: 655 - RNA, 19 nt, Artificial Sequence
  Description of Artificial Sequence Synthetic oligonucleotide
cuuucuugaagcgaacaac

SEQ ID NO: 656 - RNA, 19 nt, Artificial Sequence
  Description of Artificial Sequence Synthetic oligonucleotide
cugaugguacuucuuacac

SEQ ID NO: 657 - RNA, 19 nt, Artificial Sequence
  Description of Artificial Sequence Synthetic oligonucleotide
ucguaggcgauccuuuuga

SEQ ID NO: 658 - RNA, 19 nt, Artificial Sequence
  Description of Artificial Sequence Synthetic oligonucleotide
auacccacgauuggcaagg

SEQ ID NO: 659 - RNA, 19 nt, Artificial Sequence
  Description of Artificial Sequence Synthetic oligonucleotide
uagaguuguuccagaaucu

SEQ ID NO: 660 - RNA, 19 nt, Artificial Sequence
  Description of Artificial Sequence Synthetic oligonucleotide
accgugugcugcaagauac

SEQ ID NO: 661 - RNA, 19 nt, Artificial Sequence
  Description of Artificial Sequence Synthetic oligonucleotide
uuccugauucauacccacg

SEQ ID NO: 662 - RNA, 19 nt, Artificial Sequence
  Description of Artificial Sequence Synthetic oligonucleotide
ucuggaaggaaugaaggua

SEQ ID NO: 663 - RNA, 19 nt, Artificial Sequence
  Description of Artificial Sequence Synthetic oligonucleotide
uugaaagcgggcgucuggg

SEQ ID NO: 664 - RNA, 19 nt, Artificial Sequence
  Description of Artificial Sequence Synthetic oligonucleotide
uguccaacccuguuugucu

SEQ ID NO: 665 - RNA, 19 nt, Artificial Sequence
  Description of Artificial Sequence Synthetic oligonucleotide
uguagaguuguuccagaau

SEQ ID NO: 666 - RNA, 19 nt, Artificial Sequence
  Description of Artificial Sequence Synthetic oligonucleotide
aguucaggagcaugugcuc

SEQ ID NO: 667 - RNA, 19 nt, Artificial Sequence
  Description of Artificial Sequence Synthetic oligonucleotide
ugugagauagcuggaccag

SEQ ID NO: 668 - RNA, 19 nt, Artificial Sequence
  Description of Artificial Sequence Synthetic oligonucleotide
cucgucaggggugacaucc

SEQ ID NO: 669 - RNA, 19 nt, Artificial Sequence
  Description of Artificial Sequence Synthetic oligonucleotide
uucggucugguuccagggg

SEQ ID NO: 670 - RNA, 19 nt, Artificial Sequence
  Description of Artificial Sequence Synthetic oligonucleotide
ugaguacauccacagccug

SEQ ID NO: 671 - RNA, 19 nt, Artificial Sequence
  Description of Artificial Sequence Synthetic oligonucleotide
gugugugguagccaugucc

SEQ ID NO: 672 - RNA, 19 nt, Artificial Sequence
  Description of Artificial Sequence Synthetic oligonucleotide
aacaacugcaugcuccguu

SEQ ID NO: 673 - RNA, 19 nt, Artificial Sequence
  Description of Artificial Sequence Synthetic oligonucleotide
aaagcgggcgucugggcca

SEQ ID NO: 674 - RNA, 19 nt, Artificial Sequence
  Description of Artificial Sequence Synthetic oligonucleotide
uuaaaggaagguuccugau

SEQ ID NO: 675 - RNA, 19 nt, Artificial Sequence
  Description of Artificial Sequence Synthetic oligonucleotide
agaguuguuccagaaucuc

SEQ ID NO: 676 - RNA, 19 nt, Artificial Sequence
  Description of Artificial Sequence Synthetic oligonucleotide
cuucuugccaaaguauaag

SEQ ID NO: 677 - RNA, 19 nt, Artificial Sequence
  Description of Artificial Sequence Synthetic oligonucleotide
uucugaugguacuucuuac

SEQ ID NO: 678 - RNA, 19 nt, Artificial Sequence
  Description of Artificial Sequence Synthetic oligonucleotide
ucuuauagagggccugguc

SEQ ID NO: 679 - RNA, 19 nt, Artificial Sequence
  Description of Artificial Sequence Synthetic oligonucleotide
cuucggucugguuccaggg

SEQ ID NO: 680 - RNA, 19 nt, Artificial Sequence
  Description of Artificial Sequence Synthetic oligonucleotide
gcgccugugagauagcugg

SEQ ID NO: 681 - RNA, 19 nt, Artificial Sequence
  Description of Artificial Sequence Synthetic oligonucleotide
uuacccacccaagccagcg

SEQ ID NO: 682 - RNA, 19 nt, Artificial Sequence
  Description of Artificial Sequence Synthetic oligonucleotide
guugaaagcgggcgucugg

SEQ ID NO: 683 - RNA, 19 nt, Artificial Sequence
  Description of Artificial Sequence Synthetic oligonucleotide
cuucgauggucucaucacc

SEQ ID NO: 684 - RNA, 19 nt, Artificial Sequence
  Description of Artificial Sequence Synthetic oligonucleotide
uaaucaaacuuggccagga

SEQ ID NO: 685 - RNA, 19 nt, Artificial Sequence
  Description of Artificial Sequence Synthetic oligonucleotide
aaugaagguaagaguguca

SEQ ID NO: 686 - RNA, 19 nt, Artificial Sequence
  Description of Artificial Sequence Synthetic oligonucleotide
uagccucuuauagagggcc

SEQ ID NO: 687 - RNA, 19 nt, Artificial Sequence
  Description of Artificial Sequence Synthetic oligonucleotide
gucccguuuggucaucugg

SEQ ID NO: 688 - RNA, 19 nt, Artificial Sequence
  Description of Artificial Sequence Synthetic oligonucleotide
cugcauugauguucagucg

SEQ ID NO: 689 - RNA, 19 nt, Artificial Sequence
  Description of Artificial Sequence Synthetic oligonucleotide
ugucacagaguugugcaga

SEQ ID NO: 690 - RNA, 19 nt, Artificial Sequence
  Description of Artificial Sequence Synthetic oligonucleotide
ucggucugguuccagggga

SEQ ID NO: 691 - RNA, 19 nt, Artificial Sequence
  Description of Artificial Sequence Synthetic oligonucleotide
guaagagugucacagaguu

SEQ ID NO: 692 - RNA, 19 nt, Artificial Sequence
  Description of Artificial Sequence Synthetic oligonucleotide
gauuggguccacuuuggaa

SEQ ID NO: 693 - RNA, 19 nt, Artificial Sequence
  Description of Artificial Sequence Synthetic oligonucleotide
gauagcuggaccaggggca

SEQ ID NO: 694 - RNA, 19 nt, Artificial Sequence
  Description of Artificial Sequence Synthetic oligonucleotide
ucgcuugaauucuuccuca

SEQ ID NO: 695 - RNA, 19 nt, Artificial Sequence
  Description of Artificial Sequence Synthetic oligonucleotide
uuuagccuuaaaggaaggu

SEQ ID NO: 696 - RNA, 19 nt, Artificial Sequence
  Description of Artificial Sequence Synthetic oligonucleotide
aagugcauguuuucuugua

SEQ ID NO: 697 - RNA, 19 nt, Artificial Sequence
  Description of Artificial Sequence Synthetic oligonucleotide
gaauuggccagcaggugug

SEQ ID NO: 698 - RNA, 19 nt, Artificial Sequence
  Description of Artificial Sequence Synthetic oligonucleotide
aaucaaacuuggccaggaa

SEQ ID NO: 699 - RNA, 19 nt, Artificial Sequence
  Description of Artificial Sequence Synthetic oligonucleotide
uccccuucggucugguucc

SEQ ID NO: 700 - RNA, 19 nt, Artificial Sequence
  Description of Artificial Sequence Synthetic oligonucleotide
acccuguuugucuugagua

SEQ ID NO: 701 - RNA, 19 nt, Artificial Sequence
  Description of Artificial Sequence Synthetic oligonucleotide
aagauaccagucaccagcu

SEQ ID NO: 702 - RNA, 19 nt, Artificial Sequence
  Description of Artificial Sequence Synthetic oligonucleotide
guugagugaguaguuggcc

SEQ ID NO: 703 - RNA, 19 nt, Artificial Sequence
  Description of Artificial Sequence Synthetic oligonucleotide
uuguagaguuguuccagaa

SEQ ID NO: 704 - RNA, 19 nt, Artificial Sequence
  Description of Artificial Sequence Synthetic oligonucleotide
guaaucaaacuuggccagg

SEQ ID NO: 705 - RNA, 19 nt, Artificial Sequence
  Description of Artificial Sequence Synthetic oligonucleotide
gucgcuugaauucuuccuc

SEQ ID NO: 706 - RNA, 19 nt, Artificial Sequence
  Description of Artificial Sequence Synthetic oligonucleotide
caguaaagcccaugucccg

SEQ ID NO: 707 - RNA, 19 nt, Artificial Sequence
  Description of Artificial Sequence Synthetic oligonucleotide
uggacgguacggugaaugc

SEQ ID NO: 708 - RNA, 19 nt, Artificial Sequence
  Description of Artificial Sequence Synthetic oligonucleotide
aagcgaacaacugcaugcu

SEQ ID NO: 709 - RNA, 19 nt, Artificial Sequence
  Description of Artificial Sequence Synthetic oligonucleotide
cccacgauuggcaaggccc

SEQ ID NO: 710 - RNA, 19 nt, Artificial Sequence
  Description of Artificial Sequence Synthetic oligonucleotide
guaggcgauccuuuugaug

SEQ ID NO: 711 - RNA, 19 nt, Artificial Sequence
  Description of Artificial Sequence Synthetic oligonucleotide
ugccaaaguauaagauggu

SEQ ID NO: 712 - RNA, 19 nt, Artificial Sequence
  Description of Artificial Sequence Synthetic oligonucleotide
uaacaggcauguaguaauc

SEQ ID NO: 713 - RNA, 19 nt, Artificial Sequence
  Description of Artificial Sequence Synthetic oligonucleotide
ugcaugcuccguuuuggga

SEQ ID NO: 714 - RNA, 19 nt, Artificial Sequence
  Description of Artificial Sequence Synthetic oligonucleotide
auguccaacccuguuuguc

SEQ ID NO: 715 - RNA, 19 nt, Artificial Sequence
  Description of Artificial Sequence Synthetic oligonucleotide
cugauuaaagaagccaaga

SEQ ID NO: 716 - RNA, 19 nt, Artificial Sequence
  Description of Artificial Sequence Synthetic oligonucleotide
acccacgauuggcaaggcc

SEQ ID NO: 717 - RNA, 19 nt, Artificial Sequence
  Description of Artificial Sequence Synthetic oligonucleotide
gacgguacggugaaugcca

SEQ ID NO: 718 - RNA, 19 nt, Artificial Sequence
  Description of Artificial Sequence Synthetic oligonucleotide
cugugagauagcuggacca

SEQ ID NO: 719 - RNA, 19 nt, Artificial Sequence
  Description of Artificial Sequence Synthetic oligonucleotide
gaccacauucaccgugugc

SEQ ID NO: 720 - RNA, 19 nt, Artificial Sequence
  Description of Artificial Sequence Synthetic oligonucleotide
uacggugaaugccacuuuu

SEQ ID NO: 721 - RNA, 19 nt, Artificial Sequence
  Description of Artificial Sequence Synthetic oligonucleotide
uggacgugcagcucuacuu

SEQ ID NO: 722 - RNA, 19 nt, Artificial Sequence
  Description of Artificial Sequence Synthetic oligonucleotide
agcgaacaacugcaugcuc

SEQ ID NO: 723 - RNA, 19 nt, Artificial Sequence
  Description of Artificial Sequence Synthetic oligonucleotide
ugcagcccgcgauaacagg

SEQ ID NO: 724 - RNA, 19 nt, Artificial Sequence
  Description of Artificial Sequence Synthetic oligonucleotide
uugaccccgaauucuugcu

SEQ ID NO: 725 - RNA, 19 nt, Artificial Sequence
  Description of Artificial Sequence Synthetic oligonucleotide
gacgugcagcucuacuuug

SEQ ID NO: 726 - RNA, 19 nt, Artificial Sequence
  Description of Artificial Sequence Synthetic oligonucleotide
ucauacccacgauuggcaa

SEQ ID NO: 727 - RNA, 19 nt, Artificial Sequence
  Description of Artificial Sequence Synthetic oligonucleotide
acaagauccacaaccucgu

SEQ ID NO: 728 - RNA, 19 nt, Artificial Sequence
  Description of Artificial Sequence Synthetic oligonucleotide
ggguugguggcgcauacag

SEQ ID NO: 729 - RNA, 19 nt, Artificial Sequence
  Description of Artificial Sequence Synthetic oligonucleotide
uggcgcauacagcacagua

SEQ ID NO: 730 - RNA, 19 nt, Artificial Sequence
  Description of Artificial Sequence Synthetic oligonucleotide
gauuggcaaggccccuggg

SEQ ID NO: 731 - RNA, 19 nt, Artificial Sequence
  Description of Artificial Sequence Synthetic oligonucleotide
uagcguacuuccacauaca

SEQ ID NO: 732 - RNA, 19 nt, Artificial Sequence
  Description of Artificial Sequence Synthetic oligonucleotide
uugguaugcuuuguagagu

SEQ ID NO: 733 - RNA, 19 nt, Artificial Sequence
  Description of Artificial Sequence Synthetic oligonucleotide
cuuguugaaagcgggcguc

SEQ ID NO: 734 - RNA, 19 nt, Artificial Sequence
  Description of Artificial Sequence Synthetic oligonucleotide
uguagcguacuuccacaua

SEQ ID NO: 735 - RNA, 19 nt, Artificial Sequence
  Description of Artificial Sequence Synthetic oligonucleotide
cugcaugcuccguuuuggg

SEQ ID NO: 736 - RNA, 19 nt, Artificial Sequence
  Description of Artificial Sequence Synthetic oligonucleotide
acauucaccgugugcugca

SEQ ID NO: 737 - RNA, 19 nt, Artificial Sequence
  Description of Artificial Sequence Synthetic oligonucleotide
ggcuuguugaaagcgggcg

SEQ ID NO: 738 - RNA, 19 nt, Artificial Sequence
  Description of Artificial Sequence Synthetic oligonucleotide
ggucucaucaccagccagg

SEQ ID NO: 739 - RNA, 19 nt, Artificial Sequence
  Description of Artificial Sequence Synthetic oligonucleotide
gauccacaaccucgucagg

SEQ ID NO: 740 - RNA, 19 nt, Artificial Sequence
  Description of Artificial Sequence Synthetic oligonucleotide
uugaagcgaacaacugcau

SEQ ID NO: 741 - RNA, 19 nt, Artificial Sequence
  Description of Artificial Sequence Synthetic oligonucleotide
gucgucuguguugagugag

SEQ ID NO: 742 - RNA, 19 nt, Artificial Sequence
  Description of Artificial Sequence Synthetic oligonucleotide
ggggauuggguccacuuug

SEQ ID NO: 743 - RNA, 19 nt, Artificial Sequence
  Description of Artificial Sequence Synthetic oligonucleotide
cacaguauggaccggaccu

SEQ ID NO: 744 - RNA, 19 nt, Artificial Sequence
  Description of Artificial Sequence Synthetic oligonucleotide
aaacucguaggcgauccuu

SEQ ID NO: 745 - RNA, 19 nt, Artificial Sequence
  Description of Artificial Sequence Synthetic oligonucleotide
aggguggacuugaagauga

SEQ ID NO: 746 - RNA, 19 nt, Artificial Sequence
  Description of Artificial Sequence Synthetic oligonucleotide
ugguagccauguccaaccc

SEQ ID NO: 747 - RNA, 19 nt, Artificial Sequence
  Description of Artificial Sequence Synthetic oligonucleotide
ugaccccgaauucuugcuc

SEQ ID NO: 748 - RNA, 19 nt, Artificial Sequence
  Description of Artificial Sequence Synthetic oligonucleotide
cacauucaccgugugcugc

SEQ ID NO: 749 - RNA, 19 nt, Artificial Sequence
  Description of Artificial Sequence Synthetic oligonucleotide
cacauacaccacacccucc

SEQ ID NO: 750 - RNA, 19 nt, Artificial Sequence
  Description of Artificial Sequence Synthetic oligonucleotide
gggucgucuguguugagug

SEQ ID NO: 751 - RNA, 19 nt, Artificial Sequence
  Description of Artificial Sequence Synthetic oligonucleotide
agugugugguagccauguc

SEQ ID NO: 752 - RNA, 19 nt, Artificial Sequence
  Description of Artificial Sequence Synthetic oligonucleotide
uuggguugguggcgcauac

SEQ ID NO: 753 - RNA, 19 nt, Artificial Sequence
  Description of Artificial Sequence Synthetic oligonucleotide
cgcgauaacaggcauguag

SEQ ID NO: 754 - RNA, 19 nt, Artificial Sequence
  Description of Artificial Sequence Synthetic oligonucleotide
cuuguaguagccucuuaua

SEQ ID NO: 755 - RNA, 19 nt, Artificial Sequence
  Description of Artificial Sequence Synthetic oligonucleotide
agagggucgucuguguuga

SEQ ID NO: 756 - RNA, 19 nt, Artificial Sequence
  Description of Artificial Sequence Synthetic oligonucleotide
uguugaaagcgggcgucug

SEQ ID NO: 757 - RNA, 19 nt, Artificial Sequence
  Description of Artificial Sequence Synthetic oligonucleotide
cauggugccgagugugcac

SEQ ID NO: 758 - RNA, 19 nt, Artificial Sequence
  Description of Artificial Sequence Synthetic oligonucleotide
aguacauccacagccuguu

SEQ ID NO: 759 - RNA, 19 nt, Artificial Sequence
  Description of Artificial Sequence Synthetic oligonucleotide
aaugucacuucgggaggga

SEQ ID NO: 760 - RNA, 19 nt, Artificial Sequence
  Description of Artificial Sequence Synthetic oligonucleotide
ugggcccuagccagaacac

SEQ ID NO: 761 - RNA, 19 nt, Artificial Sequence
  Description of Artificial Sequence Synthetic oligonucleotide
gcgaacaacugcaugcucc

SEQ ID NO: 762 - RNA, 19 nt, Artificial Sequence
  Description of Artificial Sequence Synthetic oligonucleotide
agcucccucauaggcuugc

SEQ ID NO: 763 - RNA, 19 nt, Artificial Sequence
  Description of Artificial Sequence Synthetic oligonucleotide
caugcuccguuuugggauc

SEQ ID NO: 764 - RNA, 19 nt, Artificial Sequence
  Description of Artificial Sequence Synthetic oligonucleotide
ucgucuguguugagugagu

SEQ ID NO: 765 - RNA, 19 nt, Artificial Sequence
  Description of Artificial Sequence Synthetic oligonucleotide
ugagagggucgucuguguu

SEQ ID NO: 766 - RNA, 19 nt, Artificial Sequence
  Description of Artificial Sequence Synthetic oligonucleotide
uuuggucaucugguaguca

SEQ ID NO: 767 - RNA, 19 nt, Artificial Sequence
  Description of Artificial Sequence Synthetic oligonucleotide
aggcuugcacauguccugg

SEQ ID NO: 768 - RNA, 19 nt, Artificial Sequence
  Description of Artificial Sequence Synthetic oligonucleotide
acggugaaugccacuuuuc

SEQ ID NO: 769 - RNA, 19 nt, Artificial Sequence
  Description of Artificial Sequence Synthetic oligonucleotide
uugguggcgcauacagcac

SEQ ID NO: 770 - RNA, 19 nt, Artificial Sequence
  Description of Artificial Sequence Synthetic oligonucleotide
auagcuggaccaggggcag

SEQ ID NO: 771 - RNA, 19 nt, Artificial Sequence
  Description of Artificial Sequence Synthetic oligonucleotide
uacacagcuccaacaccuc

SEQ ID NO: 772 - RNA, 19 nt, Artificial Sequence
  Description of Artificial Sequence Synthetic oligonucleotide
ucgggagggaaugucccug

SEQ ID NO: 773 - RNA, 19 nt, Artificial Sequence
  Description of Artificial Sequence Synthetic oligonucleotide
cuuacccacccaagccagc

SEQ ID NO: 774 - RNA, 19 nt, Artificial Sequence
  Description of Artificial Sequence Synthetic oligonucleotide
ucauaggcuugcacauguc

SEQ ID NO: 775 - RNA, 19 nt, Artificial Sequence
  Description of Artificial Sequence Synthetic oligonucleotide
augguacuucuuacacagc

SEQ ID NO: 776 - RNA, 19 nt, Artificial Sequence
  Description of Artificial Sequence Synthetic oligonucleotide
uucgggagggaaugucccu

SEQ ID NO: 777 - RNA, 19 nt, Artificial Sequence
  Description of Artificial Sequence Synthetic oligonucleotide
cagccuguugcacaaccuc

SEQ ID NO: 778 - RNA, 19 nt, Artificial Sequence
  Description of Artificial Sequence Synthetic oligonucleotide
uuguugaaagcgggcgucu

SEQ ID NO: 779 - RNA, 19 nt, Artificial Sequence
  Description of Artificial Sequence Synthetic oligonucleotide
guaugcuuuguagaguugu

SEQ ID NO: 780 - RNA, 19 nt, Artificial Sequence
  Description of Artificial Sequence Synthetic oligonucleotide
gugccgagugugcaccccg

SEQ ID NO: 781 - RNA, 19 nt, Artificial Sequence
  Description of Artificial Sequence Synthetic oligonucleotide
agcauggacgguacgguga

SEQ ID NO: 782 - RNA, 19 nt, Artificial Sequence
  Description of Artificial Sequence Synthetic oligonucleotide
cagcauggggccccaagac

SEQ ID NO: 783 - RNA, 19 nt, Artificial Sequence
  Description of Artificial Sequence Synthetic oligonucleotide
aacucguaggcgauccuuu

SEQ ID NO: 784 - RNA, 19 nt, Artificial Sequence
  Description of Artificial Sequence Synthetic oligonucleotide
ugcgcagccccuccacugu

SEQ ID NO: 785 - RNA, 19 nt, Artificial Sequence
  Description of Artificial Sequence Synthetic oligonucleotide
cauccacaggcugaggcuc

SEQ ID NO: 786 - RNA, 19 nt, Artificial Sequence
  Description of Artificial Sequence Synthetic oligonucleotide
cgaacaacugcaugcuccg

SEQ ID NO: 787 - RNA, 19 nt, Artificial Sequence
  Description of Artificial Sequence Synthetic oligonucleotide
ccuucgauggucucaucac

SEQ ID NO: 788 - RNA, 19 nt, Artificial Sequence
  Description of Artificial Sequence Synthetic oligonucleotide
cuucaggggaccugcccuc

SEQ ID NO: 789 - RNA, 19 nt, Artificial Sequence
  Description of Artificial Sequence Synthetic oligonucleotide
cagcauggacgguacggug

SEQ ID NO: 790 - RNA, 19 nt, Artificial Sequence
  Description of Artificial Sequence Synthetic oligonucleotide
guucaggagcaugugcuca

SEQ ID NO: 791 - RNA, 19 nt, Artificial Sequence
  Description of Artificial Sequence Synthetic oligonucleotide
aucaccagccaggucgaug

SEQ ID NO: 792 - RNA, 19 nt, Artificial Sequence
  Description of Artificial Sequence Synthetic oligonucleotide
acaccugaucagaggacag

SEQ ID NO: 793 - RNA, 19 nt, Artificial Sequence
  Description of Artificial Sequence Synthetic oligonucleotide
guagccucuuauagagggc

SEQ ID NO: 794 - RNA, 19 nt, Artificial Sequence
  Description of Artificial Sequence Synthetic oligonucleotide
augcuccguuuugggaucc

SEQ ID NO: 795 - RNA, 19 nt, Artificial Sequence
  Description of Artificial Sequence Synthetic oligonucleotide
cuacuuugggcuuguugaa

SEQ ID NO: 796 - RNA, 19 nt, Artificial Sequence
  Description of Artificial Sequence Synthetic oligonucleotide
aaccucgucaggggugaca

SEQ ID NO: 797 - RNA, 19 nt, Artificial Sequence
  Description of Artificial Sequence Synthetic oligonucleotide
ugucccguuuggucaucug

SEQ ID NO: 798 - RNA, 19 nt, Artificial Sequence
  Description of Artificial Sequence Synthetic oligonucleotide
ggaauuggccagcaggugu

SEQ ID NO: 799 - RNA, 19 nt, Artificial Sequence
  Description of Artificial Sequence Synthetic oligonucleotide
gcagaccucaaagugcaug

SEQ ID NO: 800 - RNA, 19 nt, Artificial Sequence
  Description of Artificial Sequence Synthetic oligonucleotide
ccugaucagaggacagacc

SEQ ID NO: 801 - RNA, 19 nt, Artificial Sequence
  Description of Artificial Sequence Synthetic oligonucleotide
aguaauaaagcgcugagcc

SEQ ID NO: 802 - RNA, 19 nt, Artificial Sequence
  Description of Artificial Sequence Synthetic oligonucleotide
uuuaauauaucaaaaggcc

SEQ ID NO: 803 - RNA, 19 nt, Artificial Sequence
  Description of Artificial Sequence Synthetic oligonucleotide
auacauaugcaucuuagcc

SEQ ID NO: 804 - RNA, 19 nt, Artificial Sequence
  Description of Artificial Sequence Synthetic oligonucleotide
uugaguuacaaugagacag

SEQ ID NO: 805 - RNA, 19 nt, Artificial Sequence
  Description of Artificial Sequence Synthetic oligonucleotide
aguaauacggaccuugggg

SEQ ID NO: 806 - RNA, 19 nt, Artificial Sequence
  Description of Artificial Sequence Synthetic oligonucleotide
aauaaaagcaagauuaacu

SEQ ID NO: 807 - RNA, 19 nt, Artificial Sequence
  Description of Artificial Sequence Synthetic oligonucleotide
aaccccauaaaccccaccc

SEQ ID NO: 808 - RNA, 19 nt, Artificial Sequence
  Description of Artificial Sequence Synthetic oligonucleotide
ucaacccagauauacaugg

SEQ ID NO: 809 - RNA, 19 nt, Artificial Sequence
  Description of Artificial Sequence Synthetic oligonucleotide
aaguacauuugcuugaugu

SEQ ID NO: 810 - RNA, 19 nt, Artificial Sequence
  Description of Artificial Sequence Synthetic oligonucleotide
uuuagugacaugcuagucc

SEQ ID NO: 811 - RNA, 19 nt, Artificial Sequence
  Description of Artificial Sequence Synthetic oligonucleotide
uauucugaccaauaaaagc

SEQ ID NO: 812 - RNA, 19 nt, Artificial Sequence
  Description of Artificial Sequence Synthetic oligonucleotide
uaggaaaggcucaagauca

SEQ ID NO: 813 - RNA, 19 nt, Artificial Sequence
  Description of Artificial Sequence Synthetic oligonucleotide
ugcggauuccuucacaggg

SEQ ID NO: 814 - RNA, 19 nt, Artificial Sequence
  Description of Artificial Sequence Synthetic oligonucleotide
uuaaccaagcucuucaaac

SEQ ID NO: 815 - RNA, 19 nt, Artificial Sequence
  Description of Artificial Sequence Synthetic oligonucleotide
ucauugagaaggcaugugc

SEQ ID NO: 816 - RNA, 19 nt, Artificial Sequence
  Description of Artificial Sequence Synthetic oligonucleotide
gauguuggacguuucgugg

SEQ ID NO: 817 - RNA, 19 nt, Artificial Sequence
  Description of Artificial Sequence Synthetic oligonucleotide
uaauaaagcgcugagcccc

SEQ ID NO: 818 - RNA, 19 nt, Artificial Sequence
  Description of Artificial Sequence Synthetic oligonucleotide
ucuuagccuaagcacaggg

SEQ ID NO: 819 - RNA, 19 nt, Artificial Sequence
  Description of Artificial Sequence Synthetic oligonucleotide
agugguuacguucccuucc

SEQ ID NO: 820 - RNA, 19 nt, Artificial Sequence
  Description of Artificial Sequence Synthetic oligonucleotide
agugccuuaaauuggucuc

SEQ ID NO: 821 - RNA, 19 nt, Artificial Sequence
  Description of Artificial Sequence Synthetic oligonucleotide
uguaaaguuagaaacccua

SEQ ID NO: 822 - RNA, 19 nt, Artificial Sequence
  Description of Artificial Sequence Synthetic oligonucleotide
aaagguugcuauuacccca

SEQ ID NO: 823 - RNA, 19 nt, Artificial Sequence
  Description of Artificial Sequence Synthetic oligonucleotide
uucacaggguccuauuugg

SEQ ID NO: 824 - RNA, 19 nt, Artificial Sequence
  Description of Artificial Sequence Synthetic oligonucleotide
uacauaugcaucuuagccu

SEQ ID NO: 825 - RNA, 19 nt, Artificial Sequence
  Description of Artificial Sequence Synthetic oligonucleotide
cuaucaguaacaauguuca

SEQ ID NO: 826 - RNA, 19 nt, Artificial Sequence
  Description of Artificial Sequence Synthetic oligonucleotide
gggauuaagaacuugacuc

SEQ ID NO: 827 - RNA, 19 nt, Artificial Sequence
  Description of Artificial Sequence Synthetic oligonucleotide
uuuaacuccucaccuaacu

SEQ ID NO: 828 - RNA, 19 nt, Artificial Sequence
  Description of Artificial Sequence Synthetic oligonucleotide
uuuguauuggaagggcucg

SEQ ID NO: 829 - RNA, 19 nt, Artificial Sequence
  Description of Artificial Sequence Synthetic oligonucleotide
agauuaacugggcacgagg

SEQ ID NO: 830 - RNA, 19 nt, Artificial Sequence
  Description of Artificial Sequence Synthetic oligonucleotide
cagaguauacuccuguucc

SEQ ID NO: 831 - RNA, 19 nt, Artificial Sequence
  Description of Artificial Sequence Synthetic oligonucleotide
auugagaaggcaugugcgg

SEQ ID NO: 832 - RNA, 19 nt, Artificial Sequence
  Description of Artificial Sequence Synthetic oligonucleotide
uggaauagaaaguugguuu

SEQ ID NO: 833 - RNA, 19 nt, Artificial Sequence
  Description of Artificial Sequence Synthetic oligonucleotide
cucgaugcaagaugaaacg

SEQ ID NO: 834 - RNA, 19 nt, Artificial Sequence
  Description of Artificial Sequence Synthetic oligonucleotide
aaagguacuagagccauca

SEQ ID NO: 835 - RNA, 19 nt, Artificial Sequence
  Description of Artificial Sequence Synthetic oligonucleotide
ucgaaugugguaccggugc

SEQ ID NO: 836 - RNA, 19 nt, Artificial Sequence
  Description of Artificial Sequence Synthetic oligonucleotide
uuauccguuuacacgagug

SEQ ID NO: 837 - RNA, 19 nt, Artificial Sequence
  Description of Artificial Sequence Synthetic oligonucleotide
ugcaucuuagccuaagcac

SEQ ID NO: 838 - RNA, 19 nt, Artificial Sequence
  Description of Artificial Sequence Synthetic oligonucleotide
auaggggaauugucaaucc

SEQ ID NO: 839 - RNA, 19 nt, Artificial Sequence
  Description of Artificial Sequence Synthetic oligonucleotide
uguaaucuugaccccagca

SEQ ID NO: 840 - RNA, 19 nt, Artificial Sequence
  Description of Artificial Sequence Synthetic oligonucleotide
accaucaacccagauauac

SEQ ID NO: 841 - RNA, 19 nt, Artificial Sequence
  Description of Artificial Sequence Synthetic oligonucleotide
auacaugucacuguuggag

SEQ ID NO: 842 - RNA, 19 nt, Artificial Sequence
  Description of Artificial Sequence Synthetic oligonucleotide
aaugugguaccggugccgc

SEQ ID NO: 843 - RNA, 19 nt, Artificial Sequence
  Description of Artificial Sequence Synthetic oligonucleotide
gaaaguugguuuuaccuga

SEQ ID NO: 844 - RNA, 19 nt, Artificial Sequence
  Description of Artificial Sequence Synthetic oligonucleotide
uuuggaauagaaaguuggu

SEQ ID NO: 845 - RNA, 19 nt, Artificial Sequence
  Description of Artificial Sequence Synthetic oligonucleotide
agaaaugaggguugggugu

SEQ ID NO: 846 - RNA, 19 nt, Artificial Sequence
  Description of Artificial Sequence Synthetic oligonucleotide
guguauagggauuaagaac

SEQ ID NO: 847 - RNA, 19 nt, Artificial Sequence
  Description of Artificial Sequence Synthetic oligonucleotide
ucugaccaauaaaagcaag

SEQ ID NO: 848 - RNA, 19 nt, Artificial Sequence
  Description of Artificial Sequence Synthetic oligonucleotide
ugauguuggacguuucgug

SEQ ID NO: 849 - RNA, 19 nt, Artificial Sequence
  Description of Artificial Sequence Synthetic oligonucleotide
uauuuggaauucccacugg

SEQ ID NO: 850 - RNA, 19 nt, Artificial Sequence
  Description of Artificial Sequence Synthetic oligonucleotide
acucgaugcaagaugaaac

SEQ ID NO: 851 - RNA, 19 nt, Artificial Sequence
  Description of Artificial Sequence Synthetic oligonucleotide
accccuaucaguaacaaug

SEQ ID NO: 852 - RNA, 19 nt, Artificial Sequence
  Description of Artificial Sequence Synthetic oligonucleotide
acauacucgaugcaagaug

SEQ ID NO: 853 - RNA, 19 nt, Artificial Sequence
  Description of Artificial Sequence Synthetic oligonucleotide
cuugauauuccauccuuug

SEQ ID NO: 854 - RNA, 19 nt, Artificial Sequence
  Description of Artificial Sequence Synthetic oligonucleotide
uuacaaugagacagcuggg

SEQ ID NO: 855 - RNA, 19 nt, Artificial Sequence
  Description of Artificial Sequence Synthetic oligonucleotide
aguaacuaggaugguuucc

SEQ ID NO: 856 - RNA, 19 nt, Artificial Sequence
  Description of Artificial Sequence Synthetic oligonucleotide
agauaaccaccuuuccugg

SEQ ID NO: 857 - RNA, 19 nt, Artificial Sequence
  Description of Artificial Sequence Synthetic oligonucleotide
uauccguuuacacgagugc

SEQ ID NO: 858 - RNA, 19 nt, Artificial Sequence
  Description of Artificial Sequence Synthetic oligonucleotide
uucaagugggaacuugcug

SEQ ID NO: 859 - RNA, 19 nt, Artificial Sequence
  Description of Artificial Sequence Synthetic oligonucleotide
aacuaggaugguuuccuca

SEQ ID NO: 860 - RNA, 19 nt, Artificial Sequence
  Description of Artificial Sequence Synthetic oligonucleotide
gaugcaagaugaaacgggc

SEQ ID NO: 861 - RNA, 19 nt, Artificial Sequence
  Description of Artificial Sequence Synthetic oligonucleotide
uauuggaagggcucgucgc

SEQ ID NO: 862 - RNA, 19 nt, Artificial Sequence
  Description of Artificial Sequence Synthetic oligonucleotide
gaagcagugguuacguucc

SEQ ID NO: 863 - RNA, 19 nt, Artificial Sequence
  Description of Artificial Sequence Synthetic oligonucleotide
aaaccccauaaaccccacc

SEQ ID NO: 864 - RNA, 19 nt, Artificial Sequence
  Description of Artificial Sequence Synthetic oligonucleotide
guugauguuggacguuucg

SEQ ID NO: 865 - RNA, 19 nt, Artificial Sequence
  Description of Artificial Sequence Synthetic oligonucleotide
ggaauugucaauccaagca

SEQ ID NO: 866 - RNA, 19 nt, Artificial Sequence
  Description of Artificial Sequence Synthetic oligonucleotide
uuugaguuacaaugagaca

SEQ ID NO: 867 - RNA, 19 nt, Artificial Sequence
  Description of Artificial Sequence Synthetic oligonucleotide
uggacaugcauuccugaug

SEQ ID NO: 868 - RNA, 19 nt, Artificial Sequence
  Description of Artificial Sequence Synthetic oligonucleotide
uauaggggaauugucaauc

SEQ ID NO: 869 - RNA, 19 nt, Artificial Sequence
  Description of Artificial Sequence Synthetic oligonucleotide
aaaauuccacucagguaac

SEQ ID NO: 870 - RNA, 19 nt, Artificial Sequence
  Description of Artificial Sequence Synthetic oligonucleotide
guaauaaagcgcugagccc

SEQ ID NO: 871 - RNA, 19 nt, Artificial Sequence
  Description of Artificial Sequence Synthetic oligonucleotide
ucgaugcaagaugaaacgg

SEQ ID NO: 872 - RNA, 19 nt, Artificial Sequence
  Description of Artificial Sequence Synthetic oligonucleotide
cuugcuuaguuucucgaug

SEQ ID NO: 873 - RNA, 19 nt, Artificial Sequence
  Description of Artificial Sequence Synthetic oligonucleotide
auaaaccccacccaccccu

SEQ ID NO: 874 - RNA, 19 nt, Artificial Sequence
  Description of Artificial Sequence Synthetic oligonucleotide
aguaccaucaacccagaua

SEQ ID NO: 875 - RNA, 19 nt, Artificial Sequence
  Description of Artificial Sequence Synthetic oligonucleotide
ugaaacgggccacccagag

SEQ ID NO: 876 - RNA, 19 nt, Artificial Sequence
  Description of Artificial Sequence Synthetic oligonucleotide
cauacucgaugcaagauga

SEQ ID NO: 877 - RNA, 19 nt, Artificial Sequence
  Description of Artificial Sequence Synthetic oligonucleotide
uacuagagccaucaaaauu

SEQ ID NO: 878 - RNA, 19 nt, Artificial Sequence
  Description of Artificial Sequence Synthetic oligonucleotide
uggaugaacuaggaaaggc

SEQ ID NO: 879 - RNA, 19 nt, Artificial Sequence
  Description of Artificial Sequence Synthetic oligonucleotide
acuaggaaaggcucaagau

SEQ ID NO: 880 - RNA, 19 nt, Artificial Sequence
  Description of Artificial Sequence Synthetic oligonucleotide
cccacauaguaauaaagcg

SEQ ID NO: 881 - RNA, 19 nt, Artificial Sequence
  Description of Artificial Sequence Synthetic oligonucleotide
ggauuaagaacuugacucc

SEQ ID NO: 882 - RNA, 19 nt, Artificial Sequence
  Description of Artificial Sequence Synthetic oligonucleotide
guauuggaagggcucgucg

SEQ ID NO: 883 - RNA, 19 nt, Artificial Sequence
  Description of Artificial Sequence Synthetic oligonucleotide
caugcggauuccuucacag

SEQ ID NO: 884 - RNA, 19 nt, Artificial Sequence
  Description of Artificial Sequence Synthetic oligonucleotide
auuaccccaaagucuucac

SEQ ID NO: 885 - RNA, 19 nt, Artificial Sequence
  Description of Artificial Sequence Synthetic oligonucleotide
uagggauuaagaacuugac

SEQ ID NO: 886 - RNA, 19 nt, Artificial Sequence
  Description of Artificial Sequence Synthetic oligonucleotide
uauaucaaaaggccugcuu

SEQ ID NO: 887 - RNA, 19 nt, Artificial Sequence
  Description of Artificial Sequence Synthetic oligonucleotide
gaugaacuaggaaaggcuc

SEQ ID NO: 888 - RNA, 19 nt, Artificial Sequence
  Description of Artificial Sequence Synthetic oligonucleotide
uggcaaacccaguaacuag

SEQ ID NO: 889 - RNA, 19 nt, Artificial Sequence
  Description of Artificial Sequence Synthetic oligonucleotide
uuccgguuguacuugaaaa

SEQ ID NO: 890 - RNA, 19 nt, Artificial Sequence
  Description of Artificial Sequence Synthetic oligonucleotide
ugaccucagcauuuguucc

SEQ ID NO: 891 - RNA, 19 nt, Artificial Sequence
  Description of Artificial Sequence Synthetic oligonucleotide
cacgagugccuuaaauugg

SEQ ID NO: 892 - RNA, 19 nt, Artificial Sequence
  Description of Artificial Sequence Synthetic oligonucleotide
ugaggguuggguguauagg

SEQ ID NO: 893 - RNA, 19 nt, Artificial Sequence
  Description of Artificial Sequence Synthetic oligonucleotide
uccacgauaucccugccau

SEQ ID NO: 894 - RNA, 19 nt, Artificial Sequence
  Description of Artificial Sequence Synthetic oligonucleotide
cucgucgccagucucauug

SEQ ID NO: 895 - RNA, 19 nt, Artificial Sequence
  Description of Artificial Sequence Synthetic oligonucleotide
uaauacggaccuuggggcc

SEQ ID NO: 896 - RNA, 19 nt, Artificial Sequence
  Description of Artificial Sequence Synthetic oligonucleotide
gaugguuuccucaauuaga

SEQ ID NO: 897 - RNA, 19 nt, Artificial Sequence
  Description of Artificial Sequence Synthetic oligonucleotide
augcaucuuagccuaagca

SEQ ID NO: 898 - RNA, 19 nt, Artificial Sequence
  Description of Artificial Sequence Synthetic oligonucleotide
uccgguuguacuugaaaac

SEQ ID NO: 899 - RNA, 19 nt, Artificial Sequence
  Description of Artificial Sequence Synthetic oligonucleotide
auagggauuaagaacuuga

SEQ ID NO: 900 - RNA, 19 nt, Artificial Sequence
  Description of Artificial Sequence Synthetic oligonucleotide
cacauaguaauaaagcgcu

SEQ ID NO: 901 - RNA, 19 nt, Artificial Sequence
  Description of Artificial Sequence Synthetic oligonucleotide
aaguugguuuuaccugaag

SEQ ID NO: 902 - RNA, 19 nt, Artificial Sequence
  Description of Artificial Sequence Synthetic oligonucleotide
cacgauaucccugccauag

SEQ ID NO: 903 - RNA, 19 nt, Artificial Sequence
  Description of Artificial Sequence Synthetic oligonucleotide
ugaucucccaugcggauuc

SEQ ID NO: 904 - RNA, 19 nt, Artificial Sequence
  Description of Artificial Sequence Synthetic oligonucleotide
ugcagcgcaguccuucucc

SEQ ID NO: 905 - RNA, 19 nt, Artificial Sequence
  Description of Artificial Sequence Synthetic oligonucleotide
acaauuggcagaggggcgg

SEQ ID NO: 906 - RNA, 19 nt, Artificial Sequence
  Description of Artificial Sequence Synthetic oligonucleotide
ucuaccuauaggggaauug

SEQ ID NO: 907 - RNA, 19 nt, Artificial Sequence
  Description of Artificial Sequence Synthetic oligonucleotide
ggagcccucagacugaaag

SEQ ID NO: 908 - RNA, 19 nt, Artificial Sequence
  Description of Artificial Sequence Synthetic oligonucleotide
uuggacguuucguggaacc

SEQ ID NO: 909 - RNA, 19 nt, Artificial Sequence
  Description of Artificial Sequence Synthetic oligonucleotide
aagcugaacuuguuuugcu

SEQ ID NO: 910 - RNA, 19 nt, Artificial Sequence
  Description of Artificial Sequence Synthetic oligonucleotide
uuacacgagugccuuaaau

SEQ ID NO: 911 - RNA, 19 nt, Artificial Sequence
  Description of Artificial Sequence Synthetic oligonucleotide
acugcgauuggcgacacca

SEQ ID NO: 912 - RNA, 19 nt, Artificial Sequence
  Description of Artificial Sequence Synthetic oligonucleotide
cuuuccugguacugcaccc

SEQ ID NO: 913 - RNA, 19 nt, Artificial Sequence
  Description of Artificial Sequence Synthetic oligonucleotide
gcuaaaguugguauggcag

SEQ ID NO: 914 - RNA, 19 nt, Artificial Sequence
  Description of Artificial Sequence Synthetic oligonucleotide
uuaacuccucaccuaacuu

SEQ ID NO: 915 - RNA, 19 nt, Artificial Sequence
  Description of Artificial Sequence Synthetic oligonucleotide
ccuaucaguaacaauguuc

SEQ ID NO: 916 - RNA, 19 nt, Artificial Sequence
  Description of Artificial Sequence Synthetic oligonucleotide
guauacuccuguuccauuc

SEQ ID NO: 917 - RNA, 19 nt, Artificial Sequence
  Description of Artificial Sequence Synthetic oligonucleotide
uuaacugggcacgaggaau

SEQ ID NO: 918 - RNA, 19 nt, Artificial Sequence
  Description of Artificial Sequence Synthetic oligonucleotide
gauuaacugggcacgagga

SEQ ID NO: 919 - RNA, 19 nt, Artificial Sequence
  Description of Artificial Sequence Synthetic oligonucleotide
uuaccccaaagucuucaca

SEQ ID NO: 920 - RNA, 19 nt, Artificial Sequence
  Description of Artificial Sequence Synthetic oligonucleotide
uacuggacaugcauuccug

SEQ ID NO: 921 - RNA, 19 nt, Artificial Sequence
  Description of Artificial Sequence Synthetic oligonucleotide
aagcagugguuacguuccc

SEQ ID NO: 922 - RNA, 19 nt, Artificial Sequence
  Description of Artificial Sequence Synthetic oligonucleotide
uauuaccccaaagucuuca

SEQ ID NO: 923 - RNA, 19 nt, Artificial Sequence
  Description of Artificial Sequence Synthetic oligonucleotide
ugcgauuggcgacaccagc

SEQ ID NO: 924 - RNA, 19 nt, Artificial Sequence
  Description of Artificial Sequence Synthetic oligonucleotide
uaggggaauugucaaucca

SEQ ID NO: 925 - RNA, 19 nt, Artificial Sequence
  Description of Artificial Sequence Synthetic oligonucleotide
uccguuuacacgagugccu

SEQ ID NO: 926 - RNA, 19 nt, Artificial Sequence
  Description of Artificial Sequence Synthetic oligonucleotide
guaaaguuagaaacccuac

SEQ ID NO: 927 - RNA, 19 nt, Artificial Sequence
  Description of Artificial Sequence Synthetic oligonucleotide
cacaauuggcagaggggcg

SEQ ID NO: 928 - RNA, 19 nt, Artificial Sequence
  Description of Artificial Sequence Synthetic oligonucleotide
aagauuaacugggcacgag

SEQ ID NO: 929 - RNA, 19 nt, Artificial Sequence
  Description of Artificial Sequence Synthetic oligonucleotide
uccuccguuccuccaagag

SEQ ID NO: 930 - RNA, 19 nt, Artificial Sequence
  Description of Artificial Sequence Synthetic oligonucleotide
agggugcggguuuugcagc

SEQ ID NO: 931 - RNA, 19 nt, Artificial Sequence
  Description of Artificial Sequence Synthetic oligonucleotide
cuacucaagagauccuuuc

SEQ ID NO: 932 - RNA, 19 nt, Artificial Sequence
  Description of Artificial Sequence Synthetic oligonucleotide
gcaagauuaacugggcacg

SEQ ID NO: 933 - RNA, 19 nt, Artificial Sequence
  Description of Artificial Sequence Synthetic oligonucleotide
agacugaaagguacuagag

SEQ ID NO: 934 - RNA, 19 nt, Artificial Sequence
  Description of Artificial Sequence Synthetic oligonucleotide
cugaccaauaaaagcaaga

SEQ ID NO: 935 - RNA, 19 nt, Artificial Sequence
  Description of Artificial Sequence Synthetic oligonucleotide
uagccuaagcacagggaca

SEQ ID NO: 936 - RNA, 19 nt, Artificial Sequence
  Description of Artificial Sequence Synthetic oligonucleotide
ggcaacagggcugagauac

SEQ ID NO: 937 - RNA, 19 nt, Artificial Sequence
  Description of Artificial Sequence Synthetic oligonucleotide
uguccauuauccguuuaca

SEQ ID NO: 938 - RNA, 19 nt, Artificial Sequence
  Description of Artificial Sequence Synthetic oligonucleotide
cuuaguuucucgauggccu

SEQ ID NO: 939 - RNA, 19 nt, Artificial Sequence
  Description of Artificial Sequence Synthetic oligonucleotide
acaggguccuauuuggaau

SEQ ID NO: 940 - RNA, 19 nt, Artificial Sequence
  Description of Artificial Sequence Synthetic oligonucleotide
uugauguuggacguuucgu

SEQ ID NO: 941 - RNA, 19 nt, Artificial Sequence
  Description of Artificial Sequence Synthetic oligonucleotide
augcaagaugaaacgggcc

SEQ ID NO: 942 - RNA, 19 nt, Artificial Sequence
  Description of Artificial Sequence Synthetic oligonucleotide
auaaaccccauaaacccca

SEQ ID NO: 943 - RNA, 19 nt, Artificial Sequence
  Description of Artificial Sequence Synthetic oligonucleotide
uuggaauagaaaguugguu

SEQ ID NO: 944 - RNA, 19 nt, Artificial Sequence
  Description of Artificial Sequence Synthetic oligonucleotide
gguguauagggauuaagaa

SEQ ID NO: 945 - RNA, 19 nt, Artificial Sequence
  Description of Artificial Sequence Synthetic oligonucleotide
ggaugguuuccucaauuag

SEQ ID NO: 946 - RNA, 19 nt, Artificial Sequence
  Description of Artificial Sequence Synthetic oligonucleotide
cagacugaaagguacuaga

SEQ ID NO: 947 - RNA, 19 nt, Artificial Sequence
  Description of Artificial Sequence Synthetic oligonucleotide
agaguauacuccuguucca

SEQ ID NO: 948 - RNA, 19 nt, Artificial Sequence
  Description of Artificial Sequence Synthetic oligonucleotide
uaaccaccuuuccugguac

SEQ ID NO: 949 - RNA, 19 nt, Artificial Sequence
  Description of Artificial Sequence Synthetic oligonucleotide
accagcagaaaagucguug

SEQ ID NO: 950 - RNA, 19 nt, Artificial Sequence
  Description of Artificial Sequence Synthetic oligonucleotide
agccuaagcacagggacag

SEQ ID NO: 951 - RNA, 19 nt, Artificial Sequence
  Description of Artificial Sequence Synthetic oligonucleotide
ucaaguugaccagccaacu

SEQ ID NO: 952 - RNA, 19 nt, Artificial Sequence
  Description of Artificial Sequence Synthetic oligonucleotide
gguacuagagccaucaaaa

SEQ ID NO: 953 - RNA, 19 nt, Artificial Sequence
  Description of Artificial Sequence Synthetic oligonucleotide
guccauuauccguuuacac

SEQ ID NO: 954 - RNA, 19 nt, Artificial Sequence
  Description of Artificial Sequence Synthetic oligonucleotide
ugaacuaggaaaggcucaa

SEQ ID NO: 955 - RNA, 19 nt, Artificial Sequence
  Description of Artificial Sequence Synthetic oligonucleotide
uaccuauaggggaauuguc

SEQ ID NO: 956 - RNA, 19 nt, Artificial Sequence
  Description of Artificial Sequence Synthetic oligonucleotide
acccagucagacgacgggc

SEQ ID NO: 957 - RNA, 19 nt, Artificial Sequence
  Description of Artificial Sequence Synthetic oligonucleotide
cagggugcggguuuugcag

SEQ ID NO: 958 - RNA, 19 nt, Artificial Sequence
  Description of Artificial Sequence Synthetic oligonucleotide
uccucuauucugaccaaua

SEQ ID NO: 959 - RNA, 19 nt, Artificial Sequence
  Description of Artificial Sequence Synthetic oligonucleotide
cacaaguacauuugcuuga

SEQ ID NO: 960 - RNA, 19 nt, Artificial Sequence
  Description of Artificial Sequence Synthetic oligonucleotide
gaucguaggcucgaaugug

SEQ ID NO: 961 - RNA, 19 nt, Artificial Sequence
  Description of Artificial Sequence Synthetic oligonucleotide
uaacugggcacgaggaaua

SEQ ID NO: 962 - RNA, 19 nt, Artificial Sequence
  Description of Artificial Sequence Synthetic oligonucleotide
auuaacugggcacgaggaa

SEQ ID NO: 963 - RNA, 19 nt, Artificial Sequence
  Description of Artificial Sequence Synthetic oligonucleotide
aucguaggcucgaaugugg

SEQ ID NO: 964 - RNA, 19 nt, Artificial Sequence
  Description of Artificial Sequence Synthetic oligonucleotide
gggaucguaggcucgaaug

SEQ ID NO: 965 - RNA, 19 nt, Artificial Sequence
  Description of Artificial Sequence Synthetic oligonucleotide
acaggcaauucuguaaagu

SEQ ID NO: 966 - RNA, 19 nt, Artificial Sequence
  Description of Artificial Sequence Synthetic oligonucleotide
cauuggaaggccaaccaaa

SEQ ID NO: 967 - RNA, 19 nt, Artificial Sequence
  Description of Artificial Sequence Synthetic oligonucleotide
guuucguggaacccaguca

SEQ ID NO: 968 - RNA, 19 nt, Artificial Sequence
  Description of Artificial Sequence Synthetic oligonucleotide
cauuauccguuuacacgag

SEQ ID NO: 969 - RNA, 19 nt, Artificial Sequence
  Description of Artificial Sequence Synthetic oligonucleotide
auuauccguuuacacgagu

SEQ ID NO: 970 - RNA, 19 nt, Artificial Sequence
  Description of Artificial Sequence Synthetic oligonucleotide
auaucccugccauaggcuu

SEQ ID NO: 971 - RNA, 19 nt, Artificial Sequence
  Description of Artificial Sequence Synthetic oligonucleotide
ggcaaacccaguaacuagg

SEQ ID NO: 972 - RNA, 19 nt, Artificial Sequence
  Description of Artificial Sequence Synthetic oligonucleotide
auacaagcaggcgcgguag

SEQ ID NO: 973 - RNA, 19 nt, Artificial Sequence
  Description of Artificial Sequence Synthetic oligonucleotide
gacaguccggggaaugcgg

SEQ ID NO: 974 - RNA, 19 nt, Artificial Sequence
  Description of Artificial Sequence Synthetic oligonucleotide
acaugcauuccugaugaga

SEQ ID NO: 975 - RNA, 19 nt, Artificial Sequence
  Description of Artificial Sequence Synthetic oligonucleotide
uguccuccguuccuccaag

SEQ ID NO: 976 - RNA, 19 nt, Artificial Sequence
  Description of Artificial Sequence Synthetic oligonucleotide
uggguguauagggauuaag

SEQ ID NO: 977 - RNA, 19 nt, Artificial Sequence
  Description of Artificial Sequence Synthetic oligonucleotide
accugaagaacuagcagcu

SEQ ID NO: 978 - RNA, 19 nt, Artificial Sequence
  Description of Artificial Sequence Synthetic oligonucleotide
gacguuucguggaacccag

SEQ ID NO: 979 - RNA, 19 nt, Artificial Sequence
  Description of Artificial Sequence Synthetic oligonucleotide
agucagacgacgggcauug

SEQ ID NO: 980 - RNA, 19 nt, Artificial Sequence
  Description of Artificial Sequence Synthetic oligonucleotide
acccaccccuaucaguaac

SEQ ID NO: 981 - RNA, 19 nt, Artificial Sequence
  Description of Artificial Sequence Synthetic oligonucleotide
uguuggacguuucguggaa

SEQ ID NO: 982 - RNA, 19 nt, Artificial Sequence
  Description of Artificial Sequence Synthetic oligonucleotide
acgauaucccugccauagg

SEQ ID NO: 983 - RNA, 19 nt, Artificial Sequence
  Description of Artificial Sequence Synthetic oligonucleotide
cugggcacgaggaauaaaa

SEQ ID NO: 984 - RNA, 19 nt, Artificial Sequence
  Description of Artificial Sequence Synthetic oligonucleotide
gcuaacucuguguggauac

SEQ ID NO: 985 - RNA, 19 nt, Artificial Sequence
  Description of Artificial Sequence Synthetic oligonucleotide
aaagaccuugauauuccau

SEQ ID NO: 986 - RNA, 19 nt, Artificial Sequence
  Description of Artificial Sequence Synthetic oligonucleotide
aguugguauggcagccugc

SEQ ID NO: 987 - RNA, 19 nt, Artificial Sequence
  Description of Artificial Sequence Synthetic oligonucleotide
cugagccccagauaaccac

SEQ ID NO: 988 - RNA, 19 nt, Artificial Sequence
  Description of Artificial Sequence Synthetic oligonucleotide
ccgaaacauggcaacaggg

SEQ ID NO: 989 - RNA, 19 nt, Artificial Sequence
  Description of Artificial Sequence Synthetic oligonucleotide
ugcaccauuccaguuccca

SEQ ID NO: 990 - RNA, 19 nt, Artificial Sequence
  Description of Artificial Sequence Synthetic oligonucleotide
aggaugguuuccucaauua

SEQ ID NO: 991 - RNA, 19 nt, Artificial Sequence
  Description of Artificial Sequence Synthetic oligonucleotide
uaggcucgaaugugguacc

SEQ ID NO: 992 - RNA, 19 nt, Artificial Sequence
  Description of Artificial Sequence Synthetic oligonucleotide
gaaggggucccgaaacaug

SEQ ID NO: 993 - RNA, 19 nt, Artificial Sequence
  Description of Artificial Sequence Synthetic oligonucleotide
gugguuacguucccuuccc

SEQ ID NO: 994 - RNA, 19 nt, Artificial Sequence
  Description of Artificial Sequence Synthetic oligonucleotide
gagugccuuaaauuggucu

SEQ ID NO: 995 - RNA, 19 nt, Artificial Sequence
  Description of Artificial Sequence Synthetic oligonucleotide
caugcauuccugaugagau

SEQ ID NO: 996 - RNA, 19 nt, Artificial Sequence
  Description of Artificial Sequence Synthetic oligonucleotide
gugagccuccacgauaucc

SEQ ID NO: 997 - RNA, 19 nt, Artificial Sequence
  Description of Artificial Sequence Synthetic oligonucleotide
acguucccuuccccuaccc

SEQ ID NO: 998 - RNA, 19 nt, Artificial Sequence
  Description of Artificial Sequence Synthetic oligonucleotide
aauaaagcgcugagcccca

SEQ ID NO: 999 - RNA, 19 nt, Artificial Sequence
  Description of Artificial Sequence Synthetic oligonucleotide
cuuagccuaagcacaggga

SEQ ID NO: 1000 - RNA, 19 nt, Artificial Sequence
  Description of Artificial Sequence Synthetic oligonucleotide
ucugccuacuggacaugca

SEQ ID NO: 1001 - RNA, 19 nt, Artificial Sequence
  Description of Artificial Sequence Synthetic oligonucleotide
aauguugaagggcacaccc

SEQ ID NO: 1002 - RNA, 19 nt, Artificial Sequence
  Description of Artificial Sequence Synthetic oligonucleotide
auaaccuucaaucugaaag

SEQ ID NO: 1003 - RNA, 19 nt, Artificial Sequence
  Description of Artificial Sequence Synthetic oligonucleotide
auaaacugggcccaggucc

SEQ ID NO: 1004 - RNA, 19 nt, Artificial Sequence
  Description of Artificial Sequence Synthetic oligonucleotide
aguucuuuagcauuugugg

SEQ ID NO: 1005 - RNA, 19 nt, Artificial Sequence
  Description of Artificial Sequence Synthetic oligonucleotide
uggauuauaaccuucaauc

SEQ ID NO: 1006 - RNA, 19 nt, Artificial Sequence
  Description of Artificial Sequence Synthetic oligonucleotide
ugugcuaucauguagguaa

SEQ ID NO: 1007 - RNA, 19 nt, Artificial Sequence
  Description of Artificial Sequence Synthetic oligonucleotide
uuggauguggauuauaacc

SEQ ID NO: 1008 - RNA, 19 nt, Artificial Sequence
  Description of Artificial Sequence Synthetic oligonucleotide
uuucgaaggauuuugagcu

SEQ ID NO: 1009 - RNA, 19 nt, Artificial Sequence
  Description of Artificial Sequence Synthetic oligonucleotide
uaugauucagauaaauaug

SEQ ID NO: 1010 - RNA, 19 nt, Artificial Sequence
  Description of Artificial Sequence Synthetic oligonucleotide
gaaaccauaaacugggccc

SEQ ID NO: 1011 - RNA, 19 nt, Artificial Sequence
  Description of Artificial Sequence Synthetic oligonucleotide
aaucucgggacccauuggc

SEQ ID NO: 1012 - RNA, 19 nt, Artificial Sequence
  Description of Artificial Sequence Synthetic oligonucleotide
aaaguauggacaaaaucac

SEQ ID NO: 1013 - RNA, 19 nt, Artificial Sequence
  Description of Artificial Sequence Synthetic oligonucleotide
acauagaauugacagaggg

SEQ ID NO: 1014 - RNA, 19 nt, Artificial Sequence
  Description of Artificial Sequence Synthetic oligonucleotide
aaaacucccuuccagaaca

SEQ ID NO: 1015 - RNA, 19 nt, Artificial Sequence
  Description of Artificial Sequence Synthetic oligonucleotide
aaaccauagcaacuccucc

SEQ ID NO: 1016 - RNA, 19 nt, Artificial Sequence
  Description of Artificial Sequence Synthetic oligonucleotide
aaaaccgcagcgcauaaug

SEQ ID NO: 1017 - RNA, 19 nt, Artificial Sequence
  Description of Artificial Sequence Synthetic oligonucleotide
ugaaagagcgcuaaacagc

SEQ ID NO: 1018 - RNA, 19 nt, Artificial Sequence
  Description of Artificial Sequence Synthetic oligonucleotide
uuuucgaaggauuuugagc

SEQ ID NO: 1019 - RNA, 19 nt, Artificial Sequence
  Description of Artificial Sequence Synthetic oligonucleotide
cucgauaugauucagauaa

SEQ ID NO: 1020 - RNA, 19 nt, Artificial Sequence
  Description of Artificial Sequence Synthetic oligonucleotide
aauugucucaacuuuucga

SEQ ID NO: 1021 - RNA, 19 nt, Artificial Sequence
  Description of Artificial Sequence Synthetic oligonucleotide
aaaaucucgggacccauug

SEQ ID NO: 1022 - RNA, 19 nt, Artificial Sequence
  Description of Artificial Sequence Synthetic oligonucleotide
uuugguugugagcagagga

SEQ ID NO: 1023 - RNA, 19 nt, Artificial Sequence
  Description of Artificial Sequence Synthetic oligonucleotide
gaucuuuuggguuccaggc

SEQ ID NO: 1024 - RNA, 19 nt, Artificial Sequence
  Description of Artificial Sequence Synthetic oligonucleotide
uucuuuagcauuuguggag

SEQ ID NO: 1025 - RNA, 19 nt, Artificial Sequence
  Description of Artificial Sequence Synthetic oligonucleotide
ucuugguucucgcugaagc

SEQ ID NO: 1026 - RNA, 19 nt, Artificial Sequence
  Description of Artificial Sequence Synthetic oligonucleotide
aggauuuugagcuuuggga

SEQ ID NO: 1027 - RNA, 19 nt, Artificial Sequence
  Description of Artificial Sequence Synthetic oligonucleotide
acccgaguaaucugaaucc

SEQ ID NO: 1028 - RNA, 19 nt, Artificial Sequence
  Description of Artificial Sequence Synthetic oligonucleotide
cauaaugugcuccaccugc

SEQ ID NO: 1029 - RNA, 19 nt, Artificial Sequence
  Description of Artificial Sequence Synthetic oligonucleotide
uuuuaaucguuggaugugg

SEQ ID NO: 1030 - RNA, 19 nt, Artificial Sequence
  Description of Artificial Sequence Synthetic oligonucleotide
aaguauggacaaaaucacc

SEQ ID NO: 1031 - RNA, 19 nt, Artificial Sequence
  Description of Artificial Sequence Synthetic oligonucleotide
auucucacucccuuggagg

SEQ ID NO: 1032 - RNA, 19 nt, Artificial Sequence
  Description of Artificial Sequence Synthetic oligonucleotide
ucaggggaagaucuuuugg

SEQ ID NO: 1033 - RNA, 19 nt, Artificial Sequence
  Description of Artificial Sequence Synthetic oligonucleotide
uugauaaaccauagcaacu

SEQ ID NO: 1034 - RNA, 19 nt, Artificial Sequence
  Description of Artificial Sequence Synthetic oligonucleotide
gguaaaguuggcaagacag

SEQ ID NO: 1035 - RNA, 19 nt, Artificial Sequence
  Description of Artificial Sequence Synthetic oligonucleotide
auucagauaaauaugugca

SEQ ID NO: 1036 - RNA, 19 nt, Artificial Sequence
  Description of Artificial Sequence Synthetic oligonucleotide
agaucuuuuggguuccagg

SEQ ID NO: 1037 - RNA, 19 nt, Artificial Sequence
  Description of Artificial Sequence Synthetic oligonucleotide
gaaagagcgcuaaacagcc

SEQ ID NO: 1038 - RNA, 19 nt, Artificial Sequence
  Description of Artificial Sequence Synthetic oligonucleotide
uucuuaaaaccgcagcgca

SEQ ID NO: 1039 - RNA, 19 nt, Artificial Sequence
  Description of Artificial Sequence Synthetic oligonucleotide
cauagaauugacagagggc

SEQ ID NO: 1040 - RNA, 19 nt, Artificial Sequence
  Description of Artificial Sequence Synthetic oligonucleotide
uaaccuucaaucugaaagu

SEQ ID NO: 1041 - RNA, 19 nt, Artificial Sequence
  Description of Artificial Sequence Synthetic oligonucleotide
ucugaauccauaucuuugu

SEQ ID NO: 1042 - RNA, 19 nt, Artificial Sequence
  Description of Artificial Sequence Synthetic oligonucleotide
cuggucuacuccuugaccc

SEQ ID NO: 1043 - RNA, 19 nt, Artificial Sequence
  Description of Artificial Sequence Synthetic oligonucleotide
cuaucauguagguaagcag

SEQ ID NO: 1044 - RNA, 19 nt, Artificial Sequence
  Description of Artificial Sequence Synthetic oligonucleotide
aucaggauugguuuugaug

SEQ ID NO: 1045 - RNA, 19 nt, Artificial Sequence
  Description of Artificial Sequence Synthetic oligonucleotide
uuuuccacauagaauugac

SEQ ID NO: 1046 - RNA, 19 nt, Artificial Sequence
  Description of Artificial Sequence Synthetic oligonucleotide
auuauaaccuucaaucuga

SEQ ID NO: 1047 - RNA, 19 nt, Artificial Sequence
  Description of Artificial Sequence Synthetic oligonucleotide
aaucguuggauguggauua

SEQ ID NO: 1048 - RNA, 19 nt, Artificial Sequence
  Description of Artificial Sequence Synthetic oligonucleotide
ugauucagauaaauaugug

SEQ ID NO: 1049 - RNA, 19 nt, Artificial Sequence
  Description of Artificial Sequence Synthetic oligonucleotide
auaucuuuguagucugcuc

SEQ ID NO: 1050 - RNA, 19 nt, Artificial Sequence
  Description of Artificial Sequence Synthetic oligonucleotide
acuggaaaccauaaacugg

SEQ ID NO: 1051 - RNA, 19 nt, Artificial Sequence
  Description of Artificial Sequence Synthetic oligonucleotide
aucccauauucucacuccc

SEQ ID NO: 1052 - RNA, 19 nt, Artificial Sequence
  Description of Artificial Sequence Synthetic oligonucleotide
uccuucuuaaaaccgcagc

SEQ ID NO: 1053 - RNA, 19 nt, Artificial Sequence
  Description of Artificial Sequence Synthetic oligonucleotide
auaugugcaucucccaaag

SEQ ID NO: 1054 - RNA, 19 nt, Artificial Sequence
  Description of Artificial Sequence Synthetic oligonucleotide
aguauggacaaaaucaccu

SEQ ID NO: 1055 - RNA, 19 nt, Artificial Sequence
  Description of Artificial Sequence Synthetic oligonucleotide
uaaaccauagcaacuccuc

SEQ ID NO: 1056 - RNA, 19 nt, Artificial Sequence
  Description of Artificial Sequence Synthetic oligonucleotide
augccgaacaccgacaagg

SEQ ID NO: 1057 - RNA, 19 nt, Artificial Sequence
  Description of Artificial Sequence Synthetic oligonucleotide
uaaaguuggcaagacagcu

SEQ ID NO: 1058 - RNA, 19 nt, Artificial Sequence
  Description of Artificial Sequence Synthetic oligonucleotide
gaugauucuucugucauca

SEQ ID NO: 1059 - RNA, 19 nt, Artificial Sequence
  Description of Artificial Sequence Synthetic oligonucleotide
cagaacacucuuuugguug

SEQ ID NO: 1060 - RNA, 19 nt, Artificial Sequence
  Description of Artificial Sequence Synthetic oligonucleotide
ucugucaucaggauugguu

SEQ ID NO: 1061 - RNA, 19 nt, Artificial Sequence
  Description of Artificial Sequence Synthetic oligonucleotide
augauucagauaaauaugu

SEQ ID NO: 1062 - RNA, 19 nt, Artificial Sequence
  Description of Artificial Sequence Synthetic oligonucleotide
cagcaucuuugaaagagcg

SEQ ID NO: 1063 - RNA, 19 nt, Artificial Sequence
  Description of Artificial Sequence Synthetic oligonucleotide
uuuguagucugcuccaaaa

SEQ ID NO: 1064 - RNA, 19 nt, Artificial Sequence
  Description of Artificial Sequence Synthetic oligonucleotide
ucaauugucucaacuuuuc

SEQ ID NO: 1065 - RNA, 19 nt, Artificial Sequence
  Description of Artificial Sequence Synthetic oligonucleotide
aagagcgcuaaacagccau

SEQ ID NO: 1066 - RNA, 19 nt, Artificial Sequence
  Description of Artificial Sequence Synthetic oligonucleotide
aaccuucaaucugaaaguc

SEQ ID NO: 1067 - RNA, 19 nt, Artificial Sequence
  Description of Artificial Sequence Synthetic oligonucleotide
uccauuuuaaucguuggau

SEQ ID NO: 1068 - RNA, 19 nt, Artificial Sequence
  Description of Artificial Sequence Synthetic oligonucleotide
aaucaucaauugucucaac

SEQ ID NO: 1069 - RNA, 19 nt, Artificial Sequence
  Description of Artificial Sequence Synthetic oligonucleotide
caucucucaggcuguaucg

SEQ ID NO: 1070 - RNA, 19 nt, Artificial Sequence
  Description of Artificial Sequence Synthetic oligonucleotide
ucuuuagcauuuguggagc

SEQ ID NO: 1071 - RNA, 19 nt, Artificial Sequence
  Description of Artificial Sequence Synthetic oligonucleotide
aaaguuggcaagacagcuc

SEQ ID NO: 1072 - RNA, 19 nt, Artificial Sequence
  Description of Artificial Sequence Synthetic oligonucleotide
gaauugacagagggcaugg

SEQ ID NO: 1073 - RNA, 19 nt, Artificial Sequence
  Description of Artificial Sequence Synthetic oligonucleotide
ugugcaucucccaaaguau

SEQ ID NO: 1074 - RNA, 19 nt, Artificial Sequence
  Description of Artificial Sequence Synthetic oligonucleotide
auggacaaaaucaccuggc

SEQ ID NO: 1075 - RNA, 19 nt, Artificial Sequence
  Description of Artificial Sequence Synthetic oligonucleotide
uuuccacauagaauugaca

SEQ ID NO: 1076 - RNA, 19 nt, Artificial Sequence
  Description of Artificial Sequence Synthetic oligonucleotide
gacccgaguaaucugaauc

SEQ ID NO: 1077 - RNA, 19 nt, Artificial Sequence
  Description of Artificial Sequence Synthetic oligonucleotide
uccacauagaauugacaga

SEQ ID NO: 1078 - RNA, 19 nt, Artificial Sequence
  Description of Artificial Sequence Synthetic oligonucleotide
ugaauuuucaguggcucga

SEQ ID NO: 1079 - RNA, 19 nt, Artificial Sequence
  Description of Artificial Sequence Synthetic oligonucleotide
aacaccgacaaggugccag

SEQ ID NO: 1080 - RNA, 19 nt, Artificial Sequence
  Description of Artificial Sequence Synthetic oligonucleotide
acuggcauagcggagaagu

SEQ ID NO: 1081 - RNA, 19 nt, Artificial Sequence
  Description of Artificial Sequence Synthetic oligonucleotide
uaaacugggcccagguccc

SEQ ID NO: 1082 - RNA, 19 nt, Artificial Sequence
  Description of Artificial Sequence Synthetic oligonucleotide
uuugggaaaggucuugguu

SEQ ID NO: 1083 - RNA, 19 nt, Artificial Sequence
  Description of Artificial Sequence Synthetic oligonucleotide
cuucuuaaaaccgcagcgc

SEQ ID NO: 1084 - RNA, 19 nt, Artificial Sequence
  Description of Artificial Sequence Synthetic oligonucleotide
uuuaaucguuggaugugga

SEQ ID NO: 1085 - RNA, 19 nt, Artificial Sequence
  Description of Artificial Sequence Synthetic oligonucleotide
ucucccaaaguauggacaa

SEQ ID NO: 1086 - RNA, 19 nt, Artificial Sequence
  Description of Artificial Sequence Synthetic oligonucleotide
ucaucucucaggcuguauc

SEQ ID NO: 1087 - RNA, 19 nt, Artificial Sequence
  Description of Artificial Sequence Synthetic oligonucleotide
aaaccauaaacugggccca

SEQ ID NO: 1088 - RNA, 19 nt, Artificial Sequence
  Description of Artificial Sequence Synthetic oligonucleotide
caauugucucaacuuuucg

SEQ ID NO: 1089 - RNA, 19 nt, Artificial Sequence
  Description of Artificial Sequence Synthetic oligonucleotide
uuagcauuuguggagcccu

SEQ ID NO: 1090 - RNA, 19 nt, Artificial Sequence
  Description of Artificial Sequence Synthetic oligonucleotide
auuuugagcuuugggaaag

SEQ ID NO: 1091 - RNA, 19 nt, Artificial Sequence
  Description of Artificial Sequence Synthetic oligonucleotide
guauggacaaaaucaccug

SEQ ID NO: 1092 - RNA, 19 nt, Artificial Sequence
  Description of Artificial Sequence Synthetic oligonucleotide
accgacaaggugccagugc

SEQ ID NO: 1093 - RNA, 19 nt, Artificial Sequence
  Description of Artificial Sequence Synthetic oligonucleotide
aauugacagagggcauggc

SEQ ID NO: 1094 - RNA, 19 nt, Artificial Sequence
  Description of Artificial Sequence Synthetic oligonucleotide
guucuuuagcauuugugga

SEQ ID NO: 1095 - RNA, 19 nt, Artificial Sequence
  Description of Artificial Sequence Synthetic oligonucleotide
uggacaaaaucaccuggcu

SEQ ID NO: 1096 - RNA, 19 nt, Artificial Sequence
  Description of Artificial Sequence Synthetic oligonucleotide
agauaaauaugugcaucuc

SEQ ID NO: 1097 - RNA, 19 nt, Artificial Sequence
  Description of Artificial Sequence Synthetic oligonucleotide
uuucaguggcucgauauga

SEQ ID NO: 1098 - RNA, 19 nt, Artificial Sequence
  Description of Artificial Sequence Synthetic oligonucleotide
augugcaucucccaaagua

SEQ ID NO: 1099 - RNA, 19 nt, Artificial Sequence
  Description of Artificial Sequence Synthetic oligonucleotide
ccuucuuaaaaccgcagcg

SEQ ID NO: 1100 - RNA, 19 nt, Artificial Sequence
  Description of Artificial Sequence Synthetic oligonucleotide
agguaagcagggcauagcu

SEQ ID NO: 1101 - RNA, 19 nt, Artificial Sequence
  Description of Artificial Sequence Synthetic oligonucleotide
agucuucaacuuugaaauc

SEQ ID NO: 1102 - RNA, 19 nt, Artificial Sequence
  Description of Artificial Sequence Synthetic oligonucleotide
uugguugugagcagaggaa

SEQ ID NO: 1103 - RNA, 19 nt, Artificial Sequence
  Description of Artificial Sequence Synthetic oligonucleotide
gagcagaggaaauucaucu

SEQ ID NO: 1104 - RNA, 19 nt, Artificial Sequence
  Description of Artificial Sequence Synthetic oligonucleotide
uacuccuugacccgaguaa

SEQ ID NO: 1105 - RNA, 19 nt, Artificial Sequence
  Description of Artificial Sequence Synthetic oligonucleotide
caggcacacaugaugauuc

SEQ ID NO: 1106 - RNA, 19 nt, Artificial Sequence
  Description of Artificial Sequence Synthetic oligonucleotide
ccgaacaccgacaaggugc

SEQ ID NO: 1107 - RNA, 19 nt, Artificial Sequence
  Description of Artificial Sequence Synthetic oligonucleotide
ggaaaccauaaacugggcc

SEQ ID NO: 1108 - RNA, 19 nt, Artificial Sequence
  Description of Artificial Sequence Synthetic oligonucleotide
ucccuuggaggacaguucu

SEQ ID NO: 1109 - RNA, 19 nt, Artificial Sequence
  Description of Artificial Sequence Synthetic oligonucleotide
agaacacucuuuugguugu

SEQ ID NO: 1110 - RNA, 19 nt, Artificial Sequence
  Description of Artificial Sequence Synthetic oligonucleotide
cacucuuuugguugugagc

SEQ ID NO: 1111 - RNA, 19 nt, Artificial Sequence
  Description of Artificial Sequence Synthetic oligonucleotide
cucugguaaaguuggcaag

SEQ ID NO: 1112 - RNA, 19 nt, Artificial Sequence
  Description of Artificial Sequence Synthetic oligonucleotide
agaggaaauucaucucuca

SEQ ID NO: 1113 - RNA, 19 nt, Artificial Sequence
  Description of Artificial Sequence Synthetic oligonucleotide
uuuagcauuuguggagccc

SEQ ID NO: 1114 - RNA, 19 nt, Artificial Sequence
  Description of Artificial Sequence Synthetic oligonucleotide
aaggugccagugccgguac

SEQ ID NO: 1115 - RNA, 19 nt, Artificial Sequence
  Description of Artificial Sequence Synthetic oligonucleotide
uaaucguuggauguggauu

SEQ ID NO: 1116 - RNA, 19 nt, Artificial Sequence
  Description of Artificial Sequence Synthetic oligonucleotide
cuuccugucgagcagagaa

SEQ ID NO: 1117 - RNA, 19 nt, Artificial Sequence
  Description of Artificial Sequence Synthetic oligonucleotide
ugucgagcagagaauccca

SEQ ID NO: 1118 - RNA, 19 nt, Artificial Sequence
  Description of Artificial Sequence Synthetic oligonucleotide
acacucuuuugguugugag

SEQ ID NO: 1119 - RNA, 19 nt, Artificial Sequence
  Description of Artificial Sequence Synthetic oligonucleotide
gauuuugagcuuugggaaa

SEQ ID NO: 1120 - RNA, 19 nt, Artificial Sequence
  Description of Artificial Sequence Synthetic oligonucleotide
caguggcucgauaugauuc

SEQ ID NO: 1121 - RNA, 19 nt, Artificial Sequence
  Description of Artificial Sequence Synthetic oligonucleotide
ccacauagaauugacagag

SEQ ID NO: 1122 - RNA, 19 nt, Artificial Sequence
  Description of Artificial Sequence Synthetic oligonucleotide
agaauugacagagggcaug

SEQ ID NO: 1123 - RNA, 19 nt, Artificial Sequence
  Description of Artificial Sequence Synthetic oligonucleotide
agcuggucuacuccuugac

SEQ ID NO: 1124 - RNA, 19 nt, Artificial Sequence
  Description of Artificial Sequence Synthetic oligonucleotide
ucuuaaaaccgcagcgcau

SEQ ID NO: 1125 - RNA, 19 nt, Artificial Sequence
  Description of Artificial Sequence Synthetic oligonucleotide
aguggcucgauaugauuca

SEQ ID NO: 1126 - RNA, 19 nt, Artificial Sequence
  Description of Artificial Sequence Synthetic oligonucleotide
agagaaucccaggcugucc

SEQ ID NO: 1127 - RNA, 19 nt, Artificial Sequence
  Description of Artificial Sequence Synthetic oligonucleotide
uuguagucugcuccaaaau

SEQ ID NO: 1128 - RNA, 19 nt, Artificial Sequence
  Description of Artificial Sequence Synthetic oligonucleotide
aggggaagaucuuuugggu

SEQ ID NO: 1129 - RNA, 19 nt, Artificial Sequence
  Description of Artificial Sequence Synthetic oligonucleotide
uuauaaccuucaaucugaa

SEQ ID NO: 1130 - RNA, 19 nt, Artificial Sequence
  Description of Artificial Sequence Synthetic oligonucleotide
uuaaucguuggauguggau

SEQ ID NO: 1131 - RNA, 19 nt, Artificial Sequence
  Description of Artificial Sequence Synthetic oligonucleotide
cacauagaauugacagagg

SEQ ID NO: 1132 - RNA, 19 nt, Artificial Sequence
  Description of Artificial Sequence Synthetic oligonucleotide
ucuuuugguugugagcaga

SEQ ID NO: 1133 - RNA, 19 nt, Artificial Sequence
  Description of Artificial Sequence Synthetic oligonucleotide
ucaccuggcuucaggcccg

SEQ ID NO: 1134 - RNA, 19 nt, Artificial Sequence
  Description of Artificial Sequence Synthetic oligonucleotide
aggacaguucuuuagcauu

SEQ ID NO: 1135 - RNA, 19 nt, Artificial Sequence
  Description of Artificial Sequence Synthetic oligonucleotide
aauucaucucucaggcugu

SEQ ID NO: 1136 - RNA, 19 nt, Artificial Sequence
  Description of Artificial Sequence Synthetic oligonucleotide
cuuuagcauuuguggagcc

SEQ ID NO: 1137 - RNA, 19 nt, Artificial Sequence
  Description of Artificial Sequence Synthetic oligonucleotide
guuggauguggauuauaac

SEQ ID NO: 1138 - RNA, 19 nt, Artificial Sequence
  Description of Artificial Sequence Synthetic oligonucleotide
auuuuccacauagaauuga

SEQ ID NO: 1139 - RNA, 19 nt, Artificial Sequence
  Description of Artificial Sequence Synthetic oligonucleotide
ggauuuugagcuuugggaa

SEQ ID NO: 1140 - RNA, 19 nt, Artificial Sequence
  Description of Artificial Sequence Synthetic oligonucleotide
auuuguggagcccuugaua

SEQ ID NO: 1141 - RNA, 19 nt, Artificial Sequence
  Description of Artificial Sequence Synthetic oligonucleotide
gagaaucccaggcugucca

SEQ ID NO: 1142 - RNA, 19 nt, Artificial Sequence
  Description of Artificial Sequence Synthetic oligonucleotide
cauuuuaaucguuggaugu

SEQ ID NO: 1143 - RNA, 19 nt, Artificial Sequence
  Description of Artificial Sequence Synthetic oligonucleotide
acacaugaugauucuucug

SEQ ID NO: 1144 - RNA, 19 nt, Artificial Sequence
  Description of Artificial Sequence Synthetic oligonucleotide
aagaucuuuuggguuccag

SEQ ID NO: 1145 - RNA, 19 nt, Artificial Sequence
  Description of Artificial Sequence Synthetic oligonucleotide
uuaaaaccgcagcgcauaa

SEQ ID NO: 1146 - RNA, 19 nt, Artificial Sequence
  Description of Artificial Sequence Synthetic oligonucleotide
agcgcuaaacagccauuuc

SEQ ID NO: 1147 - RNA, 19 nt, Artificial Sequence
  Description of Artificial Sequence Synthetic oligonucleotide
ugugugcuaucauguaggu

SEQ ID NO: 1148 - RNA, 19 nt, Artificial Sequence
  Description of Artificial Sequence Synthetic oligonucleotide
cuguaucgcgccugcaugc

SEQ ID NO: 1149 - RNA, 19 nt, Artificial Sequence
  Description of Artificial Sequence Synthetic oligonucleotide
ucccaaaguauggacaaaa

SEQ ID NO: 1150 - RNA, 19 nt, Artificial Sequence
  Description of Artificial Sequence Synthetic oligonucleotide
ugaugauucuucugucauc

SEQ ID NO: 1151 - RNA, 19 nt, Artificial Sequence
  Description of Artificial Sequence Synthetic oligonucleotide
uugugagcagaggaaauuc

SEQ ID NO: 1152 - RNA, 19 nt, Artificial Sequence
  Description of Artificial Sequence Synthetic oligonucleotide
gauguggauuauaaccuuc

SEQ ID NO: 1153 - RNA, 19 nt, Artificial Sequence
  Description of Artificial Sequence Synthetic oligonucleotide
auaaaccauagcaacuccu

SEQ ID NO: 1154 - RNA, 19 nt, Artificial Sequence
  Description of Artificial Sequence Synthetic oligonucleotide
uccuugacccgaguaaucu

SEQ ID NO: 1155 - RNA, 19 nt, Artificial Sequence
  Description of Artificial Sequence Synthetic oligonucleotide
uuuugcagcuggucuacuc

SEQ ID NO: 1156 - RNA, 19 nt, Artificial Sequence
  Description of Artificial Sequence Synthetic oligonucleotide
caaaaugucuccacuggaa

SEQ ID NO: 1157 - RNA, 19 nt, Artificial Sequence
  Description of Artificial Sequence Synthetic oligonucleotide
uggaaaccauaaacugggc

SEQ ID NO: 1158 - RNA, 19 nt, Artificial Sequence
  Description of Artificial Sequence Synthetic oligonucleotide
aauuuucaguggcucgaua

SEQ ID NO: 1159 - RNA, 19 nt, Artificial Sequence
  Description of Artificial Sequence Synthetic oligonucleotide
ucucaggcuguaucgcgcc

SEQ ID NO: 1160 - RNA, 19 nt, Artificial Sequence
  Description of Artificial Sequence Synthetic oligonucleotide
gcgcuaaacagccauuucc

SEQ ID NO: 1161 - RNA, 19 nt, Artificial Sequence
  Description of Artificial Sequence Synthetic oligonucleotide
gguauucacagcaucuuug

SEQ ID NO: 1162 - RNA, 19 nt, Artificial Sequence
  Description of Artificial Sequence Synthetic oligonucleotide
cugucgagcagagaauccc

SEQ ID NO: 1163 - RNA, 19 nt, Artificial Sequence
  Description of Artificial Sequence Synthetic oligonucleotide
gauugguuuugaugguguc

SEQ ID NO: 1164 - RNA, 19 nt, Artificial Sequence
  Description of Artificial Sequence Synthetic oligonucleotide
aaggaggcagugccaucag

SEQ ID NO: 1165 - RNA, 19 nt, Artificial Sequence
  Description of Artificial Sequence Synthetic oligonucleotide
uauaaccuucaaucugaaa

SEQ ID NO: 1166 - RNA, 19 nt, Artificial Sequence
  Description of Artificial Sequence Synthetic oligonucleotide
uuggcaagacagcucccca

SEQ ID NO: 1167 - RNA, 19 nt, Artificial Sequence
  Description of Artificial Sequence Synthetic oligonucleotide
ucuuugaaagagcgcuaaa

SEQ ID NO: 1168 - RNA, 19 nt, Artificial Sequence
  Description of Artificial Sequence Synthetic oligonucleotide
uaucgcgccugcaugccga

SEQ ID NO: 1169 - RNA, 19 nt, Artificial Sequence
  Description of Artificial Sequence Synthetic oligonucleotide
ucccauauucucacucccu

SEQ ID NO: 1170 - RNA, 19 nt, Artificial Sequence
  Description of Artificial Sequence Synthetic oligonucleotide
cagaggaaauucaucucuc

SEQ ID NO: 1171 - RNA, 19 nt, Artificial Sequence
  Description of Artificial Sequence Synthetic oligonucleotide
cccuugauaaaccauagca

SEQ ID NO: 1172 - RNA, 19 nt, Artificial Sequence
  Description of Artificial Sequence Synthetic oligonucleotide
aucuccugaccucugguaa

SEQ ID NO: 1173 - RNA, 19 nt, Artificial Sequence
  Description of Artificial Sequence Synthetic oligonucleotide
agcagcggagugcagcuug

SEQ ID NO: 1174 - RNA, 19 nt, Artificial Sequence
  Description of Artificial Sequence Synthetic oligonucleotide
uagaauugacagagggcau

SEQ ID NO: 1175 - RNA, 19 nt, Artificial Sequence
  Description of Artificial Sequence Synthetic oligonucleotide
ccauuuuaaucguuggaug

SEQ ID NO: 1176 - RNA, 19 nt, Artificial Sequence
  Description of Artificial Sequence Synthetic oligonucleotide
guagguaagcagggcauag

SEQ ID NO: 1177 - RNA, 19 nt, Artificial Sequence
  Description of Artificial Sequence Synthetic oligonucleotide
ccaacgacuggcauagcgg

SEQ ID NO: 1178 - RNA, 19 nt, Artificial Sequence
  Description of Artificial Sequence Synthetic oligonucleotide
uggcaucccauauucucac

SEQ ID NO: 1179 - RNA, 19 nt, Artificial Sequence
  Description of Artificial Sequence Synthetic oligonucleotide
ugcaugccgaacaccgaca

SEQ ID NO: 1180 - RNA, 19 nt, Artificial Sequence
  Description of Artificial Sequence Synthetic oligonucleotide
uuugagcuuugggaaaggu

SEQ ID NO: 1181 - RNA, 19 nt, Artificial Sequence
  Description of Artificial Sequence Synthetic oligonucleotide
gccaacgacuggcauagcg

SEQ ID NO: 1182 - RNA, 19 nt, Artificial Sequence
  Description of Artificial Sequence Synthetic oligonucleotide
aacacucuuuugguuguga

SEQ ID NO: 1183 - RNA, 19 nt, Artificial Sequence
  Description of Artificial Sequence Synthetic oligonucleotide
ccaaaguauggacaaaauc

SEQ ID NO: 1184 - RNA, 19 nt, Artificial Sequence
  Description of Artificial Sequence Synthetic oligonucleotide
cagauaaauaugugcaucu

SEQ ID NO: 1185 - RNA, 19 nt, Artificial Sequence
  Description of Artificial Sequence Synthetic oligonucleotide
accauagcaacuccuccaa

SEQ ID NO: 1186 - RNA, 19 nt, Artificial Sequence
  Description of Artificial Sequence Synthetic oligonucleotide
ucucgcugaagcugaauuu

SEQ ID NO: 1187 - RNA, 19 nt, Artificial Sequence
  Description of Artificial Sequence Synthetic oligonucleotide
uaugugcaucucccaaagu

SEQ ID NO: 1188 - RNA, 19 nt, Artificial Sequence
  Description of Artificial Sequence Synthetic oligonucleotide
auauucucacucccuugga

SEQ ID NO: 1189 - RNA, 19 nt, Artificial Sequence
  Description of Artificial Sequence Synthetic oligonucleotide
guuggcaagacagcucccc

SEQ ID NO: 1190 - RNA, 19 nt, Artificial Sequence
  Description of Artificial Sequence Synthetic oligonucleotide
agcggagugcagcuuggag

SEQ ID NO: 1191 - RNA, 19 nt, Artificial Sequence
  Description of Artificial Sequence Synthetic oligonucleotide
gggaagaucuuuuggguuc

SEQ ID NO: 1192 - RNA, 19 nt, Artificial Sequence
  Description of Artificial Sequence Synthetic oligonucleotide
aaacucccuuccagaacac

SEQ ID NO: 1193 - RNA, 19 nt, Artificial Sequence
  Description of Artificial Sequence Synthetic oligonucleotide
cuccuucuuaaaaccgcag

SEQ ID NO: 1194 - RNA, 19 nt, Artificial Sequence
  Description of Artificial Sequence Synthetic oligonucleotide
aggucuugguucucgcuga

SEQ ID NO: 1195 - RNA, 19 nt, Artificial Sequence
  Description of Artificial Sequence Synthetic oligonucleotide
ugaugugugcuaucaugua

SEQ ID NO: 1196 - RNA, 19 nt, Artificial Sequence
  Description of Artificial Sequence Synthetic oligonucleotide
cugcaugccgaacaccgac

SEQ ID NO: 1197 - RNA, 19 nt, Artificial Sequence
  Description of Artificial Sequence Synthetic oligonucleotide
ugguugugagcagaggaaa

SEQ ID NO: 1198 - RNA, 19 nt, Artificial Sequence
  Description of Artificial Sequence Synthetic oligonucleotide
aacagccauuuccauuuua

SEQ ID NO: 1199 - RNA, 19 nt, Artificial Sequence
  Description of Artificial Sequence Synthetic oligonucleotide
aaaccgcagcgcauaaugu

SEQ ID NO: 1200 - RNA, 19 nt, Artificial Sequence
  Description of Artificial Sequence Synthetic oligonucleotide
ucgauaugauucagauaaa

SEQ ID NO: 1201 - RNA, 19 nt, Artificial Sequence
  Description of Artificial Sequence Synthetic oligonucleotide
uguucaauaaguuuuaagg

SEQ ID NO: 1202 - RNA, 19 nt, Artificial Sequence
  Description of Artificial Sequence Synthetic oligonucleotide
uuuaauauaaccugguuag

SEQ ID NO: 1203 - RNA, 19 nt, Artificial Sequence
  Description of Artificial Sequence Synthetic oligonucleotide
uuuagaauuauacaggggc

SEQ ID NO: 1204 - RNA, 19 nt, Artificial Sequence
  Description of Artificial Sequence Synthetic oligonucleotide
auagacuucaaauuuauac

SEQ ID NO: 1205 - RNA, 19 nt, Artificial Sequence
  Description of Artificial Sequence Synthetic oligonucleotide
ucaaauuuauacuugaugc

SEQ ID NO: 1206 - RNA, 19 nt, Artificial Sequence
  Description of Artificial Sequence Synthetic oligonucleotide
auuguuuagaauuauacag

SEQ ID NO: 1207 - RNA, 19 nt, Artificial Sequence
  Description of Artificial Sequence Synthetic oligonucleotide
uauauuuaauauaaccugg

SEQ ID NO: 1208 - RNA, 19 nt, Artificial Sequence
  Description of Artificial Sequence Synthetic oligonucleotide
aaguacuugaauucguucc

SEQ ID NO: 1209 - RNA, 19 nt, Artificial Sequence
  Description of Artificial Sequence Synthetic oligonucleotide
uuaacaguagcuauuaugc

SEQ ID NO: 1210 - RNA, 19 nt, Artificial Sequence
  Description of Artificial Sequence Synthetic oligonucleotide
augaaaauaauucuaauug

SEQ ID NO: 1211 - RNA, 19 nt, Artificial Sequence
  Description of Artificial Sequence Synthetic oligonucleotide
aguuuaguaagcaauaucc

SEQ ID NO: 1212 - RNA, 19 nt, Artificial Sequence
  Description of Artificial Sequence Synthetic oligonucleotide
uacuuauucaucuagcucc

SEQ ID NO: 1213 - RNA, 19 nt, Artificial Sequence
  Description of Artificial Sequence Synthetic oligonucleotide
agaacuuuauggcaaaugg

SEQ ID NO: 1214 - RNA, 19 nt, Artificial Sequence
  Description of Artificial Sequence Synthetic oligonucleotide
uguuuagaauuauacaggg

SEQ ID NO: 1215 - RNA, 19 nt, Artificial Sequence
  Description of Artificial Sequence Synthetic oligonucleotide
uuguuuagaauuauacagg

SEQ ID NO: 1216 - RNA, 19 nt, Artificial Sequence
  Description of Artificial Sequence Synthetic oligonucleotide
aaacggagaaguuaaaugu

SEQ ID NO: 1217 - RNA, 19 nt, Artificial Sequence
  Description of Artificial Sequence Synthetic oligonucleotide
uuuaaaacccauuucugcc

SEQ ID NO: 1218 - RNA, 19 nt, Artificial Sequence
  Description of Artificial Sequence Synthetic oligonucleotide
gaucuaauuuucuuuaaag

SEQ ID NO: 1219 - RNA, 19 nt, Artificial Sequence
  Description of Artificial Sequence Synthetic oligonucleotide
uuaauucuguccuuuaaag

SEQ ID NO: 1220 - RNA, 19 nt, Artificial Sequence
  Description of Artificial Sequence Synthetic oligonucleotide
uucaaauuuauacuugaug

SEQ ID NO: 1221 - RNA, 19 nt, Artificial Sequence
  Description of Artificial Sequence Synthetic oligonucleotide
agcucugcuguuuaaaacc

SEQ ID NO: 1222 - RNA, 19 nt, Artificial Sequence
  Description of Artificial Sequence Synthetic oligonucleotide
ucagucucuacuuugaucu

SEQ ID NO: 1223 - RNA, 19 nt, Artificial Sequence
  Description of Artificial Sequence Synthetic oligonucleotide
ucucuaaggagcacaaugg

SEQ ID NO: 1224 - RNA, 19 nt, Artificial Sequence
  Description of Artificial Sequence Synthetic oligonucleotide
gaaaaugagcuccuugugg

SEQ ID NO: 1225 - RNA, 19 nt, Artificial Sequence
  Description of Artificial Sequence Synthetic oligonucleotide
aaguuuaguaagcaauauc

SEQ ID NO: 1226 - RNA, 19 nt, Artificial Sequence
  Description of Artificial Sequence Synthetic oligonucleotide
uugaauucguuccugagca

SEQ ID NO: 1227 - RNA, 19 nt, Artificial Sequence
  Description of Artificial Sequence Synthetic oligonucleotide
ugcagaauaauucagucuc

SEQ ID NO: 1228 - RNA, 19 nt, Artificial Sequence
  Description of Artificial Sequence Synthetic oligonucleotide
aaauaauucuaauuguuua

SEQ ID NO: 1229 - RNA, 19 nt, Artificial Sequence
  Description of Artificial Sequence Synthetic oligonucleotide
uuaccuuccacugaggagg

SEQ ID NO: 1230 - RNA, 19 nt, Artificial Sequence
  Description of Artificial Sequence Synthetic oligonucleotide
aauauaaccugguuagacu

SEQ ID NO: 1231 - RNA, 19 nt, Artificial Sequence
  Description of Artificial Sequence Synthetic oligonucleotide
ucucaauucauuaucucug

SEQ ID NO: 1232 - RNA, 19 nt, Artificial Sequence
  Description of Artificial Sequence Synthetic oligonucleotide
aauaaguuuuaaggcaucg

SEQ ID NO: 1233 - RNA, 19 nt, Artificial Sequence
  Description of Artificial Sequence Synthetic oligonucleotide
cugaagaugagaaacggag

SEQ ID NO: 1234 - RNA, 19 nt, Artificial Sequence
  Description of Artificial Sequence Synthetic oligonucleotide
aaauuuauacuugaugccu

SEQ ID NO: 1235 - RNA, 19 nt, Artificial Sequence
  Description of Artificial Sequence Synthetic oligonucleotide
gaaacggagaaguuaaaug

SEQ ID NO: 1236 - RNA, 19 nt, Artificial Sequence
  Description of Artificial Sequence Synthetic oligonucleotide
guucaauaaguuuuaaggc

SEQ ID NO: 1237 - RNA, 19 nt, Artificial Sequence
  Description of Artificial Sequence Synthetic oligonucleotide
aguauauuuaauauaaccu

SEQ ID NO: 1238 - RNA, 19 nt, Artificial Sequence
  Description of Artificial Sequence Synthetic oligonucleotide
ggcauugagaacuuuaugg

SEQ ID NO: 1239 - RNA, 19 nt, Artificial Sequence
  Description of Artificial Sequence Synthetic oligonucleotide
aauucuuaacaguagcuau

SEQ ID NO: 1240 - RNA, 19 nt, Artificial Sequence
  Description of Artificial Sequence Synthetic oligonucleotide
gucacuuucaaauuccugc

SEQ ID NO: 1241 - RNA, 19 nt, Artificial Sequence
  Description of Artificial Sequence Synthetic oligonucleotide
aacuuuauggcaaauggug

SEQ ID NO: 1242 - RNA, 19 nt, Artificial Sequence
  Description of Artificial Sequence Synthetic oligonucleotide
uaucucugaauucuuaaca

SEQ ID NO: 1243 - RNA, 19 nt, Artificial Sequence
  Description of Artificial Sequence Synthetic oligonucleotide
uacccuaugcaucugcugg

SEQ ID NO: 1244 - RNA, 19 nt, Artificial Sequence
  Description of Artificial Sequence Synthetic oligonucleotide
uaaaggucgauucuucuca

SEQ ID NO: 1245 - RNA, 19 nt, Artificial Sequence
  Description of Artificial Sequence Synthetic oligonucleotide
cuuaacaguagcuauuaug

SEQ ID NO: 1246 - RNA, 19 nt, Artificial Sequence
  Description of Artificial Sequence Synthetic oligonucleotide
caaauuuauacuugaugcc

SEQ ID NO: 1247 - RNA, 19 nt, Artificial Sequence
  Description of Artificial Sequence Synthetic oligonucleotide
ucucaauuuacccauuuuc

SEQ ID NO: 1248 - RNA, 19 nt, Artificial Sequence
  Description of Artificial Sequence Synthetic oligonucleotide
uaaggcaucguccagacuu

SEQ ID NO: 1249 - RNA, 19 nt, Artificial Sequence
  Description of Artificial Sequence Synthetic oligonucleotide
aacagaacucugcucagag

SEQ ID NO: 1250 - RNA, 19 nt, Artificial Sequence
  Description of Artificial Sequence Synthetic oligonucleotide
ugagaaacggagaaguuaa

SEQ ID NO: 1251 - RNA, 19 nt, Artificial Sequence
  Description of Artificial Sequence Synthetic oligonucleotide
uaucugcuaacucugguug

SEQ ID NO: 1252 - RNA, 19 nt, Artificial Sequence
  Description of Artificial Sequence Synthetic oligonucleotide
ucaauaaguuuuaaggcau

SEQ ID NO: 1253 - RNA, 19 nt, Artificial Sequence
  Description of Artificial Sequence Synthetic oligonucleotide
ucaauuuacccauuuucug

SEQ ID NO: 1254 - RNA, 19 nt, Artificial Sequence
  Description of Artificial Sequence Synthetic oligonucleotide
uuuggaaguacuugaauuc

SEQ ID NO: 1255 - RNA, 19 nt, Artificial Sequence
  Description of Artificial Sequence Synthetic oligonucleotide
uuaaaacccauuucugccc

SEQ ID NO: 1256 - RNA, 19 nt, Artificial Sequence
  Description of Artificial Sequence Synthetic oligonucleotide
uucauuaucucugaauucu

SEQ ID NO: 1257 - RNA, 19 nt, Artificial Sequence
  Description of Artificial Sequence Synthetic oligonucleotide
uuuaguaagcaauauccau

SEQ ID NO: 1258 - RNA, 19 nt, Artificial Sequence
  Description of Artificial Sequence Synthetic oligonucleotide
ugucugagugaacagaacu

SEQ ID NO: 1259 - RNA, 19 nt, Artificial Sequence
  Description of Artificial Sequence Synthetic oligonucleotide
ucuccaaaucaauuucugg

SEQ ID NO: 1260 - RNA, 19 nt, Artificial Sequence
  Description of Artificial Sequence Synthetic oligonucleotide
aucucugaauucuuaacag

SEQ ID NO: 1261 - RNA, 19 nt, Artificial Sequence
  Description of Artificial Sequence Synthetic oligonucleotide
ugaugugcagaauaauuca

SEQ ID NO: 1262 - RNA, 19 nt, Artificial Sequence
  Description of Artificial Sequence Synthetic oligonucleotide
ugagcauuggccagggaag

SEQ ID NO: 1263 - RNA, 19 nt, Artificial Sequence
  Description of Artificial Sequence Synthetic oligonucleotide
uguacuuauucaucuagcu

SEQ ID NO: 1264 - RNA, 19 nt, Artificial Sequence
  Description of Artificial Sequence Synthetic oligonucleotide
ucaucuagcuccuaucucu

SEQ ID NO: 1265 - RNA, 19 nt, Artificial Sequence
  Description of Artificial Sequence Synthetic oligonucleotide
aauuauacaggggcugggg

SEQ ID NO: 1266 - RNA, 19 nt, Artificial Sequence
  Description of Artificial Sequence Synthetic oligonucleotide
aaccugguuagacuaauga

SEQ ID NO: 1267 - RNA, 19 nt, Artificial Sequence
  Description of Artificial Sequence Synthetic oligonucleotide
agaaccagguuuuccggcc

SEQ ID NO: 1268 - RNA, 19 nt, Artificial Sequence
  Description of Artificial Sequence Synthetic oligonucleotide
uuaguaagcaauauccauu

SEQ ID NO: 1269 - RNA, 19 nt, Artificial Sequence
  Description of Artificial Sequence Synthetic oligonucleotide
cauauucugggacacggcg

SEQ ID NO: 1270 - RNA, 19 nt, Artificial Sequence
  Description of Artificial Sequence Synthetic oligonucleotide
ugguuguucaauaaguuuu

SEQ ID NO: 1271 - RNA, 19 nt, Artificial Sequence
  Description of Artificial Sequence Synthetic oligonucleotide
aacaguagcuauuaugcuu

SEQ ID NO: 1272 - RNA, 19 nt, Artificial Sequence
  Description of Artificial Sequence Synthetic oligonucleotide
agaccuuauauaauccucc

SEQ ID NO: 1273 - RNA, 19 nt, Artificial Sequence
  Description of Artificial Sequence Synthetic oligonucleotide
aaacggaacugccuccaac

SEQ ID NO: 1274 - RNA, 19 nt, Artificial Sequence
  Description of Artificial Sequence Synthetic oligonucleotide
uccaaaucaauuucuggga

SEQ ID NO: 1275 - RNA, 19 nt, Artificial Sequence
  Description of Artificial Sequence Synthetic oligonucleotide
aauauccauucucaauuca

SEQ ID NO: 1276 - RNA, 19 nt, Artificial Sequence
  Description of Artificial Sequence Synthetic oligonucleotide
agaaaggaccccuggguac

SEQ ID NO: 1277 - RNA, 19 nt, Artificial Sequence
  Description of Artificial Sequence Synthetic oligonucleotide
uuggaaguacuugaauucg

SEQ ID NO: 1278 - RNA, 19 nt, Artificial Sequence
  Description of Artificial Sequence Synthetic oligonucleotide
uuauauaauccuccaccug

SEQ ID NO: 1279 - RNA, 19 nt, Artificial Sequence
  Description of Artificial Sequence Synthetic oligonucleotide
aacggaacugccuccaacu

SEQ ID NO: 1280 - RNA, 19 nt, Artificial Sequence
  Description of Artificial Sequence Synthetic oligonucleotide
uaauauaaccugguuagac

SEQ ID NO: 1281 - RNA, 19 nt, Artificial Sequence
  Description of Artificial Sequence Synthetic oligonucleotide
cucacugaagaugagaaac

SEQ ID NO: 1282 - RNA, 19 nt, Artificial Sequence
  Description of Artificial Sequence Synthetic oligonucleotide
cugaauucuuaacaguagc

SEQ ID NO: 1283 - RNA, 19 nt, Artificial Sequence
  Description of Artificial Sequence Synthetic oligonucleotide
ugagaacuauauuaauucu

SEQ ID NO: 1284 - RNA, 19 nt, Artificial Sequence
  Description of Artificial Sequence Synthetic oligonucleotide
gagaacuauauuaauucug

SEQ ID NO: 1285 - RNA, 19 nt, Artificial Sequence
  Description of Artificial Sequence Synthetic oligonucleotide
uauuaugcuugccauuacu

SEQ ID NO: 1286 - RNA, 19 nt, Artificial Sequence
  Description of Artificial Sequence Synthetic oligonucleotide
uaguaagcaauauccauuc

SEQ ID NO: 1287 - RNA, 19 nt, Artificial Sequence
  Description of Artificial Sequence Synthetic oligonucleotide
ucugaugugcagaauaauu

SEQ ID NO: 1288 - RNA, 19 nt, Artificial Sequence
  Description of Artificial Sequence Synthetic oligonucleotide
uaugcuugccauuacuuaa

SEQ ID NO: 1289 - RNA, 19 nt, Artificial Sequence
  Description of Artificial Sequence Synthetic oligonucleotide
gaauaauucagucucuacu

SEQ ID NO: 1290 - RNA, 19 nt, Artificial Sequence
  Description of Artificial Sequence Synthetic oligonucleotide
uuguaaacggaacugccuc

SEQ ID NO: 1291 - RNA, 19 nt, Artificial Sequence
  Description of Artificial Sequence Synthetic oligonucleotide
aaaccauguccacuuuauc

SEQ ID NO: 1292 - RNA, 19 nt, Artificial Sequence
  Description of Artificial Sequence Synthetic oligonucleotide
ccuuauauaauccuccacc

SEQ ID NO: 1293 - RNA, 19 nt, Artificial Sequence
  Description of Artificial Sequence Synthetic oligonucleotide
uaaggagcacaauggagcc

SEQ ID NO: 1294 - RNA, 19 nt, Artificial Sequence
  Description of Artificial Sequence Synthetic oligonucleotide
cucuuguacacacacuacc

SEQ ID NO: 1295 - RNA, 19 nt, Artificial Sequence
  Description of Artificial Sequence Synthetic oligonucleotide
uauuaauucuguccuuuaa

SEQ ID NO: 1296 - RNA, 19 nt, Artificial Sequence
  Description of Artificial Sequence Synthetic oligonucleotide
gugaacagaacucugcuca

SEQ ID NO: 1297 - RNA, 19 nt, Artificial Sequence
  Description of Artificial Sequence Synthetic oligonucleotide
uuagacuaaugaaaaugaa

SEQ ID NO: 1298 - RNA, 19 nt, Artificial Sequence
  Description of Artificial Sequence Synthetic oligonucleotide
uuuaaaggucgauucuucu

SEQ ID NO: 1299 - RNA, 19 nt, Artificial Sequence
  Description of Artificial Sequence Synthetic oligonucleotide
agaacuauauuaauucugu

SEQ ID NO: 1300 - RNA, 19 nt, Artificial Sequence
  Description of Artificial Sequence Synthetic oligonucleotide
ugcuguuuaaaacccauuu

SEQ ID NO: 1301 - RNA, 19 nt, Artificial Sequence
  Description of Artificial Sequence Synthetic oligonucleotide
agaugagaaacggagaagu

SEQ ID NO: 1302 - RNA, 19 nt, Artificial Sequence
  Description of Artificial Sequence Synthetic oligonucleotide
ucauagacuucaaauuuau

SEQ ID NO: 1303 - RNA, 19 nt, Artificial Sequence
  Description of Artificial Sequence Synthetic oligonucleotide
agugaacagaacucugcuc

SEQ ID NO: 1304 - RNA, 19 nt, Artificial Sequence
  Description of Artificial Sequence Synthetic oligonucleotide
guuagacuaaugaaaauga

SEQ ID NO: 1305 - RNA, 19 nt, Artificial Sequence
  Description of Artificial Sequence Synthetic oligonucleotide
uccagaccuuauauaaucc

SEQ ID NO: 1306 - RNA, 19 nt, Artificial Sequence
  Description of Artificial Sequence Synthetic oligonucleotide
ucuagcuccuaucucuaug

SEQ ID NO: 1307 - RNA, 19 nt, Artificial Sequence
  Description of Artificial Sequence Synthetic oligonucleotide
cagacuuuuggcaagaaaa

SEQ ID NO: 1308 - RNA, 19 nt, Artificial Sequence
  Description of Artificial Sequence Synthetic oligonucleotide
aaaggucgauucuucucag

SEQ ID NO: 1309 - RNA, 19 nt, Artificial Sequence
  Description of Artificial Sequence Synthetic oligonucleotide
uuccagaccuuauauaauc

SEQ ID NO: 1310 - RNA, 19 nt, Artificial Sequence
  Description of Artificial Sequence Synthetic oligonucleotide
auuaucucugaauucuuaa

SEQ ID NO: 1311 - RNA, 19 nt, Artificial Sequence
  Description of Artificial Sequence Synthetic oligonucleotide
accuuauauaauccuccac

SEQ ID NO: 1312 - RNA, 19 nt, Artificial Sequence
  Description of Artificial Sequence Synthetic oligonucleotide
ugggacacggcgacgaugc

SEQ ID NO: 1313 - RNA, 19 nt, Artificial Sequence
  Description of Artificial Sequence Synthetic oligonucleotide
agaauuauacaggggcugg

SEQ ID NO: 1314 - RNA, 19 nt, Artificial Sequence
  Description of Artificial Sequence Synthetic oligonucleotide
uuaaaggucgauucuucuc

SEQ ID NO: 1315 - RNA, 19 nt, Artificial Sequence
  Description of Artificial Sequence Synthetic oligonucleotide
gguuagacuaaugaaaaug

SEQ ID NO: 1316 - RNA, 19 nt, Artificial Sequence
  Description of Artificial Sequence Synthetic oligonucleotide
acgugucacuuucaaauuc

SEQ ID NO: 1317 - RNA, 19 nt, Artificial Sequence
  Description of Artificial Sequence Synthetic oligonucleotide
uguaaacggaacugccucc

SEQ ID NO: 1318 - RNA, 19 nt, Artificial Sequence
  Description of Artificial Sequence Synthetic oligonucleotide
uauucugggacacggcgac

SEQ ID NO: 1319 - RNA, 19 nt, Artificial Sequence
  Description of Artificial Sequence Synthetic oligonucleotide
aagaugagaaacggagaag

SEQ ID NO: 1320 - RNA, 19 nt, Artificial Sequence
  Description of Artificial Sequence Synthetic oligonucleotide
aaaguuuaguaagcaauau

SEQ ID NO: 1321 - RNA, 19 nt, Artificial Sequence
  Description of Artificial Sequence Synthetic oligonucleotide
gaacgugucacuuucaaau

SEQ ID NO: 1322 - RNA, 19 nt, Artificial Sequence
  Description of Artificial Sequence Synthetic oligonucleotide
uccaacuauccaaaccaug

SEQ ID NO: 1323 - RNA, 19 nt, Artificial Sequence
  Description of Artificial Sequence Synthetic oligonucleotide
ucuccguucuugccgaugc

SEQ ID NO: 1324 - RNA, 19 nt, Artificial Sequence
  Description of Artificial Sequence Synthetic oligonucleotide
auaaucaccagguucuguu

SEQ ID NO: 1325 - RNA, 19 nt, Artificial Sequence
  Description of Artificial Sequence Synthetic oligonucleotide
ucuguuuaccuuccacuga

SEQ ID NO: 1326 - RNA, 19 nt, Artificial Sequence
  Description of Artificial Sequence Synthetic oligonucleotide
cuguuuaccuuccacugag

SEQ ID NO: 1327 - RNA, 19 nt, Artificial Sequence
  Description of Artificial Sequence Synthetic oligonucleotide
cuugccgaugcccauauuc

SEQ ID NO: 1328 - RNA, 19 nt, Artificial Sequence
  Description of Artificial Sequence Synthetic oligonucleotide
gucacaaagagucugagau

SEQ ID NO: 1329 - RNA, 19 nt, Artificial Sequence
  Description of Artificial Sequence Synthetic oligonucleotide
caugauccuugucacaaag

SEQ ID NO: 1330 - RNA, 19 nt, Artificial Sequence
  Description of Artificial Sequence Synthetic oligonucleotide
cuuuaaaggucgauucuuc

SEQ ID NO: 1331 - RNA, 19 nt, Artificial Sequence
  Description of Artificial Sequence Synthetic oligonucleotide
aguaucuuucuguuagccu

SEQ ID NO: 1332 - RNA, 19 nt, Artificial Sequence
  Description of Artificial Sequence Synthetic oligonucleotide
uauauuaauucuguccuuu

SEQ ID NO: 1333 - RNA, 19 nt, Artificial Sequence
  Description of Artificial Sequence Synthetic oligonucleotide
acucugguuguucaauaag

SEQ ID NO: 1334 - RNA, 19 nt, Artificial Sequence
  Description of Artificial Sequence Synthetic oligonucleotide
uacacacacuacccuaugc

SEQ ID NO: 1335 - RNA, 19 nt, Artificial Sequence
  Description of Artificial Sequence Synthetic oligonucleotide
cuaugcaucugcuggggag

SEQ ID NO: 1336 - RNA, 19 nt, Artificial Sequence
  Description of Artificial Sequence Synthetic oligonucleotide
uccuuuaaaggucgauucu

SEQ ID NO: 1337 - RNA, 19 nt, Artificial Sequence
  Description of Artificial Sequence Synthetic oligonucleotide
guuuagaauuauacagggg

SEQ ID NO: 1338 - RNA, 19 nt, Artificial Sequence
  Description of Artificial Sequence Synthetic oligonucleotide
uauucaucuagcuccuauc

SEQ ID NO: 1339 - RNA, 19 nt, Artificial Sequence
  Description of Artificial Sequence Synthetic oligonucleotide
cauucuuuggaaguacuug

SEQ ID NO: 1340 - RNA, 19 nt, Artificial Sequence
  Description of Artificial Sequence Synthetic oligonucleotide
ucacaaagagucugagaug

SEQ ID NO: 1341 - RNA, 19 nt, Artificial Sequence
  Description of Artificial Sequence Synthetic oligonucleotide
ucugguuguucaauaaguu

SEQ ID NO: 1342 - RNA, 19 nt, Artificial Sequence
  Description of Artificial Sequence Synthetic oligonucleotide
ucucuacugagaacuauau

SEQ ID NO: 1343 - RNA, 19 nt, Artificial Sequence
  Description of Artificial Sequence Synthetic oligonucleotide
uguacacacacuacccuau

SEQ ID NO: 1344 - RNA, 19 nt, Artificial Sequence
  Description of Artificial Sequence Synthetic oligonucleotide
gacuucaaauuuauacuug

SEQ ID NO: 1345 - RNA, 19 nt, Artificial Sequence
  Description of Artificial Sequence Synthetic oligonucleotide
aauucguuccugagcauug

SEQ ID NO: 1346 - RNA, 19 nt, Artificial Sequence
  Description of Artificial Sequence Synthetic oligonucleotide
ucguuccugagcauuggcc

SEQ ID NO: 1347 - RNA, 19 nt, Artificial Sequence
  Description of Artificial Sequence Synthetic oligonucleotide
ucgauucuucucaggaaug

SEQ ID NO: 1348 - RNA, 19 nt, Artificial Sequence
  Description of Artificial Sequence Synthetic oligonucleotide
gcauugagaacuuuauggc

SEQ ID NO: 1349 - RNA, 19 nt, Artificial Sequence
  Description of Artificial Sequence Synthetic oligonucleotide
auuauacaggggcugggga

SEQ ID NO: 1350 - RNA, 19 nt, Artificial Sequence
  Description of Artificial Sequence Synthetic oligonucleotide
uauacaggggcuggggaag

SEQ ID NO: 1351 - RNA, 19 nt, Artificial Sequence
  Description of Artificial Sequence Synthetic oligonucleotide
uuauacaggggcuggggaa

SEQ ID NO: 1352 - RNA, 19 nt, Artificial Sequence
  Description of Artificial Sequence Synthetic oligonucleotide
aacucugguuguucaauaa

SEQ ID NO: 1353 - RNA, 19 nt, Artificial Sequence
  Description of Artificial Sequence Synthetic oligonucleotide
uuaugcuugccauuacuua

SEQ ID NO: 1354 - RNA, 19 nt, Artificial Sequence
  Description of Artificial Sequence Synthetic oligonucleotide
guccuuuaaaggucgauuc

SEQ ID NO: 1355 - RNA, 19 nt, Artificial Sequence
  Description of Artificial Sequence Synthetic oligonucleotide
gaauuauacaggggcuggg

SEQ ID NO: 1356 - RNA, 19 nt, Artificial Sequence
  Description of Artificial Sequence Synthetic oligonucleotide
ucugggagaaguuuauauu

SEQ ID NO: 1357 - RNA, 19 nt, Artificial Sequence
  Description of Artificial Sequence Synthetic oligonucleotide
acggcgacgaugcaguuca

SEQ ID NO: 1358 - RNA, 19 nt, Artificial Sequence
  Description of Artificial Sequence Synthetic oligonucleotide
uaacaguagcuauuaugcu

SEQ ID NO: 1359 - RNA, 19 nt, Artificial Sequence
  Description of Artificial Sequence Synthetic oligonucleotide
uuccuuguaaacggaacug

SEQ ID NO: 1360 - RNA, 19 nt, Artificial Sequence
  Description of Artificial Sequence Synthetic oligonucleotide
uuuuccggcccauaaucac

SEQ ID NO: 1361 - RNA, 19 nt, Artificial Sequence
  Description of Artificial Sequence Synthetic oligonucleotide
guacuuauucaucuagcuc

SEQ ID NO: 1362 - RNA, 19 nt, Artificial Sequence
  Description of Artificial Sequence Synthetic oligonucleotide
ucuaauuguuuagaauuau

SEQ ID NO: 1363 - RNA, 19 nt, Artificial Sequence
  Description of Artificial Sequence Synthetic oligonucleotide
uggaaguacuugaauucgu

SEQ ID NO: 1364 - RNA, 19 nt, Artificial Sequence
  Description of Artificial Sequence Synthetic oligonucleotide
uauaaccugguuagacuaa

SEQ ID NO: 1365 - RNA, 19 nt, Artificial Sequence
  Description of Artificial Sequence Synthetic oligonucleotide
gaacuauauuaauucuguc

SEQ ID NO: 1366 - RNA, 19 nt, Artificial Sequence
  Description of Artificial Sequence Synthetic oligonucleotide
ucucuuguacacacacuac

SEQ ID NO: 1367 - RNA, 19 nt, Artificial Sequence
  Description of Artificial Sequence Synthetic oligonucleotide
acggagaaguuaaauguuc

SEQ ID NO: 1368 - RNA, 19 nt, Artificial Sequence
  Description of Artificial Sequence Synthetic oligonucleotide
caauauccauucucaauuc

SEQ ID NO: 1369 - RNA, 19 nt, Artificial Sequence
  Description of Artificial Sequence Synthetic oligonucleotide
cugguuagacuaaugaaaa

SEQ ID NO: 1370 - RNA, 19 nt, Artificial Sequence
  Description of Artificial Sequence Synthetic oligonucleotide
ugucacaaagagucugaga

SEQ ID NO: 1371 - RNA, 19 nt, Artificial Sequence
  Description of Artificial Sequence Synthetic oligonucleotide
uacuugaauucguuccuga

SEQ ID NO: 1372 - RNA, 19 nt, Artificial Sequence
  Description of Artificial Sequence Synthetic oligonucleotide
aauuuauacuugaugccuu

SEQ ID NO: 1373 - RNA, 19 nt, Artificial Sequence
  Description of Artificial Sequence Synthetic oligonucleotide
aacgugucacuuucaaauu

SEQ ID NO: 1374 - RNA, 19 nt, Artificial Sequence
  Description of Artificial Sequence Synthetic oligonucleotide
uuaauauaaccugguuaga

SEQ ID NO: 1375 - RNA, 19 nt, Artificial Sequence
  Description of Artificial Sequence Synthetic oligonucleotide
aucguccagacuuuuggca

SEQ ID NO: 1376 - RNA, 19 nt, Artificial Sequence
  Description of Artificial Sequence Synthetic oligonucleotide
uccuaucucuaugagcccc

SEQ ID NO: 1377 - RNA, 19 nt, Artificial Sequence
  Description of Artificial Sequence Synthetic oligonucleotide
ucuguuagccuuucuucuc

SEQ ID NO: 1378 - RNA, 19 nt, Artificial Sequence
  Description of Artificial Sequence Synthetic oligonucleotide
cuacugagaacuauauuaa

SEQ ID NO: 1379 - RNA, 19 nt, Artificial Sequence
  Description of Artificial Sequence Synthetic oligonucleotide
gaauucuuaacaguagcua

SEQ ID NO: 1380 - RNA, 19 nt, Artificial Sequence
  Description of Artificial Sequence Synthetic oligonucleotide
gcaucguccagacuuuugg

SEQ ID NO: 1381 - RNA, 19 nt, Artificial Sequence
  Description of Artificial Sequence Synthetic oligonucleotide
ugcuaacucugguuguuca

SEQ ID NO: 1382 - RNA, 19 nt, Artificial Sequence
  Description of Artificial Sequence Synthetic oligonucleotide
gagauggccuggcugauuc

SEQ ID NO: 1383 - RNA, 19 nt, Artificial Sequence
  Description of Artificial Sequence Synthetic oligonucleotide
uuguacacacacuacccua

SEQ ID NO: 1384 - RNA, 19 nt, Artificial Sequence
  Description of Artificial Sequence Synthetic oligonucleotide
uaucccuuggaaucucacu

SEQ ID NO: 1385 - RNA, 19 nt, Artificial Sequence
  Description of Artificial Sequence Synthetic oligonucleotide
uuacccauuuucugucuga

SEQ ID NO: 1386 - RNA, 19 nt, Artificial Sequence
  Description of Artificial Sequence Synthetic oligonucleotide
uagcucugcuguuuaaaac

SEQ ID NO: 1387 - RNA, 19 nt, Artificial Sequence
  Description of Artificial Sequence Synthetic oligonucleotide
uagcuauuaugcuugccau

SEQ ID NO: 1388 - RNA, 19 nt, Artificial Sequence
  Description of Artificial Sequence Synthetic oligonucleotide
guuuuaaggcaucguccag

SEQ ID NO: 1389 - RNA, 19 nt, Artificial Sequence
  Description of Artificial Sequence Synthetic oligonucleotide
agcggucgaaccaugacag

SEQ ID NO: 1390 - RNA, 19 nt, Artificial Sequence
  Description of Artificial Sequence Synthetic oligonucleotide
uauccaaaccauguccacu

SEQ ID NO: 1391 - RNA, 19 nt, Artificial Sequence
  Description of Artificial Sequence Synthetic oligonucleotide
accugguuagacuaaugaa

SEQ ID NO: 1392 - RNA, 19 nt, Artificial Sequence
  Description of Artificial Sequence Synthetic oligonucleotide
cuagcuccuaucucuauga

SEQ ID NO: 1393 - RNA, 19 nt, Artificial Sequence
  Description of Artificial Sequence Synthetic oligonucleotide
uaaccugguuagacuaaug

SEQ ID NO: 1394 - RNA, 19 nt, Artificial Sequence
  Description of Artificial Sequence Synthetic oligonucleotide
uggcaaaugguguuucuua

SEQ ID NO: 1395 - RNA, 19 nt, Artificial Sequence
  Description of Artificial Sequence Synthetic oligonucleotide
uucguuccugagcauuggc

SEQ ID NO: 1396 - RNA, 19 nt, Artificial Sequence
  Description of Artificial Sequence Synthetic oligonucleotide
acccuaugcaucugcuggg

SEQ ID NO: 1397 - RNA, 19 nt, Artificial Sequence
  Description of Artificial Sequence Synthetic oligonucleotide
auccaaaccauguccacuu

SEQ ID NO: 1398 - RNA, 19 nt, Artificial Sequence
  Description of Artificial Sequence Synthetic oligonucleotide
uacugagaacuauauuaau

SEQ ID NO: 1399 - RNA, 19 nt, Artificial Sequence
  Description of Artificial Sequence Synthetic oligonucleotide
caauaaguuuuaaggcauc

SEQ ID NO: 1400 - RNA, 19 nt, Artificial Sequence
  Description of Artificial Sequence Synthetic oligonucleotide
auuuauacuugaugccuuu

SEQ ID NO: 1401 - protein, 16 aa, Unknown
  Description of Unknown Penetratin cell permeation peptide
RQIKIWFQNRRMKWKK

SEQ ID NO: 1402 - protein, 14 aa, Human immunodeficiency virus
GRKKRRQRRRPPQC

SEQ ID NO: 1403 - protein, 27 aa, Unknown
  Description of Unknown Signal sequence-based cell permeation peptide
GALFLGWLGAAGSTMGAWSQPKKKRKV

SEQ ID NO: 1404 - protein, 18 aa, Unknown
  Description of Unknown PVEC cell permeation peptide
LLIILRRRIRKQAHAHSK

SEQ ID NO: 1405 - protein, 26 aa, Unknown
  Description of Unknown Transportan cell permeation peptide
GWTLNSAGYLLKINLKALAALAKKIL

SEQ ID NO: 1406 - protein, 18 aa, Unknown
  Description of Unknown Amphiphilic model cell permeation peptide
KLALKLALKALKAALKLA

SEQ ID NO: 1407 - protein, 9 aa, Unknown
  Description of Unknown Arg9 cell permeation peptide
RRRRRRRRR

SEQ ID NO: 1408 - protein, 10 aa, Unknown
  Description of Unknown Bacterial cell wall permeation peptide
KFFKFFKFFK

SEQ ID NO: 1409 - protein, 37 aa, Unknown
  Description of Unknown LL-37 cell permeation peptide
LLGDFFRKSKEKIGKEFKRIVQRIKDFLRNLVPRTES

SEQ ID NO: 1410 - protein, 31 aa, Unknown
  Description of Unknown Cecropin P1 cell permeation peptide
SWLSKTAKKLENSAKKRISEGIAIAIQGGPR

SEQ ID NO: 1411 - protein, 30 aa, Unknown
  Description of Unknown Alpha-defensin cell permeation peptide
ACYCRIPACIAGERRYGTCIYQGRLWAFCC

SEQ ID NO: 1412 - protein, 36 aa, Unknown
  Description of Unknown B-defensin cell permeation peptide
DHYNCVSSGGQCLYSACPIFTKIQGTCYRGKAKCCK

SEQ ID NO: 1413 - protein, 12 aa, Unknown
  Description of Unknown Bactenecin cell permeation peptide
RKCRIVVIRVCR

SEQ ID NO: 1414 - protein, 42 aa, Unknown
  Description of Unknown PR-39 cell permeation peptide
RRRPRPPYLPRPRPPPFFPPRLPPRIPPGFPPRFPPRFPGKR

SEQ ID NO: 1415 - protein, 13 aa, Unknown
  Description of Unknown Indolicidin cell permeation peptide
ILPWKWPWWPWRR

SEQ ID NO: 1416 - protein, 16 aa, Artificial Sequence
  Description of Artificial Sequence Synthetic peptide
AAVALLPAVLLALLAP

SEQ ID NO: 1417 - protein, 11 aa, Artificial Sequence
  Description of Artificial Sequence Synthetic peptide
AALLPVLLAAP

SEQ ID NO: 1418 - protein, 13 aa, Human immunodeficiency virus
GRKKRRQRRRPPQ

SEQ ID NO: 1419 - protein, 16 aa, Drosophila sp.
RQIKIWFQNRRMKWKK

SEQ ID NO: 1420 - protein, 187 aa, Cricetulus sp.
MVRPLNCIVAVSQNMGIGKNGDFPWPMLRNEFKYFQRMTTTSSVEGKQNLVIMGRKTWFSIPEKNRPLKDRINIVLSRELKEPPQGAHFLAKSLDDALKLIEQPELADKVDMVWIVGGSSVYKEAMNQPGHLRLFVTRIMQEFESDTFFPEIDLEKYKLLPEYPGVLSEVQEEKGIKYKFEVYEKKG

SEQ ID NO: 1421 - DNA, 564 bp, Cricetulus sp.
atggttcgaccgctgaactgcatcgtcgccgtgtcccagaatatgggcatcggcaagaacggagactttccctggccaatgctcaggaacgaattcaagtacttccaaagaatgaccaccacctcctcagtggaaggtaaacagaacctggtgattatgggccggaaaacctggttctccattcctgagaagaatcgacctttaaaggacagaattaatatagttctcagtagagagctcaaggaaccaccacaaggagctcattttcttgccaaaagtctggacgatgccttaaaacttattgaacaaccagagttagcagataaagtggacatggtttggatagttggaggcagttccgtttacaaggaagccatgaatcagccaggccatctcagactctttgtgacaaggatcatgcaggaatttgaaagtgacacgttcttcccagaaattgatttggagaaatataaacttctcccagagtacccaggggtcctttctgaagtccaggaggaaaaaggcatcaagtataaatttgaagtctatgagaagaaaggctaa

SEQ ID NO: 1422 - DNA, 5307 bp, Mus musculus
aggacgcgctggatcttaggcttcccgcagacctgaagaaccggcttagaaccgtttgcctccccgggcctgggccggcggcagtagagcctcccgacgcggatttcccgcggggcattgcagtgtgcagaagagccggcctgctaggagcgcgagcgcgcggccgcactttctcgcgcctgcgcgcgcgcacgcctcaacctgtgcgggaccggccttgggggcggagccttagctacacaaatagaatgcgcggcgggccttggtgggggcggggccttagctgcacaaataggatgcgcggcgggccttggtaggggcggagccttagctgcacaaataggatgcgcggcgggccttggtgggggcggggcctaagctgcgcaagtggtacacagctcagggctgcgatttcgcgccaaacttgacggcaatcctagcgtgaaggctggtaggattttatccccgctgccatcatggttcgaccattgaactgcatcgtcgccgtgtcccaaaatatggggattggcaagaacggagacctaccctggcctccgctcaggaacgagttcaagtacttccaaagaatgaccacaacctcttcagtggaaggtaaacagaatctggtgattatgggtaggaaaacctggttctccattcctgagaagaatcgacctttaaaggacagaattaatatagttctcagtagagaactcaaagaaccaccacgaggagctcattttcttgccaaaagtttggatgatgccttaagacttattgaacaaccggaattggcaagtaaagtagacatggtttggatagtcggaggcagttctgtttaccaggaagccatgaatcaaccaggccacctcagactctttgtgacaaggatcatgcaggaatttgaaagtgacacgtttttcccagaaattgatttggggaaatataaacttctcccagaatacccaggcgtcctctctgaggtccaggaggaaaaaggcatcaagtataagtttgaagtctacgagaagaaagactaacaggaagatgctttcaagttctctgctcccctcctaaagctatgcatttttataagaccatgggacttttgctggctttagatctatgagtaattatttctttagggaggggtagttggaagaattgtttgttttgtgatcctggggatggaacctaagacccagtgcgtgctgagcaaatgctatactgctgagccaccccaaccctagcccctatataattctaaacaatatgttgtcatttcccagtaatctaacaaggttatagtaaaagtgccttaagaaatgtcacttgctataaaggtctcagtgcccctcccatgagacctcaagtggctccccagcatatgcacagggtactgtgtgtacaagagaccccagtgatgtagagcccctggagcatgagcagatgtgtgggctcataaaagtaggagctaggcaggtaagtccaaagggcagaaacaggttttaaacagcagagctggaactcagactataaagaaaattccatcaaagtagagactggattattgtatgcacatcacacttgcagcaaagctctgctcactcagacagaaaatcagtaaatggagaactccattgtgttccatggagacgagagcaggtggaagattatgtaagatctgaaacactgaaattgtctgcttctcatcttcagtgagattccaaaggatagtacagtgacagaacaagaataggcactctctacaaaaaaaagaaagaaaaaactaagtaatagcaagcataatagctactgttaagaactcagagataatgaattgagaatggatactgcttgaaatgaaaatttaataagttagaaactaaactttataaaaataaaaaaatgagcattaaaatggctttcctcatctcagcagggtttcagatcatcaggtcagagaaagtatttctgcctggccttgtaaattagtatggtctttttttatcttttacttgacaatttcctacatgtttacgatgtgtcttaatcataccaggtcctcatccccactcccccactacctttgtatcttctttctcttttcataaccttctgaaaccagtcattgctttcttcatgttcctgcatgtgtgctgtccactggagtacgggcagcctgccagtggacacacacacacacacacacacacacacacacacacacacacacagagccatcagttaccaatagctcttcagctgggtgcagtcttaggagcctctcctccatccaagctagaatatgggtctgcagatcaccacagctgctgtgagcttgtgagtgtggtgtccatgccatgtccagaaggcagcattatatatacatggccctattccccaggcttcaggccttcattctttccaccacttcttcagtgctccctgagccttagacaagtctactgatagaaatggtctgttcagagctgaatagtaaacagtcacttagtctctacactttgatcagccttgtgtctgtaagctggatgctctctactgaaaaaaaaaaaaaaaaaaacagaacttcttctccaaccagggttaagagcactaatacagggtataaacacaggtatttagaaagcactctgaaaacctagccatttaacaaagcagcagtagtaggtttgtccttagaacccatgacttccacaccacaggcatttgaccagttttgcagtactagacatgaattgtctcctatagagccggcctcaaatccaactaggaagcattgacaggcatgccattgttgttcagtagaaactgtaacatgcagggcctaccaccaactaagtccataaaagcttttctccctggtgatgtgctcggcaccttctggcgctgaaaactgtccagtgaaggaggaagtttccaggcagctccagattgattctctatgtcctgcaagtaaatgtgttgtaaatgtgccttcaacagtaggggcttgtcatctggctacgacaggcaaccaagagcgatggcaaaagatgtgttgttttgggagcctccttaactagttacttttaagggtgccccacacctggcactgagagtttcatttaataacctaggtcttcatttagtaagcattgtccaccagtgtgtaggatgcctccattcaaacttttttctttagttataattacctgataaccatgaacaaagagatgaactcaaagaggcagagagtctgctgagctagcccatgaagcaggtgagccagtttgtgttatgtagcagtttcctctaaggtcaaaacctgtcttagaaggaagctgttttgacataagacggtatagaggagctagaagaagagaatattctaagggaagctgggaacttccttaagctcagcaaatacacaacttagaagatcttaaaattgaccaggtacactagactcctccctccccaagcatagataagcaatgattgccgaggggctctgagacacgctgagctgtctagaagaccaactgagcctcctggcaaagcagagaccagcagagctgtctgaaggaggcttggacctaccgaactccctagaaagagcactggaacctgttgagctgcctgtaggttgtctaatacactccaggtttcctgagctgtgactctgctggaacgggcttttggtgatgcaattttgatggtctccttcctttgaacgtccatatctggggttaacagacatttgctcctgtctctccaggaggtgtgttatacaatgatttggatccctggtgctcaggcagaggcaagtacagtagcatatgtttgtaaccctggcagtggtgtggtatgggagagactggcagtgtatttgcaagccagactgacccaagaaagaaagaaaaaatttaattgtttcagcatcttttaaatttggaaaaaagcataaatgtcaaagaaataattactatgtctataaaatacaatatttgcagcacttgaaaaggatgtggccctaacaggaaagttcttcaggaaaagaaacacacacacaaaatctagtattagtatgtctaagagtaatatatattaaaaatagttgacaatatcagacttagtgttattttctgtttgtcttctatgattaggattttttggaaacttttatagagaaattatttaaactattaagtcacagcctttgttttcaacaggaaaatgagggtatagagcattgagaggaaaaatgattttccaaaagtcatccatcaagagagagagaccagaccatacaaaagaaaaaagtcaattctatagaacagttgcacatcaaataaagcaatagcgctttcttgcaatgaacataacctgaaagccaccaatatccagaatttgtaggactcagtgactcagtcagaaaaagaaacaagttatttacagaggaaattgaagtggccaatgcagagctgaaccttctaccagaaaggagagaggcaggggaggaaattacatgtgccagtctcaccatctttagactaaaatgttgacatctcccagagctagggatgctatgctcagcacgcagcagtttcactgtggataagaccaagagaacacctcatacatgccaaaacgaaagacaaaagcaccctggaaatctagacacaaggaaattgtcgtctggctattttagtatgagttctctccaatgtgtattctgaaacaattcagtgaaagtggtgtgtgtggtaggatgcaccagaaagcacagttgggatatgggaaaacaagaaacctagttcaaggtcattaagcagattcccattgacaacccgggaaactgagggaggaagaaagctggagtgtgtagatcctgatcgttgtttgcacgatgttccacactgccagcttgttgctctgtgtaaaccggacacactccaactgccaaggtcccaagctgctaacataaatgcaaagaatacgaaacactagcaattccatgtttctgctactttctacataaaaaaaagtgcacagcccagtaatttatttgaaatataatatccatcagggtgagggtaagggtgataatcatagcttccaccaaagcattgtgtatactgaaaaggagacatgatacgtttttgtgttagaaggcgaggtttcaggtgggctttgaatttggtttgactgagactcattgagtttgaggtgtctttaggaaaggaagaaagaagggaacaaaaaataaaaagcaatggaaacatgcaaaaaaaaaaaaaaaaa

SEQ ID NO: 1423 - protein, 307 aa, Mus musculus
MLVVGSELQSDAQQLSAEAPRHGELQYLRQVEHILRCGFKKEDRTGTGTLSVFGMQARYSLRDEFPLLTTKRVFWKGVLEELLWFIKGSTNAKELSSKGVRIWDANGSRDFLDSLGFSARQEGDLGPVYGFQWRHFGAEYKDMDSDYSGQGVDQLQKVIDTIKTNPDDRRIIMCAWNPKDLPLMALPPCHALCQFYVVNGELSCQLYQRSGDMGLGVPFNIASYALLTYMIAHITGLQPGDFVHTLGDAHIYLNHIEPLKIQLQREPRPFPKLKILRKVETIDDFKVEDFQIEGYNPHPTIKMEMAV

SEQ ID NO: 1424 - DNA, 3798 bp, Mus musculus
gggctggtgttggaggaaaagagcgccaggaaggtcctggttttgtcgctgactacactgctgccagactgctccgttatgctggtggttggctccgagctgcagtccgatgctcagcagctgagcgcggaagccccacggcatggagaactccagtacctgaggcaggtggagcacattttgcgctgcggcttcaagaaggaggaccgcacgggcactggcaccctgtcggtgttcggcatgcaggcacgatacagcctgagagatgaatttcctctgctcacaaccaaacgagtgttctggaagggtgttttggaggagttgttgtggtttatcaagggatccacaaatgctaaagaattgtcctcaaagggagtgagaatctgggatgccaatggatcccgagattttctggacagcttgggattttctgcccgacaggaaggggacctgggcccagtttatggtttccaatggaggcattttggagcagagtacaaagatatggattcagattactcgggacaaggagtagaccagctgcaaaaagtgattgacaccatcaaaaccaaccctgatgacagaagaatcatcatgtgtgcctggaacccaaaagatcttcccctgatggcactgcctccttgccatgccctctgtcagttctatgtggtgaatggggaactgtcttgccagctttaccagaggtcaggagatatgggtctgggcgtgcccttcaacattgccagctatgctctgctcacctacatgattgcacatatcacaggcctgcagccaggtgattttgtccacactttgggagatgcacatatttacctgaatcatatagagccgctgaaaattcagctacagcgagaaccaagacctttcccaaagctcaaaatccttcgaaaagttgagacaatcgatgatttcaaagttgaagactttcagattgaagggtataatccacatccaacgattaaaatggaaatggctgtttagagtgctttcagtgatgctatgaatatcaccgtttttttttttttaattaggtattttcctcatttacatttccaatgctataccaaaagtcccccataccctcccctccactcccctacccacccactcccacttcttggccctggcattcccttgtactggggcatataaagtttgcaagtccaatgggcctctctttccagtgatggccgactaggccatcttttgatacatatgcagctagagtcaagagctccggggtactggttagttcataatgttgttccacctatagggttgcagatccctttagctccttgggtactttctctagctcctccattgggggccctgtgatccatccaatagctgacagtgagcatccacttctgtgtacacctccaagagtagccattacagtcactcatggagtagcgtacgaagtacttcaggtagaagcacttgcagtccctcaaggcagaaccctcgaagtccttcaaggatgagccctcacattccctcatagaggattctatgcagtccctcaaggaggagccctcacagtcactcaatgaccacacacagtccatcaaggaaaagtcctcgcagtcgttcaaggaggaaccctctaagtccttcaaggatgagccctcacagtccctcatggaggaccccatgcagtccctcaaggaggagccctcacagttactcaaggacccaacacagcccatcaaggagaagtctttgtagttcctcaatgtggaacccacaaagtccttcaggatgagccctcacagttcctcatggaggacctcttgtagtcactcaaggaggagccctcacagtcacttaagcaccccacacagcacatcaaggagaagtccacccagtccttccagaaggaaccctcgaagtccttcaagtatgagccctcacagtccctcatggaggaccccatgcagtaacttgttgtgagccagctcatggctcacatggacagggtacacctgagtggcaggccagggctgaaagagaggggaactaggtggtcgagagaaaaatggagctaagtcaagattcctgatcaaagctcaaatgtttaatggcacatgctcttataaaggggggagggggaggcccattcccgccaattcattctaggagcccagctgcagttgacgacgtgcaggatagggtgaagttccagaatagctcagcagcctcccagcaggtagcagcatcttgaaggggaacagtggcagtggctgagcaatagagtgatctagggaggaaggctccaccctaggtaatctccttagtggcagaaaggtcaatgtctggctcagcctgcttcaggctgggggaggttacaatttcctccgttattaatattaaataagctggactgaggcataaaatttatgctctatagttctgcctgatttctttgggtctagtgaagaaacctgtctcagtgggattccctaactttttctcattgtaaacccacctattcgctaggccatggtgaatacccttgtctgggtaagactggctactgtcccaagtaaatactctgtagactagccctgagctattctatctcattctttgtaatgcctaattagtttcattgtctcttccaagctcctcggcttcaagcattttctaggacattggaacagtggccaaggcttagctatgtcagaattcaatcttaaaaggcacttataataagataatactaaaagagagcacgtggagccatacaccagactaacgtggggatagggtatgagtatacaggttatgagaacactaaagttccaggaggtgagtttctgtgaaaaactctgcctcgtgagtgctcccaggcacctctgcctgccaagcaaacttcactgtagtgtgtgtagcattctccccctttctttttatattcaggagtgctctaaacacaacgatttactaatagaatagtgccaataatagcaagttgaatatatatatctcccaatctaggagattcaatgcacttaggttttttttgtttgtttgtttgttttgttttttatctctggccaattcatcaatgttccaagtgtccaagtgacttttgctaatatctgaaacttctgcctttctagccttaatatatgctatgtcctgccctgttgttaagactgctacttataagacattaacctttgcctccaatttttttacatggctctgtggtgtattattagtaaaccacggcctcttggcatgtgcctctttttagactgatatgattccgagatctggatgcccgtcttagactgtctggccctcatagcaaaccttattcccgattcagaccaaaaccacaatatgtacttagaatgcttcttcatgaagatttaataactgccacctgtgctgttggaggtgagggtgcacacctgctgcagtaaggatgaagggcaggactgacctgcttgaaaaacaaagtggagacagtgaaaagcaacagtacaaaagcttctttgggggaagtccaggtactccattcaattgcaaattagcaatggtcatgcttagacttagagctcccgttgtggggaggtgacttcgagaatcacagaaaggctgtaactttctaggagaggaggctgtgctccaaattaacttttcggttaaagaggatttttagctttaataagagtcatacttactggaggattcaggatctttatccagttgacaaactaatctttcaggcaggcagccatcctgctccatcttctttttgtg

SEQ ID NO: 1425 - DNA, 1603 bp, Homo sapiens
cgggacggccgcgggaaaaggcgcgcggaaggggtcctgccaccgcgccacttggcctgcctccgtcccgccgcgccacttggcctgcctccgtcccgccgcgccacttcgcctgcctccgtcccccgcccgccgcgccatgcctgtggccggctcggagctgccgcgccggcccttgccccccgccgcacaggagcgggacgccgagccgcgtccgccgcacggggagctgcagtacctggggcagatccaacacatcctccgctgcggcgtcaggaaggacgaccgcacgggcaccggcaccctgtcggtattcggcatgcaggcgcgctacagcctgagagatgaattccctctgctgacaaccaaacgtgtgttctggaagggtgttttggaggagttgctgtggtttatcaagggatccacaaatgctaaagagctgtcttccaagggagtgaaaatctgggatgccaatggatcccgagactttttggacagcctgggattctccaccagagaagaaggggacttgggcccagtttatggcttccagtggaggcattttggggcagaatacagagatatggaatcagattattcaggacagggagttgaccaactgcaaagagtgattgacaccatcaaaaccaaccctgacgacagaagaatcatcatgtgcgcttggaatccaagagatcttcctctgatggcgctgcctccatgccatgccctctgccagttctatgtggtgaacagtgagctgtcctgccagctgtaccagagatcgggagacatgggcctcggtgtgcctttcaacatcgccagctacgccctgctcacgtacatgattgcgcacatcacgggcctgaagccaggtgactttatacacactttgggagatgcacatatttacctgaatcacatcgagccactgaaaattcagcttcagcgagaacccagacctttcccaaagctcaggattcttcgaaaagttgagaaaattgatgacttcaaagctgaagactttcagattgaagggtacaatccgcatccaactattaaaatggaaatggctgtttagggtgctttcaaaggagctcgaaggatattgtcagtctttaggggttgggctggatgccgaggtaaaagttctttttgctctaaaagaaaaaggaactaggtcaaaaatctgtccgtgacctatcagttattaatttttaaggatgttgccactggcaaatgtaactgtgccagttctttccataataaaaggctttgagttaactcactgagggtatctgacaatgctgaggttatgaacaaagtgaggagaatgaaatgtatgtgctcttagcaaaaacatgtatgtgcatttcaatcccacgtacttataaagaaggttggtgaatttcacaagctatttttggaatatttttagaatattttaagaatttcacaagctattccctcaaatctgagggagctgagtaacaccatcgatcatgatgtagagtgtggttatgaactttaaagttatagttgttttatatgttgctataataaagaagtgttctgcattcgtcaaaaaaaaaaaaaaaaaaaa

SEQ ID NO: 1426 - protein, 373 aa, Mus musculus
MATSASSHLNKGIKQMYMSLPQGEKVQAMYIWVDGTGEGLRCKTRTLDCEPKCVEELPEWNFDGSSTFQSEGSNSDMYLHPVAMFRDPFRKDPNKLVLCEVFKYNRKPAETNLRHICKRIMDMVSNQHPWFGMEQEYTLMGTDGHPFGWPSNGFPGPQGPYYCGVGADKAYGRDIVEAHYRACLYAGVKITGTNAEVMPAQWEFQIGPCEGIRMGDHLWIARFILHRVCEDFGVIATFDPKPIPGNWNGAGCHTNFSTKAMREENGLKCIEEAIDKLSKRHQYHIRAYDPKGGLDNARRLTGFHETSNINDFSAGVANRGASIRIPRTVGQEKKGYFEDRRPSANCDPYAVTEAIVRTCLLNETGDEPFQYKN

SEQ ID NO: 1427 - DNA, 2782 bp, Mus musculus
cagagcggagaatgggagtagagcagagtgtctgaacagcacgctcacccatctcctctccgcctcgctctcctgacctgttcacccatccatcatccggccggccaccgctctgaacaccttccaccatggccacctcagcaagttcccacttgaacaaaggcatcaagcaaatgtacatgtccctgccccagggtgagaaagtccaagccatgtatatctgggttgatggtaccggagaaggactgcgctgcaagacccgtaccctggactgtgagcccaagtgtgtggaagagttacctgagtggaactttgatggctctagtacctttcagtctgaaggctccaacagcgacatgtacctccatcctgttgccatgtttcgagaccccttccgcaaagaccccaacaagctggtgctatgtgaagttttcaagtataaccggaaacctgcagagaccaacttgaggcacatctgtaaacggataatggacatggtgagcaaccagcacccctggtttggaatggagcaggaatatactcttatgggaacagacggccacccatttggttggccttccaatggcttccctggaccccaaggcccgtattactgcggtgtgggagcagacaaggcctacggcagggacatcgtggaggctcactaccgggcctgcttgtatgctggagtcaagattacggggacaaatgcggaggttatgcctgcccagtgggaattccagataggaccctgtgaggggatccgaatgggagatcatctttggatagcccgttttatcttgcatcgggtgtgcgaagactttggggtgatagcaacctttgaccccaagcccattccagggaactggaatggtgcaggctgccataccaacttcagcaccaaggccatgcgggaggagaatggtctgaagtgcattgaggaggccattgacaaactgagcaagaggcaccagtaccacattcgcgcctacgatcccaaggggggcctggacaacgcccggcgtctgactggattccacgaaacctccaacatcaacgacttttctgccggtgttgccaaccgcggtgccagtatccgcattccccggactgtcggccaggagaagaagggctactttgaagaccgtcggccttctgccaattgtgacccctatgcggtgacagaagccatcgtccgcacgtgtctcctcaacgaaacaggcgacgaacccttccaatacaagaactaagtggactagacttccagtgatccctctcccagctcttccctttcccagttgtccccactgtaactcaaaaggatggaataccaaggtctttttattcctcgtgcccagttaatcttgcttttgttggtcagaatagaggggtcaggttcttaatctctacacaccaaccctttctttcctatctagctttctagtagggagcgggaggggggaggggaagggtaacccactgcttcatctcatcgggtatgcatgtccggtaggcatagctgtcacaaagcgggtgtacttatggtgaaagaggacattttttttttcttcaggatagttgaaagggcaggcccaacggctgagattgacatttccactgttggtagagagctgttatttctaaaggggaaaccagctttctgttccaaatggaagttaggtgaggagttgaaggttggtttcttgcgctgtgcttccttggcttgggggagggggcatccgtccccctctgtgtgaacacagctcaccgcgtcacctgatggatggccctactgtgaaggaagaaaaaagttggcatttcttggtcctccgttcataacacaaagcagagtagtatttttatatttaaatgttaaaaacaaaaaagttatatatatatggatgtgtggatgtatgtctttctaattgagagaaccatcctattcactgggtgccaagtttgagtgatgaggggcttggcttagaagtgaggctcccttgaggtaggggtgaggatgcagtaccgggaaagttggttatcttggggtctcagcttcattactgcttagggtttccctgcccactctgcaggagcagatgttggacaggtagccagtgggatgccactgcttgccaccacctgtccccaggcttaggtttaggggatgcgtatacttactccacacacgagttagaagtatgagttggctggtcaacttgaacactgttactgatgggtgggtgggtgggggtttactggggttatttttttggtgggattagcatgtcactaaagcgggccttttgatatattaagttttttaaaagcaaaacaagtttagattttaatcagatttgtagggtttctaactttacagaattgcctgtttgtttcaatgtctccctccacttggctcttaggggaattaaggacaggcctagagttaaaacacttgtctcctagtgtcacctctgccagcagactgttactttccttctgaaaaagccaatagtctttttttttttcttttatagtaaacacacccccacctccatcccagcctgttgcccttcagttttctggttgtttgtgtcggcagcgggccaactgtggtttctctcttgccatgatgacttctaattgccatgtatagtatgttcggttagataactcactgtaaacagactgtaactgccaggcagcgcttataaatcaacctaacatttataagatttcctctgacttgtttctttgtggttcccaaaaaaaaaaaaaaaacctc

SEQ ID NO: 1428 - protein, 447 aa, Cricetulus griseus
DPYINIDAGTFSPYEHGEVFVLDDGGEVDLDLGNYERFLDIRLTKDNNLTTGKIYQYVINKERKGDYLGKTVHVVPHITDAIQRWVMRQALIPVDEDGLEPQVCVIELGGTVGDIESMPFIEAFRQFQFKVKRENFCNIHVSLVPQPSSTGEQKTKPTQNSVRELRGLGLSPDLVVCRCSNPLDTSVKEKISMFCHVEPEQVICVHDVSSIYRVPLLLEEQGVVDYFLRSLELPIERQSRKMLMKWKEMADRYDRLLETCSIALVGKYTKLSDSYASVIKALEHSALAINHKLEIKYIDSTDLEPSTLQEEPVRYHEAWQKLCSAHGVLVPGGFGVRGTEGKIQAIAWARKQKKPFLGVCLGMQLAVVEFSRNVLGWQDANSTEFDPKTSHPVVIDMPEHNPGQMGGTMRLGKSRTLFQTKNSVMSKLYGDTDYLEERHRHRFEVNP

SEQ ID NO: 1429 - DNA, 1341 bp, Cricetulus griseus
gacccctacattaacattgatgcaggaacattctctccttatgagcatggagaggtctttgtactggatgatggtggagaagttgaccttgacttgggaaattatgaacggttccttgatatccgcctcaccaaggacaataatctgaccacagggaagatataccagtatgttattaacaaggaacgcaaaggagattacttggggaaaactgtacacgttgtccctcacatcacagatgcaatccagcgctgggtgatgagacaggccttaatacctgtagatgaagatggcttggagcctcaagtgtgtgttattgagcttggtggcacagtgggagacattgaaagcatgccgtttattgaagccttccggcagttccagttcaaggtcaagagggaaaacttttgtaatatccatgtcagtctggttcctcagccaagttcaacaggggaacaaaagacaaaacctacccagaacagtgttcgggagcttagaggacttgggctttctccagacttggtggtgtgcaggtgctcaaatcctctggacacatctgtgaaagagaaaatatcaatgttctgccatgttgaacctgaacaagtgatctgtgtccatgatgtttcatctatctaccgtgtacccttgttgttagaagagcaaggggttgtagactattttcttcggagccttgagcttcctattgagagacagtcacgaaaaatgctgatgaaatggaaggagatggcagacagatatgatcgcttgttggagacctgctctatcgctcttgtgggcaaatacaccaaactctcagactcctatgcatctgtcattaaagctctagagcattctgcattggctattaaccacaagttagagataaagtacattgattccacagacctggagcctagcaccctgcaggaggagcctgtgcgttaccatgaggcatggcagaagctctgcagtgctcatggagtgctggttccaggaggatttggtgttcgaggaacagaaggaaaaattcaagcaattgcctgggctcggaaacagaagaagccttttttgggtgtgtgcttaggaatgcagctagcagtggtagaattctcaagaaatgtgctgggatggcaagatgccaactctacagaatttgaccctaagactagtcatcctgtggtcatagacatgccagaacataaccctgggcagatgggtggaaccatgaggctgggcaagagtagaaccctgttccagaccaagaactcagtcatgagtaaactctatggagacacagactacttggaagagaggcaccgccaccgatttgaggtgaatcca

SEQ ID NO: 1430 - protein, 363 aa, Homo sapiens
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 1431 - DNA, 1566 bp, Homo sapiens
ggcccgttaagaagagcgtggccggccgcggccaccgctggccccagggaaagccgagcggccaccgagccggcagagacccaccgagcggcggcggagggagcagcgccggggcgcacgagggcaccatggcccagacgcccgccttcgacaagcccaaagtagaactgcatgtccacctagacggatccatcaagcctgaaaccatcttatactatggcaggaggagagggatcgccctcccagctaacacagcagaggggctgctgaacgtcattggcatggacaagccgctcacccttccagacttcctggccaagtttgactactacatgcctgctatcgcgggctgccgggaggctatcaaaaggatcgcctatgagtttgtagagatgaaggccaaagagggcgtggtgtatgtggaggtgcggtacagtccgcacctgctggccaactccaaagtggagccaatcccctggaaccaggctgaaggggacctcaccccagacgaggtggtggccctagtgggccagggcctgcaggagggggagcgagacttcggggtcaaggcccggtccatcctgtgctgcatgcgccaccagcccaactggtcccccaaggtggtggagctgtgtaagaagtaccagcagcagaccgtggtagccattgacctggctggagatgagaccatcccaggaagcagcctcttgcctggacatgtccaggcctaccaggaggctgtgaagagcggcattcaccgtactgtccacgccggggaggtgggctcggccgaagtagtaaaagaggctgtggacatactcaagacagagcggctgggacacggctaccacaccctggaagaccaggccctttataacaggctgcggcaggaaaacatgcacttcgagatctgcccctggtccagctacctcactggtgcctggaagccggacacggagcatgcagtcattcggctcaaaaatgaccaggctaactactcgctcaacacagatgacccgctcatcttcaagtccaccctggacactgattaccagatgaccaaacgggacatgggctttactgaagaggagtttaaaaggctgaacatcaatgcggccaaatctagtttcctcccagaagatgaaaagagggagcttctcgacctgctctataaagcctatgggatgccaccttcagcctctgcagggcagaacctctgaagacgccactcctccaagccttcaccctgtggagtcaccccaactctgtggggctgagcaacatttttacatttattccttccaagaagaccatgatctcaatagtcagttactgatgctcctgaaccctatgtgtccatttctgcacacacgtatacctcggcatggccgcgtcacttctctgattatgtgccctggccagggaccagcgcccttgcacatgggcatggttgaatctgaaaccctccttctgtggcaacttgtactgaaaatctggtgctcaataaagaagcccatggctggtggcatgcaaaaaaaaaaaaaaaaaaaaaaaaaaaaaaaaaaa

SEQ ID NO: 1432 - protein, 352 aa, Mus musculus
MAQTPAFNKPKVELHVHLDGAIKPETILYFGKKRGIALPADTVEELRNIIGMDKPLSLPGFLAKFDYYMPVIAGCREAIKRIAYEFVEMKAKEGVVYVEVRYSPHLLANSKVDPMPWNQTEGDVTPDDVVDLVNQGLQEGEQAFGIKVRSILCCMRHQPSWSLEVLELCKKYNQKTVVAMDLAGDETIEGSSLFPGHVEAYEGAVKNGIHRTVHAGEVGSPEVVREAVDILKTERVGHGYHTIEDEALYNRLLKENMHFEVCPWSSYLTGAWDPKTTHAVVRFKNDKANYSLNTDDPLIFKSTLDTDYQMTKKDMGFTEEEFKRLNINAAKSSFLPEEEKKELLERLYREYQ

SEQ ID NO: 1433 - DNA, 1626 bp, Mus musculus
gagagccatcgggaagcgaccctgccagcgagccaacgcagacccagagagcttcggcggagagaaccgggaacacgctcggaaccatggcccagacacccgcattcaacaaacccaaagtagagttacacgtccacctggatggagccatcaagccagaaaccatcttatactttggcaagaagagaggcatcgccctcccggcagatacagtggaggagctgcgcaacattatcggcatggacaagcccctctcgctcccaggcttcctggccaagtttgactactacatgcctgtgattgcgggctgcagagaggccatcaagaggatcgcctacgagtttgtggagatgaaggcaaaggagggcgtggtctatgtggaagtgcgctatagcccacacctgctggccaattccaaggtggacccaatgccctggaaccagactgaaggggacgtcacccctgatgacgttgtggatcttgtgaaccagggcctgcaggagggagagcaagcatttggcatcaaggtccggtccattctgtgctgcatgcgccaccagcccagctggtcccttgaggtgttggagctgtgtaagaagtacaatcagaagaccgtggtggctatggacttggctggggatgagaccattgaaggaagtagcctcttcccaggccacgtggaagcctatgagggcgcagtaaagaatggcattcatcggaccgtccacgctggcgaggtgggctctcctgaggttgtgcgtgaggctgtggacatcctcaagacagagagggtgggacatggttatcacaccatcgaggatgaagctctctacaacagactactgaaagaaaacatgcactttgaggtctgcccctggtccagctacctcacaggcgcctgggatcccaaaacgacgcatgcggttgttcgcttcaagaatgataaggccaactactcactcaacacagacgaccccctcatcttcaagtccaccctagacactgactaccagatgaccaagaaagacatgggcttcactgaggaggagttcaagcgactgaacatcaacgcagcgaagtcaagcttcctcccagaggaagagaagaaggaacttctggaacggctctacagagaataccaatagccaccacagactgacgggcgggtcccctgaagatggcaaggccacttctctgagcctcatcctgtggataaagtctttacaactctgacatattgaccttcattccttccagaccttggagaggccaggtctgtcctctgattggatatcctggctaggtcccaggggacttgacaatcatgcacatgaattgaaaaccttccttctaaagctaaaattatggtgttcaataaagcagctggtgactggtatcttgcagcacatggtgaatatggtctcggggctgctggctaggatgctaagaaaggaggagccctgggccctacgctgagtgtcaggctggggagccagggtctctttcctgcagaagcgattctttcccagaggggctgttggagcagatgctcctgaactctccgcccctttaaccagtcctttggatttatttttattatttttaaatatttaattatgtttatgt

SEQ ID NO: 1434 - protein, 361 aa, Rattus norvegicus
MAAADEPKPKKLKVEAPEALSENVLFGMGNPLLDISAVVDKDFLDKYSLKPNDQILAEDKHKELFDELVKKFKVEYHAGGSTQNSMKVAQWMIQEPHRAATFFGCIGIDKFGEILKSKAADAHVDAHYYEQNEQPTGTCAACITGGNRSLVANLAAANCYKKEKHLDLENNWMLVEKARVYYIAGFFLTVSPESVLKVARYAAENNRTFTLNLSAPFISQFFKEALMEVMPYVDILFGNETEAATFAREQGFETKDIKEIARKTQALPKVNSKRQRTVIFTQGRDDTIVATGNDVTAFPVLDQNQEEIVDTNGAGDAFVGGFLSQLVSNKPLTECIRAGHYAASVIIRRTGCTFPEKPNFH

SEQ ID NO: 1435 - DNA, 1763 bp, Rattus norvegicus
gagagtgcgaagatggcagctgcggacgagccgaagcccaagaagctcaaggtggaagcgccagaagcgctgagtgaaaatgtgctgtttggaatggggaatcctcttcttgacatctctgctgtggtagacaaagatttccttgataagtattctctgaaaccaaacgaccagatcttggccgaagacaagcacaaggaattgtttgatgaacttgtaaaaaaattcaaagttgaatatcatgccggtgggtccacgcagaattcaatgaaagtggctcagtggatgattcaggagccacacagagcagcaacgttcttcggatgcattgggatagataagttcggggagatcctgaagagcaaagccgcagatgcacacgtggacgcccattactatgagcagaacgagcagcccacaggaacgtgcgctgcatgcatcaccggtggcaaccggtctcttgttgctaaccttgctgccgccaattgttataagaaagaaaagcaccttgatctggagaacaactggatgttggtagagaaagccagagtttactacatagctggcttctttctcaccgtctccccagagtcagtgttgaaagtggctcgctatgctgccgagaacaacaggaccttcactctgaacctgtccgcaccgttcattagccagttcttcaaggaagccttgatggaagtcatgccttatgttgacatcctctttggaaatgagacggaggctgccacttttgctagagagcaaggctttgagactaaagacattaaagaaatagccagaaagacgcaggctcttccaaaggtgaactcgaagaggcagaggaccgtgatcttcacccaagggagagatgacactatagtggctacaggaaatgatgtcactgctttccctgtcttggatcaaaaccaggaagagatcgttgacaccaatggagctggagatgcatttgtaggagggtttctgtctcagctggtctccaacaagcctctgactgaatgcatccgggccgggcactatgcagcgagcgtcatcattaggcgaactggctgtacttttcctgagaagccaaacttccactgacggaagaaaagcaactcaggctgtggagtggagactgcagagaccacgtcctgagcgttcctgccttgagaaagaataacattatcttctgtctttgcccaccatgattctcattattaccgtagagggctcagtgctactcctagaacctttagtctctcaaaatctggggaaaaatgtttatttccatagtctgttagtgcctcttaaatgtcacacaggaatccaacatttcagtagaaacttttaagttccttttcaattgtttataaattcatcagtatttagtaagttgatttttttttatatttctgcttctaatgcgggtgcaatttaatatgatagactttttaaagagattaatgctaacgtcattcttgagctttttatataaatgtgtttaatttggaagcgtgtgtgcacacatgtcatatatattcctgataattgtcaaataaggtacagaaacgttgacgcacaccacattgccccgcataagctcttccatgtgctgcggacacccggtggacttggggggacagctgactaatcccagctttgcgctgtgctcagattgtaacctctgcctactcatggtactctgtaatttgatacaaataaaaaactgccacatagggaaaaaaaaaaaaaaaaaaaaaaaaaaa

SEQ ID NO: 1436 - protein, 344 aa, Mus musculus
MCAVLRQPKCVKLRALHSACKFGVAARSCQELLRKGCVRFQLPMPGSRLCLYEDGTEVTDDCFPGLPNDAELLLLTAGETWHGYVSDITRFLSVFNEPHAGVIQAARQLLSDEQAPLRQKLLADLLHHVSQNITAETREQDPSWFEGLESRFRNKSGYLRYSCESRIRGYLREVSAYTSMVDEAAQEEYLRVLGSMCQKLKSVQYNGSYFDRGAEASSRLCTPEGWFSCQGPFDLESCLSKHSINPYGNRESRILFSTWNLDHIIEKKRTVVPTLAEAIQDGREVNWEYFYSLLFTAENLKLVHIACHKKTTHKLECDRSRIYRPQTGSRRKQPARKKRPARKR

SEQ ID NO: 1437 - DNA, 1038 bp, Mus musculus
atgtgcgcggtgctccgccaacccaaatgcgtcaagttgcgagccctacatagcgcctgcaagttcggcgtggcggcccggagctgccaggagctgctgcgtaagggctgcgtccgcttccagctcccgatgcccggttcccggctgtgcctgtacgaagatggcacggaggtgacggacgactgcttcccgggccttcccaacgacgctgagctcctattgctcaccgctggcgagacctggcatggctatgtgagtgacatcacacgtttcctcagtgtgtttaatgagccacatgccggcgtcatccaggctgcacggcaactgctgtcagatgagcaggccccactgaggcaaaagctgctggccgatcttctgcatcacgtgagccagaatattactgcagagacccgggagcaggacccatcctggtttgaaggtttggagtcgagattcaggaataagtcgggctatctgagatacagctgtgagagtcggatccggggttacctaagagaggtgagcgcttacacctctatggtggatgaagcagctcaagaagagtacctgcgagtccttggctccatgtgccagaagctcaaatcggtgcagtacaatggcagctatttcgacagaggtgcagaggccagcagccgcctctgtactccagaaggatggttctcctgccagggcccctttgacctggagagctgtctttccaagcactccatcaacccctatggcaacagagagagccggatcctcttcagtacctggaacctggatcatataatagagaagaagcgcaccgtggtacccacgctggctgaagccatccaggatgggagggaggtgaactgggagtacttctacagcctgctcttcactgccgagaacctgaagctggtgcacatcgcctgccacaagaagaccacacacaagctggagtgcgaccgcagtaggatctatcggcctcagacaggatccaggaggaagcagcctgctcggaagaagcgccctgctcggaagcgctagtga

SEQ ID NO: 1438 - protein, 2225 aa, Mesocricetus auratus
MAALVLEDGSVLQGRPFGAAVSTAGEVVFQTGMVGYPEALTDPSYKAQILVLTYPLIGNYGIPSDEEDEFGLSKWFESSENHVAGLVVGECCPTPSHWSATCTLHEWLQQHGIPGLQGVDTRELTKKLREQGSLLGKLVQSGTEPSTLPFVDPNARPLAPEVSIKTPRVFNAGGAPRICALDCGLKYNQIRCLCQLGAEVTVVPWNHELDSQKYDGLFLSNGPGDPASYPGVVATLNRVLSEPNPRPVFGICLGHQLLALAIGAKTYKMRYGNRGHNQPCLLVGTGRCFLTSQNHGFAVDADSLPAGWTPLFTNANDCSNEGIVHDSLPFFSVQFHPEHRAGPSDMELLFDVFLETVREAVAGNPGGQTVKERLVQRLCPPGLLIPGSGLPPPRKVLILGSGGLSIGQAGEFDYSGSQAIKALKEENIQTLLINPNIATVQTSQGLADKVYFLPITPHYVTQVIRNERPDGVLLTFGGQTALNCGVELTKAGVLARYGVRVLGTPVETIELTEDRRAFAARMAEIGEHVAPSEAANSLEQAQAAAERLGYPVLVRAAFALGGLGSGFASTKEELSALVAPAFAHTSQVLIDKSLKGWKEIEYEVVRDAYGNCVTVCNMENLDPLGIHTGESIVVAPSQTLNDREYQLLRRTAIKVTQHLGIVGECNVQYALNPESEQYYIIEVNARLSRSSALASKATGYPLAYVAAKLALGIPLPELRNSVTGGTAAFEPSLDYCVVKIPRWDLSKFLRVSTKIGSCMKSVGEVMGIGRSFEEAFQKALRMVDENCVGFDHTVKPVSDVELETPTDKRIFVVAAALWAGYSVERLYELTRIDCWFLHRMKRIVTHAQLLEQHRGQPLSQDLLHQAKCLGFSDKQIALAVLSTELAVRKLRQELGICPAVKQIDTVAAEWPAQTNYLYLTYWGNTHDLDFRTPHVLVLGSGVYRIGSSVEFDWCAVGCIQQLRKMGYKTIMVNYNPETVSTDYDMCDRLYFDEISFEVVMDIYELENPDGVILSMGGQLPNNMAMALHRQQCRVLGTSPEAIDSAENRFKFSRLLDTIGISQPQWRELSDLESARQFCQTVGYPCVVRPSYVLSGAAMNVAYTDGDLERFLSSAAAVSKEHPVVISKFIQEAKEIDVDAVACDGVVSAIAISEHVENAGVHSGDATLVTPPQDITPKTLERIKAIVHAVGQELQVTGPFNLQLIAKDDQLKVIECNVRVSRSFPFVSKTLGVDLVALATRIIMGEKVEPIGLMTGSGVVGVKVPQFSFSRLAGADVVLGVEMTSTGEVAGFGESRCEAYLKAMLSTGFKIPKKNILLTIGSYKNKSELLPTVRLLESLGYSLYASLGTADFYTEHGVKVTAVDWHFEEAVDGECPPQRSILDQLAENHFELVINLSMRGAGGRRLSSFVTKGYRTRRLAADFSVPLIIDIKCTKLFVEALGQIGPAPPLKVHVDCMTSQKLVRLPGLIDVHVHLREPGGTHKEDFASGTAAALAGGVTMVCAMPNTRPPIIDAPALALAQKLAEAGARCDFALFLGASSENAGTLGAVAGSAAGLKLYLNETFSELRLDSVAQWMEHFETWPSHLPIVAHAERQSVAAVLMVAQLTQRPVHICHVARKEEILLIKTAKAQGLPVTCEVAPHHLFLNREDLERLGPGRGEVRPELGSREDMEALWENMAVIDCFASDHAPHTLEEKCGPKPPPGFPGLETMLPLLLTAVSEGRLSLDDLLQRLHHNPRRIFHLPLQEDTYVEVDLEHEWTIPSHMPFSKARWTPFEGQKVKGTIRRVVLRGEVAYIDGQVLVPPGYGQDVRKWPQGAVPQPPPSAPATTEITTTPERPRRVIPGLPDGRFHLPPRIHRASDPGLPAEEPKEKPSRKVVEPELMGTPDGPCYPAPPVPRQASPQNLGSSGLLHPQTSPLLHSLVGQHILSVKQFTKDQMSHLFNVAHTLRMMVQKERSLDILKGKVMASMFYEVSTRTSSSFAAAMARLGGAVLSFSEATSSVQKGESLADSVQTMSCYADVVVLRHPQPGAVELAAKHCRRPVINAGDGVGEHPTQALLDIFTIREELGTVNGMTITMVGDLKHGRTVHSLACLLTQYRVSLRYVAPPSLRMPPSVWDFVASRGTKQEEFESIEEALPDTDVLYMTRIQKERFGSTQEYEACFGQFILTPHIMTRAKKKMVVMHPMPRVNEISVEVDSDPRAAYFRQAENGMYIRMALLATVLGRF

SEQ ID NO: 1439 - protein, 461 aa, Rattus norvegicus
MGSFTKEEFDCHILDEGFTAKDILDQKINEVSSSDDKDAFYVADLGDVLKKHLRWLKALPRVTPFYAVKCNDSRAIVSTLAAIGTGFDCASKTEIQLVQGLGVPPERIIYANPCKQVSQIKYAASNGVQMMTFDSEIELMKVARAHPKAKLVLRIATDDSKAVCRLSVKFGATLKTSRLLLERAKELNIDVIGVSFHVGSGCTDPETFVQAVSDARCVFDMGTEVGFSMYLLDIGGGFPGSEDTKLKFEEITSVINPALDKYFPSDSGVRIIAEPGRYYVASAFTLAVNIIAKKTVWKEQTGSDDEDESNEQTLMYYVNDGVYGSFNCILYDHAHVKALLQKRPKPDEKYYSSSIWGPTCDGLDRIVERCSLPEMHVGDWMLFENMGAYTVAAASTFNGFQRPNIYYVMSRSMWQLMKQIQSHGFPPEVEEQDVGTLPMSCAQESGMDRHPAACASASINV

SEQ ID NO: 1440 - DNA, 2506 bp, Rattus norvegicus
ccgcgcctcccggccggaaccgatcggggctggtttgagctggtgcgtctccatgacgacgtgctccgcctataagtagcggcgcgtcgcaccgtcgggctttgtcagtccctgcagccgccgccgccggccgccttcagtcagcagctcggcgccacctccggtcggcgactgcggcgggctcgacgaggcggctgacggggcggcggcgggaagacggccgggtgcgccttggggtttagtggcggcttctccatgggtccagccagccgcttccctgtgctgtgaggagacagcattcagagttgaccttgtgagagctggccataatttaattccatctctaggttttctgtcttattgtttcagaggcacatcgagaaccaaccatgggcagctttactaaggaagagtttgactgccatatcctcgatgaaggtttcactgctaaggacattctggaccaaaaaatcaatgaagtttcttcctctgatgataaggatgctttctatgttgcggacctcggagacgttctaaagaagcatctgaggtggctgaaagctcttccccgtgttactcccttctatgctgtcaagtgtaatgacagcagagccatagtgagcaccctggctgccattgggacaggatttgattgtgcaagcaagactgaaatacagttggtgcaggggcttggggtgcctccagagaggattatctatgcaaatccttgtaagcaagtgtctcagatcaagtatgctgccagtaatggagtccagatgatgacttttgacagtgaaattgagttgatgaaagttgccagagcacatccaaaggcaaagttggttttgcggattgccactgatgattccaaagcagtttgtcggctcagtgttaagtttggtgccacactgaaaaccagcaggcttctcttggaacgggcaaaagagctaaatattgatgtcattggtgtcagcttccatgtgggcagtgggtgtactgaccctgagaccttcgtgcaggcagtgtcagatgcccgctgtgtctttgacatgggaacagaagttggtttcagcatgtatctgcttgacattggtggtggctttcctgggtctgaagacacgaagcttaaatttgaggagatcaccagtgtaatcaacccagctctggacaagtacttcccatcggactctggagtgagaatcatagctgagccaggcagatactacgtcgcatcagctttcacacttgcagtgaatatcattgccaaaaaaaccgtgtggaaggagcagaccggctcggacgatgaagatgagtcaaacgagcaaactttgatgtattacgtgaatgatggagtgtatgggtcatttaactgcattctttatgaccatgcacatgtgaaggccctgctgcagaagagacccaagccagatgagaagtattactcatccagcatctggggaccaacatgtgatggccttgatcggatcgtcgagcgctgtagcctgcctgaaatgcatgtgggtgattggatgctgtttgagaacatgggtgcatacactgttgctgctgcttctactttcaatgggttccagaggccaaacatctactacgtaatgtcacggtcaatgtggcaactcatgaagcaaatccagagccatggcttcccgccagaagtggaggagcaggatgttggcactctgcccatgtcttgtgcccaggagagcgggatggaccgtcaccctgcagcctgtgcttctgctagtatcaatgtatagatgccattcttgtagctcttacctgcaagtttagcttgagttcacggcatttggggggaccatttaacttaattactgctagtttggaatgtctttgtaagagtagggttggcaccaatgcagtatggaaagactaggagatgggggtcacacttactgtgttcctatggaaactttgaatattttatatggatttttattcacttttcagacctgatactaatgagtgcccctcggctgctgagcaagcatttgtagcttgtacattggcagaatgggctaaaagcttatgttgtgacccattttgaaaataaagtatcttgaaatgattggacattggagaatgtgtgcaagtatcccttacagaaggcacaaacttctgcacaggctgtgtgttacagcagtgagtctagcccagcagagatgtggatgatacaaagctgtgccccctctgtacagcatcaatgtgcttagcccatctcaagtgtttactgtgaacttggtgcccaaagtctcttaagagtgtcatctgcctagtggcctcttgacttggccacttcctaaggagagggcatctgaggctctttgaaccttgcctgcagaaaccctgactgctccctcaacccttggcctcagctgcatcaccacctagtaacagttgaagtatcgaacagcacttggcagtcaacttcctgtaataaattcaacaacagcaaaaaaaaaaaaaaaaa

SEQ ID NO: 1441 - protein, 461 aa, Mus musculus
MSSFTKDEFDCHILDEGFTAKDILDQKINEVSSSDDKDAFYVADLGDILKKHLRWLKALPRVTPFYAVKCNDSRAIVSTLAAIGTGFDCASKTEIQLVQGLGVPAERVIYANPCKQVSQIKYAASNGVQMMTFDSEIELMKVARAHPKAKLVLRIATDDSKAVCRLSVKFGATLKTSRLLLERAKELNIDVIGVSFHVGSGCTDPETFVQAVSDARCVFDMATEVGFSMHLLDIGGGFPGSEDTKLKFEEITSVINPALDKYFPSDSGVRIIAEPGRYYVASAFTLAVNIIAKKTVWKEQPGSDDEDESNEQTFMYYVNDGVYGSFNCILYDHAHVKALLQKRPKPDEKYYSSSIWGPTCDGLDRIVERCNLPEMHVGDWMLFENMGAYTVAAASTFNGFQRPNIYYVMSRPMWQLMKQIQSHGFPPEVEEQDDGTLPMSCAQESGMDRHPAACASARINV

SEQ ID NO: 1442 - DNA, 2547 bp, Mus musculus
gcctcccggccggaaccgatcgcggctggtttgagctggtgcgtctccatgacgacgtgctcggcgtataagtagcggcgcgtcgcaccgtcgggctttgtcagtccctgcagccgccaccgccggccgccctcagccagcagctcggcgccacctccggtcggcgtctgcggcgggctcgacgaggcggctgacggggcggcggcggcgggcggacggacggacggacgggcgctcctcggggtttggcggcggcgcctccatgggtcaggccagccgggccaccgtgctgtgagtgtttccaccactccaagaaggcagcattcagagttcttggctaagtcgaccttgtgaggagctggtgataatttgattccatctccaggttccctgtaagcacatcgagaaccatgagcagctttactaaggacgagtttgactgccacatccttgatgaaggctttactgctaaggacattctggaccaaaaaatcaatgaagtctcttcctctgacgataaggatgcgttctatgttgcggacctcggagacattctaaagaagcatctgaggtggctaaaagctcttccccgcgtcactcccttttacgcagtcaagtgtaacgatagcagagccatagtgagcaccctagctgccattgggacaggatttgactgtgcaagcaagactgaaatacagttggtgcaggggcttggggtgcctgcagagagggttatctatgcaaatccttgtaaacaagtctctcaaatcaagtatgctgccagtaacggagtccagatgatgacttttgacagtgaaattgaattgatgaaagtcgccagagcacatccaaaggcaaagttggttctacggattgccactgatgattccaaagctgtctgtcgcctcagtgttaagtttggtgccacactcaaaaccagcaggcttctcttggaacgggcaaaagagctaaatattgacgtcattggtgtgagcttccatgtgggcagtggatgtactgatcctgagaccttcgttcaggcagtgtcggatgcccgctgtgtgtttgacatggcaacagaagttggtttcagcatgcatctgcttgatattggtggtggctttcctggatctgaagatacaaagcttaaatttgaagagatcaccagtgtaatcaacccagctctggacaagtacttcccatcagactctggagtgagaatcatagctgagccaggcagatactatgtcgcatcagctttcacgcttgcagtcaacatcattgccaaaaaaaccgtgtggaaggagcagcccggctctgacgatgaagatgagtcaaatgaacaaaccttcatgtattatgtgaatgatggagtatatggatcatttaactgcattctttatgatcatgcccatgtgaaggccctgctgcagaagagacccaagccagacgagaagtattactcatccagcatctggggaccaacatgtgatggccttgatcggatcgtggagcgctgtaacctgcctgaaatgcatgtgggtgattggatgctgtttgagaacatgggtgcatacactgttgctgctgcttctactttcaatgggttccagaggccaaacatctactatgtaatgtcacggccaatgtggcaactcatgaagcagatccagagccatggcttcccgccggaggtggaggagcaggatgatggcacgctgcccatgtcttgtgcccaggagagcgggatggaccgtcaccctgcagcctgtgcttctgctaggatcaatgtgtagatgccattcttgtagctcttgcctgcaagtttagcttgtgttaaggcatttggggggaccatttaacttactgctagtttgggatgtctttgtgagagtagggttggcaccaatgcagtatggaaggctaggagatggggggtcacacttactgtgttcctatggaaactttgaatatttgtattacatggatttttattcacttttcagacatgatactaacgtgtgcccctcagctgctgagcaagcgtttgtagcttgtacattggcagaatgggccagaagcttatgttgtgacccattgtgaaaataaagtatcttgaaataactgggcatcagggaatgtttgcaagtatccttaaagaaggcaccaacatctgcacaggctgctgtgtcatggagagacccactgcctgtggatctgaaggttgggctagccccgcatagcacagaggagaggtggatggcacaaggctgtgccctctctgtacagcatcagtctgcttagcccatcccaagtgtgcagttggctgagaactttgttgcccagagtctgttggtgaggaatgtcacctgcctagtgaccggttggcatggccacttcctagggaggtcatctgaagtccttgcctgcagaaaccctgactgttccctcaacccttgactcccaattgcatcaccacctagtaacagttgggagtatcatacaacatcggcagtcaacttcctgtaataaattcaacaacagcaa

SEQ ID NO: 1443 - protein, 293 aa, Cricetus cricetus
LSVKFGATLRTSRLLLERAKELNIDVIGVSFHVGSGCTDPETFVQALSDARCVFDMGTEVGFSMYLLDIGGGFPGSEDTKLKFEEITSVINPALDKYFPPDSGVRVIAEPGRYYVASAFTLAVNIIAKKIVSKGSDDEDESSEQTFMYYVNDGVYGSFNCILYDHAHVKPLLPKRPKPDEKYYSSSIWGPTCDGLDRIVERCNLPEMHVGDWMLFENMGAYTVAAASTFNGFQRPSIYYVMSRPMWQLMKQIQNHGFPPEVEEQDVGTLPISCAQESGMDRHPAACASASINV

SEQ ID NO: 1444 - DNA, 1609 bp, Cricetus cricetus
cctcagtgtaaagtttggtgccacactcagaaccagcaggcttctcttggaacgggcaaaagagctaaatattgatgtcattggtgtcagcttccacgtggggagtggatgtactgaccctgagaccttcgtccaggccttgtcggatgcccgctgtgtctttgacatgggaacagaagttggtttcagcatgtatctgcttgatattggtggtggctttcctggatctgaggataccaagcttaaatttgaagagatcaccagtgttatcaacccagctctggacaagtacttcccgccagactctggagtgagagttatagccgagccaggcagatactacgttgcctcagctttcacactggcagtcaatatcatagccaagaaaatcgtatcgaagggctctgacgatgaagatgagtccagtgagcaaaccttcatgtattatgtgaatgatggagtgtatgggtcatttaactgcattctttacgatcatgcacatgtgaagcccctgctgccgaagagacccaagccagatgagaagtattactcatccagcatctggggaccaacatgcgatggccttgaccggattgtggagcgctgtaatctgcctgaaatgcatgtgggtgattggatgctctttgagaacatgggtgcatacactgttgctgctgcatctactttcaacgggttccagaggccttctatctactatgtgatgtcgaggccaatgtggcagcttatgaagcagatccagaaccatggcttcccaccagaagtggaagagcaggatgttggcactctgcccatctcttgtgcccaggagagcgggatggaccgtcatccagcagcctgtgcttctgctagtatcaatgtgtagatgccattcttgtagctcttaccttcaagtttagcttgaattaagggatttggggggaccatttaacttaattactgttagttttgaaatgactttgtaactacagagtagggcttggcacagatgaagtatggaaggctaggagatgggtcacacttacctgtgttcctatggaaactttgaatatttgttttatatggatttttatcacttttcagacatactaacgtgtgcccctcagccactgagcaagcatttgtagcttgtacatttggcagaatgggccaaaagctaatgttgtgacccattttgaaaataaactatcttgaaataattgggcatcagggaatgtttgcaatgtcccttaaagaagggacacacttcctgcacaggctgctgtagtcatacgagtgagtccaaacatagcagagaaggaaaaggtggatgggacaaggctataccctctacacagcatcagtttgcctagccagtccttaaagtgtgtacttggctgtgaagttggtgaggaacgtcatctgcctagcgacctgatatcctggccacaccccagggaaggcatctaaggctttttgaaccttgcctgcagaagccttgaatgttcctcaacccttgacttcgcctgcatcaccacctagtaacagttggaagtatcatacagcacttggcaatcaacttcctgtaataaatctcaacagcagcaactac

SEQ ID NO: 1445 - DNA, 2214 bp, Cricetulus longicaudatus
ctaaaacatcccaagtagggagacttctcattggtcagctaggtaacctggggtagccagtgaggccctgggccgacctctgaggtcatcccgcatacagggtcacagcacctaaaagaacagaagcaagccgaattatctcctaactctcttggccccaagctgcctcttcgcgatggcctcagccccgcctggcgtcgcacgtcagcactgcgggccacacccctgcgcacgggttgggtccggcgatatgatgaaacttcccgcacgcgttacagtagcccggctgctctaaggtaagaagcagtcctctgctccttcgtgtccctgtgctgcctcctctcccgcctcctctggccctagccaccagcgagcagagtgcctgcagttctcctgcaggatgtgtggcatttgggccctctttggcagcgatgactgcctttccgtgcagtgcctgagtgcaatgaagattgcacacagagggccagatgcatttcgttttgagaatgtcaatggatataccaactgctgctttggatttcaccggttggccgtggttgaccccctgtttggaatgcagccaataagagtgaagaaatacccttatctgtggctctgttacaatggtgaaatttacaaccacaaggcgctacaacaacgctttgaattcgagtatcagaccaatgtggatggtgagattatcctgcatctctatgacaaaggaggtatcgagcaaaccatttgtatgttggatggggtgtttgcattcatcttgctggacactgccaacaagaaagtttttctgggcagagatacctatggagtcaggcccttgtttaaagccatgacagaagatggatttctggctgtgtgttcagaagctaaagggctcgtttccttaaagcactccacaactcccttcttaaaagtggagcccttccttcctggacactacgaagttttggatttaaaaccaaatggcaaagttgcatctgtggagatggtgaaataccatcactgcagagatgaaccactgcacgccctctacgacagcgtggagaaactcttccaaggctttgagttagaaacggtgaagagcaatcttcgtatcctgtttgacagtgctgtcaggaagcgcttgatgacggacaggaggatcggctgccttttatcagggggcctggactcgagcttggttgccgcctccctgctgaagcaactcaaggaggctcaagtacagtatccactccagacatttgcaattggcatggaagacagccctgatctactagccgctagaaaggtggcaaattatattggaagcgagcatcacgaagtcttatttaattctgaagaaggcattcaggccctggatgaggtcatattttccttggaaacttacgacattacaacagttcgagcatctgtaggtatgtatctaatttccaagtatattcggaagaacacagacagcgtggtgatcttctctggagaagggtcagatgaacttacacagggctacatatatttccacaaggctccttctccagagaaagcagaggaggagagtgagagactgctgaaggaactctacctgtttgatgttctccgagccgaccgaactactgctgcccacggtcttgaactgagagtcccgtttctggatcatcggttttcctcctattacttgtcactgccaccagaaatgagaattccaaaaaatgggatagaaaaacatctccttcgagagacgtttgaggactccaacctgctacccaaagagattctctggagaccaaaagaagccttcagtgatgggatcacctcagttaagaactcctggtttaagattctacaggattatgttgaacatcaggttgatgatgaaatgatggcaacagcagcccagaagtttcccttcaatactcccaaaactaaagaaggctattactaccgccagatctttgaacgccattacccaggccgggctgattggctgacccattattggatgcccaagtggatcaatgccaccgatccttctgcccgaactctcacccactataagtcagctgccaaagcttagatattctctaaactctagtgtaaaagtgttcttctcactctgaaggcagagaccgtgagacaaacacagtaatgcaaatcaaccatcaactgctcaggcttacataggcatggaaagaaataaaaaca

SEQ ID NO: 1446 - protein, 561 aa, Cricetulus longicaudatus
MCGIWALFGSDDCLSVQCLSAMKIAHRGPDAFRFENVNGYTNCCFGFHRLAVVDPLFGMQPIRVKKYPYLWLCYNGEIYNHKALQQRFEFEYQTNVDGEIILHLYDKGGIEQTICMLDGVFAFILLDTANKKVFLGRDTYGVRPLFKAMTEDGFLAVCSEAKGLVSLKHSTTPFLKVEPFLPGHYEVLDLKPNGKVASVEMVKYHHCRDEPLHALYDSVEKLFQGFELETVKSNLRILFDSAVRKRLMTDRRIGCLLSGGLDSSLVAASLLKQLKEAQVQYPLQTFAIGMEDSPDLLAARKVANYIGSEHHEVLFNSEEGIQALDEVIFSLETYDITTVRASVGMYLISKYIRKNTDSVVIFSGEGSDELTQGYIYFHKAPSPEKAEEESERLLKELYLFDVLRADRTTAAHGLELRVPFLDHRFSSYYLSLPPEMRIPKNGIEKHLLRETFEDSNLLPKEILWRPKEAFSDGITSVKNSWFKILQDYVEHQVDDEMMATAAQKFPFNTPKTKEGYYYRQIFERHYPGRADWLTHYWMPKWINATDPSARTLTHYKSAAKA

SEQ ID NO: 1447 - protein, 561 aa, Mus musculus
MCGIWALFGSDDCLSVQCLSAMKIAHRGPDAFRFENVNGYTNCCFGFHRLAVVDPLFGMQPIRVRKYPYLWLCYNGEIYNHKALQQRFEFEYQTNVDGEIILHLYDKGGIEKTICMLDGVFAFILLDTANKKVFLGRDTYGVRPLFKAMTEDGFLAVCSEAKGLVSLKHSTTPFLKVEPFLPGHYEVLDLKPNGKVASVEMVKYHHCTDEPLHAIYDSVEKLFPGFDLETVKNNLRILFDNAIKKRLMTDRRIGCLLSGGLDSSLVAASLLKQLKEAQVQYPLQTFAIGMEDSPDLLAARKVANYIGSEHHEVLFNSEEGIQALDEVIFSLETYDITTVRASVGMYLISKYIRKNTDSVVIFSGEGSDELTQGYIYFHKAPSPEKAEEESERLLKELYLFDVLRADRTTAAHGLELRVPFLDHRFSSYYLSLPPDMRIPKNGIEKHLLRETFEDCNLLPKEILWRPKEAFSDGITSVKNSWFKILQDYVEHQVDDEMMSAASQKFPFNTPKTKEGYFYRQIFERHYPGRADWLTHYWMPKWINATDPSARTLTHYKSAAKA

SEQ ID NO: 1448 - DNA, 1928 bp, Mus musculus
caagcggcctccaaccggtcttgtcactgcgctgcctctgctccaccttctctggccctggccgctagtgctcagagtgcctgcagtccgcctgtagcatgtgtggcatctgggccctcttcggcagcgatgactgcctttccgtgcagtgtctgagtgcgatgaagatcgcgcacagggggccagatgcatttcgctttgagaatgtcaatggatacaccaactgctgctttggctttcaccgcttggctgtggttgacccgctgtttggaatgcagccgataagagtgaggaaatacccttatttgtggctctgttacaatggtgaaatctacaaccacaaggcgctacagcaacgttttgaatttgaatatcagaccaatgtggatggtgagattatcctccacctctatgacaaaggaggcatcgagaaaaccatctgtatgctggacggggtgtttgcattcatcttactggacactgccaataagaaagtatttctgggcagagacacctatggagtcaggcccttgtttaaagccatgacagaagatgggtttctggctgtgtgttcagaagctaaaggccttgtttccttaaaacactccaccactcccttcttaaaagtggagcccttccttcctggacactatgaagttttggatttaaaaccaaatggcaaagttgcgtctgtggaaatggtcaaataccatcactgtacggatgaaccattgcatgccatctatgacagcgtggagaaactcttcccaggctttgacctagagaccgtgaagaacaatctgcgtatcctttttgacaacgctatcaagaaacgcttgatgacagaccggaggattggctgccttttatcagggggcctggactcgagcttggttgctgcctctctgctgaagcaactcaaggaggcccaagttcagtatcctctccagacatttgctattggcatggaggacagccccgatctcctggccgctagaaaggtggcaaattatattggaagcgagcatcatgaagtcctttttaactctgaagaaggcattcaggccctggatgaagtcatattttccttggaaacttatgatattacgacagttcgggcatctgtgggcatgtatttaatttccaagtatattcggaagaacacagacagcgtggtgatcttctccggagaggggtcagatgaacttacacagggctatatatatttccacaaggctccttcccctgagaaggccgaggaggagagtgagagactgctgaaggaactctacctgtttgatgttctccgggccgaccgcactactgccgcacatggtctcgaactgagagtcccctttctggatcatcggttttcttcctattacctgtctctgccgccagatatgagaattccaaaaaatggcatagaaaaacatctcctgagagagacttttgaggactgcaacctgctacccaaagagattctctggcgacccaaagaagccttcagtgatgggatcacctcagtcaagaactcctggttcaagattttgcaggactatgttgaacatcaggttgatgatgaaatgatgtctgcagcctcccagaagtttcccttcaatactcccaaaactaaggaaggctacttctaccgtcagatctttgaacgccattacccaggccgggctgattggctgactcattattggatgcctaagtggatcaatgctactgacccttctgcccgcactctgacccattataagtcagctgccaaagcttaggcactctctacactcttgtgtaaaagtaaatgtttcttccggctctgaaggtcgagacagcgacacaatcagaaagaatgagactcagccatcagtcacccaggcttacttaggcatgaaaagaaataaaagtttcacatctga

(19) United States
(12) Patent Application Publication
(10) Pub. No.: US 2013/0164851 A1
(43) Pub. Date: Jun. 27, 2013

(54) GENE AMPLIFICATION AND TRANSFECTION METHODS AND REAGENTS RELATED THERETO

(71) Applicants: Anthony Rossomando, Cambridge, MA (US); Gregory P. Thill, Cambridge, MA (US); Stuart Pollard, Cambridge, MA (US)

(72) Inventors: Anthony Rossomando, Cambridge, MA (US); Gregory P. Thill, Cambridge, MA (US); Stuart Pollard, Cambridge, MA (US)

(73) Assignee: ALNYLAM PHARMACEUTICALS, INC., Cambridge, MA (US)

(21) Appl. No.: 13/636,379

(22) PCT Filed: Mar. 25, 2011
(86) PCT No.: PCT/US2011/029897

§ 371 Date: Mar. 11, 2013

Related U.S. Application Data
(60) Provisional application No. 61/317,968, filed on Mar. 26, 2010.

(51) Int. Cl. C12N 15/85

(52) U.S. Cl. CPC C12N 15/85
USPC 435/465; 435/455; 435/471; 435/468; 435/325; 435/320.1; 435/254.11; 435/419; 435/358

(57) ABSTRACT

Provided herein are methods and compositions for generating a cell line capable of producing a biological product, using a gene amplification based system. Methods and compositions are provided to inhibit endogenous selectable amplifiable marker genes using RNA interference and prevent the selection of false positives during generation of a custom cell line. Such methods improve efficiency of cell line development and do not require the use of specialized substrates or cells lacking the endogenous selectable amplifiable marker gene to negate the effect of endogenously expressed levels of the selectable amplifiable marker gene in cells.

### RELATED APPLICATIONS

This application claims the benefit under 35 U.S.C. §119(e) of U.S. Provisional Patent Application No. 61/317,968, filed Mar. 26, 2010, which is herein incorporated by reference in its' entirety.

### REFERENCE TO SEQUENCES

The instant application contains a Sequence Listing which has been submitted in ASCII format via EFS-Web and is hereby incorporated by reference in its entirety. Said ASCII copy, created on Mar. 22, 2011, is named ABIO-006.txt and is 446,464 bytes in size.

### FIELD OF THE INVENTION

The field of the invention relates to production of a cell for producing a biological product.

### BACKGROUND

Cell culture techniques are used to manufacture a wide range of biological products, including biopharmaceuticals, biofuels, metabolites, vitamins and nutraceuticals. However, there have been few recent advances in the field of customized cell line generation and there is a need for new methods to rapidly customize cell lines for the production of biologics.

One method for customizing cell lines involves using genetic selection schemes to isolate cells that contain multiple copy numbers of a gene required for survival in the presence of a toxic stimulus (e.g., inhibitor, chemotherapeutic agents, lack of an essential metabolite, or removal of an important growth substrate). Known cloned amplifiable genes, whose amplification can be selected for, include those in which the gene product either (a) directly or indirectly interacts with an inhibitor of cell growth so as to render the inhibitor ineffective, or (b) is necessary for cell survival and can be inhibited by exogenously supplied substances. In both instances, the nature of the amplification process is such that increasing amounts of gene product must be produced in the presence of increasing amounts of inhibitor in order for cells to survive. Thus, the stressor is both a gene amplification-inducing agent and a selection agent. This phenomenon has been exploited to produce cells that comprise multiple copies of a transgene that encodes a biological product.

Selectable amplifiable marker genes, e.g., dihydrofolate reductase (DHFR), have been routinely used in combination with mammalian cell lines to generate cells capable of producing a biological product. Typically, a transgene is linked to a selectable amplifiable marker and introduced to cells. The cells are subsequently treated with a stimulus (e.g., a toxic metabolite) under conditions that favor survival of cells containing higher levels of the marker, which is commonly achieved when the selectable amplifiable marker has undergone gene amplification to produce multiple copies of the marker gene. These cells are then selected based on their ability to survive in the presence of the stimulus. Since the transgene is linked to the marker at the nucleic acid level, the transgene copy number is also often increased under these conditions, and the product encoded by the transgene is expressed at a higher level as a consequence of the gene amplification.

One disadvantage of this method for the production of cell lines with amplified genes is that for efficient selection the method ideally relies on the use of cells that lack an endogenously expressed amplifiable marker gene (e.g. DHFR(−)) cells. If the amplifiable marker gene is endogenous to the host cell, selection e.g., for resistance can result in amplification of the host marker gene rather than the selectable amplifiable marker gene that is linked to the transgene. This limits the number of cells that are available for making producer cells. Gene amplification or gene duplication of the endogenous amplifiable marker gene results in a high number of false positives during the selection step. False positives reduce the efficiency of these methods for developing a customized cell line, making customization of cell lines for developing biologics a tedious and inefficient process.

### SUMMARY OF THE INVENTION

Provided herein are methods and compositions for generating a cell line capable of producing a biological product. The present invention is based, in part, on the discovery that the efficiency of making custom cell lines for the production of a biological product using a gene amplification based system is improved by the administration of an RNA effector molecule that inhibits expression of an endogenously expressed selectable amplifiable marker gene. Inhibition of expression of the endogenous selectable amplifiable marker gene enables amplification of a transgene linked to an amplifiable gene that is not significantly inhibited by the RNA effector molecule, e.g. a gene that differs in its nucleic acid sequence yet encodes the same protein as the endogenous marker. The inhibition of expression of the endogenous selectable amplifiable marker genes prevents the selection of false positives during generation of a custom cell line and improves efficiency of cell line development, since only the vector-supplied marker gene and the linked transgene undergo gene duplication. In addition, the methods and compositions provided herein have the added advantage of not requiring removal of substrates from the culture medium (e.g., glutamate) or other auxotrophic mechanisms necessary to negate the effect of endogenously expressed levels of the selectable amplifiable marker gene in cells, nor does it require a cell line that lacks expression of the selectable amplifiable marker gene.

In one aspect, described herein is a method of generating a cell line capable of producing a biological product comprising: (a) providing a plurality of host cells comprising a first selectable amplifiable marker gene and a second selectable amplifiable marker gene, wherein a transgene encoding a biological product is linked to the first selectable amplifiable marker gene, and wherein the first and second selectable amplifiable marker genes each have different nucleic acid sequences and are capable of being amplified using the same amplification reagent; (b) transfecting the host cell of step (a) with an RNA effector molecule, a portion of which is complementary to the second selectable amplifiable marker gene endogenous to the host cell such that the RNA effector molecule inhibits expression of the second selectable amplifiable marker gene; and (c) contacting the transfected cells of step (b) with a progressively increasing amount of the amplification reagent to select for cells with multiple copies of the first selectable amplifiable marker gene and the transgene, thereby generating a cell line that is capable of producing the biological product.

Another aspect described herein relates to a method of generating a cell line capable of producing a biological product comprising: a) transfecting a plurality of host cells with: i) one or more vectors comprising a transgene linked to a first selectable amplifiable marker gene, wherein the transgene encodes a biological product, ii) an RNA effector molecule, a portion of which is complementary to a second selectable amplifiable marker gene endogenous to the host cell such that the RNA effector molecule inhibits expression of the second selectable amplifiable marker gene, wherein the first and second selectable amplifiable marker genes each have a different nucleic acid sequence and are capable of being amplified using an amplification reagent, b) culturing the plurality of host cells of step a) with a first concentration of the amplification reagent to select for viable transfected host cells; c) culturing the viable transfected host cells of step b) with a higher concentration of the amplification reagent than used in step b), thereby selecting for surviving cells that have an increased copy number of the transgene and the first selectable marker gene, wherein cells capable of producing a biological product are generated.

Another aspect described herein relates to methods for increasing the transfection efficiency of cells capable of producing a biological product, comprising transfecting a plurality of host cells with: i) a vector comprising a transgene that encodes a biological product; and ii) an RNA effector molecule that inhibits expression of the transgene, whereby the RNA effector molecule inhibits expression of the transgene thereby increasing the transfection efficiency as compared to the transfection efficiency observed in the absence of the RNA effector molecule.

Another aspect described herein relates to methods for generating a cell line capable of producing a biological product comprising: (a) providing a plurality of host cells comprising a modified selectable amplifiable marker gene, wherein a transgene encoding a biological product is linked to the modified selectable amplifiable marker gene and the nucleic acid sequence for the modified selectable amplifiable marker gene differs from an endogenous selectable amplifiable marker gene in the host cell by at least one nucleotide; (b) transfecting the host cell of step (a) with an RNA effector molecule, a portion of which is complementary to the endogenous selectable amplifiable marker gene such that the RNA effector molecule inhibits expression of the selectable amplifiable marker gene and wherein the RNA effector molecule does not substantially inhibit the modified selectable amplifiable marker gene; and (c) contacting the transfected cells of step (b) with a progressively increasing amount of the amplification reagent to select for cells with multiple copies of the modified selectable amplifiable marker gene and the transgene, thereby generating a cell line that is capable of producing the biological product.

In one embodiment of the aspects described herein, the RNA effector molecule does not significantly inhibit expression of the first selectable marker gene.

In another embodiment of the aspects described herein, the RNA effector molecule transiently inhibits expression of the second selectable amplifiable marker gene.

In another embodiment of the aspects described herein, the RNA effector molecule inhibits expression of the second selectable amplification gene by at least 10%, at least 20%, at least 30%, at least 40%, at least 50%, at least 60%, at least 70%, at least 80%, at least 90%, at least 95%, at least 99%, or 100%.

In another embodiment of the aspects described herein, the RNA effector molecule inhibits expression of the second amplifiable marker gene at least 10%, at least 20%, at least 30%, at least 40%, at least 50%, at least 60%, at least 70%, at least 80%, at least 90%, at least 95%, at least 99%, at least 2-fold, at least 5-fold, at least 10-fold, at least 100 fold, or at least 1000 fold more than the RNA effector molecule inhibits the first selectable amplifiable marker.

In another embodiment of the aspects described herein, the method further comprises transfecting the cell of step a) with a second RNA effector molecule, a portion of which is complementary to the transgene, such that the second RNA effector molecule inhibits expression of the transgene.

In another embodiment of the aspects described herein, the cell that has amplified the transgene is maintained in the presence of the second RNA effector molecule for a period of time before removal of the second RNA effector molecule and expression of the transgene.

In another embodiment of the aspects described herein, the RNA effector molecule inhibits expression of the transgene by an average percent inhibition of at least 10%, at least 20%, at least 30%, at least 40%, at least 50%, at least 60%, at least 70%, at least 80%, at least 90%, at least 95%, at least 99%, or 100%.

In another embodiment of the aspects described herein, the first and second selectable amplifiable marker genes encode a protein selected from the group consisting of: dihydrofolate reductase, thymidylate synthase, glutamine synthetase, adenosine deaminase, carbamoyl-phosphate synthase-aspartate transcarbamoylase-dihydroorotase (CAD), ornithine decarboxylase, and asparagine synthetase.

In another embodiment of the aspects described herein, the first and second selectable amplifiable marker genes do not encode for dihydrofolate reductase.

In another embodiment of the aspects described herein, the first and second selectable amplifiable marker genes are from different species.

In another embodiment of the aspects described herein, the amplification reagent is selected from the group consisting of: methotrexate, N-phosphonoacetyl-L-aspartic acid (PALA), 2′-deoxycoformycin (dCF), 5-fluorouracil (5FU), difluoromethylornithine (DFMO), albizziin, and β-aspartyl hydroxamate (β-AHA).

In other embodiments of the aspects described herein, the biological product is a polypeptide, a metabolite of a nutraceutical.

In other embodiments of the aspects described herein, the cell is an animal cell, a fungal cell, a plant call, or a mammalian cell. In one embodiment, the mammalian cell is a human cell. The human cell can be an adherent cell selected from the group consisting of: SH-SY5Y cells, IMR32 cells, LAN5 cells, HeLa cells, MCF1OA cells, 293T cells, and SK-BR3 cells. Alternatively, the human cell is a primary cell selected from the group consisting of: HuVEC cells, HuASMC cells, HKB-I1 cells, and hMSC cells.

In another embodiment, the human cell is selected from the group consisting of: U293 cells, HEK 293 cells, PERC6® cells, Jurkat cells, HT-29 cells, LNCap.FGC cells, A549 cells, MDA MB453 cells, HepG2 cells, THP-I cells, MCF7 cells, BxPC-3 cells, Capan-1 cells, DU145 cells, and PC-3 cells.

In another embodiment, the mammalian cell is a rodent cell selected from the group consisting of: BHK21 cells, BHK TK− cells, NS0 cells, Sp2/0 cells, EL4 cells, CHO cells, CHO cell derivatives, U293 cells, NIH/3T3 cells, 3T3 L1 cells, ES-D3 cells, H9c2 cells, C2C12 cells, and miMCD-3 cells.

In another embodiment, the CHO cell derivative is selected from the group consisting of: CHO-K1 cells, CHO-DUKX, CHO-DUKX B1, and CHO-DG44 cells.

In another embodiment, the human cell is selected from the group consisting of: PERC6 cells, HT-29 cells, LNCaP-FGC cells A549 cells, MDA MB453 cells, HepG2 cells, THP-I cells, miMCD-3 cells, HEK 293 cells, HeLaS3 cells, MCF7 cells, Cos-7 cells, BxPC-3 cells, DU145 cells, Jurkat cells, PC-3 cells, and Capan-1 cells.

In another embodiment of the aspects described herein, the RNA effector molecule is a double-stranded ribonucleic acid (dsRNA), wherein said dsRNA comprises at least two sequences that are complementary to each other and wherein a sense strand comprises a first sequence and an antisense strand comprises a second sequence comprising a region of complementarity, and wherein said region of complementarity is 15-30 nucleotides in length. In another embodiment, the RNA effector molecule comprises a modified nucleotide.

In another embodiment of the aspects described herein, the nucleic acid sequences of the first and second selectable amplifiable marker differ by at least one nucleotide.

In another embodiment of the aspects described herein, the second RNA effector molecule is transfected immediately before, simultaneously with, or immediately after the vector comprising a transgene.

In another embodiment of the aspects described herein, the transgene and first selectable marker are each provided on a separate vector and are linked co-transformationally in the host genome. Alternatively, the transgene linked to the first selectable marker is provided on a single vector.

Also described herein, in another aspect is a method for generating a cell line capable of producing a biological product, comprising: (a) transfecting a plurality of host cells with: i) a vector comprising a selectable marker and a transgene, wherein the transgene encodes a biological product, and ii) an RNA effector molecule, a portion of which is complementary to a copy of the selectable marker endogenously expressed in the plurality of host cells prior to introduction of the vector of step i), and (b) culturing the cells of step (a) under conditions that select for cells comprising the vector of step i), thereby generating a cell line capable of producing a biological product.

Also described herein are kits useful for generating a cell capable of producing a biological product comprising: a) a vector comprising a selectable amplifiable marker gene that has a nucleic acid sequence distinct from that of the marker gene endogenous to a host cell; b) an RNA effector molecule, a portion of which is complementary to the marker gene endogenous to the host cell; and c) packaging materials and instructions therefor.

In one embodiment, the kit further comprises a host cell.

In another embodiment, the nucleic acid sequence of the selectable amplifiable marker on the vector differs from the nucleic acid sequence of the endogenous marker gene by at least one nucleotide.

In another embodiment, the kit further comprises an amplification reagent.

### Definitions

For convenience, the meaning of certain terms and phrases used in the specification, examples, and appended claims, are provided below. If there is an apparent discrepancy between the usage of a term in other parts of this specification and its definition provided in this section, the definition in this section shall prevail.

“G,” “C,” “A,” “T” and “U” each generally stand for a nucleotide that contains guanine, cytosine, adenine, thymine and uracil as a base, respectively. However, it will be understood that the term “deoxyribonucleotide,” “ribonucleotide,” or “nucleotide” can also refer to a modified nucleotide, as further detailed below, or a surrogate replacement moiety. The skilled person is well aware that a ribonucleotide comprising a thymine base is also referred to as 5-methyl uridine and a deoxyribonucleotide comprising a uracil base is also referred to as deoxy-Uridine in the art. The skilled person is also well aware that guanine, cytosine, adenine, thymine and uracil can be replaced by other moieties without substantially altering the base pairing properties of an oligonucleotide comprising a nucleotide bearing such replacement moiety. For example, without limitation, a nucleotide comprising inosine as its base can base pair with nucleotides containing adenine, cytosine, or uracil. Hence, nucleotides containing uracil, guanine, or adenine can be replaced in the nucleotide sequences of dsRNA featured in the invention by a nucleotide containing, for example, inosine. In another example, adenine and cytosine anywhere in the oligonucleotide can be replaced with guanine and uracil, respectively to form G-U Wobble base pairing with the target mRNA. Sequences containing such replacement moieties are suitable for the compositions and methods featured in the invention.

As used herein, the term “transgene” refers to an exogenously supplied nucleic acid sequence e.g., that encodes a biological product or encodes for a gene product that increases production of the biological product by the cell. The term transgene also encompasses the gene once it has integrated into the host genome. A transgene can be administered by any means known in the art including e.g., vectors, plasmids, viral vectors, incorporation of a transgene into the genome of the host cell. The transgene can be under the control of an inducible promoter, if so desired.

A “biological product” can include any substance capable of being produced by a cultured host cell and recovered in useful quantities, including but not limited to, polypeptides (e.g., glycoproteins, antibodies, peptide-based growth factors), carbohydrates, lipids, fatty acids, metabolites (e.g., polyketides, macrolides), peptidomimetics, and chemical intermediates. The biological products can be used for a wide range of applications, including as biotherapeutic agents, vaccines, research or diagnostic reagents, fermented foods, food additives, nutraceuticals, biofuels, industrial enzymes (e.g., glucoamylase, lipase), industrial chemicals (e.g., lactate, fumarate, glycerol, ethanol), and the like.

In some embodiments, the biological product is a polypeptide. The polypeptide can be a recombinant polypeptide or a polypeptide endogenous to the host cell. In some embodiments, the polypeptide is a glycoprotein and the host cell is a mammalian cell. Non-limiting examples of polypeptides that can be produced according to methods provided herein include receptors, membrane proteins, cytokines, chemokines, hormones, enzymes, growth factors, growth factor receptors, antibodies, antibody derivatives and other immune effectors, interleukins, interferons, erythropoietin, integrins, soluble major histocompatibility complex antigens, binding proteins, transcription factors, translation factors, oncoproteins or proto-oncoproteins, muscle proteins, myeloproteins, neuroactive proteins, tumor growth suppressors, structural proteins, and blood proteins (e.g., thrombin, serum albumin, Factor VII, Factor VIII, Factor IX, Factor X, Protein C, von Willebrand factor, etc.). As used herein, a polypeptide encompasses glycoproteins or other polypeptides which has undergone post-translational modification, such as deamidation, glycation, and the like.

As used herein, the term “target RNA” or “target gene” refers to a nucleic acid sequence of a selectable amplifiable marker gene or a transgene that encodes a biological product or gene product that induces production of a biological product.

A “host cell,” as used herein, is any eukaryotic cell capable of being grown and maintained in cell culture under conditions allowing for production and recovery of useful quantities of a polypeptide, as defined herein. Host cells can be unmodified cells or cell lines, or cell lines which have been genetically modified (e.g., to facilitate production of a polypeptide or biological product). In some embodiments, the host cell is a cell line that has been modified to allow for growth under desired conditions, such as in serum-free media, in cell suspension culture, or in adherent cell culture. In other embodiments, the host cell can be selected from the group consisting of a plant cell, a fungal cell, an insect cell and a mammalian cell. In one embodiments, the host cell is a mammalian cell (e.g., a human cell, a hamster cell, a mouse cell, a rat cell, or a cell line derived thereof).

As used herein, the term “selectable amplifiable marker gene” refers to a gene that permits selection of cells in the presence of an amplification reagent that have undergone gene duplication to produce at least one additional copy of the gene in the host cell. Such gene duplication can occur spontaneously or in response to an amplification reagent (e.g. inhibitor) or a toxic stimulus (e.g., removal of a required growth substrate, hypoxia etc). Duplicated genes can be chromosomal or extra-chromosomal. Generally, duplicated genes present in the chromosome are stable, whereas extra-chromosomal gene duplications are unstable. The selectable amplifiable marker gene is not a gene that promotes death of the host cell. Generally, the selectable amplifiable marker gene encodes a protein necessary for the growth or survival of a host cell, and when the encoded protein is inhibited, e.g. by addition of an amplification reagent, the amplifiable marker is amplified to increase production of the encoded protein to maintain the growth and survival of the cell. A selectable gene will confer resistance to a drug or compensate for a metabolic or catabolic defect in the host cell. Some non-limiting examples of selectable amplifiable marker genes include, but are not limited to, dihydrofolate reductase (DHFR), CAD, adenosine deaminase, thymidylate synthetase, glutamine synthetase, asparagine synthetase, and ornithine decarboxylase.

As used herein the term “linked” in reference to two nucleic acid sequences (e.g., a transgene and a selectable amplifiable marker) indicates that the nucleic acid sequences are linked together using any method known in the art e.g., linked in a tandem arrangement within the host chromosome, or linked on the same integratable vector using the same or different promoters. The term “linked” also encompasses the use of a linker nucleotide or plurality of nucleotides between the two nucleic acid sequences. The term ‘linked’ is not intended to encompass or suggest that the polypeptides produced by the nucleic acid sequences are in any way tethered together (e.g., a fusion protein). In one embodiment, the nucleic acid sequences are linked together such that they are physically close to one another (e.g., within the same locus of a chromosome) and tend to stay together during meiosis, in order to permit coamplification of the two nucleic acid sequences in the host cell and its progeny. For example, in one embodiment a vector comprising a transgene and a vector comprising an amplifiable selectable marker gene are co-transformed into a host cell; upon co-transformation the transgene and selectable amplifiable marker gene become linked through recombination and integration into the host chromosome. In one embodiment, the nucleic acid sequences are linked by a chemical bond (e.g., ligated together). In another embodiment, the nucleic acid sequences are linked enzymatically using a ligase enzyme.

As used herein, the term “amplification reagent” refers to an agent that is useful in identifying duplication of a desired selectable amplifiable marker gene. The amplification reagent is often toxic to cells (especially with increasing concentrations) that lack a sufficient amount of the protein encoded by the selectable amplifiable marker gene. In the methods described herein, where the endogenous selectable amplifiable marker gene is inhibited by an RNA effector molecule, the presence of a vector-supplied selectable amplifiable marker gene permits selection of vector-transfected cells by killing cells lacking the vector. The “amplification reagent” can also be referred to herein as a “selection reagent” or an “amplification/selection reagent.” Some non-limiting examples of an amplification reagent include, but are not limited to, methotrexate, N-phosphonoacetyl-L-aspartic acid (PALA), 2′-deoxycoformycin (dCF), difluoromethylornithine (DFMO), albizziin, and β-aspartyl hydroxamate (β-AHA). The amplification reagent used herein typically induces gene duplication of a particular selectable amplifiable marker gene and the two work in concert as a pair. Thus, one of skill in the art should choose the amplification reagent necessary to produce gene duplication of the desired selectable amplifiable marker supplied in a vector to the host cell. For example, if one desires to use DHFR as the selectable amplifiable marker gene, then one would choose methotrexate or another amplification reagent that induces DHFR gene duplication and permits selection of cells having multiple copies of the DHFR gene (e.g., as supplied by a vector). Exemplary gene/amplification reagent systems are described herein in the Detailed Description.

As is understood in the art, the terms “gene duplication,” “gene amplification,” and “chromosomal duplication” are used interchangeably herein.

As used herein, the term “endogenous to the host cell” refers to any gene that is constitutively present in the host cell genome prior to the introduction of a transgene linked to a selectable amplifiable maker gene. The gene may have previously been introduced into the cell. Typically, an introduced gene will have integrated into the host cell genome and is thus constitutively present in the cell.

As used herein, the term “different nucleic acid sequences” refers to two nucleic acid sequences (e.g., a first and second selectable amplifiable marker gene) that differ in sequence by at least one nucleotide (for example, at least 2, 3, 4, 5, 6, 10, 15, 20, 30 nucleotides or more). In one embodiment, the sequences differ by at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or at least 21 nucleotides within a given 21 bp region (e.g., to confer specificity of RNA effector molecule binding). Functionally, the methods described herein require that an RNA effector molecule bind and inhibit one selectable amplifiable marker gene to a greater degree than that of the other selectable amplifiable marker gene, for example, the RNA effector molecule inhibits the endogenous selectable amplifiable marker gene to a greater extent than that of the vector-supplied selectable amplifiable marker gene (also referred to herein as the “first selectable amplifiable marker gene”). Thus, the nucleic acid sequence of the first and second selectable amplifiable marker gene have different nucleic acid sequences to confer specificity of RNA effector binding and inhibition. In one embodiment, the RNA effector molecule binds and inhibits expression of the second amplifiable marker gene and not the first amplifiable marker gene. In some embodiments, the RNA effector molecule inhibits expression of the second amplifiable marker gene at least 10%, at least 20%, at least 30%, at least 40%, at least 50%, at least 60%, at least 70%, at least 80%, at least 90%, at least 95%, at least 99%, at least 2-fold, at least 5-fold, at least 10-fold, at least 100 fold, or at least 1000 fold more than the RNA effector molecule inhibits the first selectable amplifiable marker. In one embodiment, the first and second amplifiable marker genes, while having different nucleic acid sequences by at least one nucleotide, each encode for the same protein necessary for cell growth or survival.

As used herein, the term “differs by at least one nucleotide” refers to a nucleic acid sequence for a selectable amplifiable marker gene (e.g., vector-supplied) that differs from the nucleic acid sequence for the endogenous selectable amplifiable marker gene by at least one nucleotide. Any number of differences between the two sequences can be tolerated using the methods described herein, however the difference in sequence should be enough to permit selective RNA effector molecule binding to the endogenous marker gene, while only partially or not inhibiting at all, the amplifiable marker gene exogenously added (e.g., vector supplied; “first selectable amplifiable marker) to the cell. In some examples, the nucleic acid sequences differ by at least two nucleotides, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 40, at least 50, at least 60 or at least 70 or more nucleotides, provided that each nucleic acid sequence encodes a polypeptide and can be amplified using an amplification reagent as described herein.

As used herein, “capable of being amplified using the same amplification reagent” means that the same compound or agent can induce amplification of both the first and second amplifiable marker gene e.g., increasing amounts of gene product must be produced in the presence of increasing amounts of the amplification reagent in order for cells to survive. In one embodiment, the term “amplified” refers to an increase in the copy number of the selectable amplifiable marker gene by at least 1 copy in a host cell treated with an amplification reagent, compared to the copy number of the same marker gene in a host cell not treated with the amplification reagent.

As used herein, the term “sequential increases in concentration” or “progressively increasing amount of the amplification reagent” refers to a stepwise increase in the concentration or amount of an amplification reagent administered to the cells. The time frame between each sequential increase in concentration can be hours, days or weeks, and the cells are maintained with an RNA effector molecule in an amount that inhibits expression of one of the selectable amplifiable markers. The cells should be cultured in the presence of a given concentration of the amplification reagent for a sufficient time to allow selection of cells with amplified selectable marker (and consequently make higher levels of the encoding protein) such that the cells become substantially resistant to the increased concentration of the amplification reagent. One can continue with the next sequential increase in concentration when a majority of cells are substantially resistant to the amplification reagent at the present concentration (e.g., when few cells in the culture are sensitive to the provided concentration of amplification reagent (e.g., below 5%, below 10%, below 25%).

As used herein, the term “select for cells with multiple copies” refers to selecting for viable cells at a concentration of the amplification reagent that would inhibit the growth of the input cells (e.g., when the cells are cultured in the presence of increasing amounts of an amplification reagent as described herein). Under such growth conditions, cells that retain viability despite increasing concentrations of the amplification reagent are indicative of expressing higher levels of the selectable marker gene (likely due to higher copies of the gene), as increasing amounts of the gene product are necessary for survival in a cell culture with increasing amounts of the amplification reagent. In one embodiment, the increase in copy number of the gene during each selection with a progressive increase in the concentration of the amplification reagent is monitored by RT-PCR or other conventional methods described herein.

As used herein, the term “RNA effector molecule” refers to an oligonucleotide capable of inhibiting the expression of a selectable amplifiable marker gene or a transgene, as defined herein, within a host cell, or a polynucleotide agent capable of forming an oligonucleotide that can inhibit the expression of a selectable amplifiable marker gene or a transgene upon being introduced into a host cell. The methods described herein encompasses exposure of the cell to an RNA effector molecule expressed within the cell, e.g., shRNA, or exposure by exogenous addition of the RNA effector molecule to the cell, e.g., delivery of the RNA effector molecule to the cell, optionally using an agent that facilitates uptake into the cell. A portion of an RNA effector molecule is substantially complementary to at least a portion of the target RNA (e.g., selectable amplifiable marker gene or transgene RNA), such as the coding region, the promoter region and the 3′ untranslated region (3′-UTR) of the target RNA. In one embodiment, the RNA effector molecule is not shRNA. In another embodiment, the RNA effector molecule is not vector-encoded.

In the context of this invention, the term “oligonucleotide” refers to a polymer or oligomer of nucleotide or nucleoside monomers comprising naturally occurring bases sugars and intersugar (backbone) linkages. The term “oligonucleotide” also includes polymers or oligomers comprising non-naturally occurring monomers, or portions thereof, which function similarly. Such modified or substituted oligonucleotides are often preferred over native forms because of properties such as, for example, enhanced cellular uptake, increased stability in the presence of nucleases, and the like.

Double-stranded and single-stranded oligonucleotides that are effective in inducing RNA interference are also referred to as siRNA, RNAi agent, or iRNA agent, herein. These RNA interference inducing oligonucleotides associate with a cytoplasmic multi-protein complex known as RNAi-induced silencing complex (RISC). Without wishing to be bound by theory, RNA interference leads to Argonaute-mediated post-transcriptional cleavage of target mRNA transcripts. In many embodiments, single-stranded and double-stranded RNAi agents are sufficiently long that they can be cleaved by an endogenous molecule, e.g. by Dicer, to produce smaller oligonucleotides that can enter the RISC machinery and participate in RISC mediated cleavage of a target sequence, e.g. a target mRNA.

As used herein, the term “region” or “portion,” when used in reference to an RNA effector molecule refers to a nucleic acid sequence of at least 3, at least 4, at least 5, at least 6, at least 7, at least 8, at least 9, at least 10 or more nucleotides up to and including the entire nucleic acid sequence of a strand of an RNA effector molecule. In some embodiments, the “region” or “portion” when used in reference to an RNA effector molecule includes nucleic acid sequence one nucleotide shorter than the entire nucleic acid sequence of a strand of an RNA effector molecule. Thus, the term “portion” refers to a region of an RNA effector molecule having a desired length to effect complementary binding to a region of a target RNA or a desired length of a duplex region. One of skill in the art can vary the length of the “portion” that is complementary to the target RNA or arranged in a duplex, such that an RNA effector molecule having desired characteristics (e.g., inhibition of a selectable amplifiable marker gene or a transgene) is produced. While not wishing to be bound by theory, RNA effector molecules provided herein can modulate expression of target genes by one or more of a variety of mechanisms, including but not limited to, Argonaute-mediated post-transcriptional cleavage of target mRNA transcripts (sometimes referred to in the art as RNAi) and/or other pre-transcriptional and/or pre-translational mechanisms.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person. Such conditions can, for example, be stringent conditions, where stringent conditions can include: 400 mM NaCl, 40 mM PIPES pH 6.4, 1 mM EDTA, 50° C. or 70° C. for 12-16 hours followed by washing. Other conditions, such as physiologically relevant conditions as can be encountered inside an organism, can apply. The skilled person will be able to determine the set of conditions most appropriate for a test of complementarity of two sequences in accordance with the ultimate application of the hybridized nucleotides.

Complementary sequences within an RNA effector molecule, e.g., within a dsRNA as described herein, include base-pairing of the oligonucleotide or polynucleotide comprising a first nucleotide sequence to an oligonucleotide or polynucleotide comprising a second nucleotide sequence over the entire length of one or both nucleotide sequences. Such sequences can be referred to as “fully complementary” with respect to each other herein. However, where a first sequence is referred to as “substantially complementary” with respect to a second sequence herein, the two sequences can be fully complementary, or they can form one or more, but generally not more than 5, 4, 3 or 2 mismatched base pairs upon hybridization for a duplex up to 30 base pairs, while retaining the ability to hybridize under the conditions most relevant to their ultimate application, e.g., inhibition of gene expression via a RISC pathway. However, where two oligonucleotides are designed to form, upon hybridization, one or more single stranded overhangs, such overhangs shall not be regarded as mismatches with regard to the determination of complementarity. For example, a dsRNA comprising one oligonucleotide 21 nucleotides in length and another oligonucleotide 23 nucleotides in length, wherein the longer oligonucleotide comprises a sequence of 21 nucleotides that is fully complementary to the shorter oligonucleotide, may yet be referred to as “fully complementary” for the purposes described herein.

“Complementary” sequences, as used herein, can also include, or be formed entirely from, non-Watson-Crick base pairs and/or base pairs formed from non-natural and modified nucleotides, in as far as the above requirements with respect to their ability to hybridize are fulfilled. Such non-Watson-Crick base pairs includes, but are not limited to, G:U Wobble or Hoogstein base pairing.

The terms “complementary,” “fully complementary” and “substantially complementary” herein can be used with respect to the base matching between the sense strand and the antisense strand of a dsRNA, or between the antisense strand of an RNA effector molecule agent and a target sequence, as will be understood from the context of their use.

As used herein, a polynucleotide that is “substantially complementary to at least part of a target RNA refers to a polynucleotide that is substantially complementary to a contiguous portion of a target RNA of interest (e.g., an mRNA encoded by a selectable amplifiable marker gene or a transgene, the target gene's promoter region or 3′ UTR). For example, a polynucleotide is complementary to at least a part of a target mRNA if the sequence is substantially complementary to a non-interrupted portion of an mRNA encoded by a target gene.

As used herein the term “multiple copies” refers to a plurality of copies of a selectable amplifiable marker gene and/or a transgene.

As used herein, the term “plurality” refers to at least two, for example a plurality of host cells refers to at least 2 host cells. The term “plurality” also encompasses at least 3, at least 4, at least 5, at least 6, at least 7, at least 8, at least 9, at least 10, at least 20, at least 30, at least 40, at least 50, at least 60, at least 70, at least 80, at least 90, at least 100, at least 500, at least 1000, at least 1×10⁴, at least 1×10⁵, at least 1×10⁶, at least 1×10⁷, at least 1×10⁸, at least 1×10⁹, at least 1×10¹⁰ or more.

As used herein the term “culturing a cell” or “contacting a cell” refers to the treatment of a cell in culture with an agent e.g., at least one RNA effector molecule, often prepared in a composition comprising a reagent that facilitates uptake of the RNA effector molecule into the cell (e.g., Lipofectamine) or an amplification reagent. The step of contacting a cell with an RNA effector molecule(s) can be repeated more than once (e.g., twice, 3×, 4×, 5×, 6×, 7×, 8×, 9×, 10×, 11×, 12×, 13×, 14×, 15×, 16×, 17×, 18×, 19×, 20×, 30×, 40×, 50×, 60×, 70×, 80×, 90×, 100× or more). In one embodiment, the cell is contacted such that the selectable amplifiable marker or transgene is modulated only transiently, e.g., by addition of an RNA effector molecule composition to the cell culture medium used for the production of the polypeptide where the presence of the RNA effector molecule dissipates over time, i.e., the RNA effector molecule is not constitutively expressed in the cell.

Cells can also be “contacted” with an amplification reagent. In one embodiment, the cells are contacted with the reagent by addition of the reagent to the cell medium or growth medium. In another embodiment, the amplification reagent is administered as a slow release formulation or is embedded in a matrix forming the surface on which the cells grow (e.g., fibronectin, gelatin, polymer matrix etc).

As used herein, the term “transfecting a host cell” refers to the process of introducing a nucleic acid (e.g., an RNA effector molecule, vector etc.). Means for facilitating or effecting uptake or absorption into the cell, are understood by those skilled in the art. Absorption or uptake of an RNA effector molecule or vector can occur through unaided diffusive or active cellular processes, or by auxiliary agents or devices. In vitro introduction into a cell includes methods known in the art such as electroporation and lipofection. Further approaches are described herein below or known in the art. As used herein, “effective amount” refers to that amount of an RNA effector molecule effective to produce an inhibitory effect on expression of a selectable amplifiable marker gene or a transgene.

As used herein, the phrase “reagent that facilitates RNA effector molecule uptake” or “transfection reagent” refers to any agent that enhances uptake of an RNA effector molecule into a host cell by at least 10%, at least 20%, at least 30%, at least 40%, at least 50%, at least 60%, at least 70%, at least 80%, at least 90%, at least 95%, at least 99%, at least 1-fold, at least 2-fold, at least 5-fold, at least 10-fold, at least 100-fold, at least 1000-fold or more compared to an RNA effector molecule administered in the absence of such a reagent. In one embodiment, a cationic or non-cationic lipid molecule useful for preparing a composition or for co-administration with an RNA effector molecule is used as a reagent that facilitates RNA effector molecule uptake. In other embodiments, the reagent that facilitates RNA effector molecule uptake comprises a chemical linkage to attach e.g., a ligand, a peptide group, a lipophillic group, a targeting moiety etc, as described throughout the application herein. In other embodiments, the reagent that facilitates RNA effector molecule uptake comprises a charged lipid, an emulsion, a liposome, a cationic or non-cationic lipid, an anionic lipid, a transfection reagent or a penetration enhancer as described throughout the application herein. In one embodiment, the reagent that facilitates RNA effector molecule uptake used herein comprises a charged lipid as described in U.S. Ser. No. 61/267,419 filed on Dec. 7, 2009, which is herein incorporated by reference in its entirety. Some non-limiting examples of transfection reagents useful with the methods described herein include, but are not limited to, DODAP, DOPE, DOTMA, Lipofectamine™ (Invitrogen; Carlsbad, Calif.), Lipofectamine 2000™ (Invitrogen; Carlsbad, Calif.), 293fectin™ (Invitrogen; Carlsbad, Calif.), Cellfectin™ (Invitrogen; Carlsbad, Calif.), DMRIE-C™ (Invitrogen; Carlsbad, Calif.), FreeStyle™ MAX (Invitrogen; Carlsbad, Calif.), Lipofectamine™ 2000 CD (Invitrogen; Carlsbad, Calif.), Lipofectamine™ (Invitrogen; Carlsbad, Calif.), RNAiMAX (Invitrogen; Carlsbad, Calif.), Oligofectamine™ (Invitrogen; Carlsbad, Calif.), Optifect™ (Invitrogen; Carlsbad, Calif.), X-tremeGENE Q2 Transfection Reagent (Roche; Grenzacherstrasse, Switzerland), DOTAP Liposomal Transfection Reagent (Grenzacherstrasse, Switzerland), DOSPER Liposomal Transfection Reagent (Grenzacherstrasse, Switzerland), or Fugene (Grenzacherstrasse, Switzerland), Transfectam® Reagent (Promega; Madison, Wis.), TransFast™ Transfection Reagent (Promega; Madison, Wis.), Tfx™-20 Reagent (Promega; Madison, Wis.), Tfx™-50 Reagent (Promega; Madison, Wis.), DreamFect™ (OZ Biosciences; Marseille, France), EcoTransfect (OZ Biosciences; Marseille, France), TransPass^{a} D1 Transfection Reagent (New England Biolabs; Ipswich, Mass., USA), LyoVec™/LipoGen™ (Invitrogen; San Diego, Calif., USA), PerFectin Transfection Reagent (Genlantis; San Diego, Calif., USA), NeuroPORTER Transfection Reagent (Genlantis; San Diego, Calif., USA), GenePORTER Transfection reagent (Genlantis; San Diego, Calif., USA), GenePORTER 2 Transfection reagent (Genlantis; San Diego, Calif., USA), Cytofectin Transfection Reagent (Genlantis; San Diego, Calif., USA), BaculoPORTER Transfection Reagent (Genlantis; San Diego, Calif., USA), TroganPORTER™ transfection Reagent (Genlantis; San Diego, Calif., USA), RiboFect (Bioline; Taunton, Mass., USA), PlasFect (Bioline; Taunton, Mass., USA), UniFECTOR (B-Bridge International; Mountain View, Calif., USA), SureFECTOR (B-Bridge International; Mountain View, Calif., USA), or HiFect™ (B-Bridge International, Mountain View, Calif., USA), among others.

The term “expression” as used herein is intended to mean the transcription to an RNA and/or translation to one or more polypeptides from a gene coding for the sequence of the RNA and/or the polypeptide.

The term “inhibits expression of,” and the like, in so far as it refers to a target gene, herein refer to the inhibition of expression of a target gene, as manifested by a decrease in the amount of the target RNA which can be isolated from or detected in a first cell or group of cells in which a target gene (e.g., selectable amplifiable marker or transgene) is transcribed and which has or have been treated such that the expression of a target gene is inhibited, as compared to a second cell or group of cells substantially identical to the first cell or group of cells but which has or have not been so treated (control cells). In one example, expression of a target gene is inhibited by at least 10%, at least 15%, at least 20%, at least 25%, at least 30%, at least 35%, at least 40%, at least 45%, or at least 50% by administration of an RNA effector molecule provided herein. In some embodiments, expression of a selectable amplifiable marker or transgene is inhibited by at least 60%, at least 70%, or at least 80% by administration of an RNA effector molecule to a host cell. In some embodiments, expression of a target gene (e.g., a selectable amplifiable marker or a transgene) is inhibited by at least 85%, at least 90%, or at least 95% or more by administration of an RNA effector molecule as described herein. In one embodiment, expression of the target gene is inhibited by 99% or even 100% (e.g., below detectable limits).

An RNA effector molecule as described herein can be transfected into a host cell immediately before, simultaneously with or immediately after transfection of the vector comprising a transgene. As used herein, the term “immediately before” encompasses transfection with an RNA effector molecule at least 5 minutes before transfection with the vector-supplied transgene e.g., at least 10 minutes before, at least 15 minutes before, at least 20 minutes before, at least 25 minutes before, at least 30 minutes before, at least 45 minutes before, at least 1 hour before, at least 1.5 h before, at least 2 hours before, at least 3 hours before, at least 5 hours before, at least 6 hours before, at least 12 hours before, at least 18 hours before, at least 24 hours before, at least 48 hours before, at least 1 week before, at least 2 weeks before or even earlier before transfection with the vector comprising the transgene. For longer intervals between administration of the RNA effector molecule and the vector, one of skill in the art will appreciate that the half-life of an RNA effector molecule in a host cell will vary and that to maintain an effective amount of the RNA effector molecule one will either need to perform repeated transfections or administer the RNA effector molecule by continuous infusion. As used herein, the term “simultaneously with” refers to transfection of the RNA effector molecule at the same time or within 5 minutes of the transfection with the vector, e.g., 5 minutes before, at least 4 minutes before, at least 3 minutes before, at least 2 minutes before, a least 1 minute before, at the same time, at least 1 minute after, at least 2 minutes after, at least 3 minutes after, at least 4 minutes after, or 5 minutes after. As used herein, the term “immediately after” refers to transfection with an RNA effector molecule at least 5 minutes after transfection with the vector-supplied transgene e.g., at least 10 minutes after, at least 15 minutes after, at least 20 minutes after, at least 25 minutes after, at least 30 minutes after, at least 45 minutes after, at least 1 hour after, at least 1.5 h after, at least 2 hours after, at least 3 hours after, at least 5 hours after, at least 6 hours after, at least 12 hours after, at least 18 hours after, at least 24 hours after, at least 48 hours after, at least 72 hours after, at least 84 hours after, at least 96 hours after, at least 108 hours after, at least 1 week after, at least 2 weeks after, at least 3 weeks later, at least 1 month later, or more after transfection with the vector comprising the transgene.

As used herein, the term “transfection efficiency” refers to the number of viable cells in the population that express the transgene from a vector following transfection. An “increase in transfection efficiency” refers to an increase in the number of transformed cells by at least 10% in cells treated with an RNA effector molecule compared to cells that are not treated with the RNA effector molecule e.g., an increase of at least 20%, at least 30%, at least 40%, at least 50%, at least 60%, at least 70%, at least 80%, at least 90% at least 95%, at least 1-fold, at least 2-fold, at least 5-fold, at least 10-fold, at least 20-fold, at least 50-fold, at least 100-fold, at least 1000-fold or more in vector-transfected cells treated with an RNA effector molecule compared to untreated vector-transfected cells.

A “bioreactor,” as used herein, refers generally to any reaction vessel suitable for growing and maintaining producer cells such as those described herein, as well as producing biological products using such cells. Bioreactors described herein include cell culture systems of varying sizes, such as small culture flasks, Nunc multilayer cell factories, small high yield bioreactors (e.g., MiniPerm, INTEGRA-CELLine), spinner flasks, hollow fiber-WAVE bags (Wave Biotech, Tagelswangen, Switzerland), and industrial scale bioreactors. In some embodiments, the biological product is produced in a bioreactor having a capacity suitable for pharmaceutical or industrial scale production of polypeptides (e.g., a volume of at least 2 liters, at least 5 liters, at least 10 liters, at least 25 liters, at least 50 liters, at least 100 liters, or more) and means of monitoring pH, glucose, lactate, temperature, and/or other bioprocess parameters.

As used herein, an “RNA effector composition” comprises an effective amount of an RNA effector molecule and an acceptable carrier. In one embodiment, the RNA effector molecule composition further comprises a reagent that facilitates RNA effector molecule uptake (e.g., a transfection reagent).

As used herein, the term “inhibits” or “inhibition” encompasses the term “average percent inhibition.” As used herein, the term “average percent inhibition” refers to the average degree of inhibition of target gene expression over time that is necessary to produce the desired effect (e.g., inhibition of expression of a target RNA) and which is below the degree of inhibition that produces any unwanted or negative effects. In some embodiments, the desired average percent inhibition is at least 20%, at least 30%, at least 40%, at least 50%, at least 60%, at least 70%, at least 80%, at least 85%, at least 90%, at least 95%, at least 99%, or even 100% (i.e., absent). One of skill in the art can use routine cell death assays to determine the upper limit for desired percent inhibition (e.g., level of inhibition that produces unwanted effects). One of skill in the art can also use methods to detect target gene expression (e.g., RT-PCR) to determine an amount of an RNA effector molecule that produces target RNA inhibition. The percent inhibition is described herein as an average value over time, since the amount of inhibition is dynamic and can fluctuate slightly between doses of the RNA effector molecule.

As used herein, the term “transiently inhibited” refers to the temporary inhibition of a target gene following administration of a discrete dose of an RNA effector molecule, such that the inhibition of the target gene decreases as the RNA effector molecule is cleared from the cell. In some cases, inhibition can be completely lost in between repeated administrations of an RNA effector molecule in discrete doses. In other embodiments, there can be only a partial loss of inhibition (e.g., 10%, 20%, 30%, 40%, 50%, 60%, 70%, 80%, 90% etc) as the RNA effector molecule activity is cleared. The length of time that inhibition is maintained following treatment with a single dose of RNA effector molecule will depend on the particular RNA effector molecule and/or the target gene. One of skill in the art can easily determine using e.g., ELISA assays to determine the level of inhibition and/or the loss of inhibition over time to choose an appropriate dosing regime to (1) transiently inhibit the target RNA, (2) continuously inhibit the target RNA, or (3) maintain at least a partial inhibition of the target RNA.

As used herein, the terms “significant” or “significantly” is used to refer to a value larger or smaller than two standard deviations from the mean.

The term “acceptable carrier” refers to a carrier for administration of an RNA effector molecule to cultured eukaryotic host cells. Such carriers include, but are not limited to, saline, buffered saline, dextrose, water, glycerol, ethanol, and combinations thereof. The term specifically excludes cell culture medium.

As used herein, the term “SNALP” refers to a stable nucleic acid-lipid particle. A SNALP represents a vesicle of lipids coating a reduced aqueous interior comprising a nucleic acid such as an RNA effector molecule or a plasmid from which an RNA effector molecule is transcribed. SNALPs are described, e.g., in U.S. Patent Application Publication Nos. 2006/0240093, 2007/0135372, and U.S. patent application Ser. No. 12/343,342, filed on Dec. 23, 2008 and Ser. No. 12/424,367, filed on Apr. 15, 2009. These applications are hereby incorporated by reference in their entirety.

As used herein the term “comprising ” or “comprises” is used in reference to compositions, methods, and respective component(s) thereof, that are essential to the invention, yet open to the inclusion of unspecified elements, whether essential or not.

As used herein the term “consisting essentially of refers to those elements required for a given embodiment. The term permits the presence of elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

### DETAILED DESCRIPTION

Provided herein are methods and compositions for generating a cell line capable of producing a biological product, using a gene amplification based system. Typically, a gene amplification based system involves the amplification of gene copy number of a vector-supplied selectable amplifiable marker and a linked transgene in a host cell. Multiple copies of the transgene permits higher levels of the transgene-encoded biological product to be produced in the cell, while multiple copies of the amplifiable marker permits cell survival in the presence of an amplification reagent. However, the presence of the selectable amplifiable marker endogenous to the host cell genome can permit survival of cells lacking the vector, or lacking sufficient copy numbers of the introduced amplifiable marker gene, and leads to the selection of false positives. Thus, methods and compositions are provided herein that inhibit the endogenous selectable amplifiable marker genes using RNA interference and prevents the selection of false positives during generation of a custom cell line. Such methods improve efficiency of cell line development and do not require the use of specialized substrates or cells lacking the endogenous selectable amplifiable marker gene to negate the effect of endogenously expressed levels of the selectable amplifiable marker gene in cells.

In addition, it is known that a transgene delivered to cells will initially express at a high level, which can be toxic to the cells. Thus, methods are described herein wherein RNA effector molecules that inhibit the transgene are provided prior to, at the same time, or immediately after transfection of the host cell with the transgene linked to the amplifiable marker gene. Such methods increase the efficiency of obtaining transfected cells, when the transgene used causes transient toxicity to the cells.

### Host Cells

In one embodiment, a mammalian host cell is used to generate a cell capable of producing a biological product or polypeptide, particularly if the polypeptide is a biotherapeutic agent or is otherwise intended for administration to or consumption by humans. In some embodiments, the host cell is a Chinese Hamster Ovary (CHO) cell, which is the cell line most commonly used for the expression of many recombinant proteins. Additional mammalian cell lines often for the expression of recombinant proteins include, but are not limited to, HEK-293 cells, HeLa cells, COS cells, NIH/3T3 cells, Jurkat Cells, NSO cells and HUVEC cells.

In some embodiments, the host cell is a CHO cell derivative that has been genetically modified to facilitate production of recombinant proteins, polypeptides, or other biological products. For example, various CHO cell strains have been developed which permit stable insertion of recombinant DNA into a specific gene or expression region of the cells, amplification of the inserted DNA, and selection of cells exhibiting high level expression of the recombinant protein. Examples of CHO cell derivatives useful in the methods provided herein include, but are not limited to, CHO-K1 cells, CHO-DUKX, CHO-DUKX B1, CHO-DG44 cells, CHO-ICAM-1 cells, and CHO-hIFNγ cells. Methods for expressing recombinant proteins in CHO cells are known in the art and are described, e.g., in U.S. Pat. Nos. 4,816,567 and 5,981,214, herein incorporated by reference in their entirety.

Examples of human cell lines useful in methods provided herein include, but are not limited to, 293T (embryonic kidney), 786-0 (renal), A498 (renal), A549 (alveolar basal epithelial), ACHN (renal), BT-549 (breast), BxPC-3 (pancreatic), CAKI-1 (renal), Capan-1 (pancreatic), CCRF-CEM (leukemia), COLO 205 (colon), DLD-1 (colon), DMS 114 (small cell lung), DU145 (prostate), EKVX (non-small cell lung), HCC-2998 (colon), HCT-15 (colon), HCT-116 (colon), HT29 (colon), HT-1080 (fibrosarcoma), HEK 293 (embryonic kidney), HeLa (cervical carcinoma), HepG2 (hepatocellular carcinoma), HL-60(TB) (leukemia), HOP-62 (non-small cell lung), HOP-92 (non-small cell lung), HS 578T (breast), HT-29 (colon adenocarcinoma), IGR-OV1 (ovarian), IMR32 (neuroblastoma), Jurkat (T lymphocyte), K-562 (leukemia), KM12 (colon), KM20L2 (colon), LAN5 (neuroblastoma), LNCap.FGC (Caucasian prostate adenocarcinoma), LOX IMVI (melanoma), LXFL 529 (non-small cell lung), M14 (melanoma), M19-MEL (melanoma), MALME-3M (melanoma), MCF1OA (mammary epithelial), MCF7 (mammary), MDA-MB-453 (mammary epithelial), MDA-MB-468 (breast), MDA-MB-231 (breast), MDA-N (breast), MOLT-4 (leukemia), NCI/ADR-RES (ovarian), NCI-H226 (non-small cell lung), NCI-H23 (non-small cell lung), NCI-H322M (non-small cell lung), NCI-H460 (non-small cell lung), NCI-H522 (non-small cell lung), OVCAR-3 (ovarian), OVCAR-4 (ovarian), OVCAR-5 (ovarian), OVCAR-8 (ovarian), P388 (leukemia), P388/ADR (leukemia), PC-3 (prostate), PERC6® (E1-transformed embryonal retina), RPMI-7951 (melanoma), RPMI-8226 (leukemia), RXF 393 (renal), RXF-631 (renal), Saos-2 (bone), SF-268 (CNS), SF-295 (CNS), SF-539 (CNS), SHP-77 (small cell lung), SH-SY5Y (neuroblastoma), SK-BR3 (breast), SK-MEL-2 (melanoma), SK-MEL-5 (melanoma), SK-MEL-28 (melanoma), SK-OV-3 (ovarian), SN12K1 (renal), SN12C (renal), SNB-19 (CNS), SNB-75 (CNS) SNB-78 (CNS), SR (leukemia), SW-620 (colon), T-47D (breast), THP-1 (monocyte-derived macrophages), TK-10 (renal), U87 (glioblastoma), U293 (kidney), U251 (CNS), UACC-257 (melanoma), UACC-62 (melanoma), UO-31 (renal), W138 (lung), and XF 498 (CNS).

Examples of rodent cell lines useful in methods provided herein include, but are not limited to, baby hamster kidney (BHK) cells (e.g., BHK21 cells, BHK TK− cells), mouse Sertoli (TM4) cells, buffalo rat liver (BRL 3A) cells, mouse mammary tumor (MMT) cells, rat hepatoma (HTC) cells, mouse myeloma (NS0) cells, murine hybridoma (Sp2/0) cells, mouse thymoma (EL4) cells, Chinese Hamster Ovary (CHO) cells and CHO cell derivatives, murine embryonic (NIH/3T3, 3T3 L1) cells, rat myocardial (H9c2) cells, mouse myoblast (C2C12) cells, and mouse kidney (miMCD-3) cells.

Examples of non-human primate cell lines useful in methods provided herein include, but are not limited to, monkey kidney (CVI-76) cells, African green monkey kidney (VERO-76) cells, green monkey fibroblast (Cos-1) cells, and monkey kidney (CVI) cells transformed by SV40 (Cos-7). Additional mammalian cell lines are known to those of ordinary skill in the art and are catalogued at the American Type Culture Collection catalog (ATCC®, Mamassas, Va.).

In some embodiments, the host cells are suitable for growth in suspension cultures. Suspension-competent host cells are generally monodisperse or grow in loose aggregates without substantial aggregation. Suspension-competent host cells include cells that are suitable for suspension culture without adaptation or manipulation (e.g., hematopoietic cells, lymphoid cells) and cells that have been made suspension-competent by modification or adaptation of attachment-dependent cells (e.g., epithelial cells, fibroblasts).

In some embodiments, the host cell is an attachment dependent cell which is grown and maintained in adherent culture. Examples of human adherent cell lines useful in methods provided herein include, but are not limited to, human neuroblastoma (SH-SY5Y, IMR32 and LAN5) cells, human cervical carcinoma (HeLa) cells, human breast epithelial (MCF1OA) cells, human embryonic kidney (293T) cells, and human breast carcinoma (SK-BR3) cells.

In some embodiments, the host cell is a multipotent stem cell or progenitor cell. Examples of multipotent cells useful in methods provided herein include, but are not limited to, murine embryonic stem (ES-D3) cells, human umbilical vein endothelial (HuVEC) cells, human umbilical artery smooth muscle (HuASMC) cells, human differentiated stem (HKB-I1) cells, and human mesenchymal stem (hMSC) cells.

In some embodiments, the host cell is a plant cell, such as a tobacco plant cell.

In some embodiments, the host cell is a fungal cell, such as a cell from Pichia pastoris, a Rhizopus cell, or a Aspergillus cell.

In some embodiments, the host cell is an insect cell, such as SF9 or SF-21 cells from Spodoptera frugiperda or S2 cells from Drosophila melanogaster.

### Gene Amplification

One method for obtaining high transgene copy number in a host cell involves gene amplification. Gene amplification occurs naturally in eukaryotic cells at a relatively low frequency (see e.g., Schimke, J. Biol. Chem., 263:5989 (1988)). However, gene amplification can also be induced, or at least selected for, by exposing host cells to appropriate selective pressure. For example, in many cases it is possible to introduce a product gene together with an amplifiable gene into a host cell and subsequently select for amplification of the marker gene by exposing the cotransfected cells to sequentially increasing concentrations of a selective agent. Typically the product gene will be coamplified with the marker gene under such conditions.

As but one example, the DHFR/methotrexate gene amplification system is known in the art for the generation of cells capable of producing a biological product. A vector containing DHFR and a transgene is first transfected into cells. Treating such transfected cells with increasing concentrations of methotrexate results in selection of cells with increased levels of the target enzyme dihydrofolate reductase (DHFR) (as a consequence of a proportional increase in the DHFR gene copy number), since methotrexate leads to cell death in the absence of DHFR. The methotrexate resistant cells may contain thousands of DHFR gene copies and thus express high levels of DHFR. Since the nucleic acid sequence of a transgene is linked to the nucleic acid sequence of DHFR, the transgene is often also amplified to produce a cell comprising e.g., hundreds or thousands of copies of the transgene.

However, amplification of DHFR endogenous to the host cell genome can also occur under sequentially increasing concentrations of methotrexate, causing an increase in selection of false positives, or the requirement for the use of DHFR(−) cell lines. In addition, higher concentrations of methotrexate are necessary to distinguish cells lacking a vector to those comprising a vector having a copy of DHFR. Thus, in one embodiment, the present methods and compositions permit inhibition of the endogenous DHFR using RNA interference, which permits non-transfected cells to be selected against at very low doses of methotrexate. The methods and compositions described herein permit efficient early selection of transfected vs. untransfected cells and can speed up the process of generating a cell capable of producing a biological product. Treatment of the cells with sequentially increasing concentrations of methotrexate can also induce gene duplication of the vector-supplied DHFR gene and the transgene to produce cells having multiple transgene copies, while eliminating or greatly reducing the number of false-positives that arise through amplification of the DHFR endogenous to the host cell genome.

Gene amplification can be enhanced by increasing DNA synthesis and/or cell growth, thus it is also contemplated herein that methods for enhancing DNA synthesis or cell growth are combined with the methods and compositions described herein for generating a cell capable of producing a biological product. Such methods for enhancing DNA synthesis and/or cell growth include e.g., hydroxyurea, aphidicolin, UV gamma irradiation, hypoxia, carcinogens, arsenate, phorbal esters, insulin.

The selection of host cells that express high levels of a desired selectable amplifiable marker is generally a multi-step process. In the first step, initial transfectants are selected that have incorporated the transgene and the selectable amplifiable marker gene. In subsequent steps, the initial transfectants are subject to further selection for high-level expression of the selectable gene and then random screening for high-level expression of the transgene.

In one embodiment, the gene amplification system described herein requires stepwise increases in the concentration of an amplification reagent to select for cells having multiple copies of the selectable amplifiable marker gene and the transgene. Transformed cells should be cultured for sufficient time to allow amplification to occur, that is, until the copy number of the amplifiable gene (and preferably also the copy number of the product gene) in the host cells has increased relative to the transformed cells prior to this culturing.

Gene amplification and/or expression can be measured in a sample directly, for example, by conventional Southern blotting, Northern blotting to quantitate the transcription of mRNA (Thomas, Proc. Natl. Acad. Sci. U.S.A., 77:5201-5205 [1980]), dot blotting (DNA analysis), or in situ hybridization, using an appropriately labeled probe, based on the sequences provided herein. Various labels can be employed, most commonly radioisotopes, particularly ³²P. However, other techniques can also be employed, such as using biotin-modified nucleotides for introduction into a polynucleotide. The biotin then serves as the site for binding to avidin or antibodies, which can be labeled with a wide variety of labels, such as radionuclides, fluorescence, enzymes, or the like. Alternatively, antibodies can be employed that can recognize specific duplexes, including DNA duplexes, RNA duplexes, and DNA-RNA hybrid duplexes or DNA-protein duplexes. The antibodies in turn can be labeled and the assay can be carried out where the duplex is bound to a surface, so that upon the formation of duplex on the surface, the presence of antibody bound to the duplex can be detected.

Gene expression, alternatively, can be measured by immunological methods, such as immunohistochemical staining of tissue sections and assay of cell culture or body fluids, to quantitate directly the expression of gene product. With immunohistochemical staining techniques, a cell sample is prepared, typically by dehydration and fixation, followed by reaction with labeled antibodies specific for the gene product coupled, where the labels are usually visually detectable, such as enzymatic labels, fluorescent labels, luminescent labels, and the like. A particularly sensitive staining technique suitable for use in the present invention is described by Hsu et al., Am. J. Clin. Path., 75:734-738 (1980). In one embodiment, gene expression is measured by RT-PCR or immunoblotting (e.g., Western blotting).

### Exemplary Gene Amplification Systems

The DHFR/methotrexate system is one model system, however several other selectable amplifiable marker gene/amplification reagent systems can also be used, which are described in e.g., Kaufman, R J. Methods in Enzymology (1990) 185:537-566, which is herein incorporated by reference in its entirety.

For example, another system involves the use of the selectable amplifiable marker gene carbamoyl-phosphate synthase-aspartate transcarbamoylase-dihydroorotase (CAD), which can be amplified by sequentially increasing the concentration of N-phosphonoacetyl-L-aspartic acid (PALA).

Another system utilizes the selectable amplifiable marker gene adenosine deaminase, wherein gene amplification is induced with the amplification reagent 2′-deoxycoformycin. Adenosine deaminase is not an essential enzyme for cell growth under normal conditions, however adenosine deaminase is required for cell survival when cells are cultured in cytotoxic adenine nucleosides (e.g., 9-β-D-xylofuranosyl adenine). Once adenosine deaminase is required for cell survival, the cells can be treated with 2′-deoxycoformycin to select for amplification of the adenosine deaminase gene.

In another system, the selectable amplifiable marker gene used is thymidylate synthetase and the amplification reagent is 5′fluorodeoxyuridine.

Another system that can be used with the methods and compositions described herein utilizes the selectable amplifiable marker gene glutamine synthase and the amplification reagent methionine sulfoximine. Methionine sulfoximine permits amplification of the glutamine synthetase gene.

Another exemplary system uses the selectable amplifiable marker gene ornithine decarboxylase and the amplification reagent difluoromethylornithine (DFMO). Ornithine decarboxylase is an essential enzyme in the synthesis of polyamines and thus is essential for cell growth. Treatment of cells with increasing concentrations of DFMO permit selection of cells with amplification of the ornithine decarboxylase gene.

In another embodiment, the system involves the use of asparagine synthetase as the selectable amplifiable marker in combination with the amplification reagent β-aspartyl hydroxamate (β-AHA) or albizziin.

Conditions for selection and amplification for using these systems are well known to those of skill in the art and are described in e.g. Kaufman, R J. Methods in Enzymology (1990) 185:537-566. Amplification reagents can be added at concentrations ranging from about 0.005 μM to about 100 mM, in a stepwise manner to select for multiple copies of the amplification gene. For example, MTX used in the DHFR/MTX system is typically added to culture medium at a concentration range of about 0.005 to about 0.02 μM, after selection for 1-2 weeks, the concentration is increased 2 to 5-fold. Multiple selection steps can be performed, each time increasing the concentration of amplification reagent 2 to 5-fold. PALA used in the CAD/PALA system is typically added to selection media at a concentration of 100 μM and the concentration is increased to 250 μM and 1 mM at each selection step. 2′-deoxycoformycin (dCF) to select for amplification of the adenosine deaminase gene is typically added to selection medium at a concentration of about 0.03 or 0.1 mM and after 10-14 days cells are sequentially grown in 3-fold increasing concentrations of dCF. Suicide substrate inhibitor difluoromethylornithine (DFMO) is used to select for Ornithine decarboxylase, typically, at a concentration of 160 μM, and cells are selected sequentially with 600 μM, 1 mM, 3 mM, 9 mM, and 15 mM DFMO. Increasing concentrations of β-AHA are used to amplify asparagine synthase, for example starting at 0.2 mM in successive steps up to 1.5 mM, then 1 mM incremental steps from 5 mM to about 50 mM. Methionine sulfoximine permits amplification of the glutamine synthetase gene and is provided at a concentration range of about 1 uM to about 5 mM, stepwise.

### Selectable Marker Genes

Essentially any selectable amplifiable marker gene, as that term is used herein, that is known in the art can be used with the methods described herein. Some non-limiting examples of such selectable amplifiable marker genes include dihydrofolate reductase (DHFR) (e.g. GenBank: AAA36971.1 (SEQID NO: 1420), M317124.1 (SEQ ID NO: 1421), NM_{—}010049.3 (SEQ ID NO: 1422)); thymidylate synthase (e.g. GeneBank NM_{—}021288.4 (SEQ ID NO:1423), NM_{—}021288.4 (SEQ ID NO: 1424), NM_{—}001071 (SEQ ID NO: 1425)), glutamine synthetase (e.g. GenBank: NP_{—}032157 (SEQ ID NO: 1426), NM_{—}008131 (SEQ ID NO: 1427), AAB35189.2 (SEQ ID NO:1428), 579193.1 (SEQ ID NO: 1429)), adenosine deaminase (e.g. GenBank: NP_{—}000013 (SEQ ID NO: 1430), NM_{—}000022.2 (SEQ ID NO: 1431), NP_{—}031424.1 (SEQ ID NO: 1432), NM_{—}007398.3 (SEQ ID NO: 1433), NP_{—}037027 (SEQ ID NO: 1434), NM_{—}012895.3 (SEQ ID NO: 1435)), carbamoyl-phosphate synthase-aspartate transcarbamoylase-dihydroorotase (CAD) (e.g. GenBank: BAA24977.1 (SEQ ID NO: 1436), AB009377.1 (SEQ ID NO: 1437), P08955.4 (SEQ ID NO: 1438)), ornithine decarboxylase (e.g GenBank: NP_{—}036747.1 (SEQ ID NO: 1439), NM_{—}012615.2 (SEQ ID NO: 1440), NP_{—}038642.2 (SEQ ID NO: 1441), NM_{—}013614.2 (SEQ ID NO: 1442), AAA36963.1 (SEQ ID NO: 1443), J02813.1 (SEQ ID NO: 1444), and asparagine synthetase (e.g. M27838.1 (SEQ ID NO: 1445), AAA36977.1 (SEQ ID NO: 1446), AAA85125.1 (SEQ ID NO: 1447), U38940.1 (SEQ ID NO: 1448)).

In one embodiment, the methods provided herein permit enhanced transfection efficiency of cells by administering an RNA effector molecule that transiently inhibits the initial expression of the transgene (e.g., the transgene encoding a biological product to be produced), which can be toxic to cells. In one embodiment, the RNA effector molecule that transiently inhibits expression of a transgene is administered immediately before, simultaneously with, or immediately after transfection with the RNA effector that inhibits the selectable amplifiable marker that is endogenous to a host cell. In another embodiment, the RNA effector molecule is administered immediately before, simultaneously with, or immediately after the vector encoding the transgene is transfected into the host cell. In such embodiments where gene amplification is not necessary any selectable marker known in the art, in addition to those recited above, can be used with the methods described herein, such as antibiotic resistance genes (e.g., Tet^{R}, Neo^{R}), reporter gene (e.g., GFP), cell surface marker (e.g., CD proteins) or any other selectable marker known in the art.

### Co-Amplification

Described herein are methods and compositions for generating a cell line capable of producing a biological product. The method involves introduction of a transgene and a selectable amplifiable marker gene, such that the nucleic acid sequence for the transgene is linked to the nucleic acid sequence of the marker gene to permit coamplification of both genes.

In one embodiment, the transgene and the selectable amplifiable marker gene are linked together and provided on the same vector. This method ensures that the two nucleic acid sequences integrate into the same region of the host genome and that the transgene will be duplicated as the marker gene is duplicated.

In another embodiment, the transgene and the selectable amplifiable marker gene are provided on separate vectors and are linked co-transformationally. The term “co-transformationally” refers to a process by which separate DNA molecules are ligated together inside the cell and subsequently cointegrate into the host genome as a unit (e.g., via a non-homologous recombination event). This can be achieved by co-transfecting two vectors at the same time. When separate DNA molecules are sequentially introduced into cells, the molecules may not become linked and will not cointegrate into the same chromosomal position. Thus, if one desires to use multiple vectors (e.g. one vector comprising a transgene and one vector comprising a first selectable amplifiable marker gene) with the methods described herein, the vectors must be transfected at substantially the same time to effect coamplification of the transgene and the selectable amplifiable marker gene. Methods for generating recombinant vectors are well known to those of skill in the art and can be found in e.g. Sambrook, et al. Molecular Cloning: Sambrook, et al. Molecular Cloning: By Joe Sambrook, Peter MacCallum, David Russell, CSHL Press, 2001.

### Modified Selectable Amplifiable Marker Genes

The methods described herein rely, in part, on an RNA effector molecule that can inhibit a selectable amplifiable marker gene endogenous to the cell, without reducing expression or amplification of a modified selectable amplifiable marker gene that is linked to a transgene and transfected into a host cell.

The nucleic acid sequences for the endogenous marker gene and the vector-supplied marker gene should be sufficiently different from each other to permit selective inhibition of one selectable amplifiable marker gene. This can be achieved by modifying the host cell selectable amplifiable marker gene by PCR techniques prior to incorporation into the vector. Alternatively, this can be achieved by using a selectable amplifiable marker gene from a different host (e.g., a different species or a recombinantly produced selectable amplifiable marker gene). For example, one can use a human selectable amplifiable marker gene in a vector used to transform CHO cells, provided that the sequences are sufficiently different to permit selective RNA effector molecule binding. RNA effector molecules can be designed within regions of the selectable amplifiable marker gene that are not well conserved among species etc. to prevent inhibition of the vector supplied amplifiable marker gene.

Alternatively, a selectable amplifiable marker gene from prokaryotic cell (e.g., E. coli) can be used. Any modifications made to the selectable amplifiable marker gene should not render the gene unable to produce the gene product as this will likely result in death of the cells in the presence of the amplification/selection reagent.

### Increasing Transfection Efficiency

Methods are also provided herein for increasing the transfection efficiency of a vector in a population of host cells. Typically, transient transgene expression occurs shortly following transfection of host cells. Expression of the transgene can be toxic to some cells, particularly shortly after transfection and can result in reduced transfection efficiency. Thus, methods are provided herein that reduces the initial transgene expression by transfecting an RNA effector molecule that targets the transgene. The RNA effector molecule can be administered immediately before (e.g., up to 2 days before), simultaneously with, or immediately after (e.g., up to 2 days after) transfection of the vector encoding the transgene. One of skill in the art will appreciate that the timing of this initial increase in expression can vary with each transgene and can determine the appropriate timing for treatment with an RNA effector molecule to attenuate the increased expression (as measured using e.g., RT-PCR or Western Blotting).

Transfected cells cultured in the presence of an RNA effector molecule to inhibit transgene expression can be selected using e.g., a selectable marker also supplied on the vector (e.g., a reporter gene or an antibiotic resistance gene) and grown to a density necessary or desired for production of the biological product. Once the desired growth conditions are reached, the concentration of the RNA effector molecule inhibiting transgene expression is reduced, or removed altogether, to permit expression of the transgene. These methods permit the production of biological products that induce transient or mild to severe toxicity of the host cells in which it is produced.

### Incompatible Cell/Vector Systems

One advantage of the methods and compositions described herein is that essentially any selectable amplifiable marker gene can be used with any desired cell type (i.e., the cell does not need to be engineered to lack the selectable amplifiable marker gene in its genome). As described elsewhere herein, an RNA effector molecule can be designed such that it inhibits an endogenously expressed selectable amplifiable marker gene in the host cell but does not substantially inhibit the selectable amplifiable marker gene administered to the cells in a vector. Thus, one can use any cell line without the need to change the vector system used to supply the transgene to the cells. Therefore, in another aspect, a method for transfecting a cell with a vector is described. The vector would be otherwise incompatible with the host cell due to the presence on the vector of a selectable marker that is also present in the host cell. In this aspect, selection for the presence of the marker present on the vector can be achieved by administering an RNA effector molecule that inhibits expression of a selectable marker endogenous to the host cell. The RNA effector molecule is administered immediately before, simultaneously with, or immediately after transfection of the host cell with the vector. As described elsewhere, the selectable markers on the vector and in the host cell need to have different nucleic acid sequences (e.g., at least one nucleotide difference), to allow selective inhibition of the host cell marker.

### Biological Products

The methods and compositions described herein are useful in the production of a biological product in a cell. Essentially any biological product can be made using the methods described herein including, but not limited to polypeptides (e.g., glycoproteins, antibodies, peptide-based growth factors), carbohydrates, lipids, fatty acids, metabolites (e.g., polyketides, macrolides), peptidomimetics, and chemical intermediates. The biological products can be used for a wide range of applications, including as biotherapeutic agents, vaccines, research or diagnostic reagents, fermented foods, food additives, nutraceuticals, biofuels, industrial enzymes (e.g., glucoamylase, lipase), industrial chemicals (e.g., lactate, fumarate, glycerol, ethanol), and the like.

In one embodiment, the biological product comprises a mutation relative to the endogenously expressed version of the polypeptide commonly observed in a standard population of individuals. Mutations can be in the nucleic acid sequence (e.g., genomic or mRNA sequence), or alternatively can comprise an amino acid substitution. Such amino acid substitutions can be conserved mutations or non-conserved mutations. As well-known in the art, a “conservative substitution” of an amino acid or a “conservative substitution variant” of a polypeptide refers to an amino acid substitution which maintains: 1) the structure of the backbone of the polypeptide (e.g. a beta sheet or alpha-helical structure); 2) the charge or hydrophobicity of the amino acid; or 3) the bulkiness of the side chain. More specifically, the well-known terminologies “hydrophilic residues” relate to serine or threonine. “Hydrophobic residues” refer to leucine, isoleucine, phenylalanine, valine or alanine. “Positively charged residues” relate to lysine, arginine or histidine. “Negatively charged residues” refer to aspartic acid or glutamic acid. Residues having “bulky side chains” refer to phenylalanine, tryptophan or tyrosine. To avoid doubt as to nomenclature, the term “D144N” or similar terms specifying other specific amino acid substitutions means that the Asp (D) at position 144 is substituted with Asn (N). A “conservative substitution variant” of D144N would substitute a conservative amino acid variant of Asn (N) that is not D.

In some embodiments, the polypeptide is further modified to be secreted into the cell culture medium following production in a host cell. Such modifications can include e.g., removal or inhibition of a mannose 6 phosphate group, which prevents uptake into lysosomes of the host cell via a mannose 6 phosphate receptor mediated mechanism.

In one embodiment, the modified biological product (e.g., polypeptide, recombinant polypeptide or peptidomimetic) substantially retains the activity of the wildtype biological product. By “substantially retain” is meant that the modified biological product retains at least 60% of the activity of the unmodified biological product. In some embodiments, the modified biological product retains at least 70%, at least 80%, at least 90%, at least 95%, at least 99%, or even 100% of the activity of the unmodified biological product. The term “substantially retains” also encompasses an increase in the activity of the modified biological product of at least 10% compared to the unmodified biological product; in some embodiments the increase in activity of the modified biological product is at least 20%, at least 30%, at least 40%, at least 50%, at least 60%, at least 70%, at least 80%, at least 90%, at least 95%, at least 99%, at least 1-fold, at least 2-fold, at least 5-fold, at least 10-fold, at least 50-fold, at least 100-fold, at least 1000-fold or more compared to the unmodified biological product.

### RNA Effector Molecules

Essentially any RNA effector molecule capable of inhibiting expression of a target RNA, as that term is used herein, in a mammalian cell can be used with the methods described herein. RNA effector molecules can comprise a single strand or more than one strand of RNA. The RNA effector molecule can be single-stranded or double-stranded. A single-stranded RNA effector can have double-stranded regions and a double-stranded RNA effector can have single-stranded regions. Without limitations, RNA effector molecules can include, double stranded RNA (dsRNA), microRNA (miRNA), short interfering RNA (siRNA), antisense RNA, promoter-directed RNA (pdRNA), Piwi-interacting RNA (piRNA), expressed interfering RNA (eiRNA), short hairpin RNA (shRNA), antagomirs, decoy RNA, DNA, plasmids and aptamers.

As used herein, the term “double-stranded” refers to an oligonucleotide having a hybridized duplex region that comprises two anti-parallel and substantially complementary nucleic acid strands. The duplex region can be of any length that permits specific degradation of a desired target RNA through a RISC pathway, but will typically range from 9 to 36 base pairs in length, e.g., 15-30 base pairs in length. Considering a duplex between 9 and 36 base pairs, the duplex can be any length in this range, for example, 9, 10, 11, 12, 13, 14, 15, 16, 17, 18, 19, 20, 21, 22, 23, 24, 25, 26, 27, 28, 29, 30, 31, 32, 33, 34, 35, or 36 and any sub-range there between, including, but not limited to 10-15 base pairs, 10-14 base pairs, 10-13 base pairs, 10-12 base pairs, 10-11 base pairs, 15-30 base pairs, 15-26 base pairs, 15-23 base pairs, 15-22 base pairs, 15-21 base pairs, 15-20 base pairs, 15-19 base pairs, 15-18 base pairs, 15-17 base pairs, 18-30 base pairs, 18-26 base pairs, 18-23 base pairs, 18-22 base pairs, 18-21 base pairs, 18-20 base pairs, 19-30 base pairs, 19-26 base pairs, 19-23 base pairs, 19-22 base pairs, 19-21 base pairs, 19-20 base pairs, 20-30 base pairs, 20-26 base pairs, 20-25 base pairs, 20-24 base pairs, 20-23 base pairs, 20-22 base pairs, 20-21 base pairs, 21-30 base pairs, 21-26 base pairs, 21-25 base pairs, 21-24 base pairs, 21-23 base pairs, or 21-22 base pairs. Double-stranded oligonucleotides, e.g., dsRNAs, generated in the cell by processing with Dicer and similar enzymes are generally in the range of 19-22 base pairs in length. One strand, antisense strand, of the duplex region of a double-stranded oligonucleotide comprises a sequence that is substantially complementary to a region of a target RNA. The two strands forming the duplex structure can be from a single oligonucleotide molecule having at least one self-complementary region, or can be formed from two or more separate oligonucleotide molecules. Where the duplex region is formed from two complementary regions of a single molecule, the molecule can have a duplex region separated by a single stranded chain of nucleotides (herein referred to as a “hairpin loop”) between the 3′-end of one strand and the 5′-end of the respective other strand forming the duplex structure. The hairpin loop can comprise at least one unpaired nucleotide; in some embodiments the hairpin loop can comprise at least 2, at least 3, at least 4, at least 5, at least 6, at least 7, at least 8, at least 9, at least 10, at least 20, at least 23 or more unpaired nucleotides. In some embodiments, the hairpin loop comprises 3, 4, 5, 6, or 7 unpaired nucleotides. Where the two substantially complementary strands of a double-stranded oligonucleotide are comprised by separate molecules, those molecules need not, but can be covalently connected. Where the two strands are connected covalently by means other than a hairpin loop, the connecting structure is referred to as a “linker.” The term “siRNA effector molecule” is also used herein to refer to a dsRNA as described above.

In some embodiments, the RNA effector molecule is a promoter-directed RNA (pdRNA) which is substantially complementary to at least a portion of a noncoding region of an mRNA transcript of a target gene. In one embodiment, the pdRNA is substantially complementary to at least a portion of the promoter region of a target gene mRNA at a site located upstream from the transcription start site, e.g., more than 100, more than 200, or more than 1,000 bases upstream from the transcription start site. In another embodiment, the pdRNA is substantially complementary to at least a portion of the 3′-UTR of a target gene mRNA transcript. In one embodiment, the pdRNA comprises dsRNA of 18-28 bases optionally having 3′ di- or tri-nucleotide overhangs on each strand. The dsRNA is substantially complementary to at least a portion of the promoter region or the 3′-UTR region of a target gene mRNA transcript. In another embodiment, the pdRNA comprises a gapmer consisting of a single stranded polynucleotide comprising a DNA sequence which is substantially complementary to at least a portion of the promoter or the 3′-UTR of a target gene mRNA transcript, and flanking the polynucleotide sequences (e.g., comprising the 5 terminal bases at each of the 5′ and 3′ ends of the gapmer) comprising one or more modified nucleotides, such as 2′ MOE, 2′OMe, or Locked Nucleic Acid bases (LNA), which protect the gapmer from cellular nucleases.

pdRNA can be used to selectively increase, decrease, or otherwise modulate expression of a target gene. Without being limited to a particular theory, it is believed that pdRNAs modulate expression of target genes by binding to endogenous antisense RNA transcripts which overlap with noncoding regions of a target gene mRNA transcript, and recruiting Argonaute proteins (in the case of dsRNA) or host cell nucleases (e.g., RNase H) (in the case of gapmers) to selectively degrade the endogenous antisense RNAs. In some embodiments, the endogenous antisense RNA negatively regulates expression of the target gene and the pdRNA effector molecule activates expression of the target gene. Thus, in some embodiments, pdRNAs can be used to selectively activate the expression of a target gene by inhibiting the negative regulation of target gene expression by endogenous antisense RNA. Methods for identifying antisense transcripts encoded by promoter sequences of target genes and for making and using promoter-directed RNAs are described, e.g., in International Publication No. WO 2009/046397, herein incorporated by reference in its entirety.

Expressed interfering RNA (eiRNA) can be used to selectively increase, decrease, or otherwise modulate expression of a target gene. Typically, eiRNA, (e.g., expressed dsRNA) is expressed in the first transfected cell from an expression vector. In such a vector, the sense strand and the antisense strand of the dsRNA can be transcribed from the same nucleic acid sequence using e.g., two convergent promoters at either end of the nucleic acid sequence or separate promoters transcribing either a sense or antisense sequence. Alternatively, two plasmids can be cotransfected, with one of the plasmids designed to transcribe one strand of the dsRNA while the other is designed to transcribe the other strand. Methods for making and using eiRNA effector molecules are described, for example, in International Publication No. WO 2006/033756, and in U.S. Pat. Pub. Nos. 2005/0239728 and 2006/0035344, which are incorporated by reference in their entirety.

In some embodiments, the RNA effector molecule comprises a small single-stranded Piwi-interacting RNA (piRNA effector molecule) which is substantially complementary to at least a portion of a target gene, as defined herein, and which selectively binds to proteins of the Piwi or Aubergine subclasses of Argonaute proteins. Without being limited to a particular theory, it is believed that piRNA effector molecules interact with RNA transcripts of target genes and recruit Piwi and/or Aubergine proteins to form a ribonucleoprotein (RNP) complex that induces transcriptional and/or post-transcriptional gene silencing of target genes. A piRNA effector molecule can be about 25-50 nucleotides in length, about 25-39 nucleotides in length, or about 26-31 nucleotides in length. Methods for making and using piRNA effector molecules are described, e.g., in U.S. Pat. Pub. No. 2009/0062228, herein incorporated by reference in its entirety.

In some embodiments, the RNA effector molecule is an siRNA or shRNA effector molecule introduced into an animal host cell by contacting the cell with an invasive bacterium containing one or more siRNA or shRNA effector molecules or DNA encoding one or more siRNA or shRNA effector molecules (a process sometimes referred to as transkingdom RNAi (tkRNAi)). The invasive bacterium can be an attenuated strain of a bacterium selected from the group consisting of Listeria, Shigella, Salmonella, E. coli, and Bifidobacteriae, or a non-invasive bacterium that has been genetically modified to increase its invasive properties, e.g., by introducing one or more genes that enable invasive bacteria to access the cytoplasm of host cells. Examples of such cytoplasm-targeting genes include listeriolysin O of Listeria and the invasin protein of Yersinia pseudotuberculosis. Methods for delivering RNA effector molecules to animal cells to induce transkingdom RNAi (tkRNAi) are described, e.g., in U.S. Pat. Pub. Nos. 20080311081 to Fruehauf et al. and 20090123426 to Li et al., both of which are herein incorporated by reference in their entirety. In one embodiment, the RNA effector molecule is an siRNA molecule. In one embodiment, the RNA effector molecule is not an shRNA molecule.

In some embodiments, the RNA effector molecule comprises a microRNA (miRNA). MicroRNAs are a highly conserved class of small RNA molecules that are transcribed from DNA in the genomes of plants and animals, but are not translated into protein. Pre-microRNAs are processed into miRNAs. Processed microRNAs are single stranded ˜17-25 nucleotide (nt) RNA molecules that become incorporated into the RNA-induced silencing complex (RISC) and have been identified as key regulators of development, cell proliferation, apoptosis and differentiation. They are believed to play a role in regulation of gene expression by binding to the 3′-untranslated region of specific mRNAs. MicroRNAs cause post-transcriptional silencing of specific target genes, e.g., by inhibiting translation or initiating degradation of the targeted mRNA. In some embodiments, the miRNA is completely complementary with the target nucleic acid. In other embodiments, the miRNA has a region of noncomplementarity with the target nucleic acid, resulting in a “bulge” at the region of non-complementarity. In some embodiments, the region of noncomplementarity (the bulge) is flanked by regions of sufficient complementarity, e.g., complete complementarity, to allow duplex formation. Preferably, the regions of complementarity are at least 8 to 10 nucleotides long (e.g., 8, 9, or 10 nucleotides long). miRNA can inhibit gene expression by, e.g., repressing translation, such as when the miRNA is not completely complementary to the target nucleic acid, or by causing target RNA degradation, when the miRNA binds its target with perfect or a high degree of complementarity.

In further embodiments, the RNA effector molecule can comprise an oligonucleotide agent which targets an endogenous miRNA or pre-miRNA. For example, the RNA effector can target an endogenous miRNA which negatively regulates expression of a target gene, such that the RNA effector alleviates miRNA-based inhibition of the target gene. The oligonucleotide agent can include naturally occurring nucleobases, sugars, and covalent internucleotide (backbone) linkages and/or oligonucleotides having one or more non-naturally-occurring features that confer desirable properties, such as enhanced cellular uptake, enhanced affinity for the endogenous miRNA target, and/or increased stability in the presence of nucleases. In some embodiments, an oligonucleotide agent designed to bind to a specific endogenous miRNA has substantial complementarity, e.g., at least 70, 80, 90, or 100% complementary, with at least 10, 20, or 25 or more bases of the target miRNA. Exemplary oligonucleotide agents that target miRNAs and pre-miRNAs are described, for example, in U.S. Pat. Pub. Nos.: 20090317907, 20090298174, 20090291907, 20090291906, 20090286969, 20090236225, 20090221685, 20090203893, 20070049547, 20050261218, 20090275729, 20090043082, 20070287179, 20060212950, 20060166910, 20050227934, 20050222067, 20050221490, 20050221293, 20050182005, and 20050059005, contents of all of which are herein incorporated by reference.

An miRNA or pre-miRNA can be 16-100 nucleotides in length, and more preferably from 16-80 nucleotides in length. Mature miRNAs can have a length of 16-30 nucleotides, preferably 21-25 nucleotides, particularly 21, 22, 23, 24, or 25 nucleotides. miRNA precursors can have a length of 70-100 nucleotides and can have a hairpin conformation. In some embodiments, miRNAs are generated in vivo from pre-miRNAs by the enzymes cDicer and Drosha. miRNAs or pre-miRNAs can be synthesized in vivo by a cell-based system or can be chemically synthesized. miRNAs can comprise modifications which impart one or more desired properties, such as improved stability, hybridization thermodynamics with a target nucleic acid, targeting to a particular tissue or cell-type, and/or cell permeability, e.g., by an endocytosis-dependent or -independent mechanism. Modifications can also increase sequence specificity, and consequently decrease off-site targeting.

In some embodiments, the RNA effector molecule comprises a single-stranded oligonucleotide that interacts with and directs the cleavage of RNA transcripts of a target gene. It is particularly preferred that single stranded RNA effector molecules comprise a 5′ modification including one or more phosphate groups or analogs thereof to protect the effector molecule from nuclease degradation.

In some embodiments, the RNA effector molecule comprises an antagomir. Antagomirs are single stranded, double stranded, partially double stranded or hairpin structures that target a microRNA. An antagomir consisting essentially of or comprises at least 12 or more contiguous nucleotides substantially complementary to an endogenous miRNA and more particularly a target sequence of an miRNA or pre-miRNA nucleotide sequence. Antagomirs preferably have a nucleotide sequence sufficiently complementary to a miRNA target sequence of about 12 to 25 nucleotides, preferably about 15 to 23 nucleotides, to allow the antagomir to hybridize to the target sequence. More preferably, the target sequence differs by no more than 1, 2, or 3 nucleotides from the sequence of the antagomir. In some embodiments, the antagomir includes a non-nucleotide moiety, e.g., a cholesterol moiety, which can be attached, e.g., to the 3′ or 5′ end of the oligonucleotide agent.

In some embodiments, antagomirs are stabilized against nucleolytic degradation by the incorporation of a modification, e.g., a nucleotide modification. For example, in some embodiments, antagomirs contain a phosphorothioate comprising at least the first, second, and/or third internucleotide linkages at the 5′ or 3′ end of the nucleotide sequence. In further embodiments, antagomirs include a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O-N-methylacetamido (2′-O-NMA). In some preferred embodiments, antagomirs include at least one 2′-O-methyl-modified nucleotide.

In some embodiments, the RNA effector molecule comprises an aptamer which binds to a non-nucleic acid ligand, such as a small organic molecule or protein, e.g., a transcription or translation factor, and subsequently inhibits activity. An aptamer can fold into a specific structure that directs the recognition of a targeted binding site on the non-nucleic acid ligand. Aptamers can contain any of the modifications described herein.

In some embodiments, the RNA effector molecule is a single-stranded “antisense” nucleic acid having a nucleotide sequence that is complementary to at least a portion of a “sense” nucleic acid of a target gene, e.g., the coding strand of a double-stranded cDNA molecule or an RNA sequence, e.g., a pre-mRNA, mRNA, miRNA, or pre-miRNA. Accordingly, an antisense nucleic acid can form hydrogen bonds with a sense nucleic acid target. In an alternative embodiment, the RNA effector molecule comprises a duplex region of at least 9 nucleotides in length.

Given a coding strand sequence (e.g., the sequence of a sense strand of a cDNA molecule), antisense nucleic acids can be designed according to the rules of Watson and Crick base pairing. The antisense nucleic acid can be complementary to a portion of the coding or noncoding region of an RNA, e.g., the region surrounding the translation start site of a pre-mRNA or mRNA, e.g., the 5′ UTR. An antisense oligonucleotide can be, for example, about 10 to 25 nucleotides in length (e.g., 11, 12, 13, 14, 15, 16, 18, 19, 20, 21, 22, 23, or 24 nucleotides in length). In some embodiments, the antisense oligonucleotide comprises one or more modified nucleotides, e.g., phosphorothioate derivatives and/or acridine substituted nucleotides, designed to increase the biological stability of the molecule and/or the physical stability of the duplexes formed between the antisense and target nucleic acids. Antisense oligonucleotides can comprise ribonucleotides only, deoxyribonucleotides only (e.g., oligodeoxynucleotides), or both deoxyribonucleotides and ribonucleotides. For example, an antisense agent consisting only of ribonucleotides can hybridize to a complementary RNA and prevent access of the translation machinery to the target RNA transcript, thereby preventing protein synthesis. An antisense molecule including only deoxyribonucleotides, or deoxyribonucleotides and ribonucleotides, can hybridize to a complementary RNA and the RNA target can be subsequently cleaved by an enzyme, e.g., RNAse H, to prevent translation. The flanking RNA sequences can include 2′-O-methylated nucleotides, and phosphorothioate linkages, and the internal DNA sequence can include phosphorothioate internucleotide linkages. The internal DNA sequence is preferably at least five nucleotides in length when targeting by RNAseH activity is desired.

The skilled artisan will recognize that the term “oligonucleotide” or “nucleic acid molecule” encompasses not only nucleic acid molecules as expressed or found in nature, but also analogs and derivatives of nucleic acids comprising one or more ribo- or deoxyribo-nucleotide/nucleoside analogs or derivatives as described herein or as known in the art. Strictly speaking, a “nucleoside” includes a nucleoside base and a ribose or a 2′-deoxyribose sugar, and a “nucleotide” is a nucleoside with one, two or three phosphate moieties. However, the terms “nucleoside” and “nucleotide” can be considered to be equivalent as used herein. An oligonucleotide can be modified in the nucleobase structure or in the ribose-phosphate backbone structure, e.g., as described herein below. However, the molecules comprising nucleoside analogs or derivatives must retain the ability to form a duplex. As non-limiting examples, an oligonucleotide can also include at least one modified nucleoside including but not limited to a 2′-O-methyl modified nucleoside, a nucleoside comprising a 5′ phosphorothioate group, a terminal nucleoside linked to a cholesterol derivative or dodecanoic acid bisdecylamide group, a locked nucleoside, an abasic nucleoside, a 2′-deoxy-2′-fluoro modified nucleoside, a 2′-amino-modified nucleoside, 2′-alkyl-modified nucleoside, morpholino nucleoside, a phosphoramidate or a non-natural base comprising nucleoside, or any combination thereof Alternatively, an oligonucleotide can comprise at least two modified nucleosides, at least 3, at least 4, at least 5, at least 6, at least 7, at least 8, at least 9, at least 10, at least 15, at least 20 or more, up to the entire length of the oligonucleotide. The modifications need not be the same for each of such a plurality of modified nucleosides in an oligonucleotide. When RNA effector molecule is double stranded, each strand can be independently modified as to number, type and/or location of the modified nucleosides. In one embodiment, modified oligonucleotides contemplated for use in methods and compositions described herein are peptide nucleic acids (PNAs) that have the ability to form the required duplex structure and that permit or mediate the specific degradation of a target RNA via a RISC pathway.

A double-stranded oligonucleotide can include one or more single-stranded nucleotide overhangs. As used herein, the term “nucleotide overhang” refers to at least one unpaired nucleotide that protrudes from the duplex structure of a double-stranded oligonucleotide, e.g., a dsRNA. For example, when a 3′-end of one strand of double-stranded oligonucleotide extends beyond the 5′-end of the other strand, or vice versa, there is a nucleotide overhang. A double-stranded oligonucleotide can comprise an overhang of at least one nucleotide; alternatively the overhang can comprise at least two nucleotides, at least three nucleotides, at least four nucleotides, at least five nucleotides or more. A nucleotide overhang can comprise or consist of a nucleotide/nucleoside analog. The overhang(s) can be on the sense strand, the antisense strand or any combination thereof Furthermore, the nucleotide(s) of an overhang can be present on the 5′ end, 3′ end or both ends of either an antisense or sense strand of a dsRNA.

In one embodiment, the antisense strand of a double-stranded oligonucleotide has a 1-10 nucleotide overhang at the 3′ end and/or the 5′ end. In one embodiment, the sense strand of a double-stranded oligonucleotide has a 1-10 nucleotide overhang at the 3′ end and/or the 5′ end. In another embodiment, one or more of the internucleoside linkages in the overhang is replaced with a phosphorothioate. In some embodiments, the overhang comprises one or more deoxyribonucleoside. In some embodiments, overhang comprises the sequence 5′-dTdT-3. In some embodiments, overhang comprises the sequence 5′-dT*dT-3, wherein * is a phosphorothioate internucleoside linkage.

The terms “blunt” or “blunt ended” as used herein in reference to double-stranded oligonucleotide mean that there are no unpaired nucleotides or nucleotide analogs at a given terminal end of a double-stranded oligonucleotide, i.e., no nucleotide overhang. One or both ends of a double-stranded oligonucleotide can be blunt. Where both ends are blunt, the oligonucleotide is said to be double-blunt ended. To be clear, a “double-blunt ended” oligonucleotide is a double-stranded oligonucleotide that is blunt at both ends, i.e., no nucleotide overhang at either end of the molecule. Most often such a molecule will be double-stranded over its entire length. When only one end of is blunt, the oligonucleotide is said to be single-blunt ended. To be clear, a “single-blunt ended” oligonucleotide is a double-stranded oligonucleotide that is blunt at only one end, i.e., no nucleotide overhang at one end of the molecule. Generally, a single-blunt ended oligonucleotide is blunt ended at the 5′-end of sense stand.

The term “antisense strand” or “guide strand” refers to the strand of an RNA effector molecule, e.g., a dsRNA, which includes a region that is substantially complementary to a target sequence. As used herein, the term “region of complementarity” refers to the region on the antisense strand that is substantially complementary to a sequence, for example a target sequence, as defined herein. Where the region of complementarity is not fully complementary to the target sequence, the mismatches can be in the internal or terminal regions of the molecule. Generally, the most tolerated mismatches are in the terminal regions, e.g., within 5, 4, 3, or 2 nucleotides of the 5′ and/or 3′ terminus

The term “sense strand,” or “passenger strand” as used herein, refers to the strand of an RNA effector molecule that includes a region that is substantially complementary to a region of the antisense strand as that term is defined herein.

### Plurality of RNA Effector Molecules

In one embodiment, a plurality of different RNA effector molecules are contacted with the cell culture and permit inhibition of a transgene and/or a selectable amplifiable marker. In one embodiment, the RNA effector molecules are contacted with the cell culture during production of the polypeptide.

In some embodiments, RNA effector compositions comprise two or more RNA effector molecules, e.g., two, three, four or more RNA effector molecules. In one embodiment, the two or more RNA effector molecules are capable of modulating expression of a selectable amplifiable marker, a transgene or a combination thereof. In another embodiment, an RNA effector molecule that modulates expression of an additional target gene is contemplated herein.

In one embodiment, when a plurality of different RNA effector molecules or RNA effector molecule compositions are used to modulate expression of a selectable amplifiable marker and a target gene, the plurality of RNA effector molecules are contacted with the culture simultaneously or separately. In addition, each RNA effector molecule can have its own dosage regime. For example, in one embodiment one can prepare a composition comprising a plurality of RNA effector molecules that is contacted with a cell. Alternatively, one can administer one RNA effector molecule at a time to the cell culture. In this manner, one can easily tailor the average percent inhibition desired for each target RNA by altering the frequency of administration of a particular RNA effector molecule. Contacting a cell with each RNA effector molecule separately can also prevent interactions between RNA effector molecules that can reduce efficiency of target gene modulation. For ease of use and to prevent potential contamination it may be preferred to administer a cocktail of different RNA effector molecules, thereby reducing the number of doses required and minimizing the chance of introducing a contaminant to the cell culture.

dsRNA Effector Molecules

In some embodiments, RNA effector molecule is a double-stranded oligonucleotide comprising a sense strand and an antisense strand, wherein the antisense strand has a region of complementarity to at least part of a target gene RNA. The sense strand includes a region that is complementary to the antisense strand, such that the two strands hybridize and form a duplex structure when combined under suitable conditions. Typically, the region of complementarity is 30 nucleotides or less in length, generally 10-26 nucleotides in length, preferably 18-25 nucleotides in length, and most preferably 19-24 nucleotides in length. Upon contact with a cell expressing the target gene, the RNA effector molecule inhibits the expression of the target gene by at least 10% as assayed by, for example, a PCR or branched DNA (bDNA)-based method, or by a protein-based method, such as by Western blot. Expression of a target gene in cell culture, such as in COS cells, HeLa cells, CHO cells, or the like, can be assayed by measuring target gene mRNA levels, e.g., by bDNA or TaqMan assay, or by measuring protein levels, e.g., by immunofluorescence analysis.

As the ordinarily skilled person will recognize, the targeted region of an RNA targeted for cleavage will most often be part of a larger RNA molecule, often an mRNA molecule. Where relevant, a “part” of an RNA target is a contiguous sequence of an RNA target of sufficient length to be a substrate for RNAi-directed cleavage (i.e., cleavage through a RISC pathway). dsRNAs having duplexes as short as 9 base pairs can, under some circumstances, mediate RNAi-directed RNA cleavage. Most often a target will be at least 15 nucleotides in length, preferably 15-30 nucleotides in length.

One of skill in the art will also recognize that the duplex region is a primary functional portion of a double-stranded oligonucleotide, e.g., a duplex region of 9 to 36, e.g., 15-30 base pairs. Thus, in one embodiment, to the extent that it becomes processed to a functional duplex of e.g., 15-30 base pairs that targets a desired RNA for cleavage, an oligonucleotide having a duplex region greater than 30 base pairs is an RNA effector molecule.

The oligonucleotides can be synthesized by standard methods known in the art as further discussed below, e.g., by use of an automated DNA synthesizer, such as are commercially available from, for example, Biosearch, Applied Biosystems, Inc. In one embodiment, a target gene is a human target gene. As described elsewhere herein and as known in the art, the complementary sequences of a double-stranded RNA effector molecule can also be contained as self-complementary regions of a single nucleic acid molecule, as opposed to being on separate oligonucleotides (e.g., shRNA).

The skilled person is well aware that dsRNAs having a duplex structure of between 20 and 23, but specifically 21, base pairs have been hailed as particularly effective in inducing RNA interference (Elbashir et al., EMBO 2001, 20:6877-6888, herein incorporated by reference in its entirety). However, others have found that shorter or longer RNA duplex structures can be effective as well. In the embodiments described above, dsRNAs described herein can include at least one strand of a length of minimally 21 nt.

While a target sequence is generally 15-30 nucleotides in length, there is wide variation in the suitability of particular sequences in this range for directing cleavage of any given target RNA. Various software packages and the guidelines set out herein provide guidance for the identification of optimal target sequences for any given gene target, but an empirical approach can also be taken in which a “window” or “mask” of a given size (as a non-limiting example, 21 nucleotides) is literally or figuratively (including, e.g., in silico) placed on the target RNA sequence to identify sequences in the size range that can serve as target sequences. By moving the sequence “window” progressively one nucleotide upstream or downstream of an initial target sequence location, the next potential target sequence can be identified, until the complete set of possible sequences is identified for any given target size selected. This process, coupled with systematic synthesis and testing of the identified sequences (using assays as described herein or as known in the art) to identify those sequences that perform optimally can identify those RNA sequences that, when targeted with an RNA effector molecule agent, mediate the best inhibition of target gene expression. Thus, it is contemplated that further optimization of inhibition efficiency can be achieved by progressively “walking the window” one nucleotide upstream or downstream of the given sequences to identify sequences with equal or better inhibition characteristics.

Further, it is contemplated that for any sequence identified, further optimization could be achieved by systematically either adding or removing nucleotides to generate longer or shorter sequences and testing those sequences generated by walking a window of the longer or shorter size up or down the target RNA from that point. Again, coupling this approach to generating new candidate targets with testing for effectiveness of RNA effector molecules based on those target sequences in an inhibition assay as known in the art or as described herein can lead to further improvements in the efficiency of inhibition. Further still, such optimized sequences can be adjusted by, e.g., the introduction of modified nucleotides as described herein or as known in the art, addition or changes in overhang, or other modifications as known in the art and/or discussed herein to further optimize the molecule (e.g., increasing serum stability or circulating half-life, increasing thermal stability, enhancing transmembrane delivery, targeting to a particular location or cell type, increasing interaction with silencing pathway enzymes, increasing release from endosomes, etc.) as an expression inhibitor.

An RNA effector molecule as described herein can contain one or more mismatches to the target sequence. In one embodiment, an RNA effector molecule as described herein contains no more than 3 mismatches. If the antisense strand of the RNA effector molecule contains mismatches to a target sequence, it is preferable that the area of mismatch not be located in the center of the region of complementarity. If the antisense strand of the RNA effector molecule contains mismatches to the target sequence, it is preferable that the mismatch be restricted to be within the last 5 nucleotides from either the 5′ or 3′ end of the region of complementarity. For example, for a 23 nucleotide RNA effector molecule agent RNA strand which is complementary to a region of a target gene, the RNA strand generally does not contain any mismatch within the central 13 nucleotides. The methods described herein or methods known in the art can be used to determine whether an RNA effector molecule containing a mismatch to a target sequence is effective in inhibiting the expression of a target gene. Consideration of the efficacy of RNA effector molecules with mismatches in inhibiting expression of a target gene is important, especially if the particular region of complementarity in a target gene is known to have polymorphic sequence variation within the population.

In yet another embodiment, an oligonucleotide is chemically modified to enhance stability or other beneficial characteristics. Oligonucleotides can be modified to prevent rapid degradation of the oligonucleotides by endo- and exo-nucleases and avoid undesirable off-target effects. The nucleic acids featured in the invention can be synthesized and/or modified by methods well established in the art, such as those described in “Current protocols in nucleic acid chemistry,” Beaucage, S. L. et al. (Edrs.), John Wiley & Sons, Inc., New York, N.Y., USA, which is hereby incorporated herein by reference in its entirety. Modifications include, for example, (a) end modifications, e.g., 5′ end modifications (phosphorylation, conjugation, inverted linkages, etc.) 3′ end modifications (conjugation, DNA nucleotides, inverted linkages, etc.), (b) base modifications, e.g., replacement with stabilizing bases, destabilizing bases, or bases that base pair with an expanded repertoire of partners, removal of bases (abasic nucleotides), or conjugated bases, (c) sugar modifications (e.g., at the 2′ position or 4′ position) or replacement of the sugar, as well as (d) internucleoside linkage modifications, including modification or replacement of the phosphodiester linkages. Specific examples of oligonucleotide compounds useful in this invention include, but are not limited to oligonucleotides containing modified or non-natural internucleoside linkages. Oligonucleotides having modified internucleoside linkages include, among others, those that do not have a phosphorus atom in the internucleoside linkage. For the purposes of this specification, and as sometimes referenced in the art, modified oligonucleotides that do not have a phosphorus atom in their internucleoside linkage(s) can also be considered to be oligonucleosides. In particular embodiments, the modified oligonucleotides will have a phosphorus atom in its internucleoside linkage(s).

Modified internucleoside linkages include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included.

Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6, 239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. RE39464, each of which is herein incorporated by reference in its entirety.

Modified oligonucleotide internucleoside linkages that do not include a phosphorus atom therein have internucleoside linkages that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and 5,677,439, each of which is herein incorporated by reference in its entirety.

In other modified oligonucleotides suitable or contemplated for use in RNA effector molecules, both the sugar and the internucleoside linkage, i.e., the backbone, of the nucleotide units are replaced with novel groups. The base units are maintained for hybridization with an appropriate nucleic acid target compound. One such oligomeric compound, an RNA mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262, each of which is herein incorporated by reference in its entirety. Further teaching of PNA compounds can be found, for example, in Nielsen et al., Science, 1991, 254, 1497-1500, herein incorporated by reference in its entirety.

Some embodiments featured in the invention include oligonucleotides with phosphorothioate internucleoside linkages and oligonucleosides with heteroatom internucleoside linkage, and in particular —CH₂—NH—CH₂—, —CH₂—N(CH₃)—O—CH₂— [known as a methylene (methylimino) or MMI], —CH₂—O—N(CH₃)—CH₂—, —CH₂—N(CH₃)—N(CH₃)—CH₂— and —N(CH₃)—CH₂—CH₂— [wherein the native phosphodiester internucleoside linkage is represented as —O—P—O—CH₂—] of the above-referenced U.S. Pat. No. 5,489,677, and the amide backbones of the above-referenced U.S. Pat. No. 5,602,240, both of which are herein incorporated by reference in their entirety. In some embodiments, the oligonucleotides featured herein have morpholino backbone structures of the above-referenced U.S. Pat. No. 5,034,506, herein incorporated by reference in its entirety.

Modified oligonucleotides can also contain one or more substituted sugar moieties. The oligonucleotides featured herein can include one of the following at the 2′ position: H (deoxyribose); OH (ribose); F; O-, S-, or N-alkyl; O-, S-, or N-alkenyl; O-, S- or N-alkynyl; or O-alkyl-O-alkyl, wherein the alkyl, alkenyl and alkynyl can be substituted or unsubstituted C₁ to C₁₀ alkyl or C₂ to C₁₀ alkenyl and alkynyl. Exemplary suitable modifications include O[(CH₂)ₙO]ₘCH₃, O(CH₂).ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. In some embodiments, oligonucleotides include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. In some embodiments, the modification includes a 2′-methoxyethoxy (2′-O—CH₂CH₂OCH₃, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂, also described in examples herein below.

Other modifications include 2′-methoxy (2′-OCH₃), 2′-aminopropoxy (2′-OCH₂CH₂CH₂NH₂) and 2′-fluoro (2′-F). Similar modifications can also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotide can also have sugar mimetics such as cyclobutyl moieties in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, certain of which are commonly owned with the instant application, and each of which is herein incorporated by reference in its entirety.

An oligonucleotide can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as inosine, xanthine, hypoxanthine, nubularine, isoguanisine, tubercidine, 2-(halo)adenine, 2-(alkyl)adenine, 2-(propyl)adenine, 2-(amino)adenine, 2-(aminoalkyll)adenine, 2-(aminopropyl)adenine, 2-(methylthio)-N⁶-(isopentenyl)adenine, 6-(alkyl)adenine, 6-(methyl)adenine, 7-(deaza)adenine, 8-(alkenyl)adenine, 8-(alkyl)adenine, 8-(alkynyl)adenine, 8-(amino)adenine, 8-(halo)adenine, 8-(hydroxyl)adenine, 8-(thioalkyl)adenine, 8-(thiol)adenine, N⁶-(isopentyl)adenine, N⁶-(methyl)adenine, N⁶,N⁶-(dimethyl)adenine, 2-(alkyl)guanine,2-(propyl)guanine, 6-(alkyl)guanine, 6-(methyl)guanine, 7-(alkyl)guanine, 7-(methyl)guanine, 7-(deaza)guanine, 8-(alkyl)guanine, 8-(alkenyl)guanine, 8-(alkynyl)guanine, 8-(amino)guanine, 8-(halo)guanine, 8-(hydroxyl)guanine, 8-(thioalkyl)guanine, 8-(thiol)guanine, N-(methyl)guanine, 2-(thio)cytosine, 3-(deaza)-5-(aza)cytosine, 3-(alkyl)cytosine, 3-(methyl)cytosine, 5-(alkyl)cytosine, 5-(alkynyl)cytosine, 5-(halo)cytosine, 5-(methyl)cytosine, 5-(propynyl)cytosine, 5-(propynyl)cytosine, 5-(trifluoromethyl)cytosine, 6-(azo)cytosine, N⁴-(acetyl)cytosine, 3-(3-amino-3-carboxypropyl)uracil, 2-(thio)uracil, 5-(methyl)-2-(thio)uracil, 5-(methylaminomethyl)-2-(thio)uracil, 4-(thio)uracil, 5-(methyl)-4-(thio)uracil, 5-(methylaminomethyl)-4-(thio)uracil, 5-(methyl)-2,4-(dithio)uracil, 5-(methylaminomethyl)-2,4-(dithio)uracil, 5-(2-aminopropyl)uracil, 5-(alkyl)uracil, 5-(alkynyl)uracil, 5-(allylamino)uracil, 5-(aminoallyl)uracil, 5-(aminoalkyl)uracil, 5-(guanidiniumalkyl)uracil, 5-(1,3-diazole-1-alkyl)uracil, 5-(cyanoalkyl)uracil, 5-(dialkylaminoalkyl)uracil, 5-(dimethylaminoalkyl)uracil, 5-(halo)uracil, 5-(methoxy)uracil, uracil-5-oxyacetic acid, 5-(methoxycarbonylmethyl)-2-(thio)uracil, 5-(methoxycarbonyl-methyl)uracil, 5-(propynyl)uracil, 5-(propynyl)uracil, 5-(trifluoromethyl)uracil, 6-(azo)uracil, dihydrouracil, N³-(methyl)uracil, 5-uracil (i.e., pseudouracil), 2-(thio)pseudouracil,4-(thio)pseudouracil,2,4-(dithio)psuedouracil,5-(alkyl)pseudouracil, 5-(methyl)pseudouracil, 5-(alkyl)-2-(thio)pseudouracil, 5-(methyl)-2-(thio)pseudouracil, 5-(alkyl)-4-(thio)pseudouracil, 5-(methyl)-4-(thio)pseudouracil, 5-(alkyl)-2,4-(dithio)pseudouracil, 5-(methyl)-2,4-(dithio)pseudouracil, 1-substituted pseudouracil, 1-substituted 2(thio)-pseudouracil, 1-substituted 4-(thio)pseudouracil, 1-substituted 2,4-(dithio)pseudouracil, 1-(aminocarbonylethylenyl)-pseudouracil, 1-(aminocarbonylethylenyl)-2(thio)-pseudouracil, 1-(aminocarbonylethylenyl)-4-(thio)pseudouracil, 1-(aminocarbonylethylenyl)-2,4-(dithio)pseudouracil, 1-(aminoalkylaminocarbonylethylenyl)-pseudouracil, 1-(aminoalkylamino-carbonylethylenyl)-2(thio)-pseudouracil, 1-(aminoalkylaminocarbonylethylenyl)-4-(thio)pseudouracil, 1-(aminoalkylaminocarbonylethylenyl)-2,4-(dithio)pseudouracil, 1,3-(diaza)-2-(oxo)-phenoxazin-1-yl, 1-(aza)-2-(thio)-3-(aza)-phenoxazin-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted 1-(aza)-2-(thio)-3-(aza)-phenoxazin-1-yl, 7-substituted 1,3-(diaza)-2-(oxo)-phenthiazin-1-yl, 7-substituted 1-(aza)-2-(thio)-3-(aza)-phenthiazin-1-yl, 7-(aminoalkylhydroxy)-1,3-(diaza)-2-(oxo)-phenoxazin-1-yl, 7-(aminoalkylhydroxy)-1-(aza)-2-(thio)-3-(aza)-phenoxazin-1-yl, 7-(aminoalkylhydroxy)-1,3-(diaza)-2-(oxo)-phenthiazin-1-yl, 7-(aminoalkylhydroxy)-1-(aza)-2-(thio)-3-(aza)-phenthiazin-1-yl, 7-(guanidiniumalkylhydroxy)-1,3-(diaza)-2-(oxo)-phenoxazin-1-yl, 7-(guanidiniumalkylhydroxy)-1-(aza)-2-(thio)-3-(aza)-phenoxazin-1-yl, 7-(guanidiniumalkyl-hydroxy)-1,3-(diaza)-2-(oxo)-phenthiazin-1-yl, 7-(guanidiniumalkylhydroxy)-1-(aza)-2-(thio)-3-(aza)-phenthiazin-1-yl, 1,3,5-(triaza)-2,6-(dioxa)-naphthalene, inosine, xanthine, hypoxanthine, nubularine, tubercidine, isoguanisine, inosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, 3-(methyl)isocarbostyrilyl, 5-(methyl)isocarbostyrilyl, 3-(methyl)-7-(propynyl)isocarbostyrilyl, 7-(aza)indolyl, 6-(methyl)-7-(aza)indolyl, imidizopyridinyl, 9-(methyl)-imidizopyridinyl, pyrrolopyrizinyl, isocarbostyrilyl, 7-(propynyl)isocarbostyrilyl, propynyl-7-(aza)indolyl, 2,4,5-(trimethyl)phenyl, 4-(methyl)indolyl, 4,6-(dimethyl)indolyl, phenyl, napthalenyl, anthracenyl, phenanthracenyl, pyrenyl, stilbenyl, tetracenyl, pentacenyl, difluorotolyl, 4-(fluoro)-6-(methyl)benzimidazole, 4-(methyl)benzimidazole, 6-(azo)thymine, 2-pyridinone, 5-nitroindole, 3-nitropyrrole, 6-(aza)pyrimidine, 2-(amino)purine, 2,6-(diamino)purine, 5-substituted pyrimidines, N²-substituted purines, N⁶-substituted purines, O⁶-substituted purines, substituted 1,2,4-triazoles, pyrrolo-pyrimidin-2-on-3-yl, 6-phenyl-pyrrolo-pyrimidin-2-on-3-yl, para-substituted-6-phenyl-pyrrolo-pyrimidin-2-on-3-yl, ortho-substituted-6-phenyl-pyrrolo-pyrimidin-2-on-3-yl, bis-ortho-substituted-6-phenyl-pyrrolo-pyrimidin-2-on-3-yl,para-(aminoalkylhydroxy)-6-phenyl-pyrrolo-pyrimidin-2-on-3-yl, ortho-(aminoalkylhydroxy)-6-phenyl-pyrrolo-pyrimidin-2-on-3-yl, bis-ortho-(aminoalkylhydroxy)-6-phenyl-pyrrolo-pyrimidin-2-on-3-yl, pyridopyrimidin-3-yl, 2-oxo-7-amino-pyridopyrimidin-3-yl, 2-oxo-pyridopyrimidine-3-yl, or any O-alkylated or N-alkylated derivatives thereof. Modified nucleobases also include natural bases that comprise conjugated moieties, e.g. a ligand described herein.

Further nucleobases include those disclosed in U.S. Pat. No. 3,687,808, those disclosed in Modified Nucleosides in Biochemistry, Biotechnology and Medicine, Herdewijn, P. ed. Wiley-VCH, 2008; those disclosed in Int. Appl. No. PCT/US09/038425, filed Mar. 26, 2009; those disclosed in The Concise Encyclopedia Of Polymer Science And Engineering, pages 858-859, Kroschwitz, J. L, ed. John Wiley & Sons, 1990, these disclosed by Englisch et al., Angewandte Chemie, International Edition, 1991, 30, 613, and those disclosed by Sanghvi, Y S., Chapter 15, dsRNA Research and Applications, pages 289-302, Crooke, S. T. and Lebleu, B., Ed., CRC Press, 1993. Certain of these nucleobases are particularly useful for increasing the binding affinity of the oligomeric compositions featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278, herein incorporated by reference in its entirety) and are exemplary base substitutions, even more particularly when combined with 2′-O-methoxyethyl sugar modifications.

Representative U.S. patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,30; 5,134,066; 5,175,273; 5,367,066; 5,432,272; 5,457,187; 5,457,191; 5,459,255; 5,484,908; 5,502,177; 5,525,711; 5,552,540; 5,587,469; 5,594,121, 5,596,091; 5,614,617; 5,681,941; 6,015,886; 6,147,200; 6,166,197; 6,222,025; 6,235,887; 6,380,368; 6,528,640; 6,639,062; 6,617,438; 7,045,610; 7,427,672; and 7,495,088, each of which is herein incorporated by reference in its entirety, and U.S. Pat. No. 5,750,692, also herein incorporated by reference in its entirety.

The oligonucleotides can also be modified to include one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to oligonucleotides has been shown to increase oligonucleotide stability in serum, and to reduce off-target effects (see e.g., Elmen, J. et al., (2005) Nucleic Acids Research 33(1):439-447; Mook, O R. et al., (2007) Mol Canc Ther 6(3):833-843; Grunweller, A. et al., (2003) Nucleic Acids Research 31(12):3185-3193, each of which is herein incorporated by reference in its entirety).

Representative U.S. patents that teach the preparation of locked nucleic acid nucleotides include, but are not limited to, the following: U.S. Pat. Nos. 6,268,490; 6,670,461; 6,794,499; 6,998,484; 7,053,207; 7,084,125; and 7,399,845, each of which is herein incorporated by reference in its entirety.

Another modification of the oligonucleotides featured in the invention involves chemically linking to the oligonucleotide one or more ligands, moieties or conjugates that enhance the activity, cellular distribution or cellular uptake of the oligonucleotide. Such moieties include but are not limited to lipid moieties such as a cholesterol moiety (Letsinger et al., Proc. Natl. Acid. Sci. USA, 1989, 86: 6553-6556, herein incorporated by reference in its entirety), cholic acid (Manoharan et al., Biorg. Med. Chem. Let., 1994, 4:1053-1060, herein incorporated by reference in its entirety), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770, each of which is herein incorporated by reference in its entirety), a thiocholesterol (Oberhauser et al., Nucl. Acids Res., 1992, 20:533-538, herein incorporated by reference in its entirety), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54, each of which is herein incorporated by reference in its entirety), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783, each of which is herein incorporated by reference in its entirety), a polyamine or a polyethylene glycol chain (Manoharan et al., Nucleosides & Nucleotides, 1995, 14:969-973, herein incorporated by reference in its entirety), or adamantane acetic acid (Manoharan et al., Tetrahedron Lett., 1995, 36:3651-3654, herein incorporated by reference in its entirety), a palmityl moiety (Mishra et al., Biochim. Biophys. Acta, 1995, 1264:229-237, herein incorporated by reference in its entirety), or an octadecylamine or hexylamino-carbonyloxycholesterol moiety (Crooke et al., J. Pharmacol. Exp. Ther., 1996, 277:923-937, herein incorporated by reference in its entirety).

In one embodiment, a ligand alters the cellular uptake, intracellular targeting or half-life of an RNA effector molecule agent into which it is incorporated. In preferred embodiments a ligand provides an enhanced affinity for a selected target, e.g., molecule, cell or cell type, intracellular compartment, e.g., mitochondria, cytoplasm, peroxisome, lysosome, as, e.g., compared to a composition absent such a ligand. Preferred ligands will not take part in duplex pairing in a duplexed nucleic acid.

Ligands can include a naturally occurring substance, such as a protein (e.g., human serum albumin (HSA), low-density lipoprotein (LDL), or globulin); carbohydrate (e.g., a dextran, pullulan, chitin, chitosan, inulin, cyclodextrin or hyaluronic acid); or a lipid. The ligand can also be a recombinant or synthetic molecule, such as a synthetic polymer, e.g., a synthetic polyamino acid. Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

Ligands can also include targeting groups, e.g., a cell targeting agent, (e.g., a lectin, glycoprotein, lipid or protein), or an antibody, that binds to a specified cell type such as a CHO cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-glucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, or an RGD peptide or RGD peptide mimetic.

Other examples of ligands include dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), lipophilic molecules, e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid,O3-(oleoyl)lithocholic acid, O3-(oleoyl)cholenic acid, dimethoxytrityl, or phenoxazine)and peptide conjugates (e.g., antennapedia peptide, Tat peptide), alkylating agents, phosphate, amino, mercapto, PEG (e.g., PEG-40K), MPEG, [MPEG]₂, polyamino, alkyl, substituted alkyl, radiolabeled markers, enzymes, haptens (e.g. biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, or AP.

Ligands can be proteins, e.g., glycoproteins, or peptides, e.g., molecules having a specific affinity for a co-ligand, or antibodies e.g., an antibody, that binds to a specified cell type such as a CHO cell, or other cell useful in the production of polypeptides. Ligands can also include hormones and hormone receptors. They can also include non-peptidic species, such as lipids, lectins, carbohydrates, vitamins, cofactors, multivalent lactose, multivalent galactose, N-acetyl-galactosamine, N-acetyl-glucosamine multivalent mannose, or multivalent fucose. The ligand can be, for example, a lipopolysaccharide, an activator of p38 MAP kinase, or an activator of NF-κB.

The ligand can be a substance, e.g., a drug, which can increase the uptake of the RNA effector molecule agent into the cell, for example, by disrupting the cell's cytoskeleton, e.g., by disrupting the cell's microtubules, microfilaments, and/or intermediate filaments. The drug can be, for example, taxol, vincristine, vinblastine, cytochalasin, nocodazole, japlakinolide, latrunculin A, phalloidin, swinholide A, indanocine, or myoservin.

One exemplary ligand is a lipid or lipid-based molecule. A lipid or lipid-based ligand can (a) increase resistance to degradation of the conjugate, and/or (b) increase targeting or transport into a target cell or cell membrane. A lipid based ligand can be used to modulate, e.g., binding of the RNA effector molecule composition to a target cell.

In some embodiments, the ligand is a lipid or lipid-based molecule that preferably binds a serum protein, e.g., human serum albumin (HSA). An HSA binding ligand allows for distribution of the conjugate to a target tissue, e.g., a non-kidney target tissue of the body. For example, the target tissue can be the liver, including parenchymal cells of the liver. Other molecules that can bind HSA can also be used as ligands. For example, Naproxen or aspirin can be used. A lipid or lipid-based ligand can (a) increase resistance to degradation of the conjugate, (b) increase targeting or transport into a target cell or cell membrane, and/or (c) can be used to adjust binding to a serum protein, e.g., HSA.

A lipid based ligand can be used to modulate, e.g., control the binding of the conjugate to a target tissue. For example, a lipid or lipid-based ligand that binds to HSA more strongly will be less likely to be targeted to the kidney and therefore less likely to be cleared from the embryo. A lipid or lipid-based ligand that binds to HSA less strongly can be used to target the conjugate to the kidney. For example, the lipid based ligand binds HSA, or it binds HSA with a sufficient affinity such that the conjugate will be distributed to a non-kidney tissue but also be reversible. Alternatively, the lipid-based ligand binds HSA weakly or not at all, such that the conjugate will be distributed to the kidney. Other moieties that target to kidney cells can also be used in place of or in addition to the lipid-based ligand.

In another aspect, the ligand is a moiety, e.g., a vitamin, which is taken up by a host cell. Exemplary vitamins include vitamin A, E, and K. Other exemplary vitamins include are B vitamin, e.g., folic acid, B12, riboflavin, biotin, pyridoxal or other vitamins or nutrients taken up by cancer cells. Also included are HSA and low density lipoprotein (LDL).

In another aspect, the ligand is a cell-permeation agent, preferably a helical cell-permeation agent. Preferably, the agent is amphipathic. An exemplary agent is a peptide such as that or antennopedia. If the agent is a peptide, it can be modified, including a peptidylmimetic, invertomers, non-peptide or pseudo-peptide linkages, and use of D-amino acids. The helical agent is preferably an alpha-helical agent, which preferably has a lipophilic and a lipophobic phase.

The ligand can be a peptide or peptidomimetic. A peptidomimetic (also referred to herein as an oligopeptidomimetic) is a molecule capable of folding into a defined three-dimensional structure similar to a natural peptide. The attachment of peptide and peptidomimetics to oligonucleotides can affect pharmacokinetic distribution of the oligonucleotide, such as by enhancing cellular recognition and uptake. The peptide or peptidomimetic moiety can be about 5-50 amino acids long, e.g., about 5, 10, 15, 20, 25, 30, 35, 40, 45, or 50 amino acids long (see Table 1, for example).

TABLE 1
Exemplary Cell Permeation Peptides

| Cell | | |
|---|---|---|
| Permeation | | |
| Peptide | Amino acid Sequence | Reference |
| Penetratin | RQIKIWFQNRRMKWKK (SEQ ID NO: 1401) | Derossi et al., J. Biol. |
| | | Chem. 269: 10444, 1994 |
| Tat fragment | GRKKRRQRRRPPQC (SEQ ID NO: 1402) | Vives et al., J. Biol. |
| (48-60) | | Chem., 272: 16010, 1997 |
| Signal | GALFLGWLGAAGSTMGAWSQPKKKRKV | Chaloin et al., Biochem. |
| Sequence-based | (SEQ ID NO: 1403) | Biophys. Res. Commun , |
| peptide | | 243: 601, 1998 |
| PVEC | LLIILRRRIRKQAHAHSK (SEQ ID NO: 1404) | Elmquist et al., Exp. Cell |
| | | Res., 269: 237, 2001 |
| Transportan | GWTLNSAGYLLKINLKALAALAKKIL (SEQ | Pooga et al., FASEB J., |
| | ID NO: 1405) | 12: 67, 1998 |
| Amphiphilic | KLALKLALKALKAALKLA (SEQ ID NO: | Oehlke et al., Mol. Ther., |
| model peptide | 1406) | 2: 339, 2000 |
| Arg₉ | RRRRRRRRR (SEQ ID NO: 1407) | Mitchell et al., J. Pept. |
| | | Res., 56: 318, 2000 |
| Bacterial cell | KFFKFFKFFK (SEQ ID NO: 1408) | |
| wall permeating | | |
| LL-37 | LLGDFFRKSKEKIGKEFKRIVQRIKDFLRNLV | |
| | PRTES (SEQ ID NO: 1409) | |
| Cecropin P1 | SWLSKTAKKLENSAKKRISEGIAIAIQGGPR | |
| | (SEQ ID NO: 1410) | |
| α-defensin | ACYCRIPACIAGERRYGTCIYQGRLWAFCC | |
| | (SEQ ID NO: 1411) | |
| b-defensin | DHYNCVSSGGQCLYSACPIFTKIQGTCYRGK | |
| | AKCCK (SEQ ID NO: 1412) | |
| Bactenecin | RKCRIVVIRVCR (SEQ ID NO: 1413) | |
| PR-39 | RRRPRPPYLPRPRPPPFFPPRLPPRIPPGFPPRFP | |
| | PRFPGKR-NH2 (SEQ ID NO: 1414) | |
| Indolicidin | ILPWKWPWWPWRR-NH2 (SEQ ID NO: 1415) | |

A peptide or peptidomimetic can be, for example, a cell permeation peptide, cationic peptide, amphipathic peptide, or hydrophobic peptide (e.g., consisting primarily of Tyr, Trp or Phe). The peptide moiety can be a dendrimer peptide, constrained peptide or crosslinked peptide. In another alternative, the peptide moiety can include a hydrophobic membrane translocation sequence (MTS). An exemplary hydrophobic MTS-containing peptide is RFGF having the amino acid sequence AAVALLPAVLLALLAP (SEQ ID NO: 1416). An RFGF analogue (e.g., amino acid sequence AALLPVLLAAP (SEQ ID NO:1417)) containing a hydrophobic MTS can also be a targeting moiety. The peptide moiety can be a “delivery” peptide, which can carry large polar molecules including peptides, oligonucleotides, and protein across cell membranes. For example, sequences from the HIV Tat protein (GRKKRRQRRRPPQ (SEQ ID NO:1418)) and the Drosophila Antennapedia protein (RQIKIWFQNRRMKWKK (SEQ ID NO:1419)) have been found to be capable of functioning as delivery peptides. A peptide or peptidomimetic can be encoded by a random sequence of DNA, such as a peptide identified from a phage-display library, or one-bead-one-compound (OBOC) combinatorial library (Lam et al., Nature, 354:82-84, 1991). Preferably the peptide or peptidomimetic tethered to a dsRNA agent via an incorporated monomer unit is a cell targeting peptide such as an arginine-glycine-aspartic acid (RGD)-peptide, or RGD mimic. A peptide moiety can range in length from about 5 amino acids to about 40 amino acids. The peptide moieties can have a structural modification, such as to increase stability or direct conformational properties. Any of the structural modifications described below can be utilized.

An RGD peptide moiety can be used to target a host cell derived from a tumorous cell e.g., an endothelial tumor cell or a breast cancer tumor cell (Zitzmann et al., Cancer Res., 62:5139-43, 2002). The RGD peptide can be linear or cyclic, and can be modified, e.g., glycosylated or methylated to facilitate targeting to specific tissues. For example, a glycosylated RGD peptide can deliver a RNA effector molecule composition to a cell expressing α_{V}β₃ (Haubner et al., Jour. Nucl. Med., 42:326-336, 2001).

A “cell permeation peptide” is capable of permeating a cell, e.g., a microbial cell, such as a bacterial or fungal cell, or a mammalian cell, such as a human cell. A microbial cell-permeating peptide can be, for example, an a-helical linear peptide (e.g., LL-37 or Ceropin P1), a disulfide bond-containing peptide (e.g., α-defensin, β-defensin or bactenecin), or a peptide containing only one or two dominating amino acids (e.g., PR-39 or indolicidin). A cell permeation peptide can also include a nuclear localization signal (NLS). For example, a cell permeation peptide can be a bipartite amphipathic peptide, such as MPG, which is derived from the fusion peptide domain of HIV-1 gp41 and the NLS of SV40 large T antigen (Simeoni et al., Nucl. Acids Res. 31:2717-2724, 2003).

Representative U.S. patents that teach the preparation of oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941; 6,294,664; 6,320,017; 6,576,752; 6,783,931; 6,900,297; 7,037,646; each of which is herein incorporated by reference.

It is not necessary for all positions in a given oligonucleotide to be uniformly modified, and in fact more than one of the aforementioned modifications can be incorporated in a single oligonucleotide or even at a single nucleoside within an oligonucleotide. The present invention also includes oligonucleotides which are chimeric compounds. “Chimeric” oligonucleotides or “chimeras,” in the context of this invention, are oligonucleotides, preferably double-stranded oligonucleotides, which contain two or more chemically distinct regions, each made up of at least one monomer unit, i.e., a nucleotide. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide can serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of RNA effector molecule inhibition of gene expression. Consequently, comparable results can often be obtained with shorter oligonucleotides when chimeric oligonucleotides are used, compared to phosphorothioate deoxydsRNAs hybridizing to the same target region. Cleavage of the RNA target can be routinely detected by gel electrophoresis and, if necessary, associated nucleic acid hybridization techniques known in the art.

In certain instances, the oligonucleotide can be modified by a non-ligand group. A number of non-ligand molecules have been conjugated to oligonucleotides in order to enhance the activity, cellular distribution or cellular uptake of the oligonucleotide, and procedures for performing such conjugations are available in the scientific literature. Such non-ligand moieties have included lipid moieties, such as cholesterol (Kubo, T. et al., Biochem. Biophys. Res. Comm., 2007, 365(1):54-61; Letsinger et al., Proc. Natl. Acad. Sci. USA, 1989, 86:6553), cholic acid (Manoharan et al., Bioorg. Med. Chem. Lett., 1994, 4:1053), a thioether, e.g., hexyl-S-tritylthiol (Manoharan et al., Ann. N.Y. Acad. Sci., 1992, 660:306; Manoharan et al., Bioorg. Med. Chem. Let., 1993, 3:2765), a thiocholesterol (Oberhauser et al., Nucl. Acids Res., 1992, 20:533), an aliphatic chain, e.g., dodecandiol or undecyl residues (Saison-Behmoaras et al., EMBO J., 1991, 10:111; Kabanov et al., FEBS Lett., 1990, 259:327; Svinarchuk et al., Biochimie, 1993, 75:49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36:3651; Shea et al., Nucl. Acids Res., 1990, 18:3777), a polyamine or a polyethylene glycol chain (Manoharan et al., Nucleosides & Nucleotides, 1995, 14:969), or adamantane acetic acid (Manoharan et al., Tetrahedron Lett., 1995, 36:3651), a palmityl moiety (Mishra et al., Biochim. Biophys. Acta, 1995, 1264:229), or an octadecylamine or hexylamino-carbonyl-oxycholesterol moiety (Crooke et al., J. Pharmacol. Exp. Ther., 1996, 277:923). Representative United States patents that teach the preparation of such oligonucleotide conjugates have been listed above. Typical conjugation protocols involve the synthesis of an oligonucleotide bearing an aminolinker at one or more positions of the sequence. The amino group is then reacted with the molecule being conjugated using appropriate coupling or activating reagents. The conjugation reaction can be performed either with the oligonucleotide still bound to the solid support or following cleavage of the oligonucleotide, in solution phase. Purification of the oligonucleotide conjugate by HPLC typically affords the pure conjugate.

### Delivery of an RNA Effector Molecule to a Host Cell

The delivery of an RNA effector molecule to cells according to methods provided herein can be achieved in a number of different ways. Delivery can be performed directly by administering a composition comprising an RNA effector molecule, e.g. a dsRNA, to the cell culture media. Alternatively, delivery can be performed indirectly by administering one or more vectors that encode and direct the expression of the RNA effector molecule. These alternatives are discussed further below.

### Direct Delivery

RNA effector molecules can be modified by chemical conjugation to lipophilic groups such as cholesterol to enhance cellular uptake and prevent degradation. In an alternative embodiment, RNA effector molecules can be delivered using a drug delivery system such as a nanoparticle, a dendrimer, a polymer, a liposome, or a cationic delivery system. Positively charged cationic delivery systems facilitate binding of an RNA effector molecule (negatively charged oligonucleotide) and also enhance interactions at the negatively charged cell membrane to permit efficient cellular uptake. Cationic lipids, dendrimers, or polymers can either be bound to RNA effector molecules, or induced to form a vesicle or micelle (see e.g., Kim S H., et al (2008) Journal of Controlled Release 129(2):107-116) that encases the RNA effector molecule. Methods for making and using cationic-RNA effector molecule complexes are well within the abilities of those skilled in the art (see e.g., Sorensen, D R., et al (2003) J. Mol. Biol 327:761-766; Verma, U N., et al (2003) Clin. Cancer Res. 9:1291-1300; Arnold, A S et al (2007) J. Hypertens. 25:197-205, which are incorporated herein by reference in their entirety). Exemplary reagents that facilitate RNA effector molecule uptake into a cell comprising charged lipids are described in e.g., U.S. Ser. No. 61/267,419 (filed December 7, 2009), which is herein incorporated by reference in its entirety. Liposome agents and emulsions for facilitating uptake of the RNA effector molecule into the host cell are known in the art or are described herein.

### Separate and Temporal Administration

Where the RNA effector molecule is a double-stranded molecule, such as a small interfering RNA (siRNA), comprising a sense strand and an antisense strand, the sense strand and antisense strand can be separately and temporally exposed to a cell, cell lysate or cell culture. The phrase “separately and temporally” refers to the introduction of each strand of a double-stranded RNA effector molecule to a cell, cell lysate or cell culture in a single-stranded form, e.g., in the form of a non-annealed mixture of both strands or as separate, i.e., unmixed, preparations of each strand. In some embodiments, there is a time interval between the introduction of each strand which can range from seconds to several minutes to about an hour or more, e.g., 12, 24, 48, 72, 84, 96, or 108 hours or more. Separate and temporal administration can be performed with independently modified or unmodified sense and antisense strands.

It is also contemplated herein that a first and second RNA effector molecule are administered in a separate and temporal manner. Thus, each of a plurality of RNA effector molecules can be administered at a separate time or at a different frequency interval to achieve the desired average percent inhibition for the target RNA. In one embodiment, the RNA effector molecules are added at a concentration from approximately 0.01 nM to 200 nM. In another embodiment, the RNA effector molecules are added at an amount of approximately 50 molecules per cell up to and including 500,000 molecules per cell. In another embodiment, the RNA effector molecules are added at a concentration from about 0.1 fmol/10⁶ cells to about 1 pmol/10⁶ cells.

### Transient Inhibition of a Gene Product

In one embodiment, the RNA effector molecule is delivered to the cell such that expression of the gene product is modulated only transiently, e.g., by addition of an RNA effector molecule composition to the cell culture medium used for the production of the polypeptide, with or without a transfection reagent, where the presence of the RNA effector molecules dissipates over time, i.e., the RNA effector molecule is not constitutively expressed in the cell. This can be achieved by altering the timing between delivery of discrete doses of the RNA effector molecule to e.g., the cell culture medium. One of skill in the art can choose an appropriate dosing regime that permits (1) transient inhibition of the gene product, (2) constitutive inhibition of the gene product, or (3) maintenance of a partial inhibition of the gene product (e.g., 50% inhibition, 60%, 70%, 80%, 20%, 30%, 40% etc) as desired by determining the level of inhibition using e.g., ELISA assays to test for expression of the gene product.

Vector Encoded dsRNAs

In another aspect, an RNA effector molecule for modulating expression of a target gene can be expressed from transcription units inserted into DNA or RNA vectors (see, e.g., Couture, A, et al., TIG. (1996), 12:5-10; Skillern, A., et al., International PCT Publication No. WO 00/22113, Conrad, International PCT Publication No. WO 00/22114, and Conrad, U.S. Pat. No. 6,054,299). Such vectors are also useful for expressing an RNA molecule that inhibits expression of a selectable amplifiable marker gene or a transgene. Expression can be transient (on the order of hours to weeks) or sustained (weeks to months or longer), depending upon the specific construct used and the target cell type. These transgenes can be introduced as a linear construct, a circular plasmid, or a viral vector, which can be an integrating or non-integrating vector. The transgene can also be constructed to permit it to be inherited as an extra chromosomal plasmid (Gassmann, et al., Proc. Natl. Acad. Sci. USA (1995) 92:1292).

The individual strand or strands of an RNA effector molecule can be transcribed from a promoter on an expression vector. Where two separate strands are to be expressed to generate, for example a dsRNA, two separate expression vectors can be co-introduced (e.g., by transfection or infection) into a target cell. Alternatively each individual strand of a dsRNA can be transcribed by promoters, both of which are located on the same expression plasmid. In one embodiment, a dsRNA is expressed as an inverted repeat joined by a linker polynucleotide sequence such that the dsRNA has a stem and loop structure.

RNA effector molecule expression vectors are generally DNA plasmids or viral vectors. Expression vectors compatible with eukaryotic cells, preferably those compatible with vertebrate cells, can be used to produce recombinant constructs for the expression of an RNA effector molecule as described herein. Eukaryotic cell expression vectors are well known in the art and are available from a number of commercial sources. Typically, such vectors are provided containing convenient restriction sites for insertion of the desired nucleic acid segment. RNA effector molecule expressing vectors can be delivered directly to target cells using standard transfection and transduction methods.

### Transfection of RNA Effector Molecules or Plasmids

An RNA effector molecule or an expression plasmids encoding an RNA effector molecule can be transfected into target cells as a complex with cationic lipid carriers (e.g., Oligofectamine) or non-cationic lipid-based carriers (e.g., Transit-TKO™, Mirus Bio LLC, Madison, Wis.). Multiple lipid transfections for RNA effector molecule-mediated knockdowns targeting different regions of a target RNA over a period of a week or more are also contemplated by the invention. Successful introduction of vectors into host cells can be monitored using various known methods. For example, transient transfection can be signaled with a reporter, such as a fluorescent marker, such as Green Fluorescent Protein (GFP). Stable transfection of cells ex vivo can be ensured using markers that provide the transfected cell with resistance to specific environmental factors (e.g., antibiotics and drugs), such as hygromycin B resistance. Successful transfection of an RNA effector molecule can be determined by measuring the mRNA or protein expression level of the target RNA by e.g., RT-PCR, Western Blotting or Northern Blotting.

Vector systems encoding a transgene linked to a first selectable amplifiable marker can be e.g., a viral vector or a plasmid. Viral vector systems which can be utilized with the methods described herein include, but are not limited to, (a) adenovirus vectors; (b) retrovirus vectors, including but not limited to lentiviral vectors, moloney murine leukemia virus, etc.; (c) adeno-associated virus vectors; (d) herpes simplex virus vectors; (e) SV 40 vectors; (f) polyoma virus vectors; (g) papilloma virus vectors; (h) picornavirus vectors; (i) pox virus vectors such as an orthopox, e.g., vaccinia virus vectors or avipox, e.g. canary pox or fowl pox; and (j) a helper-dependent or gutless adenovirus. Replication-defective viruses can also be advantageous. Different vectors will or will not become incorporated into the cells' genome. In one embodiment, the vector encoding a transgene linked to a first selectable amplifiable marker is a vector that permits incorporation of at least the transgene and the amplifiable marker into the cells' genome. The constructs can include viral sequences for transfection, if desired.

Alternatively, the construct can be incorporated into vectors capable of episomal replication, e.g. EPV and EBV vectors. Constructs for the recombinant expression of an RNA effector molecule will generally require regulatory elements, e.g., promoters, enhancers, etc., to ensure the expression of the RNA effector molecule in target cells. Other aspects to consider for vectors and constructs are further described below.

Vectors useful for the delivery of a transgene linked to a selectable amplifiable marker gene or an RNA effector molecule will include regulatory elements (promoter, enhancer, etc.) sufficient for expression of the RNA effector molecule or biological product in the desired target cell. The regulatory elements can be chosen to provide either constitutive or regulated/inducible expression.

Expression from the vector can be precisely regulated, for example, by using an inducible regulatory sequence that is sensitive to certain physiological regulators, e.g., glucose levels (Docherty et al., 1994, FASEB J. 8:20-24). Such inducible expression systems, suitable for the control of dsRNA expression in cells include, for example, regulation by ecdysone, estrogen, progesterone, doxycycline, tetracycline, chemical inducers of dimerization, and isopropyl-beta-D1-thiogalactopyranoside (IPTG). A person skilled in the art would be able to choose the appropriate regulatory/promoter sequence based on the intended use of the transgene.

In a specific embodiment, viral vectors that contain nucleic acid sequences encoding (a) an RNA effector molecule or (b) a transgene linked to a selectable amplifiable marker gene to be modified can be used. For example, a retroviral vector can be used (see Miller et al., Meth. Enzymol. 217:581-599 (1993)). These retroviral vectors contain the components necessary for the correct packaging of the viral genome and integration into the host cell DNA. The nucleic acid sequences encoding an RNA effector molecule are cloned into one or more vectors, which facilitates delivery of the nucleic acid into a patient. More detail about retroviral vectors can be found, for example, in Boesen et al., Biotherapy 6:291-302 (1994), which describes the use of a retroviral vector to deliver the mdrl gene to hematopoietic stem cells in order to make the stem cells more resistant to chemotherapy. Other references illustrating the use of retroviral vectors in gene therapy are: Clowes et al., J. Clin. Invest. 93:644-651 (1994); Kiem et al., Blood 83:1467-1473 (1994); Salmons and Gunzberg, Human Gene Therapy 4:129-141 (1993); and Grossman and Wilson, Curr. Opin. in Genetics and Devel. 3:110-114 (1993). Lentiviral vectors contemplated for use include, for example, the HIV based vectors described in U.S. Pat. Nos. 6,143,520; 5,665,557; and 5,981,276, each of which is herein incorporated by reference in its entirety.

Adenoviruses are also contemplated for use with the methods described herein. A suitable AV vector for expressing an RNA effector molecule featured in the invention, a method for constructing the recombinant AV vector, and a method for delivering the vector into target cells, are described in Xia H et al. (2002), Nat. Biotech. 20: 1006-1010.

Use of Adeno-associated virus (AAV) vectors is also contemplated (Walsh et al., Proc. Soc. Exp. Biol. Med. 204:289-300 (1993); U.S. Pat. No. 5,436,146, herein incorporated by reference in its entirety). In one embodiment, the RNA effector molecule can be expressed as two separate, complementary single-stranded RNA molecules from a recombinant AAV vector having, for example, either the U6 or H1 RNA promoters, or the cytomegalovirus (CMV) promoter. Suitable AAV vectors for expressing the dsRNA featured in the invention, methods for constructing the recombinant AV vector, and methods for delivering the vectors into target cells are described in Samulski Ret al. (1987), J. Virol. 61: 3096-3101; Fisher K J et al. (1996), J. Virol, 70: 520-532; Samulski Ret al. (1989), J. Virol. 63: 3822-3826; U.S. Pat. No. 5,252,479; U.S. Pat. No. 5,139,941; International Patent Application No. WO 94/13788; and International Patent Application No. WO 93/24641, the entire disclosures of which are herein incorporated by reference.

Another preferred viral vector is a pox virus such as a vaccinia virus, for example an attenuated vaccinia such as Modified Virus Ankara (MVA) or NYVAC, an avipox such as fowl pox or canary pox.

The tropism of viral vectors can be modified by pseudotyping the vectors with envelope proteins or other surface antigens from other viruses, or by substituting different viral capsid proteins, as appropriate. For example, lentiviral vectors can be pseudotyped with surface proteins from vesicular stomatitis virus (VSV), rabies, Ebola, Mokola, and the like. AAV vectors can be made to target different cells by engineering the vectors to express different capsid protein serotypes; see, e.g., Rabinowitz J E et al. (2002), J Virol 76:791-801, the entire disclosure of which is herein incorporated by reference.

The pharmaceutical preparation of a vector can include the vector in an acceptable diluent, or can include a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

### Administration to Cells

Compositions described herein can be administered to cells in culture in a variety of methods known to those of skill in the art.

In one embodiment, the composition is administered to the cell using continuous infusion of at least one RNA effector molecule into a culture medium used for maintaining the cell during the selection process. In one embodiment, the continuous infusion is administered at a rate to achieve a desired average percent inhibition for the selectable amplifiable marker or transgene. In another embodiment, the addition of the RNA effector molecule is repeated throughout the production of the polypeptide. In another embodiment, addition of the RNA effector molecule is repeated at a frequency selected from the group consisting of: 6 h, 12 h, 24 h, 36 h, 48 h, 72 h, 84 h, 96 h, and 108 h. Alternatively, in one embodiment, the addition of the RNA effector molecule is repeated at least three times.

An appropriate concentration of an RNA effector molecule composition useful to achieve the generation of a cell capable of producing a biological product as described herein can be determined by one of skill in the art. In one embodiment, the at least one RNA effector molecule is added at a concentration selected from the group consisting of 1 pM, 5 pM, 10 pM, 25 pM, 50 pM, 75 pM, 0.1 nM, 0.5 nM, 0.75 nM, 1 nM, 2 nM, 5 nM, 10 nM, 20 nM, 30 nM, 40 nM, 50 nM, 60 nM, 70 nM, 80 nM, 90 nM, 0.1 μM, 0.5 μM, 0.75 μM, 1 μM, 2 μM, 5 μM, and 10 μM. The RNA effector molecule can also be added following the selection step to help maintain cells throughout the production process. The concentration will typically be lower than that used during the selection process.

### Compositions for Delivery of an RNA Effector Molecule to a Cell

In one embodiment, the invention provides compositions containing an RNA effector molecule, as described herein, and an acceptable carrier. In one embodiment, the acceptable carrier is a “reagent that facilitates RNA effector molecule uptake” as that term is used herein. The composition containing the RNA effector molecule is useful for inhibiting a selectable amplifiable marker gene endogenous to the host cell or a transgene produced in the host cell. Such compositions are formulated based on the mode of delivery. Provided herein are exemplary RNA effector molecules useful in modifying the glycosylation pattern of an expressed polypeptide. In another embodiment, the methods described herein further comprise treating a cell with a composition that inhibits the mannose 6 phosphate receptor to prevent lysosomal uptake of the produced polypeptide. In one embodiment, the RNA effector molecule is an siRNA. In another embodiment, the RNA effector molecule is not an shRNA.

In one embodiment, the composition further comprises a reagent that facilitates RNA effector uptake into a cell (transfection reagent), such as an emulsion, a liposome, a cationic lipid, a non-cationic lipid, an anionic lipid, a charged lipid, a penetration enhancer or alternatively, a modification to the RNA effector molecule to attach e.g., a ligand, peptide, lipophillic group, or targeting moiety.

In one embodiment, the compositions described herein comprise a plurality of RNA effector molecules that target the same selectable amplifiable marker gene or transgene, or a combination thereof In one embodiment of this aspect, each of the plurality of RNA effector molecules is provided at a different concentration. In another embodiment of this aspect, each of the plurality of RNA effector molecules is provided at the same concentration. In another embodiment of this aspect, at least two of the plurality of RNA effector molecules are provided at the same concentration, while at least one other RNA effector molecule in the plurality is provided at a different concentration. It is appreciated by one of skill in the art that a variety of combinations of RNA effector molecules and concentrations can be provided to a cell in culture to produce the desired effects described herein.

The compositions featured herein are administered in amounts sufficient to inhibit expression of target genes. In general, a suitable dose of RNA effector molecule will be in the range of 0.001 to 200.0 milligrams per unit volume or cell density per day. In another embodiment, the RNA effector molecule is provided in the range of 0.001 nM to 200 mM per day, generally in the range of 0.1 nM to 500 nM. For example, the dsRNA can be administered at 0.01 nM, 0.05 nM, 0.1 nM, 0.5 nM, 0.75 nM, 1 nM, 1.5 nM, 2 nM, 3 nM, 10 nM, 20 nM, 30 nM, 40 nM, 50 nM, 100 nM, 200 nM, 400 nM, or 500 nM per single dose.

The composition can be administered once daily, or the RNA effector molecule can be administered as two, three, or more sub-doses at appropriate intervals throughout the day or even using continuous infusion or delivery through a controlled release formulation. In that case, the RNA effector molecule contained in each sub dose must be correspondingly smaller in order to achieve the total daily dosage. The dosage unit can also be compounded for delivery over several days, e.g., using a conventional sustained release formulation, which provides sustained release of the RNA effector molecule e.g., over a several day period. Sustained release formulations are well known in the art and are particularly useful for delivery of agents to a cell culture, such as could be used with the compositions of the present invention. In one embodiment, an RNA effector molecule is contacted with the cells in culture at a final concentration of 1nM. It should be noted that when administering a plurality of RNA effector molecules that one should consider that the total dose of RNA effector molecules will be higher than when each is administered alone. For example, administration of three RNA effector molecules each at 1 nM (e.g., for effective inhibition of target gene expression) will necessarily result in a total dose of 3 nM to the cell culture. One of skill in the art can modify the necessary amount of each RNA effector molecule to produce effective inhibition of each target gene while preventing any unwanted toxic effects to the cell culture resulting from high concentrations of either the RNA effector molecules or delivery agent.

The effect of a single dose on target gene transcript levels can be long-lasting, such that subsequent doses are administered at not more than 3, 4, or 5 day intervals, or at not more than 1, 2, 3, or 4 week intervals.

It is also noted that, in certain embodiments, it can be beneficial to contact the cells in culture with an RNA effector molecule such that a constant number (or at least a minimum number) of RNA effector molecules per each cell is maintained. Maintaining the levels of the RNA effector molecule as such can ensure that inhibition of expression is maintained even at high cell densities.

Alternatively, the amount of an RNA effector molecule can be administered according to the cell density. In such embodiments, the RNA effector molecule(s) is added at a concentration of at least 0.01 fmol/10⁶ cells, at least 0.1 fmol/10⁶ cells, at least 0.5 fmol/10⁶ cells, at least 0.75 fmol/10⁶ cells, at least 1 fmol/10⁶ cells, at least 2 fmol/10⁶ cells, at least 5 fmol/10⁶ cells, at least 10 fmol/10⁶ cells, at least 20 fmol/10⁶ cells, at least 30 fmol/10⁶ cells, at least 40 fmol/10⁶ cells, at least 50 fmol/10⁶ cells, at least 60 fmol/10⁶ cells, at least 100 fmol/10⁶ cells, at least 200 fmol/10⁶ cells, at least 300 fmol/10⁶ cells, at least 400 fmol/10⁶ cells, at least 500 fmol/10⁶ cells, at least 700 fmol/10⁶ cells, at least 800 fmol/10⁶ cells, at least 900 fmol/10⁶ cells, or at least 1 pmol/10⁶ cells, or more.

In an alternate embodiment, the RNA effector molecule is administered at a dose of at least 10 molecules per cell, at least 20 molecules per cell, at least 30 molecules per cell, at least 40 molecules per cell, at least 50 molecules per cell, at least 60 molecules per cell, at least 70 molecules per cell, at least 80 molecules per cell, at least 90 molecules per cell at least 100 molecules per cell, at least 200 molecules per cell, at least 300 molecules per cell, at least 400 molecules per cell, at least 500 molecules per cell, at least 600 molecules per cell, at least 700 molecules per cell, at least 800 molecules per cell, at least 900 molecules per cell, at least 1000 molecules per cell, at least 2000 molecules per cell, at least 5000 molecules per cell or more. In some embodiments, the RNA effector molecule is administered at a dose within the range of 10-100 molecules/cell, 10-90 molecules/cell, 10-80 molecules/cell, 10-70 molecules/cell, 10-60 molecules/cell, 10-50 molecules/cell, 10-40 molecules/cell, 10-30 molecules/cell, 10-20 molecules/cell, 90-100 molecules/cell, 80-100 molecules/cell, 70-100 molecules/cell, 60-100 molecules/cell, 50-100 molecules/cell, 40-100 molecules/cell, 30-100 molecules/cell, 20-100 molecules/cell, 30-60 molecules/cell, 30-50 molecules/cell, 40-50 molecules/cell, 40-60 molecules/cell, or any range therebetween.

In one embodiment, during selection of cells having multiple copy numbers of the transgene and the selectable amplifiable marker gene, the RNA effector molecule is added at a concentration selected from the group consisting of 1 pM, 5 pM, 10 pM, 25 pM, 50 pM, 75 pM, 0.1 nM, 0.5 nM, 0.75 nM, 1 nM, 2 nM, 5 nM, 10 nM, 20 nM, 30 nM, 40 nM, 50 nM, 60 nM, 70 nM, 80 nM, 90 nM, 0.1 μM, 0.5 μM, 0.75 μM, 1 μM, 2 μM, 5 μM, and 10 μM. Cells can be maintained in the presence of the RNA effector molecule throughout the production process, however the concentration will typically be lower than that used during the selection process (e.g., the concentration of the RNA effector molecule used to maintain cell during the production process is at least 50% lower, at least 1-fold lower, at least 2-fold lower, at least 5-fold lower, at least 10-fold lower, at least 100-fold lower, at least 1000-fold lower or less than the concentration of the RNA effector molecule used during the cell selection process).

In one embodiment of the methods described herein, the RNA effector molecule is provided to the cells in a continuous infusion. The continuous infusion can be initiated at day zero (e.g., the first day of cell culture or day of inoculation with an RNA effector molecule) or can be initiated at any time period during the selection or polypeptide production process. Similarly, the continuous infusion can be stopped at any time point during the selection or polypeptide production process. Thus, the infusion of an RNA effector molecule or composition can be provided and/or removed at a particular phase of cell growth, a window of time in the production process, or at any other desired time point. The continuous infusion can also be provided to achieve an “average percent inhibition” for a target gene, as that term is used herein. In one embodiment, a continuous infusion can be used following an initial bolus administration of an RNA effector molecule to a cell culture. In this embodiment, the continuous infusion maintains the concentration of RNA effector molecule above a minimum level over a desired period of time. The continuous infusion can be delivered at a rate of 0.03-3 pmol/liter of culture/h, for example, at 0.03 pmol/l/h, 0.05 pmol/l/h, 0.08 pmol/l/h, 0.1 pmol/l/h, 0.2 pmol/l/h, 0.3 pmol/l/h, 0.5 pmol/l/h, 1.0 pmol/l/h, 2 pmol/l/h, or 3 pmol/l/h, or any value therebetween. In one embodiment, the RNA effector molecule is administered as a sterile aqueous solution. In another embodiment, the RNA effector molecule is formulated in a cationic or non-cationic lipid formulation. In still another embodiment, the RNA effector molecule is formulated in a cell medium suitable for culturing a host cell (e.g., a serum-free medium). In one embodiment, an initial concentration of RNA effector molecule(s) is supplemented with a continuous infusion of the RNA effector molecule to maintain modulation of expression of a target gene. In another embodiment, the RNA effector molecule is applied to cells in culture at a particular stage of cell growth (e.g., early log phase) in a bolus dosage to achieve a certain concentration (e.g., 1 nM), and provided with a continuous infusion of the RNA effector molecule.

The RNA effector molecule(s) can be administered once daily, or the RNA effector molecule treatment can be repeated (e.g., two, three, or more doses) by adding the composition to the culture medium at appropriate intervals/frequencies throughout the production of the biological product. As used herein the term “frequency” refers to the interval at which transfection or infection of the cell culture occurs and can be optimized by one of skill in the art to maintain the desired level of inhibition for each target gene. In one embodiment, RNA effector molecules are contacted with cells in culture at a frequency of every 48 hours. In other embodiments, the RNA effector molecules are administered at a frequency of e.g., every 4 h, every 6 h, every 12 h, every 18 h, every 24 h, every 36 h, every 72 h, every 84 h, every 96 h, every 5 days, every 7 days, every 10 days, every 14 days, every 3 weeks, or more during the selection process or production of the biological product. The frequency can also vary, such that the interval between each dose is different (e.g., first interval 36 h, second interval 48 h, third interval 72 h etc).

The term “frequency” can be similarly applied to nutrient feeding of a cell culture during the production of a polypeptide. The frequency of treatment with RNA effector molecule(s) and nutrient feeding need not be the same. To be clear, nutrients can be added at the time of RNA effector treatment or at an alternate time. The frequency of nutrient feeding can be a shorter interval or a longer interval than RNA effector molecule treatment. As but one example, the dose of RNA effector molecule can be applied at a 48h interval while nutrient feeding can be applied at a 24h interval. During the entire length of the interval for producing the biological product (e.g., 3 weeks) there can be more doses of nutrients than RNA effector molecules or less doses of nutrients than RNA effector molecules. Alternatively, the amount (e.g., number) of treatments with RNA effector molecule(s) is equal to that of nutrient feedings.

The frequency of RNA effector molecule treatment can be optimized to maintain an “ average percent inhibition” of a particular target gene. As used herein, the term “average percent inhibition” refers to the average degree of inhibition of target gene expression over time that is necessary to produce the desired effect and which is below the degree of inhibition that produces any unwanted or negative effects. In some embodiments, the desired average percent inhibition is at least 20%, at least 30%, at least 40%, at least 50%, at least 60%, at least 70%, at least 80%, at least 85%, at least 90%, at least 95%, at least 99%, or even 100% (i.e., absent). One of skill in the art can use routine cell death assays to determine the upper limit for desired percent inhibition (e.g., level of inhibition that produces unwanted effects). One of skill in the art can also use methods to detect target gene expression (e.g., RT-PCR) to determine an amount of an RNA effector molecule that produces inhibition of expression. The percent inhibition is described herein as an average value over time, since the amount of inhibition is dynamic and can fluctuate slightly between doses of the RNA effector molecule.

In one embodiment of the methods described herein, the RNA effector molecule is added to the culture medium of the cells in culture. The methods described herein can be applied to any size of cell culture flask and/or bioreactor. For example, the methods can be applied in bioreactors or cell cultures of 1 L, 3 L, 5 L, 10 L, 15 L, 40 L, 100 L, 500 L, 1000 L, 2000 L, 3000 L, 4000 L, 5000 L or larger. In some embodiments, the cell culture size can range from 0.01 L to 5000 L, from 0.1 L to 5000 L, from 1 L to 5000 L, from 5 L to 5000 L, from 40 L to 5000 L, from 100 L-5000 L, from 500 L to 5000 L, from 1000-5000 L, from 2000-5000 L, from 3000-5000 L, from 4000-5000 L, from 4500-5000 L, from 0.01 L to 1000 L, from 0.01-500 L, from 0.01-100 L, from 0.01-40 L, from 15-2000 L, from 40-1000 L, from 100-500 L, from 200-400 L, or any integer therebetween.

The RNA effector molecule(s) can be added during any phase of cell growth including, but not limited to, lag phase, stationary phase, early log phase, mid-log phase, late-log phase, exponential phase, or death phase. It is preferred that the cells are contacted with the RNA effector molecules prior to their entry into the death phase. In some embodiments, it may be desired to contact the cell in an earlier growth phase such as the lag phase, early log phase, mid-log phase or late-log phase. In other embodiments, it may be desired or acceptable to inhibit target gene expression at a later phase in the cell growth cycle (e.g., late-log phase or stationary phase).

RNA effector molecules featured in the invention can be encapsulated within liposomes or can form complexes thereto, in particular to cationic liposomes. Alternatively, RNA effector molecules can be complexed to lipids, in particular to cationic lipids. Suitable fatty acids and esters include but are not limited to arachidonic acid, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C₁₋₂₀ alkyl ester (e.g., isopropylmyristate IPM), monoglyceride, diglyceride or acceptable salt thereof.

In one embodiment, an RNA effector molecule featured in the invention is fully encapsulated in the lipid formulation (e.g., to form a SPLP, pSPLP, SNALP, or other nucleic acid-lipid particle). As used herein, the term “SNALP” refers to a stable nucleic acid-lipid particle, including SPLP. As used herein, the term “SPLP” refers to a nucleic acid-lipid particle comprising plasmid DNA encapsulated within a lipid vesicle. SNALPs and SPLPs typically contain a cationic lipid, a non-cationic lipid, and a lipid that prevents aggregation of the particle (e.g., a PEG-lipid conjugate). SPLPs include “pSPLP,” which include an encapsulated condensing agent-nucleic acid complex as set forth in e.g., PCT Publication No. WO 00/03683. The particles in this embodiment typically have a mean diameter of about 50 nm to about 150 nm, more typically about 60 nm to about 130 nm, more typically about 70 nm to about 110 nm, most typically about 70 to about 90 nm, and are substantially nontoxic. In addition, the nucleic acids when present in the nucleic acid-lipid particles of the present invention are resistant in aqueous solution to degradation with a nuclease. Nucleic acid-lipid particles and their method of preparation are disclosed in, e.g., U.S. Pat. Nos. 5,976,567; 5,981,501; 6,534,484; 6,586,410; 6,815,432; and PCT Publication No. WO 96/40964.

In one embodiment, the lipid to drug ratio (mass/mass ratio) (e.g., lipid to dsRNA ratio) will be in the range of from about 1:1 to about 50:1, from about 1:1 to about 25:1, from about 3:1 to about 15:1, from about 4:1 to about 10:1, from about 5:1 to about 9:1, or about 6:1 to about 9:1.

The cationic lipid of the formulation preferably comprises at least one protonatable group having a pKa of from 4 to 15. The cationic lipid can be, for example, N,N-dioleyl-N,N-dimethylammonium chloride (DODAC), N,N-distearyl-N,N-dimethylammonium bromide (DDAB), N-(I -(2,3-dioleoyloxy)propyl)-N,N,N-trimethylammonium chloride (DOTAP), N-(I-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), 1,2-Dilinoleoyl-3-trimethylaminopropane chloride salt (DLin-TAP.Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 2,2-Dilinoleyl-4-dimethylaminomethyl-[1,3]-dioxolane (DLin-K-DMA), 2,2-Dilinoleyl-4-dimethylaminoethyl[1,3]-dioxolane, or a mixture thereof. The cationic lipid can comprise from about 20 mol % to about 70 mol % or about 40 mol % to about 60 mol % of the total lipid present in the particle. In one embodiment, cationic lipid can be further conjugated to a ligand.

The non-cationic lipid can be an anionic lipid or a neutral lipid including, but not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), cholesterol, or a mixture thereof. The non-cationic lipid can be from about 5 mol % to about 90 mol %, about 10 mol %, or about 58 mol % if cholesterol is included, of the total lipid present in the particle.

The lipid that inhibits aggregation of particles can be, for example, a polyethyleneglycol (PEG)-lipid including, without limitation, a PEG-diacylglycerol (DAG), a PEG-dialkyloxypropyl (DAA), a PEG-phospholipid, a PEG-ceramide (Cer), or a mixture thereof. The PEG-DAA can be, for example, a PEG-dilauryloxypropyl (Ci₂), a PEG-dimyristyloxypropyl (C₁₄), a PEG-dipalmityloxypropyl (C₁₆), or a PEG-distearyloxypropyl (C₁₈). The lipid that prevents aggregation of particles can be from 0 mol % to about 20 mol % or about 2 mol % of the total lipid present in the particle. In one embodiment, PEG lipid can be further conjugated to a ligand.

In some embodiments, the nucleic acid-lipid particle further includes a steroid such as, cholesterol at, e.g., about 10 mol % to about 60 mol % or about 48 mol % of the total lipid present in the particle.

In one embodiment, the lipid particle comprises a steroid, a PEG lipid and a cationic lipid of formula (I):

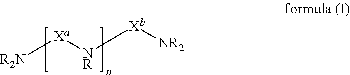

wherein
each X^{a} and X^{b}, for each occurrence, is independently C₁₋₆ alkylene;
n is 0, 1, 2, 3, 4, or 5; each R is independently H,

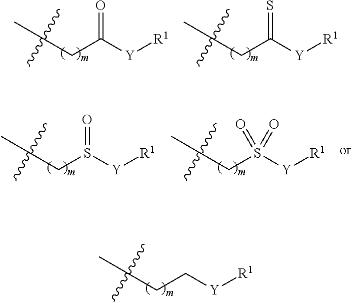

m is 0, 1, 2, 3 or 4; Y is absent, O, NR², or S;
R¹ is alkyl alkenyl or alkynyl; each of which is optionally substituted with one or more substituents; and
R² is H, alkyl alkenyl or alkynyl; each of which is optionally substituted each of which is optionally substituted with one or more substituents. In one example, the lipidoid ND98.4HCl (MW 1487) (Formula 1), Cholesterol (Sigma-Aldrich), and PEG-Ceramide C16 (Avanti Polar Lipids) can be used to prepare lipid RNA effector molecule nanoparticles (e.g., LNP01 particles). Stock solutions of each in ethanol can be prepared as follows: ND98, 133 mg/mL; Cholesterol, 25 mg/mL, PEG-Ceramide C16, 100 mg/mL. The ND98, Cholesterol, and PEG-Ceramide C16 stock solutions can then be combined in a, e.g., 42:48:10 molar ratio. The combined lipid solution can be mixed with aqueous RNA effector molecule (e.g., in sodium acetate pH 5) such that the final ethanol concentration is about 35-45% and the final sodium acetate concentration is about 100-300 mM. Lipid RNA effector molecule nanoparticles typically form spontaneously upon mixing. Depending on the desired particle size distribution, the resultant nanoparticle mixture can be extruded through a polycarbonate membrane (e.g., 100 nm cut-off) using, for example, a thermobarrel extruder, such as Lipex Extruder (Northern Lipids, Inc). In some cases, the extrusion step can be omitted. Ethanol removal and simultaneous buffer exchange can be accomplished by, for example, dialysis or tangential flow filtration. Buffer can be exchanged with, for example, phosphate buffered saline (PBS) at about pH 7, e.g., about pH 6.9, about pH 7.0, about pH 7.1, about pH 7.2, about pH 7.3, or about pH 7.4.

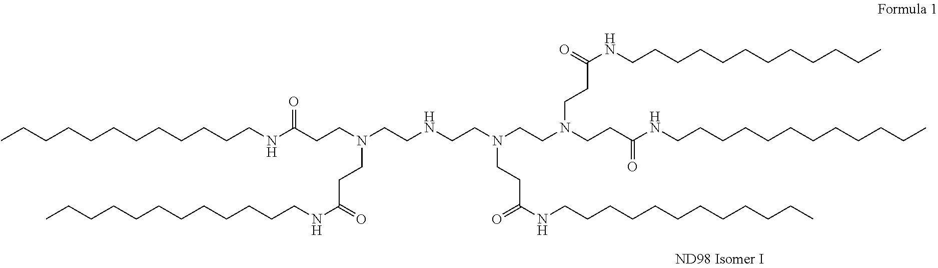 [SMILES: CCCCCCCCCCCCNC(=O)CCN(CCNCCN(CCC(=O)NCCCCCCCCCCCC)CCN(CCC(=O)NCCCCCCCCCCCC)CCC(=O)NCCCCCCCCCCCC)CCC(=O)NCCCCCCCCCCCC]

LNP01 formulations are described, e.g., in International Application Publication No. WO 2008/042973, which is hereby incorporated by reference.

Additional exemplary lipid-dsRNA formulations are as follows:

TABLE 2
Formulations

| | | cationic lipid/non-cationic lipid/ | |
|---|---|---|---|
| | | cholesterol/PEG-lipid conjugate | |
| | Cationic Lipid | Lipid:siRNA ratio | Process |
| SNALP | 1,2-Dilinolenyloxy-N,N- | DLinDMA/DPPC/Cholesterol/PEG-cDMA | |
| | dimethylaminopropane (DLinDMA) | (57.1/7.1/34.4/1.4) | |
| | | lipid:siRNA ~7:1 | |
| SNALP- | 2,2-Dilinoleyl-4-dimethylaminoethyl- | XTC/DPPC/Cholesterol/PEG-cDMA | |
| XTC | [1,3]-dioxolane (XTC) | 57.1/7.1/34.4/1.4 | |
| | | lipid:siRNA ~7:1 | |
| LNP05 | 2,2-Dilinoleyl-4-dimethylaminoethyl- | XTC/DSPC/Cholesterol/PEG-DMG | Extrusion |
| | [1,3]-dioxolane (XTC) | 57.5/7.5/31.5/3.5 | |
| | | lipid:siRNA ~6:1 | |
| LNP06 | 2,2-Dilinoleyl-4-dimethylaminoethyl- | XTC/DSPC/Cholesterol/PEG-DMG | Extrusion |
| | [1,3]-dioxolane (XTC) | 57.5/7.5/31.5/3.5 | |
| | | lipid:siRNA ~11:1 | |
| LNP07 | 2,2-Dilinoleyl-4-dimethylaminoethyl- | XTC/DSPC/Cholesterol/PEG-DMG | In-line |
| | [1,3]-dioxolane (XTC) | 60/7.5/31/1.5, | mixing |
| | | lipid:siRNA ~6:1 | |
| LNP08 | 2,2-Dilinoleyl-4-dimethylaminoethyl- | XTC/DSPC/Cholesterol/PEG-DMG | In-line |
| | [1,3]-dioxolane (XTC) | 60/7.5/31/1.5, | mixing |
| | | lipid:siRNA ~11:1 | |
| LNP09 | 2,2-Dilinoleyl-4-dimethylaminoethyl- | XTC/DSPC/Cholesterol/PEG-DMG | In-line |
| | [1,3]-dioxolane (XTC) | 50/10/38.5/1.5 | mixing |
| | | Lipid:siRNA 10:1 | |
| LNP10 | (3aR,5s,6aS)-N,N-dimethyl-2,2- | ALN100/DSPC/Cholesterol/PEG-DMG | In-line |
| | di((9Z,12Z)-octadeca-9,12- | 50/10/38.5/1.5 | mixing |
| | dienyl)tetrahydro-3aH- | Lipid:siRNA 10:1 | |
| | cyclopenta[d][1,3]dioxol-5-amine | | |
| | (ALN100) | | |
| LNP11 | (6Z,9Z,28Z,31Z)-heptatriaconta- | MC-3/DSPC/Cholesterol/PEG-DMG | In-line |
| | 6,9,28,31-tetraen-19-yl 4- | 50/10/38.5/1.5 | mixing |
| | (dimethylamino)butanoate (MC3) | Lipid:siRNA 10:1 | |
| LNP12 | 1,1′-(2-(4-(2-((2-(bis(2- | Tech G1/DSPC/Cholesterol/PEG-DMG | In-line |
| | hydroxydodecyl)amino)ethyl)(2- | 50/10/38.5/1.5 | mixing |
| | hydroxydodecyl)amino)ethyl)piperazin- | Lipid:siRNA 10:1 | |
| | 1-yl)ethylazanediyl)didodecan-2-ol | | |
| | (Tech G1) | | |

LNP09 formulations and XTC comprising formulations are described, e.g., in U.S. Provisional Ser. No. 61/239,686, filed Sep. 3, 2009, which is hereby incorporated by reference. LNP11 formulations and MC3 comprising formulations are described, e.g., in U.S. Provisional Ser. No. 61/244,834, filed Sep. 22, 2009, which is hereby incorporated by reference.

In one embodiment, the lipid particle comprises a charged lipid having the formula:

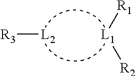

wherein:

R₁ and R₂ are each independently for each occurrence optionally substituted C₁₀-C₃₀ alkyl, optionally substituted C₁₀-C₃₀ alkoxy, optionally substituted C₁₀-C₃₀ alkenyl, optionally substituted C₁₀-C₃₀ alkenyloxy, optionally substituted C₁₀-C₃₀ alkynyl, optionally substituted C₁₀-C₃₀ alkynyloxy, or optionally substituted C₁₀-C₃₀ acyl;

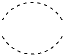

represents a connection between L₂ and L₁ which is:

(1) a single bond between one atom of L₂ and one atom of L₁, wherein
L₁ is C(Rₓ), O, S or N(Q);
L₂ is —CR₅R₆—, —O—, —S—, —N(Q)-, ═C(R₅)—, —C(O)N(Q)-, —C(O)O—, —N(Q)C(O)—, —OC(O)—, or —C(O)—;

(2) a double bond between one atom of L₂ and one atom of L₁; wherein
L₁ is C;
L₂ is —CR₅═, —N(Q)=, —N—, —O—N═, —N(Q)-N═, or —C(O)N(Q)-N═;

(3) a single bond between a first atom of L₂ and a first atom of L₁, and a single bond between a second atom of L₂ and the first atom of L₁, wherein
L₁ is C;
L₂ has the formula

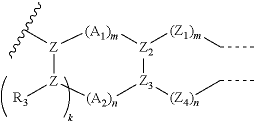

wherein

X is the first atom of L₂, Y is the second atom of L₂, - - - - - represents a single bond to the first atom of L₁, and X and Y are each, independently, selected from the group consisting of —O—, —S—, alkylene, —N(Q)-, —C(O)—, —O(CO)—, —OC(O)N(Q)-, —N(Q)C(O)O—, —C(O)O, —OC(O)O—, —OS(O)(Q₂)O—, and —OP(O)(Q₂)O—;
Z₁ and Z₄ are each, independently, —O—, —S—, —CH₂—, —CHR⁵—, or —CR⁵R⁵—;
Z₂ is CH or N;
Z₃ is CH or N;
or Z₂ and Z₃, taken together, are a single C atom;
A₁ and A₂ are each, independently, —O—, —S—, —CH₂—, —CHR⁵—, or —CR⁵R⁵—;
each Z is N, C(R₅), or C(R₃);
k is 0, 1, or 2;
each m, independently, is 0 to 5;
each n, independently, is 0 to 5;
where m and n taken together result in a 3, 4, 5, 6, 7 or 8 member ring;

(4) a single bond between a first atom of L₂ and a first atom of L₁, and a single bond between the first atom of L₂ and a second atom of L₁, wherein

(A) L₁ has the formula:

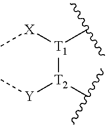

wherein
X is the first atom of L₁, Y is the second atom of L₁, - - - - - represents a single bond to the first atom of L₂, and X and Y are each, independently, selected from the group consisting of —O—, —S—, alkylene, —N(Q)-, —C(O)—, —O(CO)—, —OC(O)N(Q)-, —N(Q)C(O)O—, —C(O)O, —OC(O)O—, —OS(O)(Q₂)O—, and —OP(O)(Q₂)O—;
T₁ is CH or N;
T₂ is CH or N;
or T₁ and T₂ taken together are C═C;
L₂ is CR₅; or

(B) L₁ has the formula:

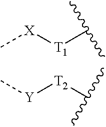

wherein
X is the first atom of L₁, Y is the second atom of L₁, - - - - - represents a single bond to the first atom of L₂, and X and Y are each, independently, selected from the group consisting of —O—, —S—, alkylene, —N(Q)-, —C(O)—, —O(CO)—, —OC(O)N(Q)-, —N(Q)C(O)O—, —C(O)O, —OC(O)O—, —OS(O)(Q2)O—, and —OP(O)(Q₂)O—;
T₁ is —CR₅R₅—, —N(Q)-, —O—, or —S—;
T₂ is —CR₅R₅—, —N(Q)-, —O—, or —S—;
L₂ is CR₅ or N;

R₃ has the formula:

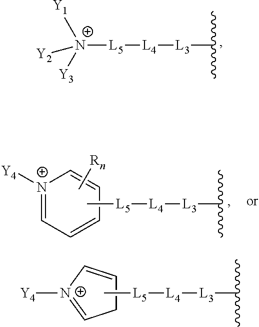

wherein

each of Y₁, Y₂, Y₃, and Y₄, independently, is alkyl, cycloalkyl, aryl, aralkyl, or alkynyl; or

any two of Y₁, Y₂, and Y₃ are taken together with the N atom to which they are attached to form a 3- to 8-member heterocycle; or

Y₁, Y₂, and Y₃ are all be taken together with the N atom to which they are attached to form a bicyclic 5- to 12-member heterocycle;

each Rₙ, independently, is H, halo, cyano, hydroxy, amino, alkyl, alkoxy, cycloalkyl, aryl, heteroaryl, or heterocyclyl;

L₃ is a bond, —N(Q)-, —O—, —S—, —(CR₅R₆)ₐ—, —C(O)—, or a combination of any two of these;

L₄ is a bond, —N(Q)-, —O—, —S—, —(CR₅R₆)ₐ—, —C(O)—, or a combination of any two of these;

L₅ is a bond, —N(Q)-, —O—, —S—, —(CR₅R₆)ₐ—, —C(O)—, or a combination of any two of these;

each occurrence of R₅ and R₆ is, independently, H, halo, cyano, hydroxy, amino, alkyl, alkoxy, cycloalkyl, aryl, heteroaryl, or heterocyclyl; or two R₅ groups on adjacent carbon atoms are taken together to form a double bond between their respective carbon atoms; or two R₅ groups on adjacent carbon atoms and two R₆ groups on the same adjacent carbon atoms are taken together to form a triple bond between their respective carbon atoms;

each a, independently, is 0, 1, 2, or 3;

wherein
an R₅ or R₆ substituent from any of L₃, L₄, or L₅ is optionally taken with an R₅ or R₆ substituent from any of L₃, L₄, or L₅ to form a 3- to 8-member cycloalkyl, heterocyclyl, aryl, or heteroaryl group; and
any one of Y₁, Y₂, or Y₃, is optionally taken together with an R₅ or R₆ group from any of L₃, L₄, and L₅, and atoms to which they are attached, to form a 3- to 8-member heterocyclyl group;

each Q, independently, is H, alkyl, acyl, cycloalkyl, alkenyl, alkynyl, aryl, heteroaryl or heterocyclyl; and

each Q₂, independently, is O, S, N(Q)(Q), alkyl or alkoxy.

In some embodiments,

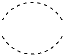

represents a connection between L₂ and L₁ which is a single bond between one atom of L₂ and one atom of L₁, wherein L₁ is C(Rₓ), O, S or N(Q); and L₂ is —CR₅R₆—, —O—, —S—, —N(Q)-, ═C(R₅)—, —C(O)N(Q)-, —C(O)O—, —N(Q)C(O)—, —OC(O)—, or —C(O)—.

In another aspect, a compound having formula I, XIII, XV, XVII, XXXIII, or XXXV:

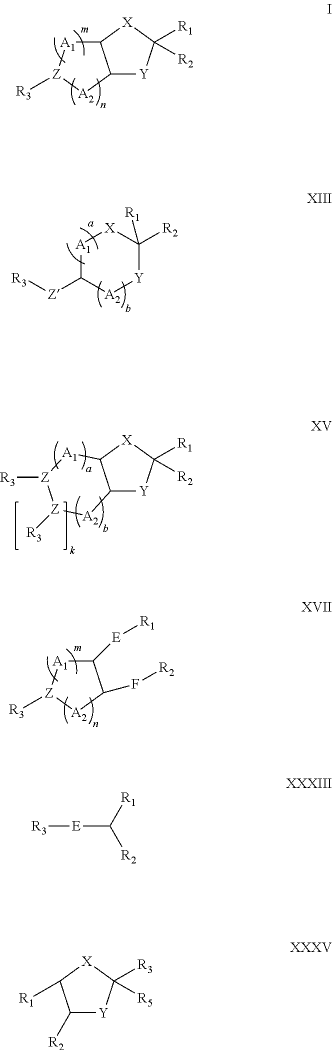

wherein:

R₁ and R₂ are each independently for each occurrence optionally substituted C₁₀-C₃₀ alkyl, optionally substituted C₁₀-C₃₀ alkoxy, optionally substituted C₁₀-C₃₀ alkenyl, optionally substituted C₁₀-C₃₀ alkenyloxy, optionally substituted C₁₀-C₃₀ alkynyl, optionally substituted C₁₀-C₃₀ alkynyloxy, or optionally substituted C₁₀-C₃₀ acyl;

R₃ is independently for each occurrence H, optionally substituted C₁-C₁₀ alkyl, optionally substituted C₂-C₁₀ alkenyl, optionally substituted C₂-C₁₀ alkynyl, optionally substituted alkylheterocycle, optionally substituted heterocyclealkyl, optionally substituted alkylphosphate, optionally substituted phosphoalkyl, optionally substituted alkylphosphorothioate, optionally substituted phosphorothioalkyl, optionally substituted alkylphosphorodithioate, optionally substituted phosphorodithioalkyl, optionally substituted alkylphosphonate, optionally substituted phosphonoalkyl, optionally substituted amino, optionally substituted alkylamino, optionally substituted di(alkyl)amino, optionally substituted aminoalkyl, optionally substituted alkylaminoalkyl, optionally substituted di(alkyl)aminoalkyl, optionally substituted hydroxyalkyl, optionally substituted polyethylene glycol (PEG, mw 100-40K), optionally substituted mPEG (mw 120-40K), optionally substituted heteroaryl, or optionally substituted heterocycle;

at least one R₃ includes a quaternary amine;

X and Y are each independently —O—, —S—, alkylene, —N(Q)—, —C(O)—, —O(CO)—, —OC(O)N(Q)-, —N(Q)C(O)—, —C(O)O, —OC(O)O—, —OS(O)(Q₂)O—, or —OP(O)(Q₂)O—;

Q is H, alkyl, ω-aminoalkyl, ω-(substituted)aminoalkyl, ω-phosphoalkyl, or ω-thiophosphoalkyl;

Q₂ is independently for each occurrence O, S, N(Q)(Q), alkyl or alkoxy;

A₁, A₂, A₃, A₄, A₅ and A₆ are each independently —O—, —S—, —CH₂—, —CHR⁵—, —CR⁵R⁵—;

A₈ is independently for each occurrence —CH₂—, —CHR⁵—, —CR⁵R⁵—;

E and F are each independently for each occurrence —CH₂—, —O—, —S—, —SS—, —CO—, —C(O)O—, —C(O)N(R′)—, —OC(O)N(R′)—, —N(R′)C(O)N(R″)—, —C(O)—N(R′)—N═C(R′″)—; —N(R′)—N═C(R″)—, —O—N═C(R″)—, —C(S)O—, —C(S)N(R′)—, —OC(S)N(R′)—, —N(R′)C(S)N(R″)—, —C(S)—N(R′)—N═C(R′″); —S—N═C(R″); —C(O)S—, —SC(O)N(R′)—, —OC(O)—, —N(R′)C(O)—, —N(R′)C(O)O—, —C(R′″)═N—N(R′)—; —C(R′″)═N—N(R′)—C(O)—, —C(R′″)═N—O—, —OC(S)—, —SC(O)—, —N(R′)C(S)—, —N(R′)C(S)O—, —N(R′)C(O)S—, —C(R′″)═N—N(R′)—C(S)—, —C(R′″)═N—S—, C[═N(R′)]O, C[═N(R′)]N(R″), —OC[═N(R′)]—, —N(R″)C[═N(R′)]N(R′″)—, —N(R″)C[═N(R′)]—,

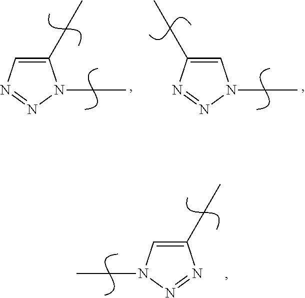

arylene, heteroarylene, cycloalkylene, or heterocyclylene;

Z is N or C(R₃);

Z′ is —O—, —S—, —N(Q)-, or alkylene;

each R′, R″, and R′″, independently, is H, alkyl, alkyl, heteroalkyl, aralkyl, cyclic alkyl, or heterocyclyl;

R⁵ is H, halo, cyano, hydroxy, amino, optionally substituted alkyl, optionally substituted alkoxy, or optionally substituted cycloalkyl;

i and j are each independently 0-10; and

a and b are each independently 0-2.

In another aspect, a compound can be selected from the group consisting of:

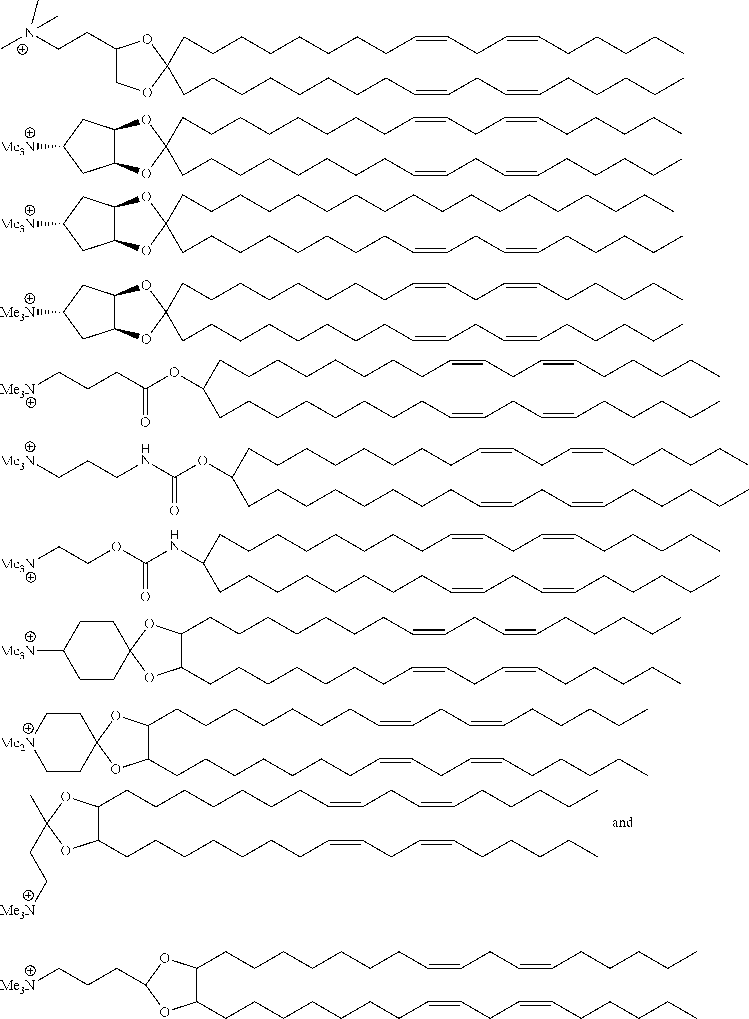

In one embodiment, the lipid particle further comprises a neutral lipid and a sterol. Neutral lipids, when present in the lipid particle, can be any of a number of lipid species which exist either in an uncharged or neutral zwitterionic form at physiological pH. Such lipids include, for example diacylphosphatidylcholine, diacylphosphatidylethanolamine, ceramide, sphingomyelin, dihydrosphingomyelin, cephalin, and cerebrosides. The selection of neutral lipids for use in the particles described herein is generally guided by consideration of, e.g., liposome size and stability of the liposomes in the bloodstream. Preferably, the neutral lipid component is a lipid having two acyl groups, (i.e., diacylphosphatidylcholine and diacylphosphatidylethanolamine). Lipids having a variety of acyl chain groups of varying chain length and degree of saturation are available or can be isolated or synthesized by well-known techniques. In one group of embodiments, lipids containing saturated fatty acids with carbon chain lengths in the range of C₁₀ to C₂₀ are preferred. In another group of embodiments, lipids with mono or diunsaturated fatty acids with carbon chain lengths in the range of C₁₀ to C₂₀ are used. Additionally, lipids having mixtures of saturated and unsaturated fatty acid chains can be used. Preferably, the neutral lipids used in the present invention are DOPE, DSPC, POPC, DPPC or any related phosphatidylcholine. The neutral lipids useful in the present invention can also be composed of sphingomyelin, dihydrosphingomyeline, or phospholipids with other head groups, such as serine and inositol.

The sterol component of the lipid mixture, when present, can be any of those sterols conventionally used in the field of liposome, lipid vesicle or lipid particle preparation. A preferred sterol is cholesterol.

Other protonatable lipids, which carry a net positive charge at about physiological pH, in addition to those specifically described above, can also be included in lipid particles of the present invention. Such protonatable lipids include, but are not limited to, N,N-dioleyl-N,N-dimethylammonium chloride (“DODAC”); N-(2,3-dioleyloxy)propyl-N,N-N-triethylammonium chloride (“DOTMA”); N,N-distearyl-N,N-dimethylammonium bromide (“DDAB”); N-(2,3-dioleoyloxy)propyl)-N,N,N-trimethylammonium chloride (“DOTAP”); 1,2-Dioleyloxy-3-trimethylaminopropane chloride salt (“DOTAP.Cl”); 3β-(N-(N′,N′-dimethylaminoethane)-carbamoyl)cholesterol (“DC-Chol”), N-(1-(2,3-dioleyloxy)propyl)-N-2-(sperminecarboxamido)ethyl)-N,N-dimethylammonium trifluoracetate (“DOSPA”), dioctadecylamidoglycyl carboxyspermine (“DOGS”), 1,2-dileoyl-sn-3-phosphoethanolamine (“DOPE”), 1,2-dioleoyl-3-dimethylammonium propane (“DODAP”), N,N-dimethyl-2,3-dioleyloxy)propylamine (“DODMA”), and N-(1,2-dimyristyloxyprop-3-yl)-N,N-dimethyl-N-hydroxyethyl ammonium bromide (“DMRIE”). Additionally, a number of commercial preparations of lipids can be used, such as, e.g., LIPOFECTIN (including DOTMA and DOPE, available from GIBCO/BRL), and LIPOFECTAMINE (comprising DOSPA and DOPE, available from GIBCO/BRL).

Anionic lipids suitable for use in lipid particles of the present invention include, but are not limited to, phosphatidylglycerol, cardiolipin, diacylphosphatidylserine, diacylphosphatidic acid, N-dodecanoyl phosphatidylethanoloamine, N-succinyl phosphatidylethanolamine, N-glutaryl phosphatidylethanolamine, lysylphosphatidylglycerol, and other anionic modifying groups joined to neutral lipids.

Additional components that can be present in a lipid particle as described herein include bilayer stabilizing components such as polyamide oligomers (see, e.g., U.S. Pat. No. 6,320,017), peptides, proteins, detergents, lipid-derivatives, such as PEG coupled to phosphatidylethanolamine and PEG conjugated to ceramides (see, U.S. Pat. No. 5,885,613).

The lipid particles described herein can further comprise one or more additional lipids and/or other components such as cholesterol.

As used herein, the term “charged lipid” is meant to include those lipids having one or two fatty acyl or fatty alkyl chains and a quaternary amino head group. The quaternary amine carries a permanent positive charge. The head group can optionally include a ionizable group, such as a primary, secondary, or tertiary amine that can be protonated at physiological pH. The presence of the quaternary amine can alter the pKa of the ionizable group relative to the pKa of the group in a structurally similar compound that lacks the quaternary amine (e.g., the quaternary amine is replaced by a tertiary amine) In some embodiments, a charged lipid is referred to as an “amino lipid.”

Other charged lipids would include those having alternative fatty acid groups and other quaternary groups, including those in which the alkyl substituents are different (e.g., N-ethyl-N-methylamino-, N-propyl-N-ethylamino- and the like). For those embodiments in which R₁ and R₂ are both long chain alkyl or acyl groups, they can be the same or different. In general, lipids (e.g., a charged lipid) having less saturated acyl chains are more easily sized, particularly when the complexes are sized below about 0.3 microns, for purposes of filter sterilization. Charged lipids containing unsaturated fatty acids with carbon chain lengths in the range of C₁₀ to C₂₀ are typical. Other scaffolds can also be used to separate the amino group (e.g., the amino group of the charged lipid) and the fatty acid or fatty alkyl portion of the charged lipid. Suitable scaffolds are known to those of skill in the art.

In certain embodiments, charged lipids of the present invention have at least one protonatable or deprotonatable group, such that the lipid is positively charged at a pH at or below physiological pH (e.g. pH 7.4), and neutral at a second pH, preferably at or above physiological pH. Such lipids are also referred to as charged lipids. It will, of course, be understood that the addition or removal of protons as a function of pH is an equilibrium process, and that the reference to a charged or a neutral lipid refers to the nature of the predominant species and does not require that all of the lipid be present in the charged or neutral form. Lipids that have more than one protonatable or deprotonatable group, or which are zwiterrionic, are not excluded from use in the invention.

In certain embodiments, protonatable lipids (i.e., charged lipids) according to the invention have a pKa of the protonatable group in the range of about 4 to about 11. Typically, lipids will have a pKa of about 4 to about 7, e.g., between about 5 and 7, such as between about 5.5 and 6.8, when incorporated into lipid particles. Such lipids will be cationic at a lower pH formulation stage, while particles will be largely (though not completely) surface neutralized at physiological pH around pH 7.4. One of the benefits of a pKa in the range of between about 4 and 7 is that at least some nucleic acid associated with the outside surface of the particle will lose its electrostatic interaction at physiological pH and be removed by simple dialysis; thus greatly reducing the particle's susceptibility to clearance. pKa measurements of lipids within lipid particles can be performed, for example, by using the fluorescent probe 2-(p-toluidino)-6-napthalene sulfonic acid (TNS), using methods described in Cullis et al., (1986) Chem Phys Lipids 40, 127-144.

Charged lipids can be prepared for use in transfection by forming into liposomes and mixing with the RNA effector molecules to be introduced into the cell. Methods of forming liposomes are well known in the art and include, but are not limited to, sonication, extrusion, extended vortexing, reverse evaporation, and homogenization, which includes microfluidization.

The reagent that facilitates uptake of an RNA effector molecule into the cell encompasses both single-layered liposomes, which are referred to as unilamellar, and multi-layered liposomes, which are referred to as multilamellar. Lipoplexes are composed of charged lipid bilayers sandwiched between nucleic acid layers, as described, e.g., in Felgner, Scientific American.

LNP01 formulations are described, e.g., in International Application Publication No. WO 2008/042973, which is hereby incorporated by reference in its entirety.

Formulations prepared by either the standard or extrusion-free method can be characterized in similar manners. For example, formulations are typically characterized by visual inspection. They should be whitish translucent solutions free from aggregates or sediment. Particle size and particle size distribution of lipid-nanoparticles can be measured by light scattering using, for example, a Malvern Zetasizer Nano ZS (Malvern, USA). Particles should be about 20-300 nm, such as e.g., 40-100 nm in size. The particle size distribution should be unimodal. The total siRNA effector molecule concentration in the formulation, as well as the entrapped fraction, is estimated using a dye exclusion assay. A sample of the formulated RNA effector molecule can be incubated with an RNA-binding dye, such as Ribogreen (Molecular Probes) in the presence or absence of a formulation disrupting surfactant, e.g., 0.5% Triton-X100. The total RNA effector molecule in the formulation can be determined by the signal from the sample containing the surfactant, relative to a standard curve. The entrapped fraction is determined by subtracting the “free” RNA effector molecule content (as measured by the signal in the absence of surfactant) from the total RNA effector molecule content. Percent entrapped RNA effector molecule is typically >85%. For lipid nanoparticle formulation, the particle size is at least 30 nm, at least 40 nm, at least 50 nm, at least 60 nm, at least 70 nm, at least 80 nm, at least 90 nm, at least 100 nm, at least 110 nm, and at least 120 nm. The suitable range is typically about at least 50 nm to about at least 110 nm, about at least 60 nm to about at least 100 nm, or about at least 80 nm to about at least 90 nm.

In some embodiments, RNA effector molecules featured in the invention are formulated in conjunction with one or more penetration enhancers, surfactants and/or chelators. Suitable surfactants include fatty acids and/or esters or salts thereof, bile acids and/or salts thereof Suitable bile acids/salts include chenodeoxycholic acid (CDCA) and ursodeoxychenodeoxycholic acid (UDCA), cholic acid, dehydrocholic acid, deoxycholic acid, glucholic acid, glycholic acid, glycodeoxycholic acid, taurocholic acid, taurodeoxycholic acid, sodium tauro-24,25-dihydro-fusidate and sodium glycodihydrofusidate. Suitable fatty acids include arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof (e.g., sodium). In some embodiments, combinations of penetration enhancers are used, for example, fatty acids/salts in combination with bile acids/salts. One exemplary combination is the sodium salt of lauric acid, capric acid and UDCA. Further penetration enhancers include polyoxyethylene-9-lauryl ether, polyoxyethylene-20-cetyl ether.

The compositions of the present invention can be formulated into any of many possible administration forms, including a sustained release form (e.g., tablets, capsules, gel capsules, liquid syrups, and soft gels). The compositions of the present invention can also be formulated as suspensions in aqueous, non-aqueous or mixed media. Aqueous suspensions can further contain substances which increase the viscosity of the suspension including, for example, sodium carboxymethylcellulose, sorbitol and/or dextran. The suspension can also contain stabilizers.

### Emulsions

The compositions of the present invention can be prepared and formulated as emulsions. Emulsions are typically heterogeneous systems of one liquid dispersed in another in the form of droplets usually exceeding 0.1 μm in diameter (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Idson, in Pharmaceutical Dosage Forms, Lieberman, Rieger and Banker (Eds.), 1988, Marcel Dekker, Inc., New York, N.Y., volume 1, p. 199; Rosoff, in Pharmaceutical Dosage Forms, Lieberman, Rieger and Banker (Eds.), 1988, Marcel Dekker, Inc., New York, N.Y., Volume 1, p. 245; Block in Pharmaceutical Dosage Forms, Lieberman, Rieger and Banker (Eds.), 1988, Marcel Dekker, Inc., New York, N.Y., volume 2, p. 335; Higuchi et al., in Remington's Pharmaceutical Sciences, Mack Publishing Co., Easton, Pa., 1985, p. 301). Emulsions are often biphasic systems comprising two immiscible liquid phases intimately mixed and dispersed with each other. In general, emulsions can be of either the water-in-oil (w/o) or the oil-in-water (o/w) variety. When an aqueous phase is finely divided into and dispersed as minute droplets into a bulk oily phase, the resulting composition is called a water-in-oil (w/o) emulsion. Alternatively, when an oily phase is finely divided into and dispersed as minute droplets into a bulk aqueous phase, the resulting composition is called an oil-in-water (o/w) emulsion. Emulsions can contain additional components in addition to the dispersed phases, and the active drug which can be present as a solution in either the aqueous phase, oily phase or itself as a separate phase. Pharmaceutical excipients such as emulsifiers, stabilizers, dyes, and anti-oxidants can also be present in emulsions as needed. Emulsions can also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise a system of oil droplets enclosed in globules of water stabilized in an oily continuous phase provides an o/w/o emulsion.

Emulsions are characterized by little or no thermodynamic stability. Often, the dispersed or discontinuous phase of the emulsion is well dispersed into the external or continuous phase and maintained in this form through the means of emulsifiers or the viscosity of the formulation. Either of the phases of the emulsion can be a semisolid or a solid, as is the case of emulsion-style ointment bases and creams. Other means of stabilizing emulsions entail the use of emulsifiers that can be incorporated into either phase of the emulsion. Emulsifiers can broadly be classified into four categories: synthetic surfactants, naturally occurring emulsifiers, absorption bases, and finely dispersed solids (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Idson, in Pharmaceutical Dosage Forms, Lieberman, Rieger and Banker (Eds.), 1988, Marcel Dekker, Inc., New York, N.Y., volume 1, p. 199).

Synthetic surfactants, also known as surface active agents, have found wide applicability in the formulation of emulsions and have been reviewed in the literature (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Rieger, in Pharmaceutical Dosage Forms, Lieberman, Rieger and Banker (Eds.), 1988, Marcel Dekker, Inc., New York, N.Y., volume 1, p. 285; Idson, in Pharmaceutical Dosage Forms, Lieberman, Rieger and Banker (Eds.), Marcel Dekker, Inc., New York, N.Y., 1988, volume 1, p. 199). Surfactants are typically amphiphilic and comprise a hydrophilic and a hydrophobic portion. The ratio of the hydrophilic to the hydrophobic nature of the surfactant has been termed the hydrophile/lipophile balance (HLB) and is a valuable tool in categorizing and selecting surfactants in the preparation of formulations. Surfactants can be classified into different classes based on the nature of the hydrophilic group: nonionic, anionic, cationic and amphoteric (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y. Rieger, in Pharmaceutical Dosage Forms, Lieberman, Rieger and Banker (Eds.), 1988, Marcel Dekker, Inc., New York, N.Y., volume 1, p. 285).

Naturally occurring emulsifiers used in emulsion formulations include lanolin, beeswax, phosphatides, lecithin and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers especially in combination with surfactants and in viscous preparations. These include polar inorganic solids, such as heavy metal hydroxides, nonswelling clays such as bentonite, attapulgite, hectorite, kaolin, montmorillonite, colloidal aluminum silicate and colloidal magnesium aluminum silicate, pigments and nonpolar solids such as carbon or glyceryl tristearate.

A large variety of non-emulsifying materials are also included in emulsion formulations and contribute to the properties of emulsions. These include fats, oils, waxes, fatty acids, fatty alcohols, fatty esters, humectants, hydrophilic colloids, preservatives and antioxidants (Block, in Pharmaceutical Dosage Forms, Lieberman, Rieger and Banker (Eds.), 1988, Marcel Dekker, Inc., New York, N.Y., volume 1, p. 335; Idson, in Pharmaceutical Dosage Forms, Lieberman, Rieger and Banker (Eds.), 1988, Marcel Dekker, Inc., New York, N.Y., volume 1, p. 199).

Hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylcellulose and carboxypropylcellulose), and synthetic polymers (for example, carbomers, cellulose ethers, and carboxyvinyl polymers). These disperse or swell in water to form colloidal solutions that stabilize emulsions by forming strong interfacial films around the dispersed-phase droplets and by increasing the viscosity of the external phase.

Since emulsions often contain a number of ingredients such as carbohydrates, proteins, sterols and phosphatides that can readily support the growth of microbes, these formulations often incorporate preservatives. Commonly used preservatives included in emulsion formulations include methyl paraben, propyl paraben, quaternary ammonium salts, benzalkonium chloride, esters of p-hydroxybenzoic acid, and boric acid. Antioxidants are also commonly added to emulsion formulations to prevent deterioration of the formulation. Antioxidants used can be free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin.

In one embodiment, the compositions of RNA effector molecules and nucleic acids are formulated as microemulsions. A microemulsion can be defined as a system of water, oil and amphiphile which is a single optically isotropic and thermodynamically stable liquid solution (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Rosoff, in Pharmaceutical Dosage Forms, Lieberman, Rieger and Banker (Eds.), 1988, Marcel Dekker, Inc., New York, N.Y., volume 1, p. 245). Typically microemulsions are systems that are prepared by first dispersing an oil in an aqueous surfactant solution and then adding a sufficient amount of a fourth component, generally an intermediate chain-length alcohol to form a transparent system. Therefore, microemulsions have also been described as thermodynamically stable, isotropically clear dispersions of two immiscible liquids that are stabilized by interfacial films of surface-active molecules (Leung and Shah, in: Controlled Release of Drugs: Polymers and Aggregate Systems, Rosoff, M., Ed., 1989, VCH Publishers, New York, pages 185-215). Microemulsions commonly are prepared via a combination of three to five components that include oil, water, surfactant, cosurfactant and electrolyte. Whether the microemulsion is of the water-in-oil (w/o) or an oil-in-water (o/w) type is dependent on the properties of the oil and surfactant used and on the structure and geometric packing of the polar heads and hydrocarbon tails of the surfactant molecules (Schott, in Remington's Pharmaceutical Sciences, Mack Publishing Co., Easton, Pa., 1985, p. 271).

The phenomenological approach utilizing phase diagrams has been extensively studied and has yielded a comprehensive knowledge, to one skilled in the art, of how to formulate microemulsions (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Rosoff, in Pharmaceutical Dosage Forms, Lieberman, Rieger and Banker (Eds.), 1988, Marcel Dekker, Inc., New York, N.Y., volume 1, p. 245; Block, in Pharmaceutical Dosage Forms, Lieberman, Rieger and Banker (Eds.), 1988, Marcel Dekker, Inc., New York, N.Y., volume 1, p. 335). Compared to conventional emulsions, microemulsions offer the advantage of solubilizing water-insoluble drugs in a formulation of thermodynamically stable droplets that are formed spontaneously.

Surfactants used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (MO310), hexaglycerol monooleate (PO310), hexaglycerol pentaoleate (PO500), decaglycerol monocaprate (MCA750), decaglycerol monooleate (MO750), decaglycerol sequioleate (SO750), decaglycerol decaoleate (DAO750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules. Microemulsions can, however, be prepared without the use of cosurfactants and alcohol-free self-emulsifying microemulsion systems are known in the art. The aqueous phase can typically be, but is not limited to, water, an aqueous solution of the drug, glycerol, PEG300, PEG400, polyglycerols, propylene glycols, and derivatives of ethylene glycol. The oil phase can include, but is not limited to, materials such as Captex 300, Captex 355, Capmul MCM, fatty acid esters, medium chain (C8-C12) mono, di, and tri-glycerides, polyoxyethylated glyceryl fatty acid esters, fatty alcohols, polyglycolized glycerides, saturated polyglycolized C8-C10 glycerides, vegetable oils and silicone oil.

Microemulsions afford advantages of improved agent solubilization, protection from enzymatic hydrolysis, possible enhancement of cellular uptake due to surfactant-induced alterations in membrane fluidity and permeability, ease of preparation, and decreased toxicity (see e.g., U.S. Pat. Nos. 6,191,105; 7,063,860; 7,070,802; 7,157,099; Constantinides et al., Pharmaceutical Research, 1994, 11, 1385; Ho et al., J. Pharm. Sci., 1996, 85, 138-143). Often microemulsions can form spontaneously when their components are brought together at ambient temperature. This can be particularly advantageous when formulating thermolabile compositions, peptides or RNA effector molecules.

Microemulsions of the present invention can also contain additional components and additives such as sorbitan monostearate (Grill 3), Labrasol, and penetration enhancers to improve the properties of the formulation and to enhance the absorption of the RNA effector molecules and nucleic acids of the present invention. Penetration enhancers used in the microemulsions of the present invention can be classified as belonging to one of five broad categories--surfactants, fatty acids, bile salts, chelating agents, and non-chelating non-surfactants (Lee et al., Critical Reviews in Therapeutic Drug Carrier Systems, 1991, p. 92). Each of these classes has been discussed above.

### Liposomes

There are many organized surfactant structures besides microemulsions that have been studied and used for the formulation of drugs. These include monolayers, micelles, bilayers and vesicles. Vesicles, such as liposomes, have attracted great interest because of their specificity and the duration of action they offer from the standpoint of drug delivery. As used in the present invention, the term “liposome” means a vesicle composed of amphiphilic lipids arranged in a spherical bilayer or bilayers.

Liposomes are unilamellar or multilamellar vesicles which have a membrane formed from a lipophilic material and an aqueous interior. The aqueous portion contains the composition to be delivered. Cationic liposomes possess the advantage of being able to fuse to the cell wall. In some embodiments, it is desirable to use a liposome which is highly deformable and able to pass through fine pores in a cell membrane or between cells grown in culture.

Further advantages of liposomes include: liposomes obtained from natural phospholipids are biocompatible and biodegradable; liposomes can incorporate a wide range of water and lipid soluble drugs; and liposomes can protect encapsulated RNA effector molecules in their internal compartments from metabolism and degradation (see e.g., Wang, B et al., Drug delivery: principles and applications, 2005, John Wiley and Sons, Hoboken, N.J.; Rosoff, in Pharmaceutical Dosage Forms, Lieberman, Rieger and Banker (Eds.), 1988, Marcel Dekker, Inc., New York, N.Y., volume 1, p. 245) in the cell culture medium. Important considerations in the preparation of liposome formulations are the lipid surface charge, vesicle size and the aqueous volume of the liposomes.

Liposomes are useful for the transfer and delivery of active ingredients to the site of action in the cell. Because the liposomal membrane is structurally similar to biological membranes, when liposomes are applied to a cell in culture, the liposomes start to merge with the cellular membranes and as the merging of the liposome and cell progresses, the liposomal contents are emptied into the cell where the RNA effector molecule acts.

Liposomal formulations have been the focus of extensive investigation as the mode of delivery for many compositions. Liposomes fall into two broad classes. Cationic liposomes are positively charged liposomes which interact with the negatively charged polynucleotide molecules to form a stable complex. The positively charged polynucleotide/liposome complex binds to the negatively charged cell surface and is internalized in an endosome. Due to the acidic pH within the endosome, the liposomes are ruptured, releasing their contents into the cell cytoplasm (Wang et al., Biochem. Biophys. Res. Commun , 1987, 147, 980-985).

Liposomes which are pH-sensitive or negatively-charged, entrap polynucleotide rather than complex with it. Since both the polynucleotide and the lipid are similarly charged, repulsion rather than complex formation occurs. Nevertheless, some polynucleotide is entrapped within the aqueous interior of these liposomes. pH-sensitive liposomes have been used to deliver DNA encoding the thymidine kinase gene to cell monolayers in culture. Expression of the exogenous gene was detected in the target cells (Zhou et al., Journal of Controlled Release, 1992, 19, 269-274).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome (A) comprises one or more glycolipids, such as monosialoganglioside G_{M1}, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES) (Allen et al., FEBS Letters, 1987, 223, 42; Wu et al., Cancer Research, 1993, 53, 3765).

Various liposomes comprising one or more glycolipids are known in the art. Papahadjopoulos et al. (Ann. N.Y. Acad. Sci., 1987, 507, 64) reported the ability of monosialoganglioside G_{M1}, galactocerebroside sulfate and phosphatidylinositol to improve blood half-lives of liposomes. These findings were expounded upon by Gabizon et al. (Proc. Natl. Acad. Sci. U.S.A., 1988, 85, 6949). U.S. Pat. No. 4,837,028 and WO 88/04924, both to Allen et al., disclose liposomes comprising (1) sphingomyelin and (2) the ganglioside G_{M1} or a galactocerebroside sulfate ester. U.S. Pat. No. 5,543,152 (Webb et al.) discloses liposomes comprising sphingomyelin. Liposomes comprising 1,2-sn-dimyristoylphosphatidylcholine are disclosed in WO 97/13499 (Lim et al).

Many liposomes comprising lipids derivatized with one or more hydrophilic polymers, and methods of preparation thereof, are known in the art. Sunamoto et al. (Bull. Chem. Soc. Jpn., 1980, 53, 2778) described liposomes comprising a nonionic detergent, 2C₁₂₁₅G, that contains a PEG moiety. Illum et al. (FEB S Lett., 1984, 167, 79) noted that hydrophilic coating of polystyrene particles with polymeric glycols results in significantly enhanced blood half-lives. Synthetic phospholipids modified by the attachment of carboxylic groups of polyalkylene glycols (e.g., PEG) are described by Sears (U.S. Pat. Nos. 4,426,330 and 4,534,899). In addition, antibodies can be conjugated to a polyakylene derivatized liposome (see e.g., PCT Application US 2008/0014255). Klibanov et al. (FEBS Lett., 1990, 268, 235) described experiments demonstrating that liposomes comprising phosphatidylethanolamine (PE) derivatized with PEG or PEG stearate have significant increases in blood circulation half-lives. Blume et al. (Biochimica et Biophysica Acta, 1990, 1029, 91) extended such observations to other PEG-derivatized phospholipids, e.g., DSPE-PEG, formed from the combination of distearoylphosphatidylethanolamine (DSPE) and PEG. Liposomes having covalently bound PEG moieties on their external surface are described in European Patent No. EP 0 445 131 B1 and WO 90/04384 to Fisher. Liposome compositions containing 1-20 mole percent of PE derivatized with PEG, and methods of use thereof, are described by Woodle et al. (U.S. Pat. Nos. 5,013,556 and 5,356,633) and Martin et al. (U.S. Pat. No. 5,213,804 and European Patent No. EP 0 496 813 B1). Liposomes comprising a number of other lipid-polymer conjugates are disclosed in WO 91/05545 and U.S. Pat. No. 5,225,212 (both to Martin et al.) and in WO 94/20073 (Zalipsky et al.) Liposomes comprising PEG-modified ceramide lipids are described in WO 96/10391 (Choi et al). U.S. Pat. No. 5,540,935 (Miyazaki et al.) and U.S. Pat. No. 5,556,948 (Tagawa et al.) describe PEG-containing liposomes that can be further derivatized with functional moieties on their surfaces. Methods and compositions relating to liposomes comprising PEG can be found in e.g., U.S. Pat. Nos. 6,049,094; 6,224,903; 6,270,806; 6,471,326; and 6,958,241.

As noted above, liposomes can optionally be prepared to contain surface groups, such as antibodies or antibody fragments, small effector molecules for interacting with cell-surface receptors, antigens, and other like compounds, and these groups can facilitate delivery of liposomes and their contents to specific cell populations. Such ligands can be included in the liposomes by including in the liposomal lipids a lipid derivatized with the targeting molecule, or a lipid having a polar-head chemical group that can be derivatized with the targeting molecule in preformed liposomes. Alternatively, a targeting moiety can be inserted into preformed liposomes by incubating the preformed liposomes with a ligand-polymer-lipid conjugate.

Also suitable for inclusion in the lipid particles of the present invention are programmable fusion lipids. Such lipid particles have little tendency to fuse with cell membranes and deliver their payload until a given signal event occurs. This allows the lipid particle to distribute more evenly after injection into an organism or disease site before it starts fusing with cells. The signal event can be, for example, a change in pH, temperature, ionic environment, or time. In the latter case, a fusion delaying or “cloaking” component, such as an ATTA-lipid conjugate or a PEG-lipid conjugate, can simply exchange out of the lipid particle membrane over time. By the time the lipid particle is suitably distributed in the body, it has lost sufficient cloaking agent so as to be fusogenic. With other signal events, it is desirable to choose a signal that is associated with the disease site or target cell, such as increased temperature at a site of inflammation.

In certain embodiments, it is desirable to target the lipid particles of this invention using targeting moieties that are specific to a cell type or tissue. Targeting of lipid particles using a variety of targeting moieties, such as ligands, cell surface receptors, glycoproteins, vitamins (e.g., riboflavin) and monoclonal antibodies, have been previously described (see, e.g., U.S. Pat. Nos. 4,957,773 and 4,603,044). The targeting moieties can comprise the entire protein or fragments thereof Targeting mechanisms generally require that the targeting agents be positioned on the surface of the lipid particle in such a manner that the target moiety is available for interaction with the target, for example, a cell surface receptor. A variety of different targeting agents and methods are known and available in the art, including those described, e.g., in Sapra, P. and Allen, T M, Prog. Lipid Res. 42(5):439-62 (2003); and Abra, R M et al., J. Liposome Res. 12:1-3, (2002).

The use of lipid particles, i.e., liposomes, with a surface coating of hydrophilic polymer chains, such as polyethylene glycol (PEG) chains, for targeting has been proposed (Allen, et al., Biochimica et Biophysica Acta 1237: 99-108 (1995); DeFrees, et al., Journal of the American Chemistry Society 118: 6101-6104 (1996); Blume, et al., Biochimica et Biophysica Acta 1149: 180-184 (1993); Klibanov, et al., Journal of Liposome Research 2: 321-334 (1992); U.S. Pat. No. 5,013556; Zalipsky, Bioconjugate Chemistry 4: 296-299 (1993); Zalipsky, FEBS Letters 353: 71-74 (1994); Zalipsky, in Stealth Liposomes Chapter 9 (Lasic and Martin, Eds) CRC Press, Boca Raton Fla. (1995). In one approach, a ligand, such as an antibody, for targeting the lipid particle is linked to the polar head group of lipids forming the lipid particle. In another approach, the targeting ligand is attached to the distal ends of the PEG chains forming the hydrophilic polymer coating (Klibanov, et al., Journal of Liposome Research 2: 321-334 (1992); Kirpotin et al., FEBS Letters 388: 115-118 (1996)).

Standard methods for coupling the target agents can be used. For example, phosphatidylethanolamine, which can be activated for attachment of target agents, or derivatized lipophilic compounds, such as lipid-derivatized bleomycin, can be used. Antibody-targeted liposomes can be constructed using, for instance, liposomes that incorporate protein A (see, Renneisen, et al., J. Bio. Chem., 265:16337-16342 (1990) and Leonetti, et al., Proc. Natl. Acad. Sci. (USA), 87:2448-2451 (1990). Other examples of antibody conjugation are disclosed in U.S. Pat. No. 6,027,726, the teachings of which are incorporated herein by reference. Examples of targeting moieties can also include other proteins, specific to cellular components, including antigens associated with neoplasms or tumors. Proteins used as targeting moieties can be attached to the liposomes via covalent bonds (see, Heath, Covalent Attachment of Proteins to Liposomes, 149 Methods in Enzymology 111-119 (Academic Press, Inc. 1987)). Other targeting methods include the biotin-avidin system.

In one exemplary embodiment, the lipid particle comprises a mixture of a charged lipid of the present invention, one or more different neutral lipids, and a sterol (e.g., cholesterol). In certain embodiments, the lipid mixture consists of or consists essentially of a charged lipid as described herein, a neutral lipid, and cholesterol. In further preferred embodiments, the lipid particle consists of or consists essentially of the above lipid mixture in molar ratios of about 50-90% charged lipid, 0-50% neutral lipid, and 0-10% cholesterol. In certain embodiments, the lipid particle can further include a PEG-modified lipid (e.g., a PEG-DMG or PEG-DMA).

In one embodiment, the lipid particle consists of a charged lipid (e.g., a quaternary nitrogen containing lipid) and a protonatable lipid, a neutral lipid or a steroid, or a combination thereof The particles can be formulated with a nucleic acid therapeutic agent so as to attain a desired N/P ratio. The N/P ratio is the ratio of number of molar equivalent of cationic nitrogen (N) atoms present in the lipid particle to the number of molar equivalent of anionic phosphate (P) of the nucleic acid backbone. For example, the N/P ratio can be in the range of about 1 to about 50. In one example, the range is about 1 to about 20, about 1 to about 10, about 1 to about 5.

In particular embodiments, the lipid particle consists of or consists essentially of a charged lipid described in paragraph [00246] herein, DOPE, and cholesterol. In particular embodiments, the particle includes lipids in the following mole percentages: charged lipid, 45-63 mol %; DOPE, 35-55 mol %; and cholesterol, 0-10 mol %. The particles can be formulated with a nucleic acid therapeutic agent so as to attain a desired N/P ratio. The N/P ratio is the ratio of number of moles cationic nitrogen (N) atoms (i.e., charged lipids) to the number of molar equivalents of anionic phosphate (P) backbone groups of the nucleic acid. For example, the N to P ratio can be in the range of about 5:1 to about 1:1. In certain embodiments, the charged lipid is chosen from those described in paragraph [00215] herein.

In another group of embodiments, the neutral lipid, DOPE, in these compositions is replaced with POPC, DPPC, DPSC or SM.

A number of liposomes comprising nucleic acids are known in the art. WO 96/40062 (Thierry et al.) discloses methods for encapsulating high molecular weight nucleic acids in liposomes. U.S. Pat. No. 5,264,221 (Tagawa et al.) discloses protein-bonded liposomes and asserts that the contents of such liposomes can include a dsRNA. U.S. Pat. No. 5,665,710 (Rahman et al.) describes certain methods of encapsulating oligodeoxynucleotides in liposomes. WO 97/04787 (Love et al.) discloses liposomes comprising dsRNAs targeted to the raf gene. In addition, methods for preparing a liposome composition comprising a nucleic acid can be found in e.g., U.S. Pat. Nos. 6,011,020; 6,074,667; 6,110,490; 6,147,204; 6, 271, 206; 6,312,956; 6,465,188; 6,506,564; 6,750,016; and 7,112,337.

Transfersomes are yet another type of liposome, and are highly deformable lipid aggregates which are attractive candidates for RNA delivery vehicles. Transfersomes can be described as lipid droplets which are so highly deformable that they are easily able to penetrate through pores which are smaller than the droplet. Transfersomes are adaptable to the environment in which they are used, e.g., they are self-optimizing, self-repairing, frequently reach their targets without fragmenting, and often self-loading. To make transfersomes it is possible to add surface edge-activators, usually surfactants, to a standard liposomal composition.

Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations (see e.g., Malmsten, M. Surfactants and polymers in drug delivery, Informa Health Care, New York, N.Y., 2002; Rieger, in Pharmaceutical Dosage Forms, Marcel Dekker, Inc., New York, N.Y., 1988, p. 285).

If the surfactant molecule is not ionized, it is classified as a nonionic surfactant. Nonionic surfactants find wide application in pharmaceutical and cosmetic products and are usable over a wide range of pH values. In general their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines and phosphatides.

The use of surfactants in drug products, formulations and in emulsions has been reviewed (see e.g., Malmsten, M. Surfactants and polymers in drug delivery, Informa Health Care, New York, N.Y., 2002; Rieger, in Pharmaceutical Dosage Forms, Marcel Dekker, Inc., New York, N.Y., 1988, p. 285).

### Penetration Enhancers

In one embodiment, the present invention employs various penetration enhancers to effect the efficient delivery of nucleic acids, particularly RNA effector molecules, to the cell in culture. Typically, only lipid soluble or lipophilic compositions readily cross cell membranes. It has been discovered that even non-lipophilic compositions can cross cell membranes if the membrane to be crossed is treated with a penetration enhancer. In addition to aiding the diffusion of non-lipophilic compositions across cell membranes, penetration enhancers also enhance the permeability of lipophilic compositions.

Agents that enhance uptake of RNA effector molecules at the cellular level can also be added to the pharmaceutical and other compositions of the present invention. For example, cationic lipids, such as lipofectin (Junichi et al, U.S. Pat. No. 5,705,188), cationic glycerol derivatives, and polycationic molecules, such as polylysine (Lollo et al., PCT Application WO 97/30731), are also known to enhance the cellular uptake of dsRNAs. Examples of commercially available transfection reagents include, for example Lipofectamine™ (Invitrogen; Carlsbad, Calif.), Lipofectamine 2000™ (Invitrogen; Carlsbad, Calif.), 293fectin™ (Invitrogen; Carlsbad, Calif.), Cellfectin™ (Invitrogen; Carlsbad, Calif.), DMRIE-C™ (Invitrogen; Carlsbad, Calif.), FreeStyle™ MAX (Invitrogen; Carlsbad, Calif.), Lipofectamine™ 2000 CD (Invitrogen; Carlsbad, Calif.), Lipofectamine™ (Invitrogen; Carlsbad, Calif.), RNAiMAX (Invitrogen; Carlsbad, Calif.), Oligofectamine™ (Invitrogen; Carlsbad, Calif.), Optifect™ (Invitrogen; Carlsbad, Calif.), X-tremeGENE Q2 Transfection Reagent (Roche; Grenzacherstrasse, Switzerland), DOTAP Liposomal Transfection Reagent (Grenzacherstrasse, Switzerland), DOSPER Liposomal Transfection Reagent (Grenzacherstrasse, Switzerland), or Fugene (Grenzacherstrasse, Switzerland), Transfectam® Reagent (Promega; Madison, Wis.), TransFast™ Transfection Reagent (Promega; Madison, Wis.), Tfx™-20 Reagent (Promega; Madison, Wis.), Tfx™-50 Reagent (Promega; Madison, Wis.), DreamFect™ (OZ Biosciences; Marseille, France), EcoTransfect (OZ Biosciences; Marseille, France), TransPass^{a} D1 Transfection Reagent (New England Biolabs; Ipswich, Mass., USA), LyoVec™/LipoGen™ (Invitrogen; San Diego, Calif., USA), PerFectin Transfection Reagent (Genlantis; San Diego, Calif., USA), NeuroPORTER Transfection Reagent (Genlantis; San Diego, Calif., USA), GenePORTER Transfection reagent (Genlantis; San Diego, Calif., USA), GenePORTER 2 Transfection reagent (Genlantis; San Diego, Calif., USA), Cytofectin Transfection Reagent (Genlantis; San Diego, Calif., USA), BaculoPORTER Transfection Reagent (Genlantis; San Diego, Calif., USA), TroganPORTER™ transfection Reagent (Genlantis; San Diego, Calif., USA), RiboFect (Bioline; Taunton, Mass., USA), PlasFect (Bioline; Taunton, Mass., USA), UniFECTOR (B-Bridge International; Mountain View, Calif., USA), SureFECTOR (B-Bridge International; Mountain View, Calif., USA), or HiFect™ (B-Bridge International, Mountain View, Calif., USA), among others.

Other agents can be utilized to enhance the penetration of the administered nucleic acids, including glycols such as ethylene glycol and propylene glycol, pyrrols such as 2-pyrrol, azones, and terpenes such as limonene and menthone.

### Carriers

Certain compositions of the present invention also incorporate carrier compounds in the formulation. As used herein, “carrier compound” or “carrier” can refer to a nucleic acid, or analog thereof, which is inert (i.e., does not possess biological activity per se) but is recognized as a nucleic acid by in vivo processes that reduce the bioavailability of a nucleic acid having biological activity by, for example, degrading the biologically active nucleic acid or promoting its removal.

### Other Components

The compositions of the present invention can additionally contain other adjunct components so long as such materials, when added, do not unduly interfere with the biological activities of the components of the compositions of the present invention. The formulations can be sterilized and, if desired, mixed with auxiliary agents which do not deleteriously interact with the nucleic acid(s) of the formulation.

Aqueous suspensions can contain substances which increase the viscosity of the suspension including, for example, sodium carboxymethylcellulose, sorbitol and/or dextran. The suspension can also contain stabilizers.

Toxicity and therapeutic efficacy of such compounds can be determined by standard cell based assays cell cultures, e.g., cell death assays for determining the level of toxicity or evaluating an LD50 (the dose lethal to 50% of the cells in the population) and the ED50 (the dose therapeutically effective in 50% of the cellular population). The dose ratio between toxic and therapeutic effects is the therapeutic index and it can be expressed as the ratio LD50/ED50. Compounds that exhibit high therapeutic indices are preferred as they are less likely to induce cell toxicity during the production of a modified polypeptide.

The data obtained from cell culture assays can be used in formulating a range of dosages for use in the instant methods. The dosage of compositions featured in the invention lies generally within a range of concentrations that includes the ED50 with little or no toxicity. The dosage can vary within this range depending upon the dosage form employed and the route of administration utilized.

### Use of Producer Cells for Industrial Production of a Biological Product

The methods and compositions described herein can be applied to any system for producing a biological product using cells capable of producing a biological product (e.g., producer cells) as described herein, including polypeptide production on an industrial scale. Following the sequential selection of cells having multiple copies of a transgene linked to a selectable amplifiable marker using an amplification reagent, the cell lines (also referred to herein as “producer cells”) can be used to produce a biological product. The producer cells described herein can be combined with any known method or composition to enhance the production of a polypeptide or biological product, such as those disclosed in e.g., U.S. Provisional No. 61/293,980 or described herein.

In one embodiment, the producer cells are used to produce a biological product on an industrial scale. A non-limiting exemplary process for the industrial-scale production of a heterologous polypeptide (e.g., a polypeptide to be modified) in cell culture (e.g., mammalian cell culture) includes the following steps:

i) inoculating mammalian host cells containing a transgene linked to a selectable amplifiable marker into a seed culture vessel containing cell culture medium and propagating the cells to reach a minimum threshold cross-seeding density;

ii) transferring the propagated seed culture cells, or a portion thereof, to a large-scale bioreactor;

iii) propagating the large-scale culture under conditions allowing for rapid growth and cell division until the cells reach a predetermined density;

iv) maintaining the culture under conditions that disfavor continued cell growth and/or cell division and facilitate expression of the heterologous protein.

The cells can be cultured in a stirred tank bioreactor system in a fed batch culture process in which the host cells and culture medium are supplied to the bioreactor initially and additional culture nutrients are fed, continuously or in discrete increments, throughout the cell culture process. The fed batch culture process can be semi-continuous, wherein periodically the entire culture (including cells and medium) is removed and replaced. Alternatively, a simple batch culture process can be used in which all components for cell culturing (including the cells and culture medium) are supplied to the culturing vessel at the start of the process. A continuous perfusion process can also be used, in which the cells are immobilized in the culture, e.g., by filtration, encapsulation, anchoring to microcarriers, or the like, and the supernatant is continuously removed from the culturing vessel and replaced with fresh medium during the process.

Steps i)-iii) of the above method generally comprise a “growth” phase, whereas step iv) generally comprises a “production” phase. In some embodiments, fed batch culture or continuous cell culture conditions are tailored to enhance growth and division of the cultured cells in the growth phase and to disfavor cell growth and/or division and facilitate expression of the heterologous protein during the production phase. For example, in some embodiments, a biological product is expressed at levels of about 1 mg/L, or about 2.5 mg/L, or about 5 mg/L or higher. The rate of cell growth and/or division can be modulated by varying culture conditions, such as temperature, pH, dissolved oxygen (dO₂) and the like. For example, suitable conditions for the growth phase can include a pH of between about 6.5 and 7.5, a temperature between about 30° C. to 38° C., and a dO₂ between about 5-90% saturation. In some embodiments, the expression of a biological product can be enhanced in the production phase by inducing a temperature shift to a lower culture temperature (e.g., from about 37° C. to about 30° C.), increasing the concentration of solutes in the cell culture medium, or adding a toxin (e.g., sodium butyrate) to the cell culture medium. A variety of additional protocols and conditions for enhancing growth during the growth phase and/or protein expression during the production phase are known in the art.

In one embodiment, after the production phase the biological product is recovered from the cell culture medium using various methods known in the art. Recovering a secreted biological product or polypeptide typically involves removal of host cells and debris from the medium, for example, by centrifugation or filtration. In some embodiments, the methods provided herein further comprise inhibition of the mannose 6 phosphate receptor such that the expressed polypeptide does not accumulate in lysosomes. In other embodiments, the polypeptide produced in a host cell does not comprise a mannose 6 phosphate group such that it is preferentially secreted rather than imported into lysosomes by mannose 6 phosphate mediated uptake.

In some cases, particularly if the protein is not secreted, protein recovery can also be performed by lysing the cultured host cells, e.g., by mechanical shear, osmotic shock, or enzymatic treatment, to release the contents of the cells into the homogenate. The polypeptide can then be separated from subcellular fragments, insoluble materials, and the like by differential centrifugation, filtration, affinity chromatography, hydrophobic interaction chromatography, ion-exchange chromatography, size exclusion chromatography, electrophoretic procedures (e.g., preparative isoelectric focusing (IEF)), ammonium sulfate precipitation, and the like. Procedures for recovering and purifying particular types of proteins are known in the art.

Methods and compositions useful for enhancing polypeptide production in cells is provided in e.g., U.S. Provisional Application 61/293,980, which is incorporated herein by reference in its entirety. Such methods are directed at e.g., increasing cell growth, increasing cell viability, decreasing apoptosis, decreasing lactate formation, decreasing reactive oxygen species production, modifying post-translational modifications, and decreasing viral contamination of cells in culture.

In another embodiment, the RNA effector molecule is added to maintain the cells during the production process at an amount of 50 molecules per cell, 100 molecules per cell, 200 molecules per cell, 300 molecules per cell, 400 molecules per cell, 500 molecules per cell, 600 molecules per cell, 700 molecules per cell, 800 molecules per cell, 900 molecules per cell, 1000 molecules per cell, 2000 molecules per cell, or 5000 molecules per cell.

In another embodiment, the at least one RNA effector molecule is added to maintain the cells during the production process at a concentration selected from the group consisting of: 0.01 fmol/10⁶ cells, 0.1 fmol/10⁶ cells, 0.5 fmol/10⁶ cells, 0.75 fmol/10⁶ cells, 1 fmol/10⁶ cells, 2 fmol/10⁶ cells, 5 fmol/10⁶ cells, 10 fmol/10⁶ cells, 20 fmol/10⁶ cells, 30 fmol/10⁶ cells, 40 fmol/10⁶ cells, 50 fmol/10⁶ cells, 60 fmol/10⁶ cells, 100 fmol/10⁶ cells, 200 fmol/10⁶ cells, 300 fmol/10⁶ cells, 400 fmol/10⁶ cells, 500 fmol/10⁶ cells, 700 fmol/10⁶ cells, 800 fmol/10⁶ cells, 900 fmol/10⁶ cells, and 1 pmol/10⁶ cells.

In another embodiment, the cells produced using the methods described herein can be cultured in the presence or the absence of the amplification reagent during the production of the biological product. Such cells can also be transfected with an RNA effector molecule that partially inhibits expression (e.g., at least 10%) of the selectable amplifiable marker such that the cell overexpresses the biological product in the absence of substantial overexpression of the selectable amplifiable marker.

### Kits for Generating a Cell Capable of Producing a Biological Product

In some embodiments, kits are provided for generating a cell capable of producing a biological, where the kits comprise at a minimum, a vector comprising a selectable amplifiable marker gene that has a nucleic acid sequence distinct from that of the same marker gene endogenous to the host cell, an RNA effector molecule, and packaging materials therefor. The kit can further comprise a host cell provided as e.g., frozen cells or cells in culture. In one embodiment, the host cell is a CHO cell.

In another embodiment, the kit comprises a substrate having one or more selection surfaces suitable for culturing host cells under conditions that allow selection of a cell based on the expression of the first amplifiable marker gene that confers resistance to an amplification reagent. In some embodiments, the exterior of the substrate comprises wells, indentations, demarcations, or the like at positions corresponding to the selection surfaces. In some preferred embodiments, the wells, indentations, demarcations, or the like retain fluid, such as cell culture media, over the surfaces.

In some embodiments, the surfaces on the substrate are sterile and are suitable for culturing host cells under conditions representative of the cell culture conditions during large-scale (e.g., industrial scale) production of the biological product. In some embodiments, one or more surfaces of the substrate comprise a concentrated test agent, such as an RNA effector molecule, such that the addition of suitable media to the assay surfaces results in a desired concentration of the RNA effector molecule surrounding the surface. In some embodiments, the RNA effector molecules can be printed or ingrained onto the surface, or provided in a lyophilized form, e.g., within wells, such that the effector molecules can be reconstituted upon addition of an appropriate amount of media. In some embodiments, the RNA effector molecules are reconstituted by plating cells onto surfaces of the substrate.

In some embodiments, kits provided herein further comprise cell culture media suitable for culturing a host cell under conditions allowing for selection of a cell capable of producing a biological product. The media can be in a ready to use form or can be concentrated (e.g., as a stock solution), lyophilized, or provided in another reconstitutable form.

In some embodiments, one or more surfaces of the substrate further comprises a reagent that facilitates uptake of RNA effector molecules by host cells. Such reagent carriers for RNA effector molecules are known in the art and/or are described herein. For example, in some embodiments, the carrier is a lipid formulation such as Lipofectamine™ (Invitrogen; Carlsbad, Calif.) or a related formulation. Examples of such carrier formulations are described herein.

In some embodiments, one or more surfaces of the substrate comprise an RNA effector molecule or series of RNA effector molecules and a carrier, each in concentrated form, such that plating host cells onto the surface(s) results in a concentration of the RNA effector molecule(s) and the carrier effective for facilitating uptake of the RNA effector molecule(s) by the host cells and modulation of the expression of one or more genes targeted by the RNA effector molecules.

In some embodiments, the substrate further comprises a matrix which facilitates three-dimensional cell growth and/or production of the biological product by host cells. In some embodiments, the matrix facilitates anchorage-independent growth of host cells. In further embodiments, the matrix facilitates anchorage-dependent growth of host cells. Non-limiting examples of matrix materials suitable for use with various kits described herein include agar, agarose, methylcellulose, alginate hydrogel (e.g., 5% alginate+5% collagen type I), chitosan, hydroactive hydrocolloid polymer gels, polyvinyl alcohol-hydrogel (PVA-H), polylactide-co-glycolide (PLGA), collagen vitrigel, PHEMA (poly(2-hydroxylmethacrylate)) hydrogels, PVP/PEO hydrogels, BD PuraMatrix™ hydrogels, and copolymers of 2-methacryloyloxyethyl phophorylcholine (MPC).

In some embodiments, the substrate comprises a microarray plate, a biochip, or the like which allows for the high-throughput, automated testing of a range of test agents, conditions, and/or combinations thereof on the production of a modified polypeptide by cultured host cells. For example, the substrate can comprise a two-dimensional microarray plate or biochip having m columns and n rows of assay surfaces (e.g., residing within wells) which allow for the testing of m×n combinations of test agents and/or conditions (e.g., on a 24, 96 or 384-well microarray plate). The microarray substrates are preferably designed such that all necessary positive and negative controls can be carried out in parallel with testing of the agents and/or conditions.

In some embodiments, kits are provided comprising one or more microarray plates or biochips seeded with a series of RNA effector molecules to test the efficacy of each RNA effector molecule alone, or in combination. In further embodiments, kits are provided that can further comprise one or more microarray substrates seeded with different concentrations of an amplification reagent.

In some embodiments, kits provided herein allow for the selection or optimization of the concentration of an amplification reagent or the amount of an RNA effector molecule adequate for inhibition of expression of an endogenous selectable amplifiable marker gene. For example, the kits can allow for the selection of an RNA effector molecule from among a series of candidate RNA effector molecules, or for the selection of a concentration or concentration range from a wider range of concentrations of a given RNA effector molecule. In some embodiments, the kits allow for selection of one or more RNA effector molecules from a series of candidate RNA effector molecules directed against a common target gene.

In another embodiment, a kit for generating a cell capable of producing a biological product from a host cell is provided comprising one or more microarray plates seeded with a range of concentrations of an RNA effector molecule.

In another embodiment, a kit for generating a cell capable of producing a biological product from a host cell is provided comprising one or more two-dimensional microarray plates seeded along one dimension (e.g., rows or columns) with a series of RNA effector molecules and along the remaining dimension with a series of concentrations of an amplification reagent.

In another embodiment, the kit further comprises a cell medium for culturing the host cell.

In other embodiments, the RNA effector molecule is provided at a concentration selected from the group consisting of 0.1 nM, 0.5 nM, 0.75 nM, 1 nM, 2 nM, 5 nM, 10 nM, 20 nM, 30 nM, 40 nM, 50 nM, and 60 nM. Alternatively, in other embodiments the RNA effector molecule is provided at an amount of 50 molecules per cell, 100 molecules per cell, 200 molecules per cell, 300 molecules per cell, 400 molecules per cell, 500 molecules per cell, 600 molecules per cell, 700 molecules per cell, 800 molecules per cell, 900 molecules per cell, 1000 molecules per cell, 2000 molecules per cell, or 5000 molecules per cell. In further embodiments, the RNA effector molecule is provided at a concentration selected from the group consisting of: 0.01 fmol/10⁶ cells, 0.1 fmol/10⁶ cells, 0.5 fmol/10⁶ cells, 0.75 fmol/10⁶ cells, 1 fmol/10⁶ cells, 2 fmol/10⁶ cells, 5 fmol/10⁶ cells, 10 fmol/10⁶ cells, 20 fmol/10⁶ cells, 30 fmol/10⁶ cells, 40 fmol/10⁶ cells, 50 fmol/10⁶ cells, 60 fmol/10⁶ cells, 100 fmol/10⁶ cells, 200 fmol/10⁶ cells, 300 fmol/10⁶ cells, 400 fmol/10⁶ cells, 500 fmol/10⁶ cells, 700 fmol/10⁶ cells, 800 fmol/10⁶ cells, 900 fmol/10⁶ cells, and 1 pmol/10⁶ cells.

In another embodiment, the kit further comprises an RNA effector molecule that inhibits expression of the mannose 6 phosphate receptor.

The present invention may be as defined in any one of the following numbered paragraphs:

1. A method of generating a cell line capable of producing a biological product comprising:(a) providing a plurality of host cells comprising a first selectable amplifiable marker gene and a second selectable amplifiable marker gene, wherein a transgene encoding a biological product is linked to the first selectable amplifiable marker gene, and wherein the first and second selectable amplifiable marker genes each have different nucleic acid sequences and are capable of being amplified using the same amplification reagent; (b) transfecting the host cell of step (a) with an RNA effector molecule, a portion of which is complementary to the second selectable amplifiable marker gene endogenous to the host cell such that the RNA effector molecule inhibits expression of the second selectable amplifiable marker gene; and (c) contacting the transfected cells of step (b) with a progressively increasing amount of the amplification reagent to select for cells with multiple copies of the first selectable amplifiable marker gene and the transgene, thereby generating a cell line that is capable of producing the biological product.

2. A method of generating a cell line capable of producing a biological product comprising: a) transfecting a plurality of host cells with: i) one or more vectors comprising a transgene linked to a first selectable amplifiable marker gene, wherein the transgene encodes a biological product, ii) an RNA effector molecule, a portion of which is complementary to a second selectable amplifiable marker gene endogenous to the host cell such that the RNA effector molecule inhibits expression of the second selectable amplifiable marker gene, wherein the first and second selectable amplifiable marker genes each have a different nucleic acid sequence and are capable of being amplified using an amplification reagent, b) culturing the plurality of host cells of step a) with a first concentration of the amplification reagent to select for viable transfected host cells; c) culturing the viable transfected host cells of step b) with a higher concentration of the amplification reagent than used in step b), thereby selecting for surviving cells that have an increased copy number of the transgene and the first selectable marker gene, wherein cells capable of producing a biological product are generated.

3. The method of paragraph 1 or 2, wherein the RNA effector molecule does not significantly inhibit expression of the first selectable marker gene.

4. The method of paragraph 1 or 2, wherein the RNA effector molecule transiently inhibits expression of the second selectable amplifiable marker gene.

5. The method of paragraph 1 or 2, wherein the RNA effector molecule inhibits expression of the second selectable amplification gene by at least 10%, at least 20%, at least 30%, at least 40%, at least 50%, at least 60%, at least 70%, at least 80%, at least 90%, at least 95%, at least 99%, or 100%.

6. The method of paragraph 1 or 2, wherein the RNA effector molecule inhibits expression of the second amplifiable marker gene at least 10%, at least 20%, at least 30%, at least 40%, at least 50%, at least 60%, at least 70%, at least 80%, at least 90%, at least 95%, at least 99%, at least 2-fold, at least 5-fold, at least 10-fold, at least 100 fold, or at least 1000 fold more than the RNA effector molecule inhibits the first selectable amplifiable marker.

7. The method of paragraph 1 or 2, further comprising transfecting the cell of step a) with a second RNA effector molecule, a portion of which is complementary to the transgene, such that the second RNA effector molecule inhibits expression of the transgene.

8. The method of paragraph 6, wherein the cell that has amplified the transgene is maintained in the presence of the second RNA effector molecule for a period of time before removal of the second RNA effector molecule and expression of the transgene.

9. The method of paragraph 7, wherein the RNA effector molecule inhibits expression of the transgene by an average percent inhibition of at least 10%, at least 20%, at least 30%, at least 40%, at least 50%, at least 60%, at least 70%, at least 80%, at least 90%, at least 95%, at least 99%, or 100%.

10. The method of paragraph 1 or 2, wherein the first and second selectable amplifiable marker genes encode a protein selected from the group consisting of: dihydrofolate reductase, thymidylate synthase, glutamine synthetase, adenosine deaminase, carbamoyl-phosphate synthase-aspartate transcarbamoylase-dihydroorotase (CAD), ornithine decarboxylase, and asparagine synthetase.

11. The method of paragraph 1 or 2, wherein the first and second selectable amplifiable marker genes do not encode for dihydrofolate reductase.

12. The method of paragraph 1 or 2, wherein the first and second selectable amplifiable marker genes are from different species.

13. The method of paragraph 1 or 2, wherein the amplification reagent is selected from the group consisting of: methotrexate, N-phosphonoacetyl-L-aspartic acid (PALA), 2′-deoxycoformycin (dCF), 5-fluorouracil (5FU), difluoromethylornithine (DFMO), albizziin, and -aspartyl hydroxamate (-AHA).

14. The method of any of paragraphs 1-13, wherein the biological product is a polypeptide.

15. The method of any of paragraphs 1-14, wherein the biological product is a metabolite.

16. The method of any of paragraphs 1-15, wherein the biological product is a nutraceutical.

17. The method of any of paragraphs 1-16, wherein the cell is an animal cell.

18. The method of any of paragraphs 1-16, wherein the cell is a fungal cell.

19. The method of any of paragraphs 1-16, wherein the cell is a plant cell.

20. The method of any of paragraphs 1-17, wherein the cell is a mammalian cell.

21. The method of paragraph 20, wherein the mammalian cell is a human cell.

22. The method of paragraph 21, wherein the human cell is an adherent cell selected from the group consisting of: SH-SY5Y cells, IMR32 cells, LAN5 cells, HeLa cells, MCF1OA cells, 293T cells, and SK-BR3 cells.

23. The method of paragraph 21, wherein the human cell is a primary cell selected from the group consisting of: HuVEC cells, HuASMC cells, HKB-I1 cells, and hMSC cells.

24. The method of paragraph 21, wherein the human cell is selected from the group consisting of: U293 cells, HEK 293 cells, PERC6® cells, Jurkat cells, HT-29 cells, LNCap.FGC cells, A549 cells, MDA MB453 cells, HepG2 cells, THP-I cells, MCF7 cells, BxPC-3 cells, Capan-1 cells, DU145 cells, and PC-3 cells.

25. The method of paragraph 21, wherein the mammalian cell is a rodent cell selected from the group consisting of: BHK21 cells, BHK TK− cells, NS0 cells, Sp2/0 cells, EL4 cells, CHO cells, CHO cell derivatives, U293 cells, NIH/3T3 cells, 3T3 L1 cells, ES-D3 cells, H9c2 cells, C2C12 cells, and miMCD-3 cells.

26. The method of paragraph 25, wherein the CHO cell derivative is selected from the group consisting of: CHO-K1 cells, CHO-DUKX, CHO-DUKX B1, and CHO-DG44 cells.

27. The method of paragraph 21, wherein the human cell is selected from the group consisting of: PERC6 cells, HT-29 cells, LNCaP-FGC cells A549 cells, MDA MB453 cells, HepG2 cells, THP-I cells, miMCD-3 cells, HEK 293 cells, HeLaS3 cells, MCF7 cells, Cos-7 cells, BxPC-3 cells, DU145 cells, Jurkat cells, PC-3 cells, and Capan-1 cells.

28. The method of any of paragraphs 1-27, wherein the RNA effector molecule is a double-stranded ribonucleic acid (dsRNA), wherein said dsRNA comprises at least two sequences that are complementary to each other and wherein a sense strand comprises a first sequence and an antisense strand comprises a second sequence comprising a region of complementarity, and wherein said region of complementarily is 15-30 nucleotides in length.

29. The method of any one of paragraphs 1-28, wherein the RNA effector molecule comprises a modified nucleotide.

30. The method of paragraph 1 or 2, wherein the nucleic acid sequences of the first and second selectable amplifiable marker differ by at least one nucleotide.

31. The method of paragraph 7, wherein the second RNA effector molecule is transfected immediately before, simultaneously with, or immediately after the vector comprising a transgene.

32. The method of paragraph 2, wherein the transgene and first selectable marker are each provided on a separate vector and are linked co-transformationally in the host genome.

33. The method of paragraph 2, wherein the transgene linked to the first selectable marker is provided on a single vector.

34. A method for increasing the transfection efficiency of cells capable of producing a biological product, comprising transfecting a plurality of host cells with: i) a vector comprising a transgene that encodes a biological product; and ii) an RNA effector molecule that inhibits expression of the transgene, wherein the RNA effector molecule inhibits expression of the transgene thereby increasing the transfection efficiency as compared to the transfection efficiency observed in the absence of the RNA effector molecule.

35. The method of paragraph 34, wherein the RNA effector molecule is transfected immediately before, simultaneously with, or immediately after the vector comprising a transgene.

36. The method paragraphs 34-35, wherein the RNA effector molecule is a double-stranded ribonucleic acid (dsRNA), wherein said dsRNA comprises at least two sequences that are complementary to each other and wherein a sense strand comprises a first sequence and an antisense strand comprises a second sequence comprising a region of complementarity, and wherein said region of complementarity is 15-30 nucleotides in length.

37. The method of paragraphs 34-36, wherein the RNA effector molecule comprises a modified nucleotide.

38. The method of paragraphs 34-37, wherein expression of the transgene is transiently inhibited.

39. The method of paragraphs 34-38, wherein the RNA effector molecule inhibits expression of the transgene by at least 10%, at least 20%, at least 30%, at least 40%, at least 50%, at least 60%, at least 70%, at least 80%, at least 90%, at least 95%, at least 99%, or 100%.

40. The method of paragraphs 34-39, wherein the cell with the transgene is maintained in the presence of the RNA effector molecule for a period of time before removal of the RNA effector molecule and expression of the transgene.

41. The method of any of paragraphs 34-40, wherein the biological product is a polypeptide.

42. The method of any of paragraphs 34-41, wherein the biological product is a metabolite.

43. The method of any of paragraphs 34-41, wherein the biological product is a nutraceutical.

44. The method of any of paragraphs 34-43, wherein the cell is an animal cell.

45. The method of any of paragraphs 34-43, wherein the cell is a fungal cell.

46. The method of any of paragraphs 34-43, wherein the cell is a plant cell.

47. The method of any of paragraphs 34-44, wherein the cell is a mammalian cell.

48. The method of paragraph 47, wherein the mammalian cell is a human cell.

49. The method of paragraph 48, wherein the human cell is an adherent cell selected from the group consisting of: SH-SY5Y cells, IMR32 cells, LAN5 cells, HeLa cells, MCF1OA cells, 293T cells, and SK-BR3 cells.

50. The method of paragraph 48, wherein the human cell is a primary cell selected from the group consisting of: HuVEC cells, HuASMC cells, HKB-I1 cells, and hMSC cells.

51. The method of paragraph 48, wherein the human cell is selected from the group consisting of: U293 cells, HEK 293 cells, PERC6® cells, Jurkat cells, HT-29 cells, LNCap.FGC cells, A549 cells, MDA MB453 cells, HepG2 cells, THP-I cells, MCF7 cells, BxPC-3 cells, Capan-1 cells, DU145 cells, and PC-3 cells.

52. The method of paragraph 48, wherein the mammalian cell is a rodent cell selected from the group consisting of: BHK21 cells, BHK TK− cells, NS0 cells, Sp2/0 cells, EL4 cells, CHO cells, CHO cell derivatives, U293 cells, NIH/3T3 cells, 3T3 L1 cells, ES-D3 cells, H9c2 cells, C2C12 cells, and miMCD-3 cells.

53. The method of paragraph 52, wherein the CHO cell derivative is selected from the group consisting of: CHO-K1 cells, CHO-DUKX, CHO-DUKX B1, and CHO-DG44 cells.

54. The method of paragraph 48, wherein the human cell is selected from the group consisting of: PERC6 cells, HT-29 cells, LNCaP-FGC cells A549 cells, MDA MB453 cells, HepG2 cells, THP-I cells, miMCD-3 cells, HEK 293 cells, HeLaS3 cells, MCF7 cells, Cos-7 cells, BxPC-3 cells, DU145 cells, Jurkat cells, PC-3 cells, and Capan-1 cells.

55. A method for generating a cell line capable of producing a biological product, comprising: (a) transfecting a plurality of host cells with: i) a vector comprising a selectable marker and a transgene, wherein the transgene encodes a biological product, and ii)an RNA effector molecule, a portion of which is complementary to a copy of the selectable marker endogenously expressed in the plurality of host cells prior to introduction of the vector of step i), and (b) culturing the cells of step (a) under conditions that select for cells comprising the vector of step i), thereby generating a cell line capable of producing a biological product.

56. A kit for generating a cell capable of producing a biological product comprising: a) a vector comprising a selectable amplifiable marker gene that has a nucleic acid sequence distinct from that of the marker gene endogenous to a host cell; b) an RNA effector molecule, a portion of which is complementary to the marker gene endogenous to the host cell; and c) packaging materials and instructions therefor.

57. The kit of paragraph 56, further comprising a host cell.

58. The kit of paragraph 56, wherein the nucleic acid sequence of the selectable amplifiable marker on the vector differs from the nucleic acid sequence of the endogenous marker gene by at least one nucleotide.

59. The kit of paragraph 56, further comprising an amplification reagent.

Unless otherwise defined, all technical and scientific terms used herein have the same meaning as commonly understood by one of ordinary skill in the art to which this invention belongs. Although methods and materials similar or equivalent to those described herein can be used in the practice or testing of the RNA effector molecules and methods featured in the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

### Example 1

### Production of a Cell Line Using Gene Amplification

### Vector Production

An expression vector containing a transgene encoding ApoE and DHFR (or other selectable amplifiable marker gene) is generated. Such expression vectors can be generated by e.g., replacing the neomycin phosphotransferase gene with a modified DHFR cDNA in a commercially available plasmid such as pcDNA 3.1(+) (INVITROGEN™). The modified DHFR cDNA does not substantially bind the RNA effector molecule used to inhibit the endogenous DHFR gene in CHO cells. The modified DHFR can include a DHFR gene from a species other than a Chinese hamster, e.g. mouse etc. Alternatively, a Chinese hamster DHFR gene can be modified, for example, to include a number of silent mutations such that a given 21 bp region can have at least one nucleotide sequence difference (e.g., at least 2, 3, 4, or more) from the unmodified DHFR gene. An RNA effector molecule is selected which does not substantially bind the modified DHFR cDNA, but is effective in inhibiting the endogenous DHFR gene in CHO cells.

### Cell Culture and Transfection

Wild-type CHO cells are maintained in standard culture conditions (e.g., 5% CO₂, 37° C.) and MEM media comprising 10% fetal bovine serum. Wild-type (e.g., CHO cells that do not lack DHFR) CHO cells are simultaneously transfected with the linearized ApoE/DHFR vector and an RNA effector molecule that inhibits expression of the endogenous DHFR gene in the CHO cells using Lipofectamine 2000 (INVITROGEN™). The expression vector is an integratable vector or can be linearized. In other embodiments, the RNA effector molecule is transfected immediately before, simultaneously with, or immediately after transfection of the vector. If desired, an siRNA against ApoE can also be administered at this time to minimize toxic effects of a high level of ApoE expression observed following transfection. To determine optimal transfection protocols, expression of the transgene is confirmed using RT-PCR for ApoE or Western Blotting using an anti-ApoE antibody.

### Gene Amplification and Selection

Transfected cells are contacted with a starting methotrexate concentration, e.g., 0.04 μM, and are maintained in a culture medium comprising 0.04 μM for a period of time sufficient to select, e.g., at least 7 days in the presence of the RNA effector molecule for endogenous DHFR and optionally an RNA effector molecule against the ApoE transgene. The concentration of methotrexate is increased step-wise from e.g., 0.04 μM to 5 μM (e.g., from 0.04 μM to 0.4 μM, then from 0.4 μM to 1 μM, then from 1 μM to 2 μM, then from 2 μM to 4 μM, then from 3 μM to 4 μM, and then from 4 μM to 5 μM) the cells are cultured in each successive concentration for a period of time sufficient to induce amplification (e.g., at least 15 days) before the methotrexate concentration is increased. Cells are cultured in the presence of the appropriate RNA effector molecules by e.g., repeated transfection or continuous infusion of the RNA effector molecules. Methods for the selection of CHO clones expressing heterologous genes are known in the art and described in for e.e. Hayduk and Lee Biotech Bioengineering, 2005, pg. 354-364, herein incorporated by reference.

Cells that survive the selection process and that are able to grow in 5 μM methotrexate are expected to have multiple copies of the DHFR gene and the ApoE transgene. At this time, the cells need not be cultured with methotrexate for further selection or amplification; however cells can be maintained in a culture comprising 5 μM methotrexate if so desired to prevent spontaneous deletion of the DHFR gene copies. The selected cells are further characterized for protein expression. Levels of secreted ApoE can be detected by Western blot analysis of proteins recovered from the cell supernatant. Clones exhibiting high levels are selected for production of ApoE (e.g., the biological product).

### Production of a Biological Product

Cells are grown in a larger volume for production of the ApoE protein, and the optional RNA effector molecule inhibiting ApoE expression is now removed from the cell culture. Cells can be further treated to enhance viability e.g., by treating with siRNA against Bax/Bak/LDH as described in e.g., U.S. Provisional No. 61/293,980, which is herein incorporated by reference in its entirety. In one embodiment, an siRNA against xylosyltransferase is administered to reduce heparin levels in cells to prevent intracellular binding of ApoE. Growth media is replaced as necessary to maintain production of the biological product by the cells.

### Example 2

### Enhancing Transfection Efficiency Using an RNA Effector Molecule Against the Transgene (with Gene Amplification)

### Cell Culture and Transfection

Vector production is as described above in Example 1. Wild-type CHO cells are maintained in standard culture conditions (e.g., 5% CO₂, 37° C.) and MEM media comprising 10% fetal bovine serum. Wild-type (e.g., DHFR(+)) CHO cells are simultaneously transfected with the ApoE/DHFR vector and an RNA effector molecule that inhibits expression of the endogenous DHFR gene in the CHO cells using Lipofectamine 2000 (INVITROGEN™). Alternatively, the RNA effector molecule is transfected immediately before, simultaneously with, or immediately after transfection of the vector. To determine optimal transfection protocols, expression of the transgene is confirmed using RT-PCR for ApoE or Western Blotting using an anti-ApoE antibody.

In one set of experiments, a second RNA effector molecule directed against the transgene is transfected into the CHO cells immediately before transfection with the ApoE/DHFR vector. In another set of experiments, a second RNA effector molecule directed against the transgene is transfected into the CHO cell simultaneously with transfection of the ApoE/DHFR vector. In another set of experiments, a second RNA effector molecule against the transgene is transfected immediately after transfection with the ApoE/DHFR vector.

Transfection with the second RNA effector molecule will enhance transfection efficiency by preventing an initial increase in transgene expression, which can be toxic to some cells, thereby increasing the number of transfected cells.

The ApoE transgene is then amplified using progressively increasing concentrations of methotrexate. Gene amplification and selection can be performed as described in Example 1. Methods for producing a biological product are also described herein in Example 1.

### Example 3

### Enhancing Transfection Efficiency Without Gene Amplification

### Cell Culture and Transfection

Wild-type CHO cells are maintained in standard culture conditions (e.g., 5% CO₂, 37° C.) and MEM media comprising 10% fetal bovine serum. Wild-type CHO cells are transfected with a vector comprising a selectable marker and the ApoE transgene using Lipofectamine 2000 (INVITROGEN™). To optimize a transfection protocol, expression of the transgene can be confirmed using RT-PCR for ApoE or Western Blotting using an anti-ApoE antibody.

The cells are further transfected with an RNA effector molecule against ApoE immediately before, simultaneously, or immediately after transfection with the vector. The RNA expression vector prevents the initial spike of ApoE concentration in the cells that can result in cell toxicity and cell death. These methods permit increased transfection efficiency (e.g., the number of transformed cells) by preventing death of cells following transfection. Once cells are selected based on the presence of the selectable marker, the RNA effector molecule can be removed to initiate transgene expression.

### Example 4

### Exemplary siRNA Compositions

Provided herein are exemplary siRNA reagents for inhibition of endogenous selectable amplifiable markers in CHO cells.

TABLE 3
RNA effector molecules for inhibition of
asparagine synthetase expression in CHO cells
(hamster)

| SEQ | | |
|---|---|---|
| ID | Start | Antisense Sequence |
| NO. | Pos. | 5′ to 3′ |
| 1 | 1688 | AAUGCUAUCAUCCAGAACU |
| 2 | 1898 | AGAGAUGCGACCCAGUUCC |
| 3 | 1057 | UUAUUUAAGGGAACGACAG |
| 4 | 2138 | AAUUCUAGAUCCAAACUGC |
| 5 | 1587 | AAUUCCAAGUUCGAGAAGG |
| 6 | 1231 | UUUCUAACUAAACAUAAGA |
| 7 | 1229 | UCUAACUAAACAUAAGACA |
| 8 | 364 | AAGUGUUCAUCAGAGAAGC |
| 9 | 667 | UGGACUCUAGAGAAGAGCG |
| 10 | 2135 | UCUAGAUCCAAACUGCAUG |
| 11 | 1589 | AAAAUUCCAAGUUCGAGAA |
| 12 | 1725 | AACCGACUCCUCUAGACGC |
| 13 | 315 | UGUGAAAUCAGGGUGACUG |
| 14 | 617 | AAGGCUACUGAACAUAACU |
| 15 | 1012 | UUUGCUACCACUGAUACAA |
| 16 | 1359 | CAUCUAAAGGAAUAUGACG |
| 17 | 1204 | UUGACUGCAACACUCAGGA |
| 18 | 931 | UUAGAAACGUAUUUCCAAG |
| 19 | 404 | UCUAAGAUUGCACAGCAAA |
| 20 | 1429 | UCUACCUUAGUAACACUCG |
| 21 | 256 | CUUGGUAUUGUUUUCUUAG |
| 22 | 1260 | UCAAAACUUCCUUUGAUAC |
| 23 | 1230 | UUCUAACUAAACAUAAGAC |
| 24 | 2205 | UCUAAGGUAGGAUUUGGAG |
| 25 | 1657 | UGAACUAAGUGGCAUAUUC |
| 26 | 1663 | AAGGGCUGAACUAAGUGGC |
| 27 | 1046 | AACGACAGGUUUUGAAAGG |
| 28 | 1401 | UCCGUUUAGUCACAAAAGC |
| 29 | 1187 | GACGUCAAUAAACUGAUGA |
| 30 | 1761 | UCUUGUAAGAUCUCACAGC |
| 31 | 1349 | AAUAUGACGAUCAGCAAGA |
| 32 | 2134 | CUAGAUCCAAACUGCAUGG |
| 33 | 607 | AACAUAACUUGAGUGUCAC |
| 34 | 423 | UGUUAUUGGGGCCCCGUCG |
| 35 | 1056 | UAUUUAAGGGAACGACAGG |
| 36 | 723 | UACCAAACCAUAAGUAAUG |
| 37 | 930 | UAGAAACGUAUUUCCAAGG |
| 38 | 1358 | AUCUAAAGGAAUAUGACGA |
| 39 | 774 | UGCCCAGAUUAUUAAAACG |
| 40 | 875 | GGAAUUGAGAUCAAUUUGG |
| 41 | 1354 | AAAGGAAUAUGACGAUCAG |
| 42 | 654 | AGAGCGACAAAAUAUCAGA |
| 43 | 345 | CUACAGAGCAGCAAAUGCC |
| 44 | 847 | GAUGCUGGAACUUCUUGCC |
| 45 | 1223 | UAAACAUAAGACACGUCUC |
| 46 | 1804 | UGCUCAUCGGCACCGAUCC |
| 47 | 1008 | CUACCACUGAUACAAACGC |
| 48 | 1059 | UCUUAUUUAAGGGAACGAC |
| 49 | 466 | AACAAACACUGAUAGUUAA |
| 50 | 1055 | AUUUAAGGGAACGACAGGU |
| 51 | 385 | UCCUCUUUCAAUUCUUUAC |
| 52 | 788 | GAGGAAGAAACUAUUGCCC |
| 53 | 657 | AGAAGAGCGACAAAAUAUC |
| 54 | 760 | AAACGCCAAAGCAAGCUAC |
| 55 | 2061 | GUAAAAUGAGCUUCUCACC |
| 56 | 313 | UGAAAUCAGGGUGACUGCG |
| 57 | 1153 | UGUUCAUCUGUAAGAAACA |
| 58 | 994 | AACGCUGGCAAUUCUGCUG |
| 59 | 1489 | ACAGUAGCACCCCAAGAGG |
| 60 | 1216 | AAGACACGUCUCUUGACUG |
| 61 | 88 | UGUUAAGGGCCUCCGUGCG |
| 62 | 1760 | CUUGUAAGAUCUCACAGCA |
| 63 | 2042 | AACCCCUCGUGGCAAAGAC |
| 64 | 1934 | ACCAAUAACUCUGUCAUCA |
| 65 | 1656 | GAACUAAGUGGCAUAUUCG |
| 66 | 1058 | CUUAUUUAAGGGAACGACA |
| 67 | 1937 | AUCACCAAUAACUCUGUCA |
| 68 | 873 | AAUUGAGAUCAAUUUGGAA |
| 69 | 1294 | AGGAUUGCAAUGUUUGCUG |
| 70 | 1753 | GAUCUCACAGCAUCCUGGG |
| 71 | 1652 | UAAGUGGCAUAUUCGAGCU |
| 72 | 2267 | UGUCACACAGUGUUGUUAC |
| 73 | 1407 | CAGUCUUCCGUUUAGUCAC |
| 74 | 1579 | GUUCGAGAAGGGUUGAUUG |
| 75 | 1824 | GACGGGAGUAACCAGCCAG |
| 76 | 1193 | ACUCAGGACGUCAAUAAAC |
| 77 | 1644 | AUAUUCGAGCUCUUCUUAG |
| 78 | 929 | AGAAACGUAUUUCCAAGGA |
| 79 | 2131 | GAUCCAAACUGCAUGGCCC |
| 80 | 715 | CAUAAGUAAUGGCUAGAGG |
| 81 | 2069 | UGCAAGGCGUAAAAUGAGC |
| 82 | 1718 | UCCUCUAGACGCAAACCAG |
| 83 | 655 | AAGAGCGACAAAAUAUCAG |
| 84 | 763 | UUAAAACGCCAAAGCAAGC |
| 85 | 1182 | CAAUAAACUGAUGAACUAC |
| 86 | 1793 | ACCGAUCCCAGUGAGAAUC |
| 87 | 1120 | CUUGAAUGGGGAACGCCGG |
| 88 | 700 | GAGGCUUGAUAAUAUAUAA |
| 89 | 1220 | ACAUAAGACACGUCUCUUG |
| 90 | 1014 | CAUUUGCUACCACUGAUAC |
| 91 | 1738 | UGGGUCACUAACCAACCGA |
| 92 | 1347 | UAUGACGAUCAGCAAGAGC |
| 93 | 497 | AACACCUCUCAAAUGAAGA |
| 94 | 1234 | UCCUUUCUAACUAAACAUA |
| 95 | 161 | UGCUUAUAUACUUUCCGAU |
| 96 | 1827 | GAUGACGGGAGUAACCAGC |
| 97 | 1053 | UUAAGGGAACGACAGGUUU |
| 98 | 1141 | AGAAACAUCGCAAGGGUCU |
| 99 | 2176 | UUAUCAGAUGCUUUCUCAU |
| 100 | 1880 | CAUUGCAAUUUCCUCAUUC |
| 101 | 651 | GCGACAAAAUAUCAGAUUC |
| 102 | 1918 | UCACGACUGAGGUUCCUGG |
| 103 | 761 | AAAACGCCAAAGCAAGCUA |
| 104 | 1532 | UGUGACUCGGUCUGGCACC |
| 105 | 1357 | UCUAAAGGAAUAUGACGAU |
| 106 | 1507 | UCAUCAAGACCCUGGGCCA |
| 107 | 242 | CUUAGUUCAUCUAAUGUCU |
| 108 | 1368 | CAAUGGGCUCAUCUAAAGG |
| 109 | 1729 | AACCAACCGACUCCUCUAG |
| 110 | 2144 | UUUUGCAAUUCUAGAUCCA |
| 111 | 1003 | ACUGAUACAAACGCUGGCA |
| 112 | 1319 | CAUGGAAUCAACACCUCCA |
| 113 | 1138 | AACAUCGCAAGGGUCUCUC |
| 114 | 1841 | CUGGAAGCGAACACGAUGA |
| 115 | 183 | AAAUACGCUGCUGUUCUGU |
| 116 | 89 | CUGUUAAGGGCCUCCGUGC |
| 117 | 1477 | CAAGAGGACUCUUCGGAAG |
| 118 | 1015 | UCAUUUGCUACCACUGAUA |
| 119 | 2143 | UUUGCAAUUCUAGAUCCAA |
| 120 | 775 | UUGCCCAGAUUAUUAAAAC |
| 121 | 408 | GUCGUCUAAGAUUGCACAG |
| 122 | 719 | AAACCAUAAGUAAUGGCUA |
| 123 | 1502 | AAGACCCUGGGCCACAGUA |
| 124 | 133 | GGUUAGAAAGUUCAUCCAC |
| 125 | 479 | AACAUGACCGGAAAACAAA |
| 126 | 1715 | UCUAGACGCAAACCAGACA |
| 127 | 1578 | UUCGAGAAGGGUUGAUUGA |
| 128 | 770 | CAGAUUAUUAAAACGCCAA |
| 129 | 1720 | ACUCCUCUAGACGCAAACC |
| 130 | 254 | UGGUAUUGUUUUCUUAGUU |
| 131 | 1191 | UCAGGACGUCAAUAAACUG |
| 132 | 1403 | CUUCCGUUUAGUCACAAAA |
| 133 | 1643 | UAUUCGAGCUCUUCUUAGU |
| 134 | 1939 | UGAUCACCAAUAACUCUGU |
| 135 | 2198 | UAGGAUUUGGAGCCUUCCA |
| 136 | 184 | AAAAUACGCUGCUGUUCUG |
| 137 | 1186 | ACGUCAAUAAACUGAUGAA |
| 138 | 860 | UUGGAAAAUUCCAGAUGCU |
| 139 | 995 | AAACGCUGGCAAUUCUGCU |
| 140 | 615 | GGCUACUGAACAUAACUUG |
| 141 | 1826 | AUGACGGGAGUAACCAGCC |
| 142 | 1726 | CAACCGACUCCUCUAGACG |
| 143 | 777 | UAUUGCCCAGAUUAUUAAA |
| 144 | 989 | UGGCAAUUCUGCUGCAGUG |
| 145 | 999 | AUACAAACGCUGGCAAUUC |
| 146 | 2136 | UUCUAGAUCCAAACUGCAU |
| 147 | 14 | CUUCGGGGCAUCUCCACGC |
| 148 | 134 | AGGUUAGAAAGUUCAUCCA |
| 149 | 1135 | AUCGCAAGGGUCUCUCUUG |
| 150 | 728 | GUCCCUACCAAACCAUAAG |
| 151 | 752 | AAGCAAGCUACGCCGGCCA |
| 152 | 2252 | UUACAGGGUGGAAUCACAU |
| 153 | 1063 | AGCAUCUUAUUUAAGGGAA |
| 154 | 1885 | AGUUCCAUUGCAAUUUCCU |
| 155 | 1577 | UCGAGAAGGGUUGAUUGAC |
| 156 | 1184 | GUCAAUAAACUGAUGAACU |
| 157 | 86 | UUAAGGGCCUCCGUGCGUG |
| 158 | 24 | UCGUACCCCACUUCGGGGC |
| 159 | 658 | GAGAAGAGCGACAAAAUAU |
| 160 | 1315 | GAAUCAACACCUCCAGAAA |
| 161 | 1385 | AGCCACAUUCAGAAGAUCA |
| 162 | 652 | AGCGACAAAAUAUCAGAUU |
| 163 | 1400 | CCGUUUAGUCACAAAAGCC |
| 164 | 656 | GAAGAGCGACAAAAUAUCA |
| 165 | 1801 | UCAUCGGCACCGAUCCCAG |
| 166 | 1423 | UUAGUAACACUCGGCCCAG |
| 167 | 2259 | AGUGUUGUUACAGGGUGGA |
| 168 | 182 | AAUACGCUGCUGUUCUGUU |
| 169 | 1487 | AGUAGCACCCCAAGAGGAC |
| 170 | 776 | AUUGCCCAGAUUAUUAAAA |
| 171 | 1790 | GAUCCCAGUGAGAAUCACC |
| 172 | 492 | CUCUCAAAUGAAGAACAUG |
| 173 | 1117 | GAAUGGGGAACGCCGGAAC |
| 174 | 1529 | GACUCGGUCUGGCACCUCC |
| 175 | 314 | GUGAAAUCAGGGUGACUGC |
| 176 | 151 | CUUUCCGAUUCUUCUUCAG |
| 177 | 998 | UACAAACGCUGGCAAUUCU |
| 178 | 286 | UCGGUUGUCUCUGUGUUUC |
| 179 | 162 | CUGCUUAUAUACUUUCCGA |
| 180 | 712 | AAGUAAUGGCUAGAGGCUU |
| 181 | 483 | GAAGAACAUGACCGGAAAA |
| 182 | 772 | CCCAGAUUAUUAAAACGCC |
| 183 | 1344 | GACGAUCAGCAAGAGCUGC |
| 184 | 1829 | ACGAUGACGGGAGUAACCA |
| 185 | 1894 | AUGCGACCCAGUUCCAUUG |
| 186 | 1060 | AUCUUAUUUAAGGGAACGA |
| 187 | 1221 | AACAUAAGACACGUCUCUU |
| 188 | 25 | CUCGUACCCCACUUCGGGG |
| 189 | 1730 | UAACCAACCGACUCCUCUA |
| 190 | 1583 | CCAAGUUCGAGAAGGGUUG |
| 191 | 779 | ACUAUUGCCCAGAUUAUUA |
| 192 | 1719 | CUCCUCUAGACGCAAACCA |
| 193 | 997 | ACAAACGCUGGCAAUUCUG |
| 194 | 90 | GCUGUUAAGGGCCUCCGUG |
| 195 | 71 | CGUGCAUCCGACUUGUAGG |
| 196 | 388 | AAAUCCUCUUUCAAUUCUU |
| 197 | 833 | UUGCCACUUGUCUGCAAGC |
| 198 | 928 | GAAACGUAUUUCCAAGGAU |
| 199 | 1468 | UCUUCGGAAGGGAUCUCAU |
| 200 | 1134 | UCGCAAGGGUCUCUCUUGA |

TABLE 4
RNA effector molecules for inhibition of ornithine
decarboxylase expression in CHO cells (hamster)

| SEQ | | |
|---|---|---|
| ID | Start | Antisense Sequence |
| NO. | Pos. | 5′ to 3′ |
| 201 | 1020 | AGUUAAAUGACCCAUACAC |
| 202 | 747 | AUAUCAAGCAGAUACAUGC |
| 203 | 751 | ACCAAUAUCAAGCAGAUAC |
| 204 | 622 | AAUGACAUCAAUAUUUAGC |
| 205 | 1263 | UCACAUAGUAGAUAGAAGG |
| 206 | 1464 | UUCAAGCUAAACUUGAAGG |
| 207 | 939 | UUCGAUACGAUUUUCUUGG |
| 208 | 1040 | UGCAUGAUCGUAAAGAAUG |
| 209 | 14 | AUGGGAUUCAGUUAUGGCC |
| 210 | 1686 | AUGUACAAGCUACAAAUGC |
| 211 | 1236 | ACCCGUUGAAAGUAGAUGC |
| 212 | 1457 | UAAACUUGAAGGUAAGAGC |
| 213 | 1208 | AGUGUAUGCACCCAUGUUC |
| 214 | 391 | CUUACAUGGAUUUGCAUAG |
| 215 | 1502 | ACUAACAGUAAGUUAAAUG |
| 216 | 867 | UCGGCUAUAACUCUCACUC |
| 217 | 8 | UUCAGUUAUGGCCAGUUCC |
| 218 | 1980 | UGUAUGAUACUUCCAACUG |
| 219 | 1252 | AUAGAAGGCCUCUGGAACC |
| 220 | 611 | UAUUUAGCUCUUUUGCCCG |
| 221 | 1499 | AACAGUAAGUUAAAUGGUC |
| 222 | 916 | GAUAUUGACUGCCAGUGUG |
| 223 | 1969 | UCCAACUGUUACUAGGUGG |
| 224 | 1415 | GAUACUAGCAGAAGCACAG |
| 225 | 94 | UUCAUCGAGGAUAUGGCAG |
| 226 | 387 | CAUGGAUUUGCAUAGAUGA |
| 227 | 1685 | UGUACAAGCUACAAAUGCU |
| 228 | 1893 | AAAGCCUUAGAUGCCUUCC |
| 229 | 381 | UUUGCAUAGAUGAUCCUCU |
| 230 | 342 | UGUACCAACUGUAUCUCAG |
| 231 | 1744 | UCAAGAUAGUUUAUUUUCA |
| 232 | 1458 | CUAAACUUGAAGGUAAGAG |
| 233 | 1419 | CAUUGAUACUAGCAGAAGC |
| 234 | 862 | UAUAACUCUCACUCCAGAG |
| 235 | 1463 | UCAAGCUAAACUUGAAGGU |
| 236 | 1498 | ACAGUAAGUUAAAUGGUCC |
| 237 | 409 | CUUGAUCUGAGACACUUGC |
| 238 | 1036 | UGAUCGUAAAGAAUGCAGU |
| 239 | 1025 | AAUGCAGUUAAAUGACCCA |
| 240 | 1017 | UAAAUGACCCAUACACUCC |
| 241 | 1511 | CAUUUCAAAACUAACAGUA |
| 242 | 1715 | CAACAUUAGCUUUUGGCCC |
| 243 | 986 | AUACAUGAAGGUUUGCUCA |
| 244 | 1290 | UCAUAAGCUGCCACAUUGG |
| 245 | 1999 | GAAGUUGAUUGCCAAGUGC |
| 246 | 1429 | GGCAUCUACACAUUGAUAC |
| 247 | 375 | UAGAUGAUCCUCUCGGGAG |
| 248 | 1894 | AAAAGCCUUAGAUGCCUUC |
| 249 | 1270 | CUCGACAUCACAUAGUAGA |
| 250 | 217 | UUUUAGCCAUCUUAGGUGU |
| 251 | 2017 | UGUUGAGAUUUAUUACAGG |
| 252 | 1136 | AAUCCGGUCAAGGCCAUCG |
| 253 | 979 | AAGGUUUGCUCACUGGACU |
| 254 | 517 | AGUGGCGAUCCGCAAAACC |
| 255 | 919 | UAUGAUAUUGACUGCCAGU |
| 256 | 1862 | CAGGAUAUCAGGUCGCUAG |
| 257 | 1983 | UGCUGUAUGAUACUUCCAA |
| 258 | 1256 | GUAGAUAGAAGGCCUCUGG |
| 259 | 1026 | GAAUGCAGUUAAAUGACCC |
| 260 | 1830 | CUCACCAACAUGACUACAG |
| 261 | 883 | GUAGUAUCUGCCUGGCUCG |
| 262 | 776 | UAUCCUCAGAUCCAGGAAA |
| 263 | 1770 | CAAACAUUCCCUGAUGCCC |
| 264 | 918 | AUGAUAUUGACUGCCAGUG |
| 265 | 1454 | ACUUGAAGGUAAGAGCUAC |
| 266 | 553 | CUUUACACUGAGUCGACAC |
| 267 | 1042 | UGUGCAUGAUCGUAAAGAA |
| 268 | 936 | GAUACGAUUUUCUUGGCUA |
| 269 | 547 | ACUGAGUCGACACACUGCU |
| 270 | 306 | AAACCUGUUCCAAUGGCAG |
| 271 | 861 | AUAACUCUCACUCCAGAGU |
| 272 | 245 | CAUAAAAGGGAGUGACCCG |
| 273 | 1783 | UUAAGGGACAUUGCAAACA |
| 274 | 748 | AAUAUCAAGCAGAUACAUG |
| 275 | 552 | UUUACACUGAGUCGACACA |
| 276 | 1418 | AUUGAUACUAGCAGAAGCA |
| 277 | 1447 | GGUAAGAGCUACAAGAAUG |
| 278 | 787 | UUUAAGCUUCGUAUCCUCA |
| 279 | 886 | AACGUAGUAUCUGCCUGGC |
| 280 | 1497 | CAGUAAGUUAAAUGGUCCC |
| 281 | 250 | GACUGCAUAAAAGGGAGUG |
| 282 | 1288 | AUAAGCUGCCACAUUGGCC |
| 283 | 256 | ACUUUUGACUGCAUAAAAG |
| 284 | 661 | AGGGUCAGUACAUCCACUC |
| 285 | 374 | AGAUGAUCCUCUCGGGAGG |
| 286 | 891 | GAGGCAACGUAGUAUCUGC |
| 287 | 15 | GAUGGGAUUCAGUUAUGGC |
| 288 | 1756 | UGCCCAAUUAUUUCAAGAU |
| 289 | 1679 | AGCUACAAAUGCUUGCUCA |
| 290 | 247 | UGCAUAAAAGGGAGUGACC |
| 291 | 528 | UUAGAAUCGUCAGUGGCGA |
| 292 | 545 | UGAGUCGACACACUGCUUU |
| 293 | 243 | UAAAAGGGAGUGACCCGGG |
| 294 | 146 | AGGAAUACACUUCAUUAAU |
| 295 | 687 | UCCGACAAGGCCUGGACGA |
| 296 | 1037 | AUGAUCGUAAAGAAUGCAG |
| 297 | 376 | AUAGAUGAUCCUCUCGGGA |
| 298 | 86 | GGAUAUGGCAGUCAAACUC |
| 299 | 1643 | GUUAGUAUGUCUGAAAAGU |
| 300 | 782 | GCUUCGUAUCCUCAGAUCC |
| 301 | 1797 | AGUGUGUCCCUUCUUUAAG |
| 302 | 887 | CAACGUAGUAUCUGCCUGG |
| 303 | 1127 | AAGGCCAUCGCAUGUUGGU |
| 304 | 1237 | AACCCGUUGAAAGUAGAUG |
| 305 | 439 | CAUCAUCUGGACUCCAUUG |
| 306 | 920 | CUAUGAUAUUGACUGCCAG |
| 307 | 363 | UCGGGAGGCACUCCAAGGC |
| 308 | 525 | GAAUCGUCAGUGGCGAUCC |
| 309 | 1859 | GAUAUCAGGUCGCUAGGCA |
| 310 | 1289 | CAUAAGCUGCCACAUUGGC |
| 311 | 978 | AGGUUUGCUCACUGGACUC |
| 312 | 1008 | CAUACACUCCAUCAUUCAC |
| 313 | 21 | UCUAGAGAUGGGAUUCAGU |
| 314 | 1574 | UAAGUGUGACCCAUCUCCU |
| 315 | 471 | ACCUUCAUUAACUCAACUU |
| 316 | 985 | UACAUGAAGGUUUGCUCAC |
| 317 | 389 | UACAUGGAUUUGCAUAGAU |
| 318 | 1093 | GAGUAAUACUUCUCAUCUG |
| 319 | 555 | AACUUUACACUGAGUCGAC |
| 320 | 43 | UCUCGGUGUGCCUACAAAA |
| 321 | 843 | UCUGGCGGGAAGUACUUGU |
| 322 | 1461 | AAGCUAAACUUGAAGGUAA |
| 323 | 394 | UUGCUUACAUGGAUUUGCA |
| 324 | 1769 | AAACAUUCCCUGAUGCCCA |
| 325 | 90 | UCGAGGAUAUGGCAGUCAA |
| 326 | 509 | UCCGCAAAACCAACUUGGC |
| 327 | 1210 | ACAGUGUAUGCACCCAUGU |
| 328 | 478 | UCUGGCGACCUUCAUUAAC |
| 329 | 519 | UCAGUGGCGAUCCGCAAAA |
| 330 | 248 | CUGCAUAAAAGGGAGUGAC |
| 331 | 1240 | UGGAACCCGUUGAAAGUAG |
| 332 | 631 | GCUGACACCAAUGACAUCA |
| 333 | 1259 | AUAGUAGAUAGAAGGCCUC |
| 334 | 1961 | UUACUAGGUGGUGAUGCAG |
| 335 | 917 | UGAUAUUGACUGCCAGUGU |
| 336 | 783 | AGCUUCGUAUCCUCAGAUC |
| 337 | 1577 | AGGUAAGUGUGACCCAUCU |
| 338 | 892 | UGAGGCAACGUAGUAUCUG |
| 339 | 365 | UCUCGGGAGGCACUCCAAG |
| 340 | 523 | AUCGUCAGUGGCGAUCCGC |
| 341 | 1264 | AUCACAUAGUAGAUAGAAG |
| 342 | 541 | UCGACACACUGCUUUAGAA |
| 343 | 548 | CACUGAGUCGACACACUGC |
| 344 | 1421 | CACAUUGAUACUAGCAGAA |
| 345 | 423 | UUGCUGGCGGCAUACUUGA |
| 346 | 1007 | AUACACUCCAUCAUUCACA |
| 347 | 1139 | CACAAUCCGGUCAAGGCCA |
| 348 | 1807 | CUGUGCAGGAAGUGUGUCC |
| 349 | 1388 | AUGACGGUCCAUCCCGCUC |
| 350 | 1439 | CUACAAGAAUGGCAUCUAC |
| 351 | 364 | CUCGGGAGGCACUCCAAGG |
| 352 | 935 | AUACGAUUUUCUUGGCUAU |
| 353 | 1503 | AACUAACAGUAAGUUAAAU |
| 354 | 668 | AGGUCUCAGGGUCAGUACA |
| 355 | 1838 | UGACGUUCCUCACCAACAU |
| 356 | 1420 | ACAUUGAUACUAGCAGAAG |
| 357 | 834 | AAGUACUUGUCCAGAGCUG |
| 358 | 1022 | GCAGUUAAAUGACCCAUAC |
| 359 | 1504 | AAACUAACAGUAAGUUAAA |
| 360 | 1673 | AAAUGCUUGCUCAGUGGCU |
| 361 | 779 | UCGUAUCCUCAGAUCCAGG |
| 362 | 1128 | CAAGGCCAUCGCAUGUUGG |
| 363 | 1257 | AGUAGAUAGAAGGCCUCUG |
| 364 | 865 | GGCUAUAACUCUCACUCCA |
| 365 | 392 | GCUUACAUGGAUUUGCAUA |
| 366 | 531 | GCUUUAGAAUCGUCAGUGG |
| 367 | 922 | GGCUAUGAUAUUGACUGCC |
| 368 | 373 | GAUGAUCCUCUCGGGAGGC |
| 369 | 736 | AUACAUGCUGAAACCAACU |
| 370 | 933 | ACGAUUUUCUUGGCUAUGA |
| 371 | 524 | AAUCGUCAGUGGCGAUCCG |
| 372 | 194 | UCAGAACGUCUCCAAGGUC |
| 373 | 1034 | AUCGUAAAGAAUGCAGUUA |
| 374 | 1979 | GUAUGAUACUUCCAACUGU |
| 375 | 894 | GCUGAGGCAACGUAGUAUC |
| 376 | 289 | AGCUAGGGUGUUCACUACA |
| 377 | 287 | CUAGGGUGUUCACUACAGC |
| 378 | 658 | GUCAGUACAUCCACUCCCC |
| 379 | 656 | CAGUACAUCCACUCCCCAC |
| 380 | 192 | AGAACGUCUCCAAGGUCCG |
| 381 | 888 | GCAACGUAGUAUCUGCCUG |
| 382 | 915 | AUAUUGACUGCCAGUGUGA |
| 383 | 1024 | AUGCAGUUAAAUGACCCAU |
| 384 | 1839 | AUGACGUUCCUCACCAACA |
| 385 | 938 | UCGAUACGAUUUUCUUGGC |
| 386 | 1046 | CACAUGUGCAUGAUCGUAA |
| 387 | 610 | AUUUAGCUCUUUUGCCCGU |
| 388 | 427 | UCCAUUGCUGGCGGCAUAC |
| 389 | 1140 | CCACAAUCCGGUCAAGGCC |
| 390 | 934 | UACGAUUUUCUUGGCUAUG |
| 391 | 890 | AGGCAACGUAGUAUCUGCC |
| 392 | 1540 | AUCUGUGCCAAGCCCUACU |
| 393 | 1719 | GUCACAACAUUAGCUUUUG |
| 394 | 608 | UUAGCUCUUUUGCCCGUUC |
| 395 | 863 | CUAUAACUCUCACUCCAGA |
| 396 | 1011 | ACCCAUACACUCCAUCAUU |
| 397 | 781 | CUUCGUAUCCUCAGAUCCA |
| 398 | 1997 | AGUUGAUUGCCAAGUGCUG |
| 399 | 1460 | AGCUAAACUUGAAGGUAAG |
| 400 | 1120 | UCGCAUGUUGGUCCCCAGA |

TABLE 5
RNA effector molecules for inhibition of CAD
expression in CHO cells (hamster)

| SEQ | | |
|---|---|---|
| ID | Start | Antisense Sequence |
| NO. | Pos. | 5′ to 3′ |
| 401 | 2469 | CUAGAAACGGCCUAGCACG |
| 402 | 2633 | AGUACUCUAGUCUGGAGCC |
| 403 | 2100 | GUAACGUAAGCUCACACGG |
| 404 | 828 | AUGACUGCCAUAUUCUCCC |
| 405 | 2369 | ACGCUUAUCUCAUUGACAC |
| 406 | 2587 | AUAUGCUUGGGCUAUCUGG |
| 407 | 2235 | CUGAAUGCGAGUCAUGUAG |
| 408 | 427 | CAUUCAAUAACUUUCAGCU |
| 409 | 1812 | AAGCGAUUCCCCUUUCUGG |
| 410 | 1853 | ACGACAUCAGCGUAGCAAC |
| 411 | 1175 | AUAUAAGCAACCUCCCCUC |
| 412 | 2015 | AUCGUCAUGCCGUUGACAG |
| 413 | 2407 | GACGGAAGUAGGCAGCUCG |
| 414 | 732 | UCCCGAUUUAAGAAGAGGU |
| 415 | 2663 | AGUAUCAGAGACAGUACCC |
| 416 | 2049 | UGUGCGUCCAUGCUUCAGG |
| 417 | 2231 | AUGCGAGUCAUGUAGAGCA |
| 418 | 1911 | UCUUCGACAAUGCUUGGCU |
| 419 | 1709 | CUCACCUCGUAGAACAUGG |
| 420 | 2596 | UGUACACAUAUAUGCUUGG |
| 421 | 2634 | GAGUACUCUAGUCUGGAGC |
| 422 | 2708 | UUGAGGUGUAAGAACGGAG |
| 423 | 1184 | UGUCCAUCGAUAUAAGCAA |
| 424 | 2295 | AGUGAGGAUGAACUGACCA |
| 425 | 1289 | GUCGUGGUUACUUCGGUGG |
| 426 | 630 | UGACAUAUGUGCACUGGGC |
| 427 | 2666 | GUAAGUAUCAGAGACAGUA |
| 428 | 1182 | UCCAUCGAUAUAAGCAACC |
| 429 | 1598 | UGAUCCUUAGUGAACUGCU |
| 430 | 1623 | UGCCACGUUGAACAAAUGA |
| 431 | 7 | CUGGCGAGCAGACUCAAGG |
| 432 | 1909 | UUCGACAAUGCUUGGCUGC |
| 433 | 1178 | UCGAUAUAAGCAACCUCCC |
| 434 | 1565 | UGUUGGCCCACUAAAGAGU |
| 435 | 2256 | AGUGGAGCCAAAUCGCUCU |
| 436 | 1921 | UGAUCACUGGUCUUCGACA |
| 437 | 1922 | UUGAUCACUGGUCUUCGAC |
| 438 | 2094 | UAAGCUCACACGGUACUGG |
| 439 | 2638 | CAGAGAGUACUCUAGUCUG |
| 440 | 2233 | GAAUGCGAGUCAUGUAGAG |
| 441 | 2098 | AACGUAAGCUCACACGGUA |
| 442 | 426 | AUUCAAUAACUUUCAGCUG |
| 443 | 2464 | AACGGCCUAGCACGGUGGC |
| 444 | 2234 | UGAAUGCGAGUCAUGUAGA |
| 445 | 829 | GAUGACUGCCAUAUUCUCC |
| 446 | 1172 | UAAGCAACCUCCCCUCGCA |
| 447 | 2410 | CUUGACGGAAGUAGGCAGC |
| 448 | 2113 | UGGGAGGUGCCACGUAACG |
| 449 | 733 | UUCCCGAUUUAAGAAGAGG |
| 450 | 2152 | AAGCCACAAAGUCGCGAAC |
| 451 | 2346 | CAUCGGAUGCAUCACCACC |
| 452 | 737 | AGUCUUCCCGAUUUAAGAA |
| 453 | 1859 | AGCACGACGACAUCAGCGU |
| 454 | 2144 | AAGUCGCGAACGCUGGGUG |
| 455 | 1811 | AGCGAUUCCCCUUUCUGGA |
| 456 | 580 | AGCAACACUCUGCCGCUCG |
| 457 | 1293 | AGGUGUCGUGGUUACUUCG |
| 458 | 2106 | UGCCACGUAACGUAAGCUC |
| 459 | 1286 | GUGGUUACUUCGGUGGUGG |
| 460 | 1217 | ACGUCUUGUCCAUAGCCUG |
| 461 | 2355 | GACACGGGGCAUCGGAUGC |
| 462 | 1826 | UGCACUGAGUCGGCAAGCG |
| 463 | 2099 | UAACGUAAGCUCACACGGU |
| 464 | 788 | GGGAACCAAGCUCAGGCCG |
| 465 | 2145 | AAAGUCGCGAACGCUGGGU |
| 466 | 1707 | CACCUCGUAGAACAUGGAG |
| 467 | 284 | UCACCAGCGUAGCAUCCCC |
| 468 | 1856 | ACGACGACAUCAGCGUAGC |
| 469 | 1924 | CAUUGAUCACUGGUCUUCG |
| 470 | 1180 | CAUCGAUAUAAGCAACCUC |
| 471 | 2637 | AGAGAGUACUCUAGUCUGG |
| 472 | 2034 | CAGGUCACCUACCAUGGUG |
| 473 | 2143 | AGUCGCGAACGCUGGGUGG |
| 474 | 1171 | AAGCAACCUCCCCUCGCAG |
| 475 | 2591 | ACAUAUAUGCUUGGGCUAU |
| 476 | 1011 | GAAGAUCCGCCGAGGAUUG |
| 477 | 235 | AGAGAUGGCGAUAGCCGAC |
| 478 | 1314 | GAUGACUCGGCGUGGUCUC |
| 479 | 1490 | GAUGCCUGUCUAGGUACCG |
| 480 | 734 | CUUCCCGAUUUAAGAAGAG |
| 481 | 1908 | UCGACAAUGCUUGGCUGCC |
| 482 | 79 | CACGUUCAUGGCAGCACCG |
| 483 | 1295 | UCAGGUGUCGUGGUUACUU |
| 484 | 2554 | CUGCUAAGUGUGCUGCCCC |
| 485 | 2122 | UGCGCAGACUGGGAGGUGC |
| 486 | 1359 | GUGGAUUCGGGGUGGCAAG |
| 487 | 2470 | CCUAGAAACGGCCUAGCAC |
| 488 | 1865 | UGCCGGAGCACGACGACAU |
| 489 | 217 | CACGAUGCCAUCGCAGGCC |
| 490 | 1917 | CACUGGUCUUCGACAAUGC |
| 491 | 1039 | CAUAGGUGUCCUCCUGGAG |
| 492 | 2409 | UUGACGGAAGUAGGCAGCU |
| 493 | 39 | ACUACGCAGGGGUACCCCA |
| 494 | 770 | GGACCUCUCCCUUUCCAGG |
| 495 | 1288 | UCGUGGUUACUUCGGUGGU |
| 496 | 2108 | GGUGCCACGUAACGUAAGC |
| 497 | 2370 | CACGCUUAUCUCAUUGACA |
| 498 | 1284 | GGUUACUUCGGUGGUGGCG |
| 499 | 1802 | CCUUUCUGGACGGAGGACG |
| 500 | 2436 | CAUGCGGAUGUACAUGCCA |
| 501 | 455 | UUUAGCCCAGCUGCAGAUG |
| 502 | 225 | AUAGCCGACACGAUGCCAU |
| 503 | 2146 | CAAAGUCGCGAACGCUGGG |
| 504 | 107 | AGCGCUCUAGGUCCCCAUC |
| 505 | 2230 | UGCGAGUCAUGUAGAGCAC |
| 506 | 1869 | AGGGUGCCGGAGCACGACG |
| 507 | 1080 | GUGGCUAGGGAUUGUCCAC |
| 508 | 1476 | UACCGGUGGUGCAGGGUAG |
| 509 | 228 | GCGAUAGCCGACACGAUGC |
| 510 | 859 | AUGGGGAGCAUGGUCUGAG |
| 511 | 787 | GGAACCAAGCUCAGGCCGG |
| 512 | 1484 | UGUCUAGGUACCGGUGGUG |
| 513 | 2592 | CACAUAUAUGCUUGGGCUA |
| 514 | 731 | CCCGAUUUAAGAAGAGGUG |
| 515 | 633 | ACGUGACAUAUGUGCACUG |
| 516 | 2569 | GAUGCCCCUGGAUGUCUGC |
| 517 | 47 | AGGGGCGCACUACGCAGGG |
| 518 | 1912 | GUCUUCGACAAUGCUUGGC |
| 519 | 1009 | AGAUCCGCCGAGGAUUGUG |
| 520 | 50 | AGGAGGGGCGCACUACGCA |
| 521 | 1149 | CACACGGCGGAUGGUACCC |
| 522 | 1223 | UUCCGUACGUCUUGUCCAU |
| 523 | 785 | AACCAAGCUCAGGCCGGAC |
| 524 | 1926 | AGCAUUGAUCACUGGUCUU |
| 525 | 1186 | CCUGUCCAUCGAUAUAAGC |
| 526 | 2688 | UGAUGCGGCAGAGGAGCCC |
| 527 | 1819 | AGUCGGCAAGCGAUUCCCC |
| 528 | 53 | CAUAGGAGGGGCGCACUAC |
| 529 | 283 | CACCAGCGUAGCAUCCCCG |
| 530 | 223 | AGCCGACACGAUGCCAUCG |
| 531 | 2556 | GUCUGCUAAGUGUGCUGCC |
| 532 | 234 | GAGAUGGCGAUAGCCGACA |
| 533 | 1014 | GUGGAAGAUCCGCCGAGGA |
| 534 | 2142 | GUCGCGAACGCUGGGUGGC |
| 535 | 1477 | GUACCGGUGGUGCAGGGUA |
| 536 | 2111 | GGAGGUGCCACGUAACGUA |
| 537 | 1637 | AUCCGUAGUGUGUGUGCCA |
| 538 | 1183 | GUCCAUCGAUAUAAGCAAC |
| 539 | 2257 | GAGUGGAGCCAAAUCGCUC |
| 540 | 1294 | CAGGUGUCGUGGUUACUUC |
| 541 | 10 | GAACUGGCGAGCAGACUCA |
| 542 | 2014 | UCGUCAUGCCGUUGACAGU |
| 543 | 2056 | AGUGCACUGUGCGUCCAUG |
| 544 | 1002 | CCGAGGAUUGUGGUGACAG |
| 545 | 1925 | GCAUUGAUCACUGGUCUUC |
| 546 | 36 | ACGCAGGGGUACCCCACAG |
| 547 | 2229 | GCGAGUCAUGUAGAGCACG |
| 548 | 2137 | GAACGCUGGGUGGCAUGCG |
| 549 | 1956 | CUGGGUAGGGUGCUCUCCA |
| 550 | 1177 | CGAUAUAAGCAACCUCCCC |
| 551 | 2640 | GCCAGAGAGUACUCUAGUC |
| 552 | 1558 | CCACUAAAGAGUGCAGCAG |
| 553 | 2047 | UGCGUCCAUGCUUCAGGUC |
| 554 | 1221 | CCGUACGUCUUGUCCAUAG |
| 555 | 642 | UUCCGAGCCACGUGACAUA |
| 556 | 2590 | CAUAUAUGCUUGGGCUAUC |
| 557 | 2754 | CUGUUUGGCUAUUUAUUAU |
| 558 | 1822 | CUGAGUCGGCAAGCGAUUC |
| 559 | 1560 | GCCCACUAAAGAGUGCAGC |
| 560 | 9 | AACUGGCGAGCAGACUCAA |
| 561 | 372 | CUGUAACCUGUAGUUCCUG |
| 562 | 1160 | CCUCGCAGGACCACACGGC |
| 563 | 2059 | GGGAGUGCACUGUGCGUCC |
| 564 | 2020 | UGGUGAUCGUCAUGCCGUU |
| 565 | 222 | GCCGACACGAUGCCAUCGC |
| 566 | 1311 | GACUCGGCGUGGUCUCUCA |
| 567 | 1716 | GCGGGUGCUCACCUCGUAG |
| 568 | 1667 | AUGUCAAGGCUCCGCUCUU |
| 569 | 786 | GAACCAAGCUCAGGCCGGA |
| 570 | 224 | UAGCCGACACGAUGCCAUC |
| 571 | 2639 | CCAGAGAGUACUCUAGUCU |
| 572 | 1570 | GGAUGUGUUGGCCCACUAA |
| 573 | 122 | CCGCACUGCUCAGAAAGCG |
| 574 | 1823 | ACUGAGUCGGCAAGCGAUU |
| 575 | 269 | CCCCGGAAUGCACACCUGC |
| 576 | 571 | CUGCCGCUCGGCAGUGGGC |
| 577 | 2109 | AGGUGCCACGUAACGUAAG |
| 578 | 1012 | GGAAGAUCCGCCGAGGAUU |
| 579 | 1954 | GGGUAGGGUGCUCUCCAAC |
| 580 | 1818 | GUCGGCAAGCGAUUCCCCU |
| 581 | 1666 | UGUCAAGGCUCCGCUCUUU |
| 582 | 1724 | CUACUGGUGCGGGUGCUCA |
| 583 | 1839 | GCAACUCAUGGUCUGCACU |
| 584 | 1155 | CAGGACCACACGGCGGAUG |
| 585 | 1713 | GGUGCUCACCUCGUAGAAC |
| 586 | 1226 | CACUUCCGUACGUCUUGUC |
| 587 | 2408 | UGACGGAAGUAGGCAGCUC |
| 588 | 1312 | UGACUCGGCGUGGUCUCUC |
| 589 | 800 | CCAUAUCCUCUCGGGAACC |
| 590 | 833 | AGUCGAUGACUGCCAUAUU |
| 591 | 1150 | CCACACGGCGGAUGGUACC |
| 592 | 286 | GGUCACCAGCGUAGCAUCC |
| 593 | 1067 | GUCCACUCAUGCUCUAGAU |
| 594 | 2124 | CAUGCGCAGACUGGGAGGU |
| 595 | 1197 | GGGUACCAACACCUGUCCA |
| 596 | 106 | GCGCUCUAGGUCCCCAUCA |
| 597 | 1077 | GCUAGGGAUUGUCCACUCA |
| 598 | 1078 | GGCUAGGGAUUGUCCACUC |
| 599 | 326 | ACCGAACUCCAGAGUUUUG |
| 600 | 45 | GGGCGCACUACGCAGGGGU |

TABLE 6
RNA effector molecules for inhibition of adenosine
deaminase expression in CHO cells (hamster)

| SEQ | | |
|---|---|---|
| ID | Start | Antisense Sequence |
| NO. | Pos. | 5′ to 3′ |
| 601 | 1344 | AAAGGAAGGUUCCUGAUUC |
| 602 | 169 | UUGCCAAAGUAUAAGAUGG |
| 603 | 1371 | UUUAUUGAACAACAGAUUU |
| 604 | 160 | UAUAAGAUGGUUUCCAGCU |
| 605 | 278 | GUAGUAAUCAAACUUGGCC |
| 606 | 1567 | UGAUUAAAGAAGCCAAGAG |
| 607 | 1331 | UGAUUCAUACCCACGAUUG |
| 608 | 476 | GUUCACAAGAUCCACAACC |
| 609 | 1518 | AAAAUGUCACUUCGGGAGG |
| 610 | 1444 | AUUCUUACCCACCCAAGCC |
| 611 | 923 | GAAGCGAACAACUGCAUGC |
| 612 | 1393 | CAAGAUACCAGUCACCAGC |
| 613 | 1131 | AUUGGUAUGCUUUGUAGAG |
| 614 | 824 | CUUAUAGAGGGCCUGGUCC |
| 615 | 1061 | UGCAUUGAUGUUCAGUCGC |
| 616 | 656 | GAAGAGGCUACUUCCUUCG |
| 617 | 1374 | UGCUUUAUUGAACAACAGA |
| 618 | 879 | UGAGAUAGCUGGACCAGGG |
| 619 | 469 | AGAUCCACAACCUCGUCAG |
| 620 | 751 | UGUUGCACAACCUCAGCAG |
| 621 | 947 | UGAGUAGUUGGCCUGGUCU |
| 622 | 1118 | GUAGAGUUGUUCCAGAAUC |
| 623 | 289 | AUAACAGGCAUGUAGUAAU |
| 624 | 981 | UGGACUUGAAGAUGAGAGG |
| 625 | 1009 | UUGGUCAUCUGGUAGUCAG |
| 626 | 1566 | GAUUAAAGAAGCCAAGAGU |
| 627 | 703 | UGAAUGCCACUUUUCACAG |
| 628 | 1334 | UCCUGAUUCAUACCCACGA |
| 629 | 982 | GUGGACUUGAAGAUGAGAG |
| 630 | 334 | ACAAACUCGUAGGCGAUCC |
| 631 | 1447 | UUCAUUCUUACCCACCCAA |
| 632 | 1443 | UUCUUACCCACCCAAGCCA |
| 633 | 849 | AGUGCAUGUUUUCUUGUAG |
| 634 | 1230 | AGUGUCACAGAGUUGUGCA |
| 635 | 597 | UCUGAUGGUACUUCUUACA |
| 636 | 380 | GUAGCGUACUUCCACAUAC |
| 637 | 172 | UUCUUGCCAAAGUAUAAGA |
| 638 | 1328 | UUCAUACCCACGAUUGGCA |
| 639 | 1370 | UUAUUGAACAACAGAUUUU |
| 640 | 931 | UCUUUCUUGAAGCGAACAA |
| 641 | 690 | UCACAGCUCCCUCAUAGGC |
| 642 | 851 | AAAGUGCAUGUUUUCUUGU |
| 643 | 1517 | AAAUGUCACUUCGGGAGGG |
| 644 | 1445 | CAUUCUUACCCACCCAAGC |
| 645 | 980 | GGACUUGAAGAUGAGAGGG |
| 646 | 1442 | UCUUACCCACCCAAGCCAG |
| 647 | 904 | UCCGUUUUGGGAUCCCAGG |
| 648 | 774 | UUGUCUUGAGUACAUCCAC |
| 649 | 339 | UCUCCACAAACUCGUAGGC |
| 650 | 1446 | UCAUUCUUACCCACCCAAG |
| 651 | 466 | UCCACAACCUCGUCAGGGG |
| 652 | 1574 | AGGACUCUGAUUAAAGAAG |
| 653 | 1398 | UGCUGCAAGAUACCAGUCA |
| 654 | 951 | UGAGUGAGUAGUUGGCCUG |
| 655 | 930 | CUUUCUUGAAGCGAACAAC |
| 656 | 596 | CUGAUGGUACUUCUUACAC |
| 657 | 328 | UCGUAGGCGAUCCUUUUGA |
| 658 | 1325 | AUACCCACGAUUGGCAAGG |
| 659 | 1117 | UAGAGUUGUUCCAGAAUCU |
| 660 | 1404 | ACCGUGUGCUGCAAGAUAC |
| 661 | 1335 | UUCCUGAUUCAUACCCACG |
| 662 | 1251 | UCUGGAAGGAAUGAAGGUA |
| 663 | 97 | UUGAAAGCGGGCGUCUGGG |
| 664 | 787 | UGUCCAACCCUGUUUGUCU |
| 665 | 1119 | UGUAGAGUUGUUCCAGAAU |
| 666 | 1550 | AGUUCAGGAGCAUGUGCUC |
| 667 | 881 | UGUGAGAUAGCUGGACCAG |
| 668 | 458 | CUCGUCAGGGGUGACAUCC |
| 669 | 437 | UUCGGUCUGGUUCCAGGGG |
| 670 | 768 | UGAGUACAUCCACAGCCUG |
| 671 | 801 | GUGUGUGGUAGCCAUGUCC |
| 672 | 917 | AACAACUGCAUGCUCCGUU |
| 673 | 94 | AAAGCGGGCGUCUGGGCCA |
| 674 | 1346 | UUAAAGGAAGGUUCCUGAU |
| 675 | 1116 | AGAGUUGUUCCAGAAUCUC |
| 676 | 173 | CUUCUUGCCAAAGUAUAAG |
| 677 | 598 | UUCUGAUGGUACUUCUUAC |
| 678 | 825 | UCUUAUAGAGGGCCUGGUC |
| 679 | 438 | CUUCGGUCUGGUUCCAGGG |
| 680 | 886 | GCGCCUGUGAGAUAGCUGG |
| 681 | 1440 | UUACCCACCCAAGCCAGCG |
| 682 | 98 | GUUGAAAGCGGGCGUCUGG |
| 683 | 642 | CUUCGAUGGUCUCAUCACC |
| 684 | 274 | UAAUCAAACUUGGCCAGGA |
| 685 | 1242 | AAUGAAGGUAAGAGUGUCA |
| 686 | 830 | UAGCCUCUUAUAGAGGGCC |
| 687 | 1016 | GUCCCGUUUGGUCAUCUGG |
| 688 | 1062 | CUGCAUUGAUGUUCAGUCG |
| 689 | 1228 | UGUCACAGAGUUGUGCAGA |
| 690 | 436 | UCGGUCUGGUUCCAGGGGA |
| 691 | 1235 | GUAAGAGUGUCACAGAGUU |
| 692 | 419 | GAUUGGGUCCACUUUGGAA |
| 693 | 876 | GAUAGCUGGACCAGGGGCA |
| 694 | 1046 | UCGCUUGAAUUCUUCCUCA |
| 695 | 1353 | UUUAGCCUUAAAGGAAGGU |
| 696 | 850 | AAGUGCAUGUUUUCUUGUA |
| 697 | 403 | GAAUUGGCCAGCAGGUGUG |
| 698 | 273 | AAUCAAACUUGGCCAGGAA |
| 699 | 442 | UCCCCUUCGGUCUGGUUCC |
| 700 | 781 | ACCCUGUUUGUCUUGAGUA |
| 701 | 1392 | AAGAUACCAGUCACCAGCU |
| 702 | 953 | GUUGAGUGAGUAGUUGGCC |
| 703 | 1120 | UUGUAGAGUUGUUCCAGAA |
| 704 | 275 | GUAAUCAAACUUGGCCAGG |
| 705 | 1047 | GUCGCUUGAAUUCUUCCUC |
| 706 | 1029 | CAGUAAAGCCCAUGUCCCG |
| 707 | 715 | UGGACGGUACGGUGAAUGC |
| 708 | 922 | AAGCGAACAACUGCAUGCU |
| 709 | 1322 | CCCACGAUUGGCAAGGCCC |
| 710 | 326 | GUAGGCGAUCCUUUUGAUG |
| 711 | 168 | UGCCAAAGUAUAAGAUGGU |
| 712 | 288 | UAACAGGCAUGUAGUAAUC |
| 713 | 911 | UGCAUGCUCCGUUUUGGGA |
| 714 | 788 | AUGUCCAACCCUGUUUGUC |
| 715 | 1568 | CUGAUUAAAGAAGCCAAGA |
| 716 | 1323 | ACCCACGAUUGGCAAGGCC |
| 717 | 713 | GACGGUACGGUGAAUGCCA |
| 718 | 882 | CUGUGAGAUAGCUGGACCA |
| 719 | 1414 | GACCACAUUCACCGUGUGC |
| 720 | 708 | UACGGUGAAUGCCACUUUU |
| 721 | 124 | UGGACGUGCAGCUCUACUU |
| 722 | 921 | AGCGAACAACUGCAUGCUC |
| 723 | 300 | UGCAGCCCGCGAUAACAGG |
| 724 | 514 | UUGACCCCGAAUUCUUGCU |
| 725 | 122 | GACGUGCAGCUCUACUUUG |
| 726 | 1327 | UCAUACCCACGAUUGGCAA |
| 727 | 472 | ACAAGAUCCACAACCUCGU |
| 728 | 551 | GGGUUGGUGGCGCAUACAG |
| 729 | 544 | UGGCGCAUACAGCACAGUA |
| 730 | 1317 | GAUUGGCAAGGCCCCUGGG |
| 731 | 379 | UAGCGUACUUCCACAUACA |
| 732 | 1130 | UUGGUAUGCUUUGUAGAGU |
| 733 | 101 | CUUGUUGAAAGCGGGCGUC |
| 734 | 381 | UGUAGCGUACUUCCACAUA |
| 735 | 912 | CUGCAUGCUCCGUUUUGGG |
| 736 | 1410 | ACAUUCACCGUGUGCUGCA |
| 737 | 103 | GGCUUGUUGAAAGCGGGCG |
| 738 | 635 | GGUCUCAUCACCAGCCAGG |
| 739 | 468 | GAUCCACAACCUCGUCAGG |
| 740 | 925 | UUGAAGCGAACAACUGCAU |
| 741 | 962 | GUCGUCUGUGUUGAGUGAG |
| 742 | 422 | GGGGAUUGGGUCCACUUUG |
| 743 | 532 | CACAGUAUGGACCGGACCU |
| 744 | 332 | AAACUCGUAGGCGAUCCUU |
| 745 | 985 | AGGGUGGACUUGAAGAUGA |
| 746 | 796 | UGGUAGCCAUGUCCAACCC |
| 747 | 513 | UGACCCCGAAUUCUUGCUC |
| 748 | 1411 | CACAUUCACCGUGUGCUGC |
| 749 | 368 | CACAUACACCACACCCUCC |
| 750 | 964 | GGGUCGUCUGUGUUGAGUG |
| 751 | 802 | AGUGUGUGGUAGCCAUGUC |
| 752 | 553 | UUGGGUUGGUGGCGCAUAC |
| 753 | 293 | CGCGAUAACAGGCAUGUAG |
| 754 | 837 | CUUGUAGUAGCCUCUUAUA |
| 755 | 967 | AGAGGGUCGUCUGUGUUGA |
| 756 | 99 | UGUUGAAAGCGGGCGUCUG |
| 757 | 77 | CAUGGUGCCGAGUGUGCAC |
| 758 | 766 | AGUACAUCCACAGCCUGUU |
| 759 | 1516 | AAUGUCACUUCGGGAGGGA |
| 760 | 1302 | UGGGCCCUAGCCAGAACAC |
| 761 | 920 | GCGAACAACUGCAUGCUCC |
| 762 | 686 | AGCUCCCUCAUAGGCUUGC |
| 763 | 909 | CAUGCUCCGUUUUGGGAUC |
| 764 | 961 | UCGUCUGUGUUGAGUGAGU |
| 765 | 969 | UGAGAGGGUCGUCUGUGUU |
| 766 | 1010 | UUUGGUCAUCUGGUAGUCA |
| 767 | 675 | AGGCUUGCACAUGUCCUGG |
| 768 | 707 | ACGGUGAAUGCCACUUUUC |
| 769 | 548 | UUGGUGGCGCAUACAGCAC |
| 770 | 875 | AUAGCUGGACCAGGGGCAG |
| 771 | 582 | UACACAGCUCCAACACCUC |
| 772 | 1507 | UCGGGAGGGAAUGUCCCUG |
| 773 | 1441 | CUUACCCACCCAAGCCAGC |
| 774 | 679 | UCAUAGGCUUGCACAUGUC |
| 775 | 593 | AUGGUACUUCUUACACAGC |
| 776 | 1508 | UUCGGGAGGGAAUGUCCCU |
| 777 | 756 | CAGCCUGUUGCACAACCUC |
| 778 | 100 | UUGUUGAAAGCGGGCGUCU |
| 779 | 1127 | GUAUGCUUUGUAGAGUUGU |
| 780 | 73 | GUGCCGAGUGUGCACCCCG |
| 781 | 719 | AGCAUGGACGGUACGGUGA |
| 782 | 1468 | CAGCAUGGGGCCCCAAGAC |
| 783 | 331 | AACUCGUAGGCGAUCCUUU |
| 784 | 216 | UGCGCAGCCCCUCCACUGU |
| 785 | 1206 | CAUCCACAGGCUGAGGCUC |
| 786 | 919 | CGAACAACUGCAUGCUCCG |
| 787 | 643 | CCUUCGAUGGUCUCAUCAC |
| 788 | 1168 | CUUCAGGGGACCUGCCCUC |
| 789 | 720 | CAGCAUGGACGGUACGGUG |
| 790 | 1549 | GUUCAGGAGCAUGUGCUCA |
| 791 | 629 | AUCACCAGCCAGGUCGAUG |
| 792 | 1287 | ACACCUGAUCAGAGGACAG |
| 793 | 831 | GUAGCCUCUUAUAGAGGGC |
| 794 | 908 | AUGCUCCGUUUUGGGAUCC |
| 795 | 111 | CUACUUUGGGCUUGUUGAA |
| 796 | 461 | AACCUCGUCAGGGGUGACA |
| 797 | 1017 | UGUCCCGUUUGGUCAUCUG |
| 798 | 404 | GGAAUUGGCCAGCAGGUGU |
| 799 | 860 | GCAGACCUCAAAGUGCAUG |
| 800 | 1284 | CCUGAUCAGAGGACAGACC |

TABLE 7
RNA effector molecules for inhibition of glutamine
synthase expression in CHO cells (hamster)

| SEQ | | |
|---|---|---|
| ID | Start | Antisense Sequence |
| NO. | Pos. | 5′ to 3′ |
| 801 | 1777 | AGUAAUAAAGCGCUGAGCC |
| 802 | 2021 | UUUAAUAUAUCAAAAGGCC |
| 803 | 1889 | AUACAUAUGCAUCUUAGCC |
| 804 | 1228 | UUGAGUUACAAUGAGACAG |
| 805 | 522 | AGUAAUACGGACCUUGGGG |
| 806 | 1284 | AAUAAAAGCAAGAUUAACU |
| 807 | 1969 | AACCCCAUAAACCCCACCC |
| 808 | 136 | UCAACCCAGAUAUACAUGG |
| 809 | 88 | AAGUACAUUUGCUUGAUGU |
| 810 | 1999 | UUUAGUGACAUGCUAGUCC |
| 811 | 1294 | UAUUCUGACCAAUAAAAGC |
| 812 | 1188 | UAGGAAAGGCUCAAGAUCA |
| 813 | 675 | UGCGGAUUCCUUCACAGGG |
| 814 | 1699 | UUAACCAAGCUCUUCAAAC |
| 815 | 1123 | UCAUUGAGAAGGCAUGUGC |
| 816 | 962 | GAUGUUGGACGUUUCGUGG |
| 817 | 1775 | UAAUAAAGCGCUGAGCCCC |
| 818 | 1878 | UCUUAGCCUAAGCACAGGG |
| 819 | 1395 | AGUGGUUACGUUCCCUUCC |
| 820 | 381 | AGUGCCUUAAAUUGGUCUC |
| 821 | 2081 | UGUAAAGUUAGAAACCCUA |
| 822 | 749 | AAAGGUUGCUAUUACCCCA |
| 823 | 665 | UUCACAGGGUCCUAUUUGG |
| 824 | 1888 | UACAUAUGCAUCUUAGCCU |
| 825 | 1946 | CUAUCAGUAACAAUGUUCA |
| 826 | 1316 | GGGAUUAAGAACUUGACUC |
| 827 | 1600 | UUUAACUCCUCACCUAACU |
| 828 | 1151 | UUUGUAUUGGAAGGGCUCG |
| 829 | 1274 | AGAUUAACUGGGCACGAGG |
| 830 | 452 | CAGAGUAUACUCCUGUUCC |
| 831 | 1121 | AUUGAGAAGGCAUGUGCGG |
| 832 | 1575 | UGGAAUAGAAAGUUGGUUU |
| 833 | 717 | CUCGAUGCAAGAUGAAACG |
| 834 | 239 | AAAGGUACUAGAGCCAUCA |
| 835 | 893 | UCGAAUGUGGUACCGGUGC |
| 836 | 396 | UUAUCCGUUUACACGAGUG |
| 837 | 1882 | UGCAUCUUAGCCUAAGCA |
| 838 | 1528 | AUAGGGGAAUUGUCAAUCC |
| 839 | 608 | UGUAAUCUUGACCCCAGCA |
| 840 | 140 | ACCAUCAACCCAGAUAUAC |
| 841 | 269 | AUACAUGUCACUGUUGGAG |
| 842 | 890 | AAUGUGGUACCGGUGCCGC |
| 843 | 1568 | GAAAGUUGGUUUUACCUGA |
| 844 | 1577 | UUUGGAAUAGAAAGUUGGU |
| 845 | 1338 | AGAAAUGAGGGUUGGGUGU |
| 846 | 1323 | GUGUAUAGGGAUUAAGAAC |
| 847 | 1291 | UCUGACCAAUAAAAGCAAG |
| 848 | 963 | UGAUGUUGGACGUUUCGUG |
| 849 | 653 | UAUUUGGAAUUCCCACUGG |
| 850 | 718 | ACUCGAUGCAAGAUGAAAC |
| 851 | 1950 | ACCCCUAUCAGUAACAAUG |
| 852 | 722 | ACAUACUCGAUGCAAGAUG |
| 853 | 1244 | CUUGAUAUUCCAUCCUUUG |
| 854 | 1223 | UUACAAUGAGACAGCUGGG |
| 855 | 1669 | AGUAACUAGGAUGGUUUCC |
| 856 | 1756 | AGAUAACCACCUUUCCUGG |
| 857 | 395 | UAUCCGUUUACACGAGUGC |
| 858 | 64 | UUCAAGUGGGAACUUGCUG |
| 859 | 1666 | AACUAGGAUGGUUUCCUCA |
| 860 | 714 | GAUGCAAGAUGAAACGGGC |
| 861 | 1147 | UAUUGGAAGGGCUCGUCGC |
| 862 | 1400 | GAAGCAGUGGUUACGUUCC |
| 863 | 1970 | AAACCCCAUAAACCCCACC |
| 864 | 965 | GUUGAUGUUGGACGUUUCG |
| 865 | 1523 | GGAAUUGUCAAUCCAAGCA |
| 866 | 1229 | UUUGAGUUACAAUGAGACA |
| 867 | 1423 | UGGACAUGCAUUCCUGAUG |
| 868 | 1529 | UAUAGGGGAAUUGUCAAUC |
| 869 | 221 | AAAAUUCCACUCAGGUAAC |
| 870 | 1776 | GUAAUAAAGCGCUGAGCCC |
| 871 | 716 | UCGAUGCAAGAUGAAACGG |
| 872 | 872 | CUUGCUUAGUUUCUCGAUG |
| 873 | 1963 | AUAAACCCCACCCACCCCU |
| 874 | 143 | AGUACCAUCAACCCAGAUA |
| 875 | 705 | UGAAACGGGCCACCCAGAG |
| 876 | 721 | CAUACUCGAUGCAAGAUGA |
| 877 | 234 | UACUAGAGCCAUCAAAAUU |
| 878 | 1197 | UGGAUGAACUAGGAAAGGC |
| 879 | 1190 | ACUAGGAAAGGCUCAAGAU |
| 880 | 1784 | CCCACAUAGUAAUAAAGCG |
| 881 | 1315 | GGAUUAAGAACUUGACUCC |
| 882 | 1148 | GUAUUGGAAGGGCUCGUCG |
| 883 | 677 | CAUGCGGAUUCCUUCACAG |
| 884 | 739 | AUUACCCCAAAGUCUUCAC |
| 885 | 1318 | UAGGGAUUAAGAACUUGAC |
| 886 | 2016 | UAUAUCAAAAGGCCUGCUU |
| 887 | 1195 | GAUGAACUAGGAAAGGCUC |
| 888 | 1679 | UGGCAAACCCAGUAACUAG |
| 889 | 355 | UUCCGGUUGUACUUGAAAA |
| 890 | 627 | UGACCUCAGCAUUUGUUCC |
| 891 | 385 | CACGAGUGCCUUAAAUUGG |
| 892 | 1333 | UGAGGGUUGGGUGUAUAGG |
| 893 | 565 | UCCACGAUAUCCCUGCCAU |
| 894 | 1136 | CUCGUCGCCAGUCUCAUUG |
| 895 | 520 | UAAUACGGACCUUGGGGCC |
| 896 | 1660 | GAUGGUUUCCUCAAUUAGA |
| 897 | 1883 | AUGCAUCUUAGCCUAAGCA |
| 898 | 354 | UCCGGUUGUACUUGAAAAC |
| 899 | 1319 | AUAGGGAUUAAGAACUUGA |
| 900 | 1782 | CACAUAGUAAUAAAGCGCU |
| 901 | 1566 | AAGUUGGUUUUACCUGAAG |
| 902 | 563 | CACGAUAUCCCUGCCAUAG |
| 903 | 685 | UGAUCUCCCAUGCGGAUUC |
| 904 | 162 | UGCAGCGCAGUCCUUCUCC |
| 905 | 1073 | ACAAUUGGCAGAGGGGCGG |
| 906 | 1535 | UCUACCUAUAGGGGAAUUG |
| 907 | 254 | GGAGCCCUCAGACUGAAAG |
| 908 | 958 | UUGGACGUUUCGUGGAACC |
| 909 | 2048 | AAGCUGAACUUGUUUUGCU |
| 910 | 388 | UUACACGAGUGCCUUAAAU |
| 911 | 995 | ACUGCGAUUGGCGACACCA |
| 912 | 1746 | CUUUCCUGGUACUGCACCC |
| 913 | 806 | GCUAAAGUUGGUAUGGCAG |
| 914 | 1599 | UUAACUCCUCACCUAACUU |
| 915 | 1947 | CCUAUCAGUAACAAUGUUC |
| 916 | 448 | GUAUACUCCUGUUCCAUUC |
| 917 | 1271 | UUAACUGGGCACGAGGAAU |
| 918 | 1273 | GAUUAACUGGGCACGAGGA |
| 919 | 738 | UUACCCCAAAGUCUUCACA |
| 920 | 1426 | UACUGGACAUGCAUUCCUG |
| 921 | 1399 | AAGCAGUGGUUACGUUCCC |
| 922 | 740 | UAUUACCCCAAAGUCUUCA |
| 923 | 993 | UGCGAUUGGCGACACCAGC |
| 924 | 1527 | UAGGGGAAUUGUCAAUCCA |
| 925 | 393 | UCCGUUUACACGAGUGCCU |
| 926 | 2080 | GUAAAGUUAGAAACCCUAC |
| 927 | 1074 | CACAAUUGGCAGAGGGGCG |
| 928 | 1275 | AAGAUUAACUGGGCACGAG |
| 929 | 2138 | UCCUCCGUUCCUCCAAGAG |
| 930 | 175 | AGGGUGCGGGUUUUGCAGC |
| 931 | 1721 | CUACUCAAGAGAUCCUUUC |
| 932 | 1277 | GCAAGAUUAACUGGGCACG |
| 933 | 245 | AGACUGAAAGGUACUAGAG |
| 934 | 1290 | CUGACCAAUAAAAGCAAGA |
| 935 | 1875 | UAGCCUAAGCACAGGGACA |
| 936 | 284 | GGCAACAGGGCUGAGAUAC |
| 937 | 402 | UGUCCAUUAUCCGUUUACA |
| 938 | 868 | CUUAGUUUCUCGAUGGCCU |
| 939 | 662 | ACAGGGUCCUAUUUGGAAU |
| 940 | 964 | UUGAUGUUGGACGUUUCGU |
| 941 | 713 | AUGCAAGAUGAAACGGGCC |
| 942 | 1972 | AUAAACCCCAUAAACCCCA |
| 943 | 1576 | UUGGAAUAGAAAGUUGGUU |
| 944 | 1324 | GGUGUAUAGGGAUUAAGAA |
| 945 | 1661 | GGAUGGUUUCCUCAAUUAG |
| 946 | 246 | CAGACUGAAAGGUACUAGA |
| 947 | 451 | AGAGUAUACUCCUGUUCCA |
| 948 | 1753 | UAACCACCUUUCCUGGUAC |
| 949 | 980 | ACCAGCAGAAAAGUCGUUG |
| 950 | 1874 | AGCCUAAGCACAGGGACAG |
| 951 | 1930 | UCAAGUUGACCAGCCAACU |
| 952 | 236 | GGUACUAGAGCCAUCAAAA |
| 953 | 401 | GUCCAUUAUCCGUUUACAC |
| 954 | 1193 | UGAACUAGGAAAGGCUCAA |
| 955 | 1533 | UACCUAUAGGGGAAUUGUC |
| 956 | 942 | ACCCAGUCAGACGACGGGC |
| 957 | 176 | CAGGGUGCGGGUUUUGCAG |
| 958 | 1299 | UCCUCUAUUCUGACCAAUA |
| 959 | 91 | CACAAGUACAUUUGCUUGA |
| 960 | 903 | GAUCGUAGGCUCGAAUGUG |
| 961 | 1270 | UAACUGGGCACGAGGAAUA |
| 962 | 1272 | AUUAACUGGGCACGAGGAA |
| 963 | 902 | AUCGUAGGCUCGAAUGUGG |
| 964 | 905 | GGGAUCGUAGGCUCGAAUG |
| 965 | 2092 | ACAGGCAAUUCUGUAAAGU |
| 966 | 492 | CAUUGGAAGGCCAACCAAA |
| 967 | 952 | GUUUCGUGGAACCCAGUCA |
| 968 | 398 | CAUUAUCCGUUUACACGAG |
| 969 | 397 | AUUAUCCGUUUACACGAGU |
| 970 | 559 | AUAUCCCUGCCAUAGGCUU |
| 971 | 1678 | GGCAAACCCAGUAACUAGG |
| 972 | 590 | AUACAAGCAGGCGCGGUAG |
| 973 | 1022 | GACAGUCCGGGGAAUGCGG |
| 974 | 1420 | ACAUGCAUUCCUGAUGAGA |
| 975 | 2140 | UGUCCUCCGUUCCUCCAAG |
| 976 | 1326 | UGGGUGUAUAGGGAUUAAG |
| 977 | 1555 | ACCUGAAGAACUAGCAGCU |
| 978 | 955 | GACGUUUCGUGGAACCCAG |
| 979 | 938 | AGUCAGACGACGGGCAUUG |
| 980 | 1954 | ACCCACCCCUAUCAGUAAC |
| 981 | 960 | UGUUGGACGUUUCGUGGAA |
| 982 | 562 | ACGAUAUCCCUGCCAUAGG |
| 983 | 1267 | CUGGGCACGAGGAAUAAAA |
| 984 | 1904 | GCUAACUCUGUGUGGAUAC |
| 985 | 1250 | AAAGACCUUGAUAUUCCAU |
| 986 | 801 | AGUUGGUAUGGCAGCCUGC |
| 987 | 1765 | CUGAGCCCCAGAUAACCAC |
| 988 | 293 | CCGAAACAUGGCAACAGGG |
| 989 | 785 | UGCACCAUUCCAGUUCCCA |
| 990 | 1662 | AGGAUGGUUUCCUCAAUUA |
| 991 | 898 | UAGGCUCGAAUGUGGUACC |
| 992 | 302 | GAAGGGGUCCCGAAACAUG |
| 993 | 1394 | GUGGUUACGUUCCCUUCCC |
| 994 | 382 | GAGUGCCUUAAAUUGGUCU |
| 995 | 1419 | CAUGCAUUCCUGAUGAGAU |
| 996 | 572 | GUGAGCCUCCACGAUAUCC |
| 997 | 1388 | ACGUUCCCUUCCCCUACCC |
| 998 | 1774 | AAUAAAGCGCUGAGCCCCA |
| 999 | 1877 | CUUAGCCUAAGCACAGGGA |
| 1000 | 1432 | UCUGCCUACUGGACAUGCA |

TABLE 8
RNA effector molecules for inhibition of thymidy-
late synthase expression in CHO cells (hamster)

| SEQ | | |
|---|---|---|
| ID | Start | Antisense Sequence |
| NO. | Pos. | 5′ to 3′ |
| 1001 | 656 | AAUGUUGAAGGGCACACCC |
| 1002 | 878 | AUAACCUUCAAUCUGAAAG |
| 1003 | 380 | AUAAACUGGGCCCAGGUCC |
| 1004 | 277 | AGUUCUUUAGCAUUUGUGG |
| 1005 | 884 | UGGAUUAUAACCUUCAAUC |
| 1006 | 691 | UGUGCUAUCAUGUAGGUAA |
| 1007 | 891 | UUGGAUGUGGAUUAUAACC |
| 1008 | 826 | UUUCGAAGGAUUUUGAGCU |
| 1009 | 759 | UAUGAUUCAGAUAAAUAUG |
| 1010 | 386 | GAAACCAUAAACUGGGCCC |
| 1011 | 324 | AAUCUCGGGACCCAUUGGC |
| 1012 | 730 | AAAGUAUGGACAAAAUCAC |
| 1013 | 583 | ACAUAGAAUUGACAGAGGG |
| 1014 | 229 | AAAACUCCCUUCCAGAACA |
| 1015 | 248 | AAACCAUAGCAACUCCUCC |
| 1016 | 110 | AAAACCGCAGCGCAUAAUG |
| 1017 | 927 | UGAAAGAGCGCUAAACAGC |
| 1018 | 827 | UUUUCGAAGGAUUUUGAGC |
| 1019 | 764 | CUCGAUAUGAUUCAGAUAA |
| 1020 | 839 | AAUUGUCUCAACUUUUCGA |
| 1021 | 326 | AAAAUCUCGGGACCCAUUG |
| 1022 | 206 | UUUGGUUGUGAGCAGAGGA |
| 1023 | 534 | GAUCUUUUGGGUUCCAGGC |
| 1024 | 275 | UUCUUUAGCAUUUGUGGAG |
| 1025 | 797 | UCUUGGUUCUCGCUGAAGC |
| 1026 | 820 | AGGAUUUUGAGCUUUGGGA |
| 1027 | 440 | ACCCGAGUAAUCUGAAUCC |
| 1028 | 98 | CAUAAUGUGCUCCACCUGC |
| 1029 | 900 | UUUUAAUCGUUGGAUGUGG |
| 1030 | 729 | AAGUAUGGACAAAAUCACC |
| 1031 | 298 | AUUCUCACUCCCUUGGAGG |
| 1032 | 543 | UCAGGGGAAGAUCUUUUGG |
| 1033 | 253 | UUGAUAAACCAUAGCAACU |
| 1034 | 615 | GGUAAAGUUGGCAAGACAG |
| 1035 | 755 | AUUCAGAUAAAUAUGUGCA |
| 1036 | 535 | AGAUCUUUUGGGUUCCAGG |
| 1037 | 926 | GAAAGAGCGCUAAACAGCC |
| 1038 | 115 | UUCUUAAAACCGCAGCGCA |
| 1039 | 582 | CAUAGAAUUGACAGAGGGC |
| 1040 | 877 | UAACCUUCAAUCUGAAAGU |
| 1041 | 430 | UCUGAAUCCAUAUCUUUGU |
| 1042 | 455 | CUGGUCUACUCCUUGACCC |
| 1043 | 687 | CUAUCAUGUAGGUAAGCAG |
| 1044 | 494 | AUCAGGAUUGGUUUUGAUG |
| 1045 | 589 | UUUUCCACAUAGAAUUGAC |
| 1046 | 881 | AUUAUAACCUUCAAUCUGA |
| 1047 | 896 | AAUCGUUGGAUGUGGAUUA |
| 1048 | 757 | UGAUUCAGAUAAAUAUGUG |
| 1049 | 421 | AUAUCUUUGUAGUCUGCUC |
| 1050 | 390 | ACUGGAAACCAUAAACUGG |
| 1051 | 305 | AUCCCAUAUUCUCACUCCC |
| 1052 | 118 | UCCUUCUUAAAACCGCAGC |
| 1053 | 745 | AUAUGUGCAUCUCCCAAAG |
| 1054 | 728 | AGUAUGGACAAAAUCACCU |
| 1055 | 249 | UAAACCAUAGCAACUCCUC |
| 1056 | 157 | AUGCCGAACACCGACAAGG |
| 1057 | 613 | UAAAGUUGGCAAGACAGCU |
| 1058 | 509 | GAUGAUUCUUCUGUCAUCA |
| 1059 | 217 | CAGAACACUCUUUUGGUUG |
| 1060 | 500 | UCUGUCAUCAGGAUUGGUU |
| 1061 | 758 | AUGAUUCAGAUAAAUAUGU |
| 1062 | 936 | CAGCAUCUUUGAAAGAGCG |
| 1063 | 416 | UUUGUAGUCUGCUCCAAAA |
| 1064 | 841 | UCAAUUGUCUCAACUUUUC |
| 1065 | 924 | AAGAGCGCUAAACAGCCAU |
| 1066 | 876 | AACCUUCAAUCUGAAAGUC |
| 1067 | 904 | UCCAUUUUAAUCGUUGGAU |
| 1068 | 846 | AAUCAUCAAUUGUCUCAAC |
| 1069 | 183 | CAUCUCUCAGGCUGUAUCG |
| 1070 | 274 | UCUUUAGCAUUUGUGGAGC |
| 1071 | 612 | AAAGUUGGCAAGACAGCUC |
| 1072 | 578 | GAAUUGACAGAGGGCAUGG |
| 1073 | 742 | UGUGCAUCUCCCAAAGUAU |
| 1074 | 725 | AUGGACAAAAUCACCUGGC |
| 1075 | 588 | UUUCCACAUAGAAUUGACA |
| 1076 | 441 | GACCCGAGUAAUCUGAAUC |
| 1077 | 586 | UCCACAUAGAAUUGACAGA |
| 1078 | 778 | UGAAUUUUCAGUGGCUCGA |
| 1079 | 151 | AACACCGACAAGGUGCCAG |
| 1080 | 1 | ACUGGCAUAGCGGAGAAGU |
| 1081 | 379 | UAAACUGGGCCCAGGUCCC |
| 1082 | 808 | UUUGGGAAAGGUCUUGGUU |
| 1083 | 116 | CUUCUUAAAACCGCAGCGC |
| 1084 | 899 | UUUAAUCGUUGGAUGUGGA |
| 1085 | 736 | UCUCCCAAAGUAUGGACAA |
| 1086 | 184 | UCAUCUCUCAGGCUGUAUC |
| 1087 | 385 | AAACCAUAAACUGGGCCCA |
| 1088 | 840 | CAAUUGUCUCAACUUUUCG |
| 1089 | 271 | UUAGCAUUUGUGGAGCCCU |
| 1090 | 817 | AUUUUGAGCUUUGGGAAAG |
| 1091 | 727 | GUAUGGACAAAAUCACCUG |
| 1092 | 148 | ACCGACAAGGUGCCAGUGC |
| 1093 | 577 | AAUUGACAGAGGGCAUGGC |
| 1094 | 276 | GUUCUUUAGCAUUUGUGGA |
| 1095 | 724 | UGGACAAAAUCACCUGGCU |
| 1096 | 751 | AGAUAAAUAUGUGCAUCUC |
| 1097 | 773 | UUUCAGUGGCUCGAUAUGA |
| 1098 | 743 | AUGUGCAUCUCCCAAAGUA |
| 1099 | 117 | CCUUCUUAAAACCGCAGCG |
| 1100 | 678 | AGGUAAGCAGGGCAUAGCU |
| 1101 | 861 | AGUCUUCAACUUUGAAAUC |
| 1102 | 205 | UUGGUUGUGAGCAGAGGAA |
| 1103 | 197 | GAGCAGAGGAAAUUCAUCU |
| 1104 | 449 | UACUCCUUGACCCGAGUAA |
| 1105 | 520 | CAGGCACACAUGAUGAUUC |
| 1106 | 154 | CCGAACACCGACAAGGUGC |
| 1107 | 387 | GGAAACCAUAAACUGGGCC |
| 1108 | 290 | UCCCUUGGAGGACAGUUCU |
| 1109 | 216 | AGAACACUCUUUUGGUUGU |
| 1110 | 212 | CACUCUUUUGGUUGUGAGC |
| 1111 | 619 | CUCUGGUAAAGUUGGCAAG |
| 1112 | 193 | AGAGGAAAUUCAUCUCUCA |
| 1113 | 272 | UUUAGCAUUUGUGGAGCCC |
| 1114 | 142 | AAGGUGCCAGUGCCGGUAC |
| 1115 | 897 | UAAUCGUUGGAUGUGGAUU |
| 1116 | 360 | CUUCCUGUCGAGCAGAGAA |
| 1117 | 355 | UGUCGAGCAGAGAAUCCCA |
| 1118 | 213 | ACACUCUUUUGGUUGUGAG |
| 1119 | 818 | GAUUUUGAGCUUUGGGAAA |
| 1120 | 770 | CAGUGGCUCGAUAUGAUUC |
| 1121 | 585 | CCACAUAGAAUUGACAGAG |
| 1122 | 579 | AGAAUUGACAGAGGGCAUG |
| 1123 | 457 | AGCUGGUCUACUCCUUGAC |
| 1124 | 114 | UCUUAAAACCGCAGCGCAU |
| 1125 | 769 | AGUGGCUCGAUAUGAUUCA |
| 1126 | 347 | AGAGAAUCCCAGGCUGUCC |
| 1127 | 415 | UUGUAGUCUGCUCCAAAAU |
| 1128 | 541 | AGGGGAAGAUCUUUUGGGU |
| 1129 | 880 | UUAUAACCUUCAAUCUGAA |
| 1130 | 898 | UUAAUCGUUGGAUGUGGAU |
| 1131 | 584 | CACAUAGAAUUGACAGAGG |
| 1132 | 209 | UCUUUUGGUUGUGAGCAGA |
| 1133 | 715 | UCACCUGGCUUCAGGCCCG |
| 1134 | 282 | AGGACAGUUCUUUAGCAUU |
| 1135 | 187 | AAUUCAUCUCUCAGGCUGU |
| 1136 | 273 | CUUUAGCAUUUGUGGAGCC |
| 1137 | 892 | GUUGGAUGUGGAUUAUAAC |
| 1138 | 590 | AUUUUCCACAUAGAAUUGA |
| 1139 | 819 | GGAUUUUGAGCUUUGGGAA |
| 1140 | 266 | AUUUGUGGAGCCCUUGAUA |
| 1141 | 346 | GAGAAUCCCAGGCUGUCCA |
| 1142 | 902 | CAUUUUAAUCGUUGGAUGU |
| 1143 | 515 | ACACAUGAUGAUUCUUCUG |
| 1144 | 536 | AAGAUCUUUUGGGUUCCAG |
| 1145 | 112 | UUAAAACCGCAGCGCAUAA |
| 1146 | 921 | AGCGCUAAACAGCCAUUUC |
| 1147 | 693 | UGUGUGCUAUCAUGUAGGU |
| 1148 | 172 | CUGUAUCGCGCCUGCAUGC |
| 1149 | 734 | UCCCAAAGUAUGGACAAAA |
| 1150 | 510 | UGAUGAUUCUUCUGUCAUC |
| 1151 | 201 | UUGUGAGCAGAGGAAAUUC |
| 1152 | 888 | GAUGUGGAUUAUAACCUUC |
| 1153 | 250 | AUAAACCAUAGCAACUCCU |
| 1154 | 446 | UCCUUGACCCGAGUAAUCU |
| 1155 | 463 | UUUUGCAGCUGGUCUACUC |
| 1156 | 402 | CAAAAUGUCUCCACUGGAA |
| 1157 | 388 | UGGAAACCAUAAACUGGGC |
| 1158 | 776 | AAUUUUCAGUGGCUCGAUA |
| 1159 | 179 | UCUCAGGCUGUAUCGCGCC |
| 1160 | 920 | GCGCUAAACAGCCAUUUCC |
| 1161 | 944 | GGUAUUCACAGCAUCUUUG |
| 1162 | 356 | CUGUCGAGCAGAGAAUCCC |
| 1163 | 489 | GAUUGGUUUUGAUGGUGUC |
| 1164 | 558 | AAGGAGGCAGUGCCAUCAG |
| 1165 | 879 | UAUAACCUUCAAUCUGAAA |
| 1166 | 608 | UUGGCAAGACAGCUCCCCA |
| 1167 | 931 | UCUUUGAAAGAGCGCUAAA |
| 1168 | 169 | UAUCGCGCCUGCAUGCCGA |
| 1169 | 304 | UCCCAUAUUCUCACUCCCU |
| 1170 | 194 | CAGAGGAAAUUCAUCUCUC |
| 1171 | 256 | CCCUUGAUAAACCAUAGCA |
| 1172 | 629 | AUCUCCUGACCUCUGGUAA |
| 1173 | 29 | AGCAGCGGAGUGCAGCUUG |
| 1174 | 580 | UAGAAUUGACAGAGGGCAU |
| 1175 | 903 | CCAUUUUAAUCGUUGGAUG |
| 1176 | 680 | GUAGGUAAGCAGGGCAUAG |
| 1177 | 7 | CCAACGACUGGCAUAGCGG |
| 1178 | 309 | UGGCAUCCCAUAUUCUCAC |
| 1179 | 160 | UGCAUGCCGAACACCGACA |
| 1180 | 815 | UUUGAGCUUUGGGAAAGGU |
| 1181 | 8 | GCCAACGACUGGCAUAGCG |
| 1182 | 214 | AACACUCUUUUGGUUGUGA |
| 1183 | 732 | CCAAAGUAUGGACAAAAUC |
| 1184 | 752 | CAGAUAAAUAUGUGCAUCU |
| 1185 | 246 | ACCAUAGCAACUCCUCCAA |
| 1186 | 790 | UCUCGCUGAAGCUGAAUUU |
| 1187 | 744 | UAUGUGCAUCUCCCAAAGU |
| 1188 | 300 | AUAUUCUCACUCCCUUGGA |
| 1189 | 609 | GUUGGCAAGACAGCUCCCC |
| 1190 | 26 | AGCGGAGUGCAGCUUGGAG |
| 1191 | 539 | GGGAAGAUCUUUUGGGUUC |
| 1192 | 228 | AAACUCCCUUCCAGAACAC |
| 1193 | 119 | CUCCUUCUUAAAACCGCAG |
| 1194 | 800 | AGGUCUUGGUUCUCGCUGA |
| 1195 | 696 | UGAUGUGUGCUAUCAUGUA |
| 1196 | 161 | CUGCAUGCCGAACACCGAC |
| 1197 | 204 | UGGUUGUGAGCAGAGGAAA |
| 1198 | 914 | AACAGCCAUUUCCAUUUUA |
| 1199 | 109 | AAACCGCAGCGCAUAAUGU |
| 1200 | 763 | UCGAUAUGAUUCAGAUAAA |

TABLE 9
RNA effector molecules for inhibition of DHFR
expression in CHO cells (hamster)

| SEQ | | |
|---|---|---|
| ID | Start | Antisense Sequence |
| NO. | Pos. | 5′ to 3′ |
| 1201 | 299 | UGUUCAAUAAGUUUUAAGG |
| 1202 | 640 | UUUAAUAUAACCUGGUUAG |
| 1203 | 592 | UUUAGAAUUAUACAGGGGC |
| 1204 | 540 | AUAGACUUCAAAUUUAUAC |
| 1205 | 533 | UCAAAUUUAUACUUGAUGC |
| 1206 | 596 | AUUGUUUAGAAUUAUACAG |
| 1207 | 644 | UAUAUUUAAUAUAACCUGG |
| 1208 | 95 | AAGUACUUGAAUUCGUUCC |
| 1209 | 1153 | UUAACAGUAGCUAUUAUGC |
| 1210 | 611 | AUGAAAAUAAUUCUAAUUG |
| 1211 | 1198 | AGUUUAGUAAGCAAUAUCC |
| 1212 | 818 | UACUUAUUCAUCUAGCUCC |
| 1213 | 675 | AGAACUUUAUGGCAAAUGG |
| 1214 | 594 | UGUUUAGAAUUAUACAGGG |
| 1215 | 595 | UUGUUUAGAAUUAUACAGG |
| 1216 | 1039 | AAACGGAGAAGUUAAAUGU |
| 1217 | 842 | UUUAAAACCCAUUUCUGCC |
| 1218 | 881 | GAUCUAAUUUUCUUUAAAG |
| 1219 | 209 | UUAAUUCUGUCCUUUAAAG |
| 1220 | 534 | UUCAAAUUUAUACUUGAUG |
| 1221 | 852 | AGCUCUGCUGUUUAAAACC |
| 1222 | 895 | UCAGUCUCUACUUUGAUCU |
| 1223 | 987 | UCUCUAAGGAGCACAAUGG |
| 1224 | 259 | GAAAAUGAGCUCCUUGUGG |
| 1225 | 1199 | AAGUUUAGUAAGCAAUAUC |
| 1226 | 89 | UUGAAUUCGUUCCUGAGCA |
| 1227 | 906 | UGCAGAAUAAUUCAGUCUC |
| 1228 | 607 | AAAUAAUUCUAAUUGUUUA |
| 1229 | 131 | UUACCUUCCACUGAGGAGG |
| 1230 | 637 | AAUAUAACCUGGUUAGACU |
| 1231 | 1177 | UCUCAAUUCAUUAUCUCUG |
| 1232 | 294 | AAUAAGUUUUAAGGCAUCG |
| 1233 | 1050 | CUGAAGAUGAGAAACGGAG |
| 1234 | 531 | AAAUUUAUACUUGAUGCCU |
| 1235 | 1040 | GAAACGGAGAAGUUAAAUG |
| 1236 | 298 | GUUCAAUAAGUUUUAAGGC |
| 1237 | 646 | AGUAUAUUUAAUAUAACCU |
| 1238 | 682 | GGCAUUGAGAACUUUAUGG |
| 1239 | 1158 | AAUUCUUAACAGUAGCUAU |
| 1240 | 429 | GUCACUUUCAAAUUCCUGC |
| 1241 | 673 | AACUUUAUGGCAAAUGGUG |
| 1242 | 1166 | UAUCUCUGAAUUCUUAACA |
| 1243 | 727 | UACCCUAUGCAUCUGCUGG |
| 1244 | 195 | UAAAGGUCGAUUCUUCUCA |
| 1245 | 1154 | CUUAACAGUAGCUAUUAUG |
| 1246 | 532 | CAAAUUUAUACUUGAUGCC |
| 1247 | 961 | UCUCAAUUUACCCAUUUUC |
| 1248 | 285 | UAAGGCAUCGUCCAGACUU |
| 1249 | 932 | AACAGAACUCUGCUCAGAG |
| 1250 | 1043 | UGAGAAACGGAGAAGUUAA |
| 1251 | 316 | UAUCUGCUAACUCUGGUUG |
| 1252 | 296 | UCAAUAAGUUUUAAGGCAU |
| 1253 | 959 | UCAAUUUACCCAUUUUCUG |
| 1254 | 100 | UUUGGAAGUACUUGAAUUC |
| 1255 | 841 | UUAAAACCCAUUUCUGCCC |
| 1256 | 1171 | UUCAUUAUCUCUGAAUUCU |
| 1257 | 1196 | UUUAGUAAGCAAUAUCCAU |
| 1258 | 942 | UGUCUGAGUGAACAGAACU |
| 1259 | 457 | UCUCCAAAUCAAUUUCUGG |
| 1260 | 1165 | AUCUCUGAAUUCUUAACAG |
| 1261 | 911 | UGAUGUGCAGAAUAAUUCA |
| 1262 | 76 | UGAGCAUUGGCCAGGGAAG |
| 1263 | 820 | UGUACUUAUUCAUCUAGCU |
| 1264 | 811 | UCAUCUAGCUCCUAUCUCU |
| 1265 | 587 | AAUUAUACAGGGGCUGGGG |
| 1266 | 632 | AACCUGGUUAGACUAAUGA |
| 1267 | 169 | AGAACCAGGUUUUCCGGCC |
| 1268 | 1195 | UUAGUAAGCAAUAUCCAUU |
| 1269 | 36 | CAUAUUCUGGGACACGGCG |
| 1270 | 303 | UGGUUGUUCAAUAAGUUUU |
| 1271 | 1151 | AACAGUAGCUAUUAUGCUU |
| 1272 | 1017 | AGACCUUAUAUAAUCCUCC |
| 1273 | 355 | AAACGGAACUGCCUCCAAC |
| 1274 | 455 | UCCAAAUCAAUUUCUGGGA |
| 1275 | 1186 | AAUAUCCAUUCUCAAUUCA |
| 1276 | 495 | AGAAAGGACCCCUGGGUAC |
| 1277 | 99 | UUGGAAGUACUUGAAUUCG |
| 1278 | 1012 | UUAUAUAAUCCUCCACCUG |
| 1279 | 354 | AACGGAACUGCCUCCAACU |
| 1280 | 638 | UAAUAUAACCUGGUUAGAC |
| 1281 | 1054 | CUCACUGAAGAUGAGAAAC |
| 1282 | 1161 | CUGAAUUCUUAACAGUAGC |
| 1283 | 220 | UGAGAACUAUAUUAAUUCU |
| 1284 | 219 | GAGAACUAUAUUAAUUCUG |
| 1285 | 1142 | UAUUAUGCUUGCCAUUACU |
| 1286 | 1194 | UAGUAAGCAAUAUCCAUUC |
| 1287 | 913 | UCUGAUGUGCAGAAUAAUU |
| 1288 | 1139 | UAUGCUUGCCAUUACUUAA |
| 1289 | 902 | GAAUAAUUCAGUCUCUACU |
| 1290 | 359 | UUGUAAACGGAACUGCCUC |
| 1291 | 331 | AAACCAUGUCCACUUUAUC |
| 1292 | 1014 | CCUUAUAUAAUCCUCCACC |
| 1293 | 983 | UAAGGAGCACAAUGGAGCC |
| 1294 | 742 | CUCUUGUACACACACUACC |
| 1295 | 211 | UAUUAAUUCUGUCCUUUAA |
| 1296 | 935 | GUGAACAGAACUCUGCUCA |
| 1297 | 625 | UUAGACUAAUGAAAAUGAA |
| 1298 | 197 | UUUAAAGGUCGAUUCUUCU |
| 1299 | 218 | AGAACUAUAUUAAUUCUGU |
| 1300 | 847 | UGCUGUUUAAAACCCAUUU |
| 1301 | 1046 | AGAUGAGAAACGGAGAAGU |
| 1302 | 542 | UCAUAGACUUCAAAUUUAU |
| 1303 | 936 | AGUGAACAGAACUCUGCUC |
| 1304 | 626 | GUUAGACUAAUGAAAAUGA |
| 1305 | 1020 | UCCAGACCUUAUAUAAUCC |
| 1306 | 808 | UCUAGCUCCUAUCUCUAUG |
| 1307 | 273 | CAGACUUUUGGCAAGAAAA |
| 1308 | 194 | AAAGGUCGAUUCUUCUCAG |
| 1309 | 1021 | UUCCAGACCUUAUAUAAUC |
| 1310 | 1168 | AUUAUCUCUGAAUUCUUAA |
| 1311 | 1015 | ACCUUAUAUAAUCCUCCAC |
| 1312 | 29 | UGGGACACGGCGACGAUGC |
| 1313 | 589 | AGAAUUAUACAGGGGCUGG |
| 1314 | 196 | UUAAAGGUCGAUUCUUCUC |
| 1315 | 627 | GGUUAGACUAAUGAAAAUG |
| 1316 | 433 | ACGUGUCACUUUCAAAUUC |
| 1317 | 358 | UGUAAACGGAACUGCCUCC |
| 1318 | 34 | UAUUCUGGGACACGGCGAC |
| 1319 | 1047 | AAGAUGAGAAACGGAGAAG |
| 1320 | 1200 | AAAGUUUAGUAAGCAAUAU |
| 1321 | 435 | GAACGUGUCACUUUCAAAU |
| 1322 | 342 | UCCAACUAUCCAAACCAUG |
| 1323 | 56 | UCUCCGUUCUUGCCGAUGC |
| 1324 | 149 | AUAAUCACCAGGUUCUGUU |
| 1325 | 136 | UCUGUUUACCUUCCACUGA |
| 1326 | 135 | CUGUUUACCUUCCACUGAG |
| 1327 | 48 | CUUGCCGAUGCCCAUAUUC |
| 1328 | 401 | GUCACAAAGAGUCUGAGAU |
| 1329 | 411 | CAUGAUCCUUGUCACAAAG |
| 1330 | 198 | CUUUAAAGGUCGAUUCUUC |
| 1331 | 567 | AGUAUCUUUCUGUUAGCCU |
| 1332 | 213 | UAUAUUAAUUCUGUCCUUU |
| 1333 | 307 | ACUCUGGUUGUUCAAUAAG |
| 1334 | 736 | UACACACACUACCCUAUGC |
| 1335 | 723 | CUAUGCAUCUGCUGGGGAG |
| 1336 | 200 | UCCUUUAAAGGUCGAUUCU |
| 1337 | 593 | GUUUAGAAUUAUACAGGGG |
| 1338 | 814 | UAUUCAUCUAGCUCCUAUC |
| 1339 | 105 | CAUUCUUUGGAAGUACUUG |
| 1340 | 400 | UCACAAAGAGUCUGAGAUG |
| 1341 | 305 | UCUGGUUGUUCAAUAAGUU |
| 1342 | 227 | UCUCUACUGAGAACUAUAU |
| 1343 | 738 | UGUACACACACUACCCUAU |
| 1344 | 537 | GACUUCAAAUUUAUACUUG |
| 1345 | 86 | AAUUCGUUCCUGAGCAUUG |
| 1346 | 83 | UCGUUCCUGAGCAUUGGCC |
| 1347 | 189 | UCGAUUCUUCUCAGGAAUG |
| 1348 | 681 | GCAUUGAGAACUUUAUGGC |
| 1349 | 586 | AUUAUACAGGGGCUGGGGA |
| 1350 | 584 | UAUACAGGGGCUGGGGAAG |
| 1351 | 585 | UUAUACAGGGGCUGGGGAA |
| 1352 | 308 | AACUCUGGUUGUUCAAUAA |
| 1353 | 1140 | UUAUGCUUGCCAUUACUUA |
| 1354 | 201 | GUCCUUUAAAGGUCGAUUC |
| 1355 | 588 | GAAUUAUACAGGGGCUGGG |
| 1356 | 476 | UCUGGGAGAAGUUUAUAUU |
| 1357 | 23 | ACGGCGACGAUGCAGUUCA |
| 1358 | 1152 | UAACAGUAGCUAUUAUGCU |
| 1359 | 363 | UUCCUUGUAAACGGAACUG |
| 1360 | 160 | UUUUCCGGCCCAUAAUCAC |
| 1361 | 819 | GUACUUAUUCAUCUAGCUC |
| 1362 | 600 | UCUAAUUGUUUAGAAUUAU |
| 1363 | 98 | UGGAAGUACUUGAAUUCGU |
| 1364 | 635 | UAUAACCUGGUUAGACUAA |
| 1365 | 217 | GAACUAUAUUAAUUCUGUC |
| 1366 | 743 | UCUCUUGUACACACACUAC |
| 1367 | 1037 | ACGGAGAAGUUAAAUGUUC |
| 1368 | 1187 | CAAUAUCCAUUCUCAAUUC |
| 1369 | 629 | CUGGUUAGACUAAUGAAAA |
| 1370 | 402 | UGUCACAAAGAGUCUGAGA |
| 1371 | 92 | UACUUGAAUUCGUUCCUGA |
| 1372 | 530 | AAUUUAUACUUGAUGCCUU |
| 1373 | 434 | AACGUGUCACUUUCAAAUU |
| 1374 | 639 | UUAAUAUAACCUGGUUAGA |
| 1375 | 279 | AUCGUCCAGACUUUUGGCA |
| 1376 | 802 | UCCUAUCUCUAUGAGCCCC |
| 1377 | 559 | UCUGUUAGCCUUUCUUCUC |
| 1378 | 224 | CUACUGAGAACUAUAUUAA |
| 1379 | 1159 | GAAUUCUUAACAGUAGCUA |
| 1380 | 281 | GCAUCGUCCAGACUUUUGG |
| 1381 | 312 | UGCUAACUCUGGUUGUUCA |
| 1382 | 387 | GAGAUGGCCUGGCUGAUUC |
| 1383 | 739 | UUGUACACACACUACCCUA |
| 1384 | 1067 | UAUCCCUUGGAAUCUCACU |
| 1385 | 954 | UUACCCAUUUUCUGUCUGA |
| 1386 | 853 | UAGCUCUGCUGUUUAAAAC |
| 1387 | 1146 | UAGCUAUUAUGCUUGCCAU |
| 1388 | 289 | GUUUUAAGGCAUCGUCCAG |
| 1389 | 5 | AGCGGUCGAACCAUGACAG |
| 1390 | 336 | UAUCCAAACCAUGUCCACU |
| 1391 | 631 | ACCUGGUUAGACUAAUGAA |
| 1392 | 807 | CUAGCUCCUAUCUCUAUGA |
| 1393 | 633 | UAACCUGGUUAGACUAAUG |
| 1394 | 666 | UGGCAAAUGGUGUUUCUUA |
| 1395 | 84 | UUCGUUCCUGAGCAUUGGC |
| 1396 | 726 | ACCCUAUGCAUCUGCUGGG |
| 1397 | 335 | AUCCAAACCAUGUCCACUU |
| 1398 | 223 | UACUGAGAACUAUAUUAAU |
| 1399 | 295 | CAAUAAGUUUUAAGGCAUC |
| 1400 | 529 | AUUUAUACUUGAUGCCUUU |

1. A method of generating a cell line capable of producing a biological product comprising:
(a) providing a plurality of host cells comprising a first selectable amplifiable marker gene and a second selectable amplifiable marker gene, wherein a transgene encoding a biological product is linked to the first selectable amplifiable marker gene, and wherein the first and second selectable amplifiable marker genes each have different nucleic acid sequences and are capable of being amplified using the same amplification reagent;
(b) transfecting the host cell of step (a) with an RNA effector molecule, a portion of which is complementary to the second selectable amplifiable marker gene endogenous to the host cell such that the RNA effector molecule inhibits expression of the second selectable amplifiable marker gene; and
(c) contacting the transfected cells of step (b) with a progressively increasing amount of the amplification reagent to select for cells with multiple copies of the first selectable amplifiable marker gene and the transgene, thereby generating a cell line that is capable of producing the biological product.

2. A method of generating a cell line capable of producing a biological product comprising:
a) transfecting a plurality of host cells with:
i) one or more vectors comprising a transgene linked to a first selectable amplifiable marker gene, wherein the transgene encodes a biological product,
ii) an RNA effector molecule, a portion of which is complementary to a second selectable amplifiable marker gene endogenous to the host cell such that the RNA effector molecule inhibits expression of the second selectable amplifiable marker gene, wherein the first and second selectable amplifiable marker genes each have a different nucleic acid sequence and are capable of being amplified using an amplification reagent,
b) culturing the plurality of host cells of step a) with a first concentration of the amplification reagent to select for viable transfected host cells;
c) culturing the viable transfected host cells of step b) with a higher concentration of the amplification reagent than used in step b), thereby selecting for surviving cells that have an increased copy number of the transgene and the first selectable marker gene, wherein cells capable of producing a biological product are generated.

3. The method of claim 1, wherein the RNA effector molecule does not significantly inhibit expression of the first selectable marker gene.

4. The method of claim 1, wherein the RNA effector molecule transiently inhibits expression of the second selectable amplifiable marker gene.

5. The method of claim 1, wherein the RNA effector molecule inhibits expression of the second selectable amplification gene by at least 10%, at least 20%, at least 30%, at least 40%, at least 50%, at least 60%, at least 70%, at least 80%, at least 90%, at least 95%, at least 99%, or 100%.

6. The method of claim 1, wherein the RNA effector molecule inhibits expression of the second amplifiable marker gene at least 10%, at least 20%, at least 30%, at least 40%, at least 50%, at least 60%, at least 70%, at least 80%, at least 90%, at least 95%, at least 99%, at least 2-fold, at least 5-fold, at least 10-fold, at least 100 fold, or at least 1000 fold more than the RNA effector molecule inhibits the first selectable amplifiable marker.

7. The method of claim 1, further comprising transfecting the cell of step a) with a second RNA effector molecule, a portion of which is complementary to the transgene, such that the second RNA effector molecule inhibits expression of the transgene.

8. The method of claim 6, wherein the cell that has amplified the transgene is maintained in the presence of the second RNA effector molecule for a period of time before removal of the second RNA effector molecule and expression of the transgene.

9. The method of claim 7, wherein the RNA effector molecule inhibits expression of the transgene by an average percent inhibition of at least 10%, at least 20%, at least 30%, at least 40%, at least 50%, at least 60%, at least 70%, at least 80%, at least 90%, at least 95%, at least 99%, or 100%.

10. The method of claim 1, wherein the first and second selectable amplifiable marker genes encode a protein selected from the group consisting of: dihydrofolate reductase, thymidylate synthase, glutamine synthetase, adenosine deaminase, carbamoyl-phosphate synthase-aspartate transcarbamoylase-dihydroorotase (CAD), ornithine decarboxylase, and asparagine synthetase.

11. The method of claim 1, wherein the first and second selectable amplifiable marker genes do not encode for dihydrofolate reductase.

12. The method of claim 1, wherein the first and second selectable amplifiable marker genes are from different species.

13. The method of claim 1, wherein the amplification reagent is selected from the group consisting of: methotrexate, N-phosphonoacetyl-L-aspartic acid (PALA), 2′-deoxycoformycin (dCF), 5-fluorouracil (5FU), difluoromethylornithine (DFMO), albizziin, and β-aspartyl hydroxamate (β-AHA).

14. The method of claim 1, wherein the biological product is selected from the group consisting of a polypeptide, a metabolite and a nutraceutical.

15. (canceled)

16. (canceled)

17. The method of claim 1, wherein the cell is selected from the group consisting of an animal cell, a fungal cell, a plant cell and a mammalian cell.

18. (canceled)

19. (canceled)

20. (canceled)

21. The method of claim 17, wherein the mammalian cell is a human cell.

22. The method of claim 21, wherein the human cell is an adherent cell selected from the group consisting of: SH-SY5Y cells, IMR32 cells, LANS cells, HeLa cells, MCF1OA cells, 293T cells, and SK-BR3 cells.

23. The method of claim 21, wherein the human cell is a primary cell selected from the group consisting of: HuVEC cells, HuASMC cells, HKB-I1 cells, and hMSC cells.

24. The method of claim 21, wherein the human cell is selected from the group consisting of: U293 cells, HEK 293 cells, PERC6® cells, Jurkat cells, HT-29 cells, LNCap.FGC cells, A549 cells, MDA MB453 cells, HepG2 cells, THP-I cells, MCF7 cells, BxPC-3 cells, Capan-1 cells, DU145 cells, and PC-3 cells.

25. The method of claim 21, wherein the mammalian cell is a rodent cell selected from the group consisting of: BHK21 cells, BHK TK− cells, NS0 cells, Sp2/0 cells, EL4 cells, CHO cells, CHO cell derivatives, U293 cells, NIH/3T3 cells, 3T3 L1 cells, ES-D3 cells, H9c2 cells, C2C12 cells, and miMCD-3 cells.

26. The method of claim 25, wherein the CHO cell derivative is selected from the group consisting of: CHO-K1 cells, CHO-DUKX, CHO-DUKX B1, and CHO-DG44 cells.

27. The method of claim 21, wherein the human cell is selected from the group consisting of: PERC6 cells, HT-29 cells, LNCaP-FGC cells A549 cells, MDA MB453 cells, HepG2 cells, THP-I cells, miMCD-3 cells, HEK 293 cells, HeLaS3 cells, MCF7 cells, Cos-7 cells, BxPC-3 cells, DU145 cells, Jurkat cells, PC-3 cells, and Capan-1 cells.

28. The method of claim 1, wherein the RNA effector molecule is a double-stranded ribonucleic acid (dsRNA), wherein said dsRNA comprises at least two sequences that are complementary to each other and wherein a sense strand comprises a first sequence and an antisense strand comprises a second sequence comprising a region of complementarity, and wherein said region of complementarity is 15-30 nucleotides in length.

29. The method of claim 1, wherein the RNA effector molecule comprises a modified nucleotide.

30. The method of claim 1, wherein the nucleic acid sequences of the first and second selectable amplifiable marker differ by at least one nucleotide.

31. The method of claim 7, wherein the second RNA effector molecule is transfected immediately before, simultaneously with, or immediately after the vector comprising a transgene.

32. The method of claim 2, wherein the transgene and first selectable marker are each provided on a separate vector and are linked co-transformationally in the host genome.

33. The method of claim 2, wherein the transgene linked to the first selectable marker is provided on a single vector.

34. A method for increasing the transfection efficiency of cells capable of producing a biological product, comprising transfecting a plurality of host cells with:
i) a vector comprising a transgene that encodes a biological product; and
ii) an RNA effector molecule that inhibits expression of the transgene,
wherein the RNA effector molecule inhibits expression of the transgene thereby increasing the transfection efficiency as compared to the transfection efficiency observed in the absence of the RNA effector molecule.

35. The method of claim 34, wherein the RNA effector molecule is transfected immediately before, simultaneously with, or immediately after the vector comprising a transgene.

36. The method of claim 34, wherein the RNA effector molecule is a double-stranded ribonucleic acid (dsRNA), wherein said dsRNA comprises at least two sequences that are complementary to each other and wherein a sense strand comprises a first sequence and an antisense strand comprises a second sequence comprising a region of complementarity, and wherein said region of complementarity is 15-30 nucleotides in length.

37. The method of claim 34, wherein the RNA effector molecule comprises a modified nucleotide.

38. The method of claim 34, wherein expression of the transgene is transiently inhibited.

39. The method of claim 34, wherein the RNA effector molecule inhibits expression of the transgene by at least 10%, at least 20%, at least 30%, at least 40%, at least 50%, at least 60%, at least 70%, at least 80%, at least 90%, at least 95%, at least 99%, or 100%.

40. The method of, wherein the cell with the transgene is maintained in the presence of the RNA effector molecule for a period of time before removal of the RNA effector molecule and expression of the transgene.

41. The method of claim 34, wherein the biological product is selected from the group consisting of a polypeptide, a metabolite, and a nutraceutical.

42. (canceled)

43. (canceled)

44. The method of claim 34, wherein the cell is selected from the group consisting of an animal cell, fungal cell, plant cell and mammalian cell.

45. (canceled)

46. (canceled)

47. (canceled)

48. The method of claim 44, wherein the mammalian cell is a human cell.

49. The method of claim 48, wherein the human cell is an adherent cell selected from the group consisting of: SH-SY5Y cells, IMR32 cells, LANS cells, HeLa cells, MCF1OA cells, 293T cells, and SK-BR3 cells.

50. The method of claim 48, wherein the human cell is a primary cell selected from the group consisting of: HuVEC cells, HuASMC cells, HKB-I1 cells, and hMSC cells.

51. The method of claim 48, wherein the human cell is selected from the group consisting of: U293 cells, HEK 293 cells, PERC6® cells, Jurkat cells, HT-29 cells, LNCap.FGC cells, A549 cells, MDA MB453 cells, HepG2 cells, THP-I cells, MCF7 cells, BxPC-3 cells, Capan-1 cells, DU145 cells, and PC-3 cells.

52. The method of claim 48, wherein the mammalian cell is a rodent cell selected from the group consisting of: BHK21 cells, BHK TK− cells, NS0 cells, Sp2/0 cells, EL4 cells, CHO cells, CHO cell derivatives, U293 cells, NIH/3T3 cells, 3T3 L1 cells, ES-D3 cells, H9c2 cells, C2C12 cells, and miMCD-3 cells.

53. The method of claim 52, wherein the CHO cell derivative is selected from the group consisting of: CHO-K1 cells, CHO-DUKX, CHO-DUKX B1, and CHO-DG44 cells.

54. The method of claim 48, wherein the human cell is selected from the group consisting of: PERC6 cells, HT-29 cells, LNCaP-FGC cells A549 cells, MDA MB453 cells, HepG2 cells, THP-I cells, miMCD-3 cells, HEK 293 cells, HeLaS3 cells, MCF7 cells, Cos-7 cells, BxPC-3 cells, DU145 cells, Jurkat cells, PC-3 cells, and Capan-1 cells.

55. A method for generating a cell line capable of producing a biological product, comprising:
(a) transfecting a plurality of host cells with:
i) a vector comprising a selectable marker and a transgene, wherein the transgene encodes a biological product, and
ii) an RNA effector molecule, a portion of which is complementary to a copy of the selectable marker endogenously expressed in the plurality of host cells prior to introduction of the vector of step i), and
(b) culturing the cells of step (a) under conditions that select for cells comprising the vector of step i), thereby generating a cell line capable of producing a biological product.

56. A kit for generating a cell capable of producing a biological product comprising:
a) a vector comprising a selectable amplifiable marker gene that has a nucleic acid sequence distinct from that of the marker gene endogenous to a host cell;
b) an RNA effector molecule, a portion of which is complementary to the marker gene endogenous to the host cell; and
c) packaging materials and instructions therefor.

57. The kit of claim 56, further comprising a host cell.

58. The kit of claim 56, wherein the nucleic acid sequence of the selectable amplifiable marker on the vector differs from the nucleic acid sequence of the endogenous marker gene by at least one nucleotide.

59. The kit of claim 56, further comprising an amplification reagent.
